(19) United States
(12) Patent Application Publication
(10) Pub. No.: US 2024/0059677 A1
(43) Pub. Date: Feb. 22, 2024

(54) SUBSTITUTED PYRIMIDINE DERIVATIVES AS NICOTINIC ACETYLCHOLINESTERASE RECEPTOR ALPHA 6 MODULATOR

(71) Applicants: Cerevance, Inc., Boston, MA (US); TAKEDA PHARMACEUTICAL COMPANY LIMITED, Osaka (JP)

(72) Inventors: Roland Werner Burli, Cambridge (GB); Martin Teall, Cambridge (GB); Nicola Brice, Cambridge (GB)

(21) Appl. No.: 18/025,315

(22) PCT Filed: Sep. 10, 2021
(86) PCT No.: PCT/GB2021/052353

§ 371 Date: Mar. 8, 2023

(30) Foreign Application Priority Data
Sep. 11, 2020 (GB) 2014341.8

(51) Int. Cl. C07D 403/12; C07D 401/12; A61K 45/06

(52) U.S. Cl. CPC C07D 403/12; C07D 401/12; A61K 45/06; C07B 2200/05; C07B 2200/07

(57) ABSTRACT

The present invention provides compounds of formula (I) and pharmaceutically acceptable salts, solvates and prodrugs thereof wherein in, R¹, R², R³, L and X are as defined in the specification, processes for their preparation, pharmaceutical compositions containing them and their use in therapy particularly for use in treating disorders associated with nicotinic acetylcholine receptor α6 (nAChRα6) activity.

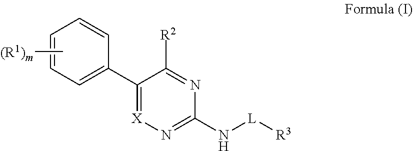

### FIELD OF THE INVENTION

The present invention relates to 5-aryl pyrimidine amines and 6-aryl 1,2,4-triazine amines, processes for their preparation, pharmaceutical compositions containing them and their use in therapy, particularly for use in treating disorders associated with nicotinic acetylcholine receptor α6 (nAChRα6) activity.

### BACKGROUND OF THE INVENTION

Acetylcholine is one of the principle neurotransmitters in the central nervous system (CNS) and mediates its effects via two classes of receptors. The first class is the muscarinic family of G-protein coupled receptors, of which there are five known members, M1, M2, M3, M4 and M5. The second class of receptors are the nicotinic ligand gated ion channels. These nicotinic receptors are expressed throughout the CNS and have been shown to have a modulatory effect on nearly all neurotransmitter systems examined, including dopamine. The receptors are cation-selective pentamers that belong to the same ligand gated ion channel superfamily as GABAa and 5HT3 receptors. In the CNS, nicotinic receptors are composed from a set of 12 different alpha (α) and beta (β) subunits (α2-10 and β2-4). Each receptor pentamer consists of 2 or 3 α subunits with β subunits (e.g. (α4)₃(β2)₂, (α6)₂(β2)₃, (α3)₂(β4)₃, α4α6β3(β2)₂) (Le et al, J Neurobiol 53: 447-456 2002). The one exception is α7, which can form a homopentamer. The different subunit compositions give rise to different biophysical and pharmacological profiles which make them suitable drug targets depending on subunit composition (Wells, Front Biosci 13: 5479-5510 2008; Campling et al, PLoS One 8: e79653 2013).

Nicotinic receptors can be found throughout the CNS, with the α4β2 being the most abundant heteroreceptor (Wada et al, J Comp Neurol 284: 314-335 1989). In contrast to other subunits, the α6 subunit expression is restricted to mid brain regions, such as the dopaminergic neurons of the substantia nigra (SN) and ventral tegmental area (VTA), as well as noradrenergic neurons of the locus coeruleus (LC). These brain regions are important in movement disorders and psychiatric disorders such as addiction (Quik et al, Biochem Pharm 82: 873-882 2011: Engle et al, Mol Pharmacol 84: 393-406 2013). The α6 subunit has also been detected in the superior colliculus (Mackey et al, J Neurosci 32: 10226-10237 2012; Allen Brain Atlas www.brain-map.org), a region important for integrating sensory information into movement, particularly eye movement. The α6 subunit has been shown to heteromultimize with both β2 and β3 subunits and potentially other a subunits such as α3 and α4 (Miller and Gotti, Neuropharmacol 56: 237-246 2009 for review).

The dopaminergic neurons from the VTA and SN project to the striatum, where they release dopamine in response to a reward signal or for locomotor control respectively. Control of this release is lost in movement disorders such as tremor, dystonia, Parkinson's disease and Huntington's disease, and psychiatric disorders including schizophrenia, attention deficit hyperactivity disorder (ADHD), autism spectrum disorder (ASD), Tourettes syndrome and addictions (Klein et al, Cellular and Molecular Neurobiol 39: 31-59 2019; Collins-Praino et al, Front Syst Neurosci 4: 49 2011; Bao et al, J Neurochem 114: 178-191 2010; Rice et al, Neurosci 198: 112-137 2011; Jakel and Maragos, TiNS 23: 239-245 2000; Howes et al, Biol Psychiatry 81: 9-20 2017; Castellanos and Tannock, Nat Rev Neurosci 3: 617-628 2002; Maia and Conceicao, Biol Psychiatry 84: 332-344 2018; Nutt et al, Nat Rev Neurosci 16: 305-312 2015).

The α6 containing receptors are pre-synaptically located on the dopaminergic neurons that project into the striatum. Their functional effect is to potentiate dopamine release in response to acetylcholine released from local cholinergic interneurons (Ztaou and Amalric, Neurochem Int 126: 1-10 2019; de Kloest et al, Biochem Pharmacol 97: 425-438 2015; Aosaki et al, Geriatr Gerontol Int S148-157 2010). In the non-human primate striatal synaptosome preparation, α6 containing receptors mediate 80% of dopamine release compared to only 20% by the α4 containing nAChR population (Quik et al, Biochemical Pharm, 82: 873-882 2011 for review). Therefore, modulators specific for the nicotinic α6 subunit would have therapeutic potential in disorders that have dysregulation of dopamine as a key pathological mechanism. Similarly, α6 containing receptors in the locus coeruleus (LC) and superior colliculus have been shown to modulate firing patterns of the noradrenergic and GABAergic neurons, respectively (Lena et al, PNAS 96: 12126-12131 1999; Mackey et al, J Neurosci 32: 10226-10237 2012), which has a consequence on noradrenaline release. The α6 containing receptors are also expressed in other discrete regions that contain monoaminergic neurons including the serotonergic neurons of the raphe nuclei. Similarly to the dopaminergic neurons, modulation of the nicotinic receptor can modulate the excitability of these neurons and therefore the release of serotonin (Galindo-Charles et al, Synapse 62(8): 601-615 2008).

Movement Disorders

Previous studies have shown the important role of nicotinic receptors in mechanisms of Parkinson's disease. The observation that nicotine appears to have neuroprotective properties led to investigation into the possible efficacy of nicotinic agonists such as TC8831, which is an agonist for both α4β2 and α6β2 and shows efficacy in the macaque MPTP model (Johnston et al, Neuropharmacology 73: 337-347 2013). One hypothesis is that the agonists desensitize the receptors, therefore acting as functional antagonists. This is supported by Bordia et al (JPET 333: 929-938 2010) who showed in 6-OHDA lesioned rats that were then chronically treated with L-DOPA to induce dyskinesias, that (1) chronic nicotine dosing is efficacious at reducing the abnormal involuntary movements (AIMs), a pre-clinical correlate of dyskinesias, whereas acute nicotine has no effect, and (2) the nAChRα6 antagonist, mecamylamine, could reduce the AIMs score when dosed alone.

Other studies using genetically manipulated mice have extended this hypothesis by showing that chronically dosed nicotine reversed dyskinesia in WT mice and not transgenic gain-of-function (α6 L9S) mice, in which the receptor shows little desensitization (Bordia et al, Neuroscience 295: 187-197 2015). Furthermore, nAChRα6 knockout (KO) mice show improvement in 6-OHDA/L-DOPA induced AIMs (reduced AIMs score) compared to WT mice. This reduction in the AIMs score seen in the KO mice was comparable to WT mice chronically treated with nicotine (Quik et al, Neuropharm 63: 450-459 2012). Although dyskinesias are often associated with Parkinson's disease, other diseases and medications also have dyskinesia as a symptom or side effect, for example, tardive dyskinesia in schizophrenia. Similar to L-DOPA induced dyskinesias, these other dyskinesias are a result of aberrant dopamine release. Indeed, pre-clinical models of tardive dyskinesia show that chronic nicotine administration reduces the dyskinesia. The molecular mechanism of this was shown to include the reduction in the α6 subunit expression (Bordia et al, JPET 340: 612-619 2012) which is a similar consequence as an α6 antagonist. This hypothesis is supported by further work by Bordia et al (Exp Neurol 286: 32-39 2016) in which the nicotinic receptor antagonist, mecamylamine, reduced haloperidol induced dyskinesias.

Tremor is the most prevalent movement disorder and can be a symptom of many underlying disorders including Parkinson's disease. Tremors can be subdivided into groups depending on characteristics such as amplitude, frequency or etiology. Categories of tremor can include resting tremor, essential tremor, drug induced tremor, dystonic tremor or psychogenic tremor. Resting tremor is seen in approximately 75% of Parkinson's patients and is poorly treated with existing medication. The monoaminergic neurons have been implicated in resting tremor (Dirkx et al, Brain 140: 721-734 2017; Isaias et al, Front Hum Neurosci, vol 5, article 179, 2012; Qamhawi et al, Brain 138: 2964-2973 2015) and all express the α6 subunit. Furthermore, tremor dominant Parkinson's patients have less noradrenergic neuronal loss in the locus coeruleus (LC) than non-tremor dominant Parkinson's patients, which leads to an imbalance of available neurotransmitter release in key brain regions such as the striatum and the thalamus with more noradrenaline being released. Further evidence for this comes from the observation that tremor is exacerbated in stressful situations when the locus coeruleus (LC) is activated and more noradrenaline is released (Zach et al, CNS Neurosci and Ther 23: 209-215 2017). An agent, such as a nicotinic α6 subunit antagonist that reduces noradrenaline release, should therefore improve symptoms of resting tremor (Lena et al, PNAS 96(21): 12126-12131 1999).

Dystonia is another movement disorder that can either be a symptom of Parkinson's and other diseases, or a syndrome in the absence of other diseases such as the DYT1 dystonia that is caused by mutations in the TOR1A gene. A common feature is an increase in dopamine release and abnormal striatal cholinergic transmission (Zimmerman et al, Front Syst Neurosci 11: 43 2017). Therefore, an α6 antagonist would reduce the dopamine tone and improve symptoms.

Huntington's disease is a fatal neurodegenerative disorder caused by a poly-glutamine expansion in the Huntington's gene (Htt). The symptoms of the disease are characterised by progressive motor, cognitive and psychiatric decline. Post-mortem analysis shows a loss of neurons in the striatum, particularly the acetylcholinergic interneurons in this region which leads to dysregulation of this system. In a study looking at the effect of varenicline, a non-specific nicotinic receptor ligand that readily desensitizes the α6 subunit, it has been shown to improve symptoms of Huntington's disease, including cognitive ability (McGregor et al, Neuropsychiatric Diseases and Treatment 12: 2381-2386 2016).

Psychiatric Disorders

Many of the symptoms of psychiatric disorders are caused by abnormal monoaminergic tone. For instance, schizophrenia, psychosis, psychotic disorder and schizoaffective disorder have hyperdopaminergic tone that is principally caused by increase in presynaptic capacity (McCutcheon et al, World Psychiatry 19: 15-33 2020). Therefore, a molecule that can reduce the release of dopamine could have efficacy in these disorders. Antagonists of α6 reduce the release of evoked dopamine via pre-synaptic mechanisms (Wickham et al, Psychopharm 229: 73-82 2013; Wang et al, J Neurochem 129: 315-327 2014) and so have the potential to be efficacious in schizophrenia and related disorders.

Lack of impulse control is a symptom of several psychiatric disorders including ADHD, schizophrenia, bipolar disorder, ASD including Fragile X, and addiction. Impulse behaviour is stimulated, in part, by increased dopamine release in the nucleus accumbens (Cole and Robbins, Behav Brain Res 33: 165-179 1989; Pattij et al, Psychopharmacology 1991: 587-598 2007). Activation of nicotinic receptors by nicotine or varenicline has been shown to induce impulsive behaviours when administered acutely (non-desensitizing) but not chronically (desensitizing) (Tsutsui-Kimura et al, Psychopharmacology 209: 351-359 2010). Both of these agonists activate α6 containing receptors when administered acutely, which would potentiate dopamine release. Therefore, an α6 antagonist that inhibits dopamine release would have utility in treating impulse control symptoms in a range of psychiatric disorders.

Tourettes syndrome (TS) is a neurodevelopmental disorder defined by characteristic involuntary movements, tics, with both motor and phonic components. Tourettes syndrome is considered a disorder of the basal ganglia and, in particular, a striatal dysfunction (Ganos et al, Neurosci Biobehav Rev 37: 1050-1062 2013; Tremblay et al, Mov Disord 30: 1155-1170 2015). This concept is based on the response of tics to treatment with dopamine antagonists, the occurrence of tics in diseases with unequivocal striatal pathology, and evidence from structural and functional imaging studies implicating basal ganglia associated neurocircuits (Worbe et al, Mov Disord 30: 1179-1183 2015). Currently, dopamine D2 receptor antagonists are the only treatments approved by the US Food and Drug Administration for tics, but they are not recommended as first line treatment due to their adverse side-effect profile (Eddy et al, Ther Adv Neurol Disord 4(1): 25-45 2011). Taken together, a striatal based mechanism that regulates synaptic dopamine output may have therapeutic utility in treating the symptoms of Tourettes syndrome.

Addiction disorders, including substance use disorder, alcohol use disorder, binge eating, and gambling disorder, all follow a similar pathology. Whatever the addictive stimuli, the addictive behaviour is caused by abnormal release of dopamine from VTA neurons projecting to the nucleus accumbens in what has been termed the ‘reward pathway’. In normal circumstances dopamine is released from these neurons in response to natural rewards such as food, social interaction or sex. However, in the addicted brain the pathway is hyperstimulated by the addictive substance or behaviour and is not stimulated by natural rewards. This leads to the feeling of craving. Agents that can decrease this hyperstimulation of the pathway and normalise dopamine transmission, reverse addictive behaviours in pre-clinical models (Volkow et al, Nat Rev Neurosci 18: 741-752 2017). Antagonists of the nAChRα6 subunit decrease dopamine release from synaptosomes of neurons originating in the VTA and projecting into the nucleus accumbens (Gotti et al, J Neurosci 30(15): 5311-5325 2010). Furthermore, multiple genetic studies have linked the nAChRα6 gene to increased risk of addictions including nicotine (Won et al, Psychiatry Investig 11(3): 307-312 2014; Wang et al, Hum Genet 133: 575-586 2014; Brunzell, Nicotine & Tobacco Res 14(11): 1258-1269 2012), cocaine (Sadler et al, Sci Rep 4: 4497 2014) and alcohol (Hoft et al, Genes Brain Behav 8: 631-637 2009).

Bipolar disorder is a complex psychiatric disorder with patients experiences both episodes of mania and depression. It is thought that the balance between acetylcholine and the catecholamines norepinephrine and dopamine determine these episodes with a hypercholinergic state during bipolar depression and increased functional catecholamines during bipolar mania (Enkhuzien et al, Eur J Pharmacol 753: 114-126 2015). The α6 subunit is expressed on the pre-synaptic terminals of both dopamine and norepinephrine neurons and modulates neurotransmitter release (Gotti et al, J Neurosci 30(15): 5311-5325 2010; Azam et al FASEB J. 24: 5113-5123 2010). Therefore, an antagonist of α6 could restore levels of catecholamines and reduce symptoms of bipolar mania. Similarly, antagonising the excess cholinergic tone during bipolar depression using a specific α6 antagonist could reduce the effects of the excess acetylcholine which would shift towards a less depressive state. Indeed, drugs currently used to treat depression such as SSRIs are nicotinic receptor antagonists (Hennings et al, Brain Res 759: 292-294 1997; Shytle et al, Mol Psychiatry 7: 525-535 2002).

There is a need for treatment of the above conditions and others described herein with compounds that are nAChRα6 antagonists. The present invention provides antagonists of nAChRα6.

### SUMMARY OF THE INVENTION

A first aspect of the present invention provides a compound of formula (I):

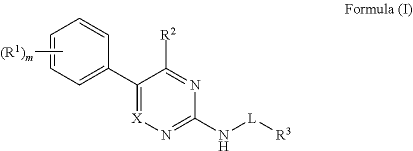

or a pharmaceutically acceptable salt, solvate or prodrug thereof, wherein:
X is N or CR⁴;
L is a bond, —C(O)—, —C(O)—C(R⁵)₂—, —C(R⁵)₂—, or —C(R⁵)₂C(R⁵)₂—;
each R¹ is independently selected from halo, cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, or C₃-C₆ halocycloalkoxy;
m is 2, 3, 4 or 5;
R² is selected from halo, cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, or C₃-C₆ halocycloalkoxy;
R³ is selected from a C₃-C₆ cycloalkyl or 4- to 10-membered heterocyclic group, wherein the cycloalkyl or heterocyclic group is optionally substituted with one, two, three, four or five substituents independently selected from halo, cyano, hydroxy, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, C₃-C₆ halocycloalkoxy, —N(R⁶)₂, or —SO₂(R⁶);
R⁴ is selected from hydrogen, halo, cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, or C₃-C₆ halocycloalkoxy;
each R⁵ is independently selected from hydrogen, CH₃, CH₂CH₃, or CF₃; and
each R⁶ is independently selected from hydrogen or C₁-C₃ alkyl, or two R⁶ together with the nitrogen to which they are attached form a 3- to 6-membered saturated heterocyclic group;
provided the compound is not:
4-methoxy-5-(2-methyl-4-(trifluoromethoxy)phenyl)-N-((1s,4s)-4-methylcyclohexyl)pyrimidin-2-amine;
4-methoxy-5-(2-methoxy-4-(trifluoromethoxy)phenyl)-N-((1s,4s)-4-methylcyclohexyl)pyrimidin-2-amine; or
N-cyclopropyl-5-(3,4-dimethoxyphenyl)-4-methyl-pyrimidin-2-amine.

In the context of the present specification, unless otherwise stated, an “alkyl” substituent group or an “alkyl” moiety in a substituent group (such as an alkoxy group) may be linear or branched.

Examples of C₁-C₅ alkyl groups/moieties include methyl, ethyl, n-propyl, iso-propyl, n-butyl, iso-butyl, sec-butyl, tert-butyl, n-pentyl, 2-methyl-1-butyl, 3-methyl-1-butyl, 2-methyl-3-butyl, and 2,2-dimethyl-1-propyl.

The term “hydrogen” encompasses ¹H, ²H (D) and ³H (T). Therefore, for the avoidance of doubt, it is noted that, for example, the terms “alkyl” and “methyl” include, for example, trideuteriomethyl.

A “cycloalkyl” substituent group or a “cycloalkyl” moiety in a substituent group refers to a saturated hydrocarbyl ring containing, for example, from 3 to 6 carbon atoms, examples of which include cyclopropyl, cyclobutyl, cyclopentyl and cyclohexyl.

A “haloalkyl” substituent group or a “haloalkyl” moiety in a substituent group refers to an alkyl group or moiety in which one or more, e.g. one, two, three, four or five, hydrogen atoms are replaced independently by halogen atoms, i.e. by fluorine, chlorine, bromine or iodine atoms. Examples of haloalkyl groups/moieties include fluoromethyl, difluoromethyl, trifluoromethyl and 2,2,2-trifluoroethyl.

The term “halogen” includes fluorine, chlorine, bromine and iodine. In one embodiment, halogen is fluorine.

A “heterocyclic” group refers to a cyclic group which comprises one or more carbon atoms and one or more (such as one, two, three or four) heteroatoms, e.g. N, O or S, in the ring structure. A heterocyclic group may be monocyclic, bicyclic (e.g. bridged, fused or spiro), or polycyclic. Typically, a heterocyclic group is a 4- to 10-membered heterocyclic group, which means it contains from 4 to 10 ring atoms. Heterocyclic groups include saturated heterocyclic groups and heteroaryl groups.

A saturated heterocyclic group may be monocyclic, bicyclic (e.g. bridged, fused or spiro), or polycyclic. Examples of saturated monocyclic heterocyclic groups are azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl, and thiomorpholinyl groups. Examples of saturated bicyclic heterocyclic groups are quinuclidinyl, 8-azabicyclo[3.2.1]octanyl, 2-azaspiro[3.3]heptanyl, 6-azaspiro[2.5]octanyl, and hexahydro-1H-pyrrolizinyl groups.

A “heteroaryl” group is an aromatic heterocyclic group. The term “heteroaryl” includes monocyclic aromatic heterocycles (such as pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, and oxadiazolyl groups) and polycyclic fused ring aromatic heterocycles. Examples of heteroaryl groups include the following:

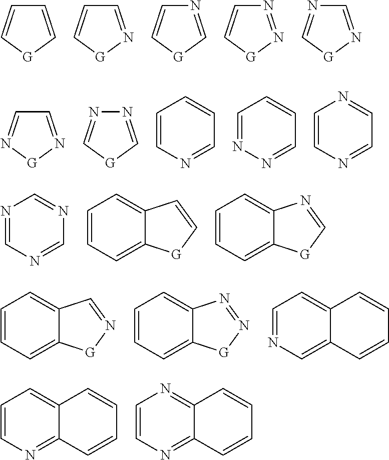

wherein G=O, S or NH.

When any chemical group or moiety is described as substituted, it will be appreciated that the number and nature of substituents will be selected so as to avoid sterically undesirable combinations.

Further, it will be appreciated that the invention does not encompass any unstable ring or other structures or any O—O or S—S bonds.

Each R¹ is independently selected from halo (such as fluoro, chloro, bromo and iodo), cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, or C₃-C₆ halocycloalkoxy.

In one embodiment, each R¹ is independently selected from halo (such as fluoro, chloro and bromo), cyano, C₁-C₃ alkyl (such as methyl, ethyl, n-propyl and iso-propyl), C₁-C₃ haloalkyl (such as fluorinated and/or chlorinated methyl, ethyl, n-propyl and iso-propyl), C₃-C₆ cycloalkyl (such as cyclopropyl, cyclobutyl, cyclopentyl and cyclohexyl), C₃-C₆ halocycloalkyl (such as fluorinated and/or chlorinated cyclopropyl, cyclobutyl, cyclopentyl and cyclohexyl), C₁-C₃ alkoxy (such as methoxy, ethoxy, n-propoxy and iso-propoxy), C₁-C₃ haloalkoxy (such as fluorinated and/or chlorinated methoxy, ethoxy, n-propoxy and iso-propoxy), C₃-C₆ cycloalkoxy (such as cyclopropoxy, cyclobutoxy, cyclopentoxy and cyclohexoxy), or C₃-C₆ halocycloalkoxy (such as fluorinated and/or chlorinated cyclopropoxy, cyclobutoxy, cyclopentoxy and cyclohexoxy).

In another embodiment, each R¹ is independently selected from halo (such as fluoro, chloro and bromo), cyano, C₁-C₃ alkyl (such as methyl, ethyl, n-propyl and iso-propyl), C₁-C₃ haloalkyl (such as fluorinated and/or chlorinated methyl, ethyl, n-propyl and iso-propyl), C₁-C₃ alkoxy (such as methoxy, ethoxy, n-propoxy and iso-propoxy), or C₁-C₃ haloalkoxy (such as fluorinated and/or chlorinated methoxy, ethoxy, n-propoxy and iso-propoxy).

In another embodiment, each R¹ is independently selected from fluoro, chloro, cyano, —CH₃, —CH₂CH₃, —CF₃, —CH₂CF₃, —CF₂CF₃, —OCH₃, —OCH₂CH₃, —OCF₃, —OCH₂CF₃, or —OCF₂CF₃.

In another embodiment, each R¹ is independently selected from fluoro, chloro, —CH₃, —CF₃, —OCH₃, or —OCF₃.

m is 2, 3, 4 or 5. In one embodiment, m is 2, 3 or 4. In another embodiment, m is 2 or 3. In another embodiment, m is 2.

In one embodiment, m is 2. When m is 2, the two substituents R¹ can be in the 2,3-, 2,4-, 2,5-, 2,6-, 3,4- or 3,5-positions. In one embodiment, m is 2 and the two substituents R¹ are in the 2,4- or 2,5-positions. In another embodiment, m is 2 and the two substituents R¹ are in the 2,4-positions.

R² is selected from halo (such as fluoro, chloro, bromo and iodo), cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, or C₃-C₆ halocycloalkoxy.

In one embodiment, R² is selected from halo (such as fluoro, chloro and bromo), cyano, C₁-C₃ alkyl (such as methyl, ethyl, n-propyl and iso-propyl), C₁-C₃ haloalkyl (such as fluorinated and/or chlorinated methyl, ethyl, n-propyl and iso-propyl), C₃-C₆ cycloalkyl (such as cyclopropyl, cyclobutyl, cyclopentyl and cyclohexyl), C₃-C₆ halocycloalkyl (such as fluorinated and/or chlorinated cyclopropyl, cyclobutyl, cyclopentyl and cyclohexyl), C₁-C₃ alkoxy (such as methoxy, ethoxy, n-propoxy and iso-propoxy), C₁-C₃ haloalkoxy (such as fluorinated and/or chlorinated methoxy, ethoxy, n-propoxy and iso-propoxy), C₃-C₆ cycloalkoxy (such as cyclopropoxy, cyclobutoxy, cyclopentoxy and cyclohexoxy), or C₃-C₆ halocycloalkoxy (such as fluorinated and/or chlorinated cyclopropoxy, cyclobutoxy, cyclopentoxy and cyclohexoxy).

In another embodiment, R² is selected from halo (such as fluoro, chloro and bromo), cyano, C₁-C₃ alkyl (such as methyl, ethyl, n-propyl and iso-propyl), C₁-C₃ haloalkyl (such as fluorinated and/or chlorinated methyl, ethyl, n-propyl and iso-propyl), cyclopropyl, C₁-C₃ alkoxy (such as methoxy, ethoxy, n-propoxy and iso-propoxy), C₁-C₃ haloalkoxy (such as fluorinated and/or chlorinated methoxy, ethoxy, n-propoxy and iso-propoxy), or cyclopropoxy.

In another embodiment, R² is selected from fluoro, chloro, cyano, cyclopropyl, cyclopropoxy, —CH₃, —CH₂CH₃, —CF₃, —CH₂CF₃, —CF₂CF₃, —OCH₃, —OCH₂CH₃, —OCF₃, —OCH₂CF₃, or —OCF₂CF₃.

In another embodiment, R² is selected from fluoro, cyano, cyclopropyl, cyclopropoxy, —CH₃, —CF₃, —OCH₃, or —OCF₃.

X is N or CR⁴. In one embodiment, X is N. In another embodiment, X is CR⁴.

R⁴ is selected from hydrogen, halo (such as fluoro, chloro, bromo and iodo), cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, or C₃-C₆ halocycloalkoxy.

In one embodiment, R⁴ is selected from hydrogen, halo (such as fluoro, chloro and bromo), cyano, C₁-C₃ alkyl (such as methyl, ethyl, n-propyl and iso-propyl), C₁-C₃ haloalkyl (such as fluorinated and/or chlorinated methyl, ethyl, n-propyl and iso-propyl), C₃-C₆ cycloalkyl (such as cyclopropyl, cyclobutyl, cyclopentyl and cyclohexyl), C₃-C₆ halocycloalkyl (such as fluorinated and/or chlorinated cyclopropyl, cyclobutyl, cyclopentyl and cyclohexyl), C₁-C₃ alkoxy (such as methoxy, ethoxy, n-propoxy and iso-propoxy), C₁-C₃ haloalkoxy (such as fluorinated and/or chlorinated methoxy, ethoxy, n-propoxy and iso-propoxy), C₃-C₆ cycloalkoxy (such as cyclopropoxy, cyclobutoxy, cyclopentoxy and cyclohexoxy), or C₃-C₆ halocycloalkoxy (such as fluorinated and/or chlorinated cyclopropoxy, cyclobutoxy, cyclopentoxy and cyclohexoxy).

In another embodiment, R⁴ is selected from hydrogen, halo (such as fluoro, chloro and bromo), cyano, C₁-C₃ alkyl (such as methyl, ethyl, n-propyl and iso-propyl), C₁-C₃ haloalkyl (such as fluorinated and/or chlorinated methyl, ethyl, n-propyl and iso-propyl), cyclopropyl, C₁-C₃ alkoxy (such as methoxy, ethoxy, n-propoxy and iso-propoxy), C₁-C₃ haloalkoxy (such as fluorinated and/or chlorinated methoxy, ethoxy, n-propoxy and iso-propoxy), or cyclopropoxy.

In another embodiment, R⁴ is selected from hydrogen, fluoro, chloro, cyano, cyclopropyl, cyclopropoxy, —CH₃, —CH₂CH₃, —CF₃, —CH₂CF₃, —CF₂CF₃, —OCH₃, —OCH₂CH₃, —OCF₃, —OCH₂CF₃, or —OCF₂CF₃.

In another embodiment, R⁴ is selected from hydrogen, fluoro, cyano, cyclopropyl, cyclopropoxy, —CH₃, —CF₃, —OCH₃, or —OCF₃.

L is a bond, —C(O)—, —C(O)—C(R⁵)₂—, —C(R⁵)₂—, or —C(R⁵)₂C(R⁵)₂—, wherein each R⁵ is independently selected from hydrogen, CH₃, CH₂CH₃, or CF₃.

In one embodiment, L is a bond, —C(O)—, —C(O)—CH₂—, —C(O)—CH(CH₃)—, —C(O)—CH(CH₂CH₃)—, —C(O)—CH(CF₃)—, —CH₂—, —CH(CH₃)—, —CH(CH₂CH₃)—, —CH(CF₃)—, —CH₂CH₂—, —CH₂—CH(CH₃)—, —CH₂—CH(CH₂CH₃)—, —CH₂—CH(CF₃)—, —CH(CH₃)—CH₂—, —CH(CH₂CH₃)—CH₂—, or —CH(CF₃)—CH₂—.

In another embodiment, L is a bond, —C(O)—, —C(O)—CH₂—, —C(O)—CH(CH₃)—, —C(O)—CH(CF₃)—, —CH₂—, —CH(CH₃)—, —CH(CF₃)—, —CH₂CH₂—, —CH₂—CH(CH₃)—, —CH₂—CH(CF₃)—, —CH(CH₃)—CH₂—, or —CH(CF₃)—CH₂—.

In another embodiment, L is a bond, —C(O)—, —CH₂—, —CH(CH₃)—, or —CH₂CH₂—.

In another embodiment, L is a bond or —CH₂—.

R³ is selected from a C₃-C₆ cycloalkyl or 4- to 10-membered heterocyclic group, wherein the cycloalkyl or heterocyclic group is optionally substituted. In one embodiment, the 4- to 10-membered heterocyclic group comprises one, two or three heteroatoms independently selected from N, O or S in the ring structure.

In one embodiment, R³ is selected from a C₃-C₆ cycloalkyl or 4- to 10-membered saturated heterocyclic group, wherein the cycloalkyl or saturated heterocyclic group is optionally substituted. In one embodiment, the 4- to 10-membered saturated heterocyclic group comprises one, two or three heteroatoms independently selected from N, O or S in the ring structure.

In one embodiment, R³ is selected from a 4- to 10-membered saturated heterocyclic group, wherein the saturated heterocyclic group is optionally substituted. In one embodiment, the 4- to 10-membered saturated heterocyclic group comprises one, two or three heteroatoms independently selected from N, O or S in the ring structure.

In another embodiment, R³ is selected from a C₃-C₆ cycloalkyl group, a 4-, 5- or 6-membered saturated monocyclic heterocyclic group (comprising, for example, one or two heteroatoms independently selected from N or O in the ring structure), a 7-, 8-, 9- or 10-membered saturated bicyclic heterocyclic group (comprising, for example, one or two heteroatoms independently selected from N or O in the ring structure), or a 5- or 6-membered heteroaryl group (comprising, for example, one or two heteroatoms independently selected from N or O in the ring structure), each of which is optionally substituted.

In another embodiment, R³ is selected from a C₃-C₆ cycloalkyl group, a 4-, 5- or 6-membered saturated monocyclic heterocyclic group (comprising, for example, one or two heteroatoms independently selected from N or O in the ring structure), or a 7-, 8-, 9- or 10-membered saturated bicyclic heterocyclic group (comprising, for example, one or two heteroatoms independently selected from N or O in the ring structure), each of which is optionally substituted.

In another embodiment, R³ is selected from a 4-, 5- or 6-membered saturated monocyclic heterocyclic group (comprising, for example, one or two heteroatoms independently selected from N or O in the ring structure), or a 7-, 8-, 9- or 10-membered saturated bicyclic heterocyclic group (comprising, for example, one or two heteroatoms independently selected from N or O in the ring structure), each of which is optionally substituted.

In another embodiment, R³ is selected from a cyclopropyl, cyclobutyl, cyclopentyl, cyclohexyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl, thiomorpholinyl, quinuclidinyl, 8-azabicyclo[3.2.1]octanyl, 2-azaspiro[3.3]heptanyl, 6-azaspiro[2.5]octanyl, hexahydro-1H-pyrrolizinyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, or oxadiazolyl group, each of which is optionally substituted.

In another embodiment, R³ is selected from a cyclopropyl, cyclobutyl, cyclopentyl, cyclohexyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl, thiomorpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group, each of which is optionally substituted.

In another embodiment, R³ is selected from an azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl, thiomorpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group, each of which is optionally substituted.

In another embodiment, R³ is selected from a cyclopropyl, cyclobutyl, cyclopentyl, cyclohexyl, azetidinyl, oxetanyl, pyrrolidinyl, tetrahydrofuranyl, pyrazolidinyl, imidazolidinyl, dioxolanyl, piperidinyl, tetrahydropyranyl, piperazinyl, dioxanyl, morpholinyl, quinuclidinyl, 8-azabicyclo[3.2.1]octanyl, 2-azaspiro[3.3]heptanyl, 6-azaspiro[2.5]octanyl, hexahydro-1H-pyrrolizinyl, pyridinyl, pyridazinyl, pyrimidinyl, pyrazinyl, pyrrolyl, furanyl, pyrazolyl, imidazolyl, oxazolyl, isoxazolyl, triazolyl, or oxadiazolyl group, each of which is optionally substituted.

In another embodiment, R³ is selected from a cyclopropyl, cyclobutyl, cyclopentyl, cyclohexyl, azetidinyl, oxetanyl, pyrrolidinyl, tetrahydrofuranyl, pyrazolidinyl, imidazolidinyl, dioxolanyl, piperidinyl, tetrahydropyranyl, piperazinyl, dioxanyl, morpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group, each of which is optionally substituted.

In another embodiment, R³ is selected from an azetidinyl, oxetanyl, pyrrolidinyl, tetrahydrofuranyl, pyrazolidinyl, imidazolidinyl, dioxolanyl, piperidinyl, tetrahydropyranyl, piperazinyl, dioxanyl, morpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group, each of which is optionally substituted.

In another embodiment, R³ is selected from a cyclopropyl, cyclobutyl, cyclopentyl, cyclohexyl, pyrrolidinyl, tetrahydrofuranyl, piperidinyl, tetrahydropyranyl, piperazinyl, dioxanyl, morpholinyl, quinuclidinyl, 8-azabicyclo[3.2.1]octanyl, 2-azaspiro[3.3]heptanyl, 6-azaspiro[2.5]octanyl, hexahydro-1H-pyrrolizinyl, pyridinyl, pyrrolyl, furanyl, pyrazolyl, or imidazolyl group, each of which is optionally substituted.

In another embodiment, R³ is selected from a cyclopropyl, cyclobutyl, cyclopentyl, cyclohexyl, pyrrolidinyl, tetrahydrofuranyl, piperidinyl, tetrahydropyranyl, piperazinyl, dioxanyl, morpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group, each of which is optionally substituted.

In another embodiment, R³ is selected from a pyrrolidinyl, tetrahydrofuranyl, piperidinyl, tetrahydropyranyl, piperazinyl, dioxanyl, morpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group, each of which is optionally substituted.

In another embodiment, R³ is selected from a cyclopropyl, cyclobutyl, cyclopentyl, cyclohexyl, pyrrolidinyl, tetrahydrofuranyl, piperidinyl, tetrahydropyranyl, morpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group, each of which is optionally substituted.

In another embodiment, R³ is selected from a pyrrolidinyl, tetrahydrofuranyl, piperidinyl, tetrahydropyranyl, morpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group, each of which is optionally substituted.

In another embodiment, R³ is selected from a pyrrolidinyl, tetrahydrofuranyl, piperidinyl, tetrahydropyranyl, or morpholinyl group, each of which is optionally substituted.

In another embodiment, R³ is selected from a pyrrolidinyl or piperidinyl group, each of which is optionally substituted.

The cycloalkyl or heterocyclic group of R³ is optionally substituted with one, two, three, four or five substituents independently selected from halo (such as fluoro, chloro, bromo and iodo), cyano, hydroxy, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, C₃-C₆ halocycloalkoxy, —N(R⁶)₂, or —SO₂(R⁶), wherein each R⁶ is independently selected from hydrogen or C₁-C₃ alkyl, or two R⁶ together with the nitrogen to which they are attached form a 3- to 6-membered saturated heterocyclic group. In one embodiment, the 3- to 6-membered saturated heterocyclic group comprises one or two heteroatoms independently selected from N, O or S in the ring structure.

In one embodiment, the substituents are independently selected from halo (such as fluoro, chloro and bromo), cyano, hydroxy, C₁-C₃ alkyl (such as methyl, ethyl, n-propyl and iso-propyl), C₁-C₃ haloalkyl (such as fluorinated and/or chlorinated methyl, ethyl, n-propyl and iso-propyl), C₃-C₆ cycloalkyl (such as cyclopropyl, cyclobutyl, cyclopentyl and cyclohexyl), C₃-C₆ halocycloalkyl (such as fluorinated and/or chlorinated cyclopropyl, cyclobutyl, cyclopentyl and cyclohexyl), C₁-C₃ alkoxy (such as methoxy, ethoxy, n-propoxy and iso-propoxy), C₁-C₃ haloalkoxy (such as fluorinated and/or chlorinated methoxy, ethoxy, n-propoxy and iso-propoxy), C₃-C₆ cycloalkoxy (such as cyclopropoxy, cyclobutoxy, cyclopentoxy and cyclohexoxy), C₃-C₆ halocycloalkoxy (such as fluorinated and/or chlorinated cyclopropoxy, cyclobutoxy, cyclopentoxy and cyclohexoxy), —N(R⁶)₂, or —SO₂(R⁶), wherein each R⁶ is independently selected from hydrogen or C₁-C₃ alkyl (such as methyl, ethyl, n-propyl and iso-propyl), or two R⁶ together with the nitrogen to which they are attached form a 3- to 6-membered saturated heterocyclic group (such as an aziridinyl, azetidinyl, pyrrolidinyl and piperidinyl group).

In another embodiment, the substituents are independently selected from halo (such as fluoro, chloro and bromo), cyano, hydroxy, C₁-C₃ alkyl (such as methyl, ethyl, n-propyl and iso-propyl), C₁-C₃ haloalkyl (such as fluorinated and/or chlorinated methyl, ethyl, n-propyl and iso-propyl), C₁-C₃ alkoxy (such as methoxy, ethoxy, n-propoxy and iso-propoxy), C₁-C₃ haloalkoxy (such as fluorinated and/or chlorinated methoxy, ethoxy, n-propoxy and iso-propoxy), —N(R⁶)₂, or —SO₂(R⁶), wherein each R⁶ is independently selected from hydrogen or C₁-C₃ alkyl (such as methyl, ethyl, n-propyl and iso-propyl).

In another embodiment, the substituents are independently selected from fluoro, chloro, cyano, hydroxy, methyl (which, for the avoidance of doubt, includes for example trideuteriomethyl), ethyl, n-propyl, iso-propyl, methoxy, ethoxy, n-propoxy, iso-propoxy, —CF₃, —CH₂CF₃, —CF₂CF₃, —OCF₃, —OCH₂CF₃, —OCF₂CF₃, —N(R⁶)₂, or —SO₂(R⁶), wherein each R⁶ is independently selected from hydrogen, methyl, or ethyl.

In another embodiment, the substituents are independently selected from fluoro, chloro, cyano, hydroxy, methyl (which, for the avoidance of doubt, includes for example trideuteriomethyl), ethyl, n-propyl, iso-propyl, methoxy, ethoxy, n-propoxy, iso-propoxy, —CF₃, —CH₂CF₃, —CF₂CF₃, —OCF₃, —OCH₂CF₃, —OCF₂CF₃, —NH₂, —NH(CH₃), —N(CH₃)₂, or —SO₂(CH₃).

In another embodiment, the substituents on a ring nitrogen atom are independently selected from methyl (which, for the avoidance of doubt, includes for example trideuteriomethyl), ethyl, n-propyl, iso-propyl, —CF₃, —CH₂CF₃, or —CF₂CF₃.

In another embodiment, the substituents on a ring carbon atom are independently selected from fluoro, hydroxy, methyl (which, for the avoidance of doubt, includes for example trideuteriomethyl), methoxy, —CF₃, —OCF₃, —NH₂, —NH(CH₃), —N(CH₃)₂, or —SO₂(CH₃).

In one specific embodiment of the first aspect, the present invention provides a compound of formula (I), or a pharmaceutically acceptable salt, solvate or prodrug thereof, wherein:
X is N or CR⁴;
L is a bond, —C(O)—, —C(O)—CH₂—, —C(O)—CH(CH₃)—, —C(O)—CH(CF₃)—, —CH₂—, —CH(CH₃)—, —CH(CF₃)—, —CH₂CH₂—, —CH₂—CH(CH₃)—, —CH₂—CH(CF₃)—, —CH(CH₃)—CH₂—, or —CH(CF₃)—CH₂—;
each R¹ is independently selected from halo, cyano, C₁-C₃ alkyl, C₁-C₃ haloalkyl, C₁-C₃ alkoxy, or C₁-C₃ haloalkoxy;
m is 2;
R² is selected from halo, cyano, C₁-C₃ alkyl, C₁-C₃ haloalkyl, cyclopropyl, C₁-C₃ alkoxy, C₁-C₃ haloalkoxy, or cyclopropoxy;
R³ is selected from a C₃-C₆ cycloalkyl group, a 4-, 5- or 6-membered saturated monocyclic heterocyclic group, a 7-, 8-, 9- or 10-membered saturated bicyclic heterocyclic group, or a 5- or 6-membered heteroaryl group (preferably a C₃-C₆ cycloalkyl group, a 4-, 5- or 6-membered saturated monocyclic heterocyclic group, or a 7-, 8-, 9- or 10-membered saturated bicyclic heterocyclic group; more preferably a 4-, 5- or 6-membered saturated monocyclic heterocyclic group, or a 7-, 8-, 9- or 10-membered saturated bicyclic heterocyclic group; more preferably a 4-, 5- or 6-membered saturated monocyclic heterocyclic group), each of which is optionally substituted with one, two, three, four or five substituents independently selected from halo, cyano, hydroxy, C₁-C₃ alkyl, C₁-C₃ haloalkyl, C₁-C₃ alkoxy, C₁-C₃ haloalkoxy, —N(R⁶)₂, or —SO₂(R⁶);
R⁴ is selected from hydrogen, halo, cyano, C₁-C₃ alkyl, C₁-C₃ haloalkyl, cyclopropyl, C₁-C₃ alkoxy, C₁-C₃ haloalkoxy, or cyclopropoxy; and
each R⁶ is independently selected from hydrogen or C₁-C₃ alkyl;
provided the compound is not:
4-methoxy-5-(2-methyl-4-(trifluoromethoxy)phenyl)-N-((1s,4s)-4-methylcyclohexyl)pyrimidin-2-amine;
4-methoxy-5-(2-methoxy-4-(trifluoromethoxy)phenyl)-N-((1s,4s)-4-methylcyclohexyl)pyrimidin-2-amine; or
N-cyclopropyl-5-(3,4-dimethoxyphenyl)-4-methyl-pyrimidin-2-amine.

In another specific embodiment of the first aspect, the present invention provides a compound of formula (I), or a pharmaceutically acceptable salt, solvate or prodrug thereof, wherein:
X is N or CR⁴;
L is a bond, —C(O), —CH₂—, —CH(CH₃)—, or —CH₂CH₂—;
each R¹ is independently selected from fluoro, chloro, cyano, —CH₃, —CH₂CH₃, —CF₃, —CH₂CF₃, —CF₂CF₃, —OCH₃, —OCH₂CH₃, —OCF₃, —OCH₂CF₃, or —OCF₂CF₃;
m is 2;
R² is selected from fluoro, chloro, cyano, cyclopropyl, cyclopropoxy, —CH₃, —CH₂CH₃, —CF₃, —CH₂CF₃, —CF₂CF₃, —OCH₃, —OCH₂CH₃, —OCF₃, —OCH₂CF₃, or —OCF₂CF₃;
R³ is selected from a cyclopropyl, cyclobutyl, cyclopentyl, cyclohexyl, azetidinyl, oxetanyl, pyrrolidinyl, tetrahydrofuranyl, pyrazolidinyl, imidazolidinyl, dioxolanyl, piperidinyl, tetrahydropyranyl, piperazinyl, dioxanyl, morpholinyl, quinuclidinyl, 8-azabicyclo[3.2.1]octanyl, 2-azaspiro[3.3]heptanyl, 6-azaspiro[2.5]octanyl, hexahydro-1H-pyrrolizinyl, pyridinyl, pyridazinyl, pyrimidinyl, pyrazinyl, pyrrolyl, furanyl, pyrazolyl, imidazolyl, oxazolyl, isoxazolyl, triazolyl, or oxadiazolyl group (preferably a cyclopropyl, cyclobutyl, cyclopentyl, cyclohexyl, azetidinyl, oxetanyl, pyrrolidinyl, tetrahydrofuranyl, pyrazolidinyl, imidazolidinyl, dioxolanyl, piperidinyl, tetrahydropyranyl, piperazinyl, dioxanyl, morpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group; more preferably an azetidinyl, oxetanyl, pyrrolidinyl, tetrahydrofuranyl, pyrazolidinyl, imidazolidinyl, dioxolanyl, piperidinyl, tetrahydropyranyl, piperazinyl, dioxanyl, morpholinyl, quinuclidinyl, 8-azabicyclo[3.2.1]octanyl, 2-azaspiro[3.3]heptanyl, 6-azaspiro[2.5]octanyl, or hexahydro-1H-pyrrolizinyl group; more preferably an azetidinyl, oxetanyl, pyrrolidinyl, tetrahydrofuranyl, pyrazolidinyl, imidazolidinyl, dioxolanyl, piperidinyl, tetrahydropyranyl, piperazinyl, dioxanyl, or morpholinyl group), each of which is optionally substituted with one, two, three, four or five substituents independently selected from fluoro, chloro, cyano, hydroxy, methyl (which, for the avoidance of doubt, includes for example trideuteriomethyl), ethyl, n-propyl, iso-propyl, methoxy, ethoxy, n-propoxy, iso-propoxy, —CF₃, —CH₂CF₃, —CF₂CF₃, —OCF₃, —OCH₂CF₃, —OCF₂CF₃, —N(R⁶)₂, or —SO₂(R⁶);
R⁴ is selected from hydrogen, fluoro, chloro, cyano, cyclopropyl, cyclopropoxy, —CH₃, —CH₂CH₃, —CF₃, —CH₂CF₃, —CF₂CF₃, —OCH₃, —OCH₂CH₃, —OCF₃, —OCH₂CF₃, or —OCF₂CF₃; and
each R⁶ is independently selected from hydrogen, methyl, or ethyl;
provided the compound is not:
4-methoxy-5-(2-methyl-4-(trifluoromethoxy)phenyl)-N-((1s,4s)-4-methylcyclohexyl)pyrimidin-2-amine;
4-methoxy-5-(2-methoxy-4-(trifluoromethoxy)phenyl)-N-((1s,4s)-4-methylcyclohexyl)pyrimidin-2-amine; or
N-cyclopropyl-5-(3,4-dimethoxyphenyl)-4-methyl-pyrimidin-2-amine.

A second aspect of the present invention provides a compound selected from:
4-methyl-N-(2-(1-methylpyrrolidin-2-yl)ethyl)-5-phenylpyrimidin-2-amine;
N-(2-(1-methylpyrrolidin-2-yl)ethyl)-5-phenylpyrimidin-2-amine;
5-(4-fluorophenyl)-N-(2-(1-methylpyrrolidin-2-yl)ethyl)pyrimidin-2-amine;
5-(4-fluorophenyl)-N-((1-methylpiperidin-3-yl)methyl)pyrimidin-2-amine;
5-(4-fluorophenyl)-N-(1-methylpiperidin-3-yl)pyrimidin-2-amine;
5-(4-fluorophenyl)-N-(1-isopropylpiperidin-3-yl)pyrimidin-2-amine;
N¹-(5-(4-fluorophenyl)pyrimidin-2-yl)-N³,N³-dimethylcyclohexane-1,3-diamine;
5-(4-fluorophenyl)-N-((1-methylpiperidin-2-yl)methyl)pyrimidin-2-amine;
5-(4-fluorophenyl)-N-(1-isopropylpiperidin-3-yl)-4-methyl-pyrimidin-2-amine;
5-(2,4-difluorophenyl)-N-((1-ethylpiperidin-2-yl)methyl)-4-methyl-pyrimidin-2-amine;
5-(2,4-difluorophenyl)-N-((1-(dimethylamino)cyclopentyl)methyl)-4-methyl-pyrimidin-2-amine;
N-(5-(2,4-difluorophenyl)-4-methyl-pyrimidin-2-yl)-8-methyl-8-azabicyclo[3.2.1]octan-3-amine;
5-(2,4-difluorophenyl)-4-methyl-N-((1-methylpyrrolidin-3-yl)methyl)pyrimidin-2-amine;
5-(2,4-difluorophenyl)-4-methyl-N-(2-(1-methylpyrrolidin-2-yl)ethyl)pyrimidin-2-amine;
6-(2,4-difluorophenyl)-5-methyl-N-((1-methylpyrrolidin-2-yl)methyl)-1,2,4-triazin-3-amine;
5-(2-fluoro-4-methoxyphenyl)-4-methyl-N-(2-(1-methylpyrrolidin-2-yl)ethyl)pyrimidin-2-amine;
5-(2-fluoro-4-methoxyphenyl)-4-methyl-N-((1-methylpiperidin-2-yl)methyl)pyrimidin-2-amine;
6-(2-fluoro-4-methoxyphenyl)-5-methyl-N-((1-methylpyrrolidin-2-yl)methyl)-1,2,4-triazin-3-amine;
5-(2,4-difluorophenyl)-2-(((1-methylpyrrolidin-2-yl)methyl)amino)pyrimidine-4-carbonitrile;
6-(2,4-difluorophenyl)-5-methyl-N-((1-methylpiperidin-2-yl)methyl)-1,2,4-triazin-3-amine;
6-(2,4-difluorophenyl)-5-methyl-N-(2-(1-methylpyrrolidin-2-yl)ethyl)-1,2,4-triazin-3-amine;
5-(2,4-difluorophenyl)-N-(1-isopropylpyrrolidin-3-yl)-4-methyl-pyrimidin-2-amine;
6-(2-fluoro-4-methoxyphenyl)-5-methyl-N-(2-(1-methylpyrrolidin-2-yl)ethyl)-1,2,4-triazin-3-amine;
5-(2,4-difluorophenyl)-4-methyl-N-((1-methylpyrrolidin-2-yl)methyl)pyrimidin-2-amine;
5-(2-fluoro-4-methoxyphenyl)-2-(((1-methylpiperidin-2-yl)methyl)amino)pyrimidine-4-carbonitrile;
6-(2-fluoro-4-methoxyphenyl)-5-methyl-N-((1-methylpiperidin-2-yl)methyl)-1,2,4-triazin-3-amine;
5-(2-fluoro-4-methoxyphenyl)-2-((2-(1-methylpyrrolidin-2-yl)ethyl)amino)pyrimidine-4-carbonitrile;
5-(2-fluoro-4-methoxyphenyl)-4-methyl-N-((1-methylpyrrolidin-2-yl)methyl)pyrimidin-2-amine;
5-(2,4-difluorophenyl)-4-methyl-N-(1-methylpyrrolidin-3-yl)pyrimidin-2-amine;
5-(2-fluoro-4-methoxyphenyl)-4-methyl-N-((1-methylpiperidin-3-yl)methyl)pyrimidin-2-amine;
5-(2-fluoro-4-methoxyphenyl)-2-(((1-methylpyrrolidin-3-yl)methyl)amino)pyrimidine-4-carbonitrile;
5-(2-fluoro-4-methoxyphenyl)-4-methyl-N-((4-methylmorpholin-3-yl)methyl)pyrimidin-2-amine;
5-(2-fluoro-5-methoxyphenyl)-4-methyl-N-((1-methylpiperidin-2-yl)methyl)pyrimidin-2-amine;
5-(2-fluoro-5-methoxyphenyl)-4-methyl-N-((1-methylpiperidin-3-yl)methyl)pyrimidin-2-amine;
5-(2-chloro-4-methoxyphenyl)-4-methyl-N-((1-methylpiperidin-2-yl)methyl)pyrimidin-2-amine;
5-(2-fluoro-4-methoxyphenyl)-4-methyl-N-((4-methylmorpholin-2-yl)methyl)pyrimidin-2-amine;
4-cyclopropyl-5-(2-fluoro-4-methoxyphenyl)-N-((1-methylpyrrolidin-3-yl)methyl)pyrimidin-2-amine;
2-((3-(dimethylamino)cyclopentyl)amino)-5-(2-fluoro-4-methoxyphenyl)pyrimidine-4-carbonitrile;
N¹-(6-(2,4-difluorophenyl)-5-methyl-1,2,4-triazin-3-yl)-N³,N³-dimethylcyclohexane-1,3-diamine;
5-(2-fluoro-4-methoxyphenyl)-4-methyl-N-(1-methylpiperidin-4-yl)pyrimidin-2-amine;
5-(2-fluoro-4-methoxyphenyl)-N-(1-isopropylpiperidin-4-yl)-4-methyl-pyrimidin-2-amine;
5-(2,4-difluorophenyl)-4-methyl-N-(pyridin-3-ylmethyl)pyrimidin-2-amine;
N¹-(5-(2-fluoro-4-methoxyphenyl)-4-(trifluoromethyl)pyrimidin-2-yl)-N³,N³-dimethylcyclopentane-1,3-diamine;
N¹-(5-(2-fluoro-4-methoxyphenyl)-4-methyl-pyrimidin-2-yl)-N³,N³-dimethylcyclopentane-1,3-diamine;
5-(2-fluoro-4-methoxyphenyl)-4-methyl-N-(tetrahydro-2H-pyran-4-yl)pyrimidin-2-amine;
5-(2-fluoro-4-methoxyphenyl)-4-methyl-N-(tetrahydro-2H-pyran-3-yl)pyrimidin-2-amine;
5-(2-chloro-4-methoxyphenyl)-4-methyl-N-((tetrahydrofuran-2-yl)methyl)pyrimidin-2-amine;
5-(2-fluoro-4-methoxyphenyl)-N-(1-isopropylpiperidin-3-yl)-4-methyl-pyrimidin-2-amine;
5-(2-fluoro-4-methoxyphenyl)-N-((1-isopropylpiperidin-3-yl)methyl)-4-methyl-pyrimidin-2-amine;
5-(2-fluoro-4-methoxyphenyl)-N-((1-isopropylpiperidin-2-yl)methyl)-4-methyl-pyrimidin-2-amine;
5-(2-fluoro-4-methoxyphenyl)-4-methyl-N-((tetrahydro-2H-pyran-3-yl)methyl)pyrimidin-2-amine;
5-(2,4-difluorophenyl)-4-methyl-N-(3-(methylsulfonyl)cyclopentyl)pyrimidin-2-amine;
5-(2-fluoro-4-methoxyphenyl)-N-(3-methoxycyclohexyl)-4-methyl-pyrimidin-2-amine;
N¹-(5-(2,4-difluorophenyl)-4-methyl-pyrimidin-2-yl)-N³,N³-dimethylcyclohexane-1,3-diamine;
2-((3-(dimethylamino)cyclohexyl)amino)-5-(2-fluoro-4-methoxyphenyl)pyrimidine-4-carbonitrile;
5-(2,4-difluorophenyl)-2-((3-(dimethylamino)cyclopentyl)amino)pyrimidine-4-carbonitrile;
5-(2,4-difluorophenyl)-2-((3-(dimethylamino)cyclohexyl)amino)pyrimidine-4-carbonitrile;
5-(2,4-difluorophenyl)-4-methyl-N-(1-(2,2,2-trifluoroethyl)piperidin-3-yl)pyrimidin-2-amine;
5-(2-fluoro-5-methoxyphenyl)-4-methyl-N-((4-methylmorpholin-3-yl)methyl)pyrimidin-2-amine;
5-(2-chloro-4-methoxyphenyl)-4-methyl-N-((4-methylmorpholin-3-yl)methyl)pyrimidin-2-amine;
5-(2-chloro-4-methoxyphenyl)-4-methyl-N-((1-methyl-1H-imidazol-5-yl)methyl)pyrimidin-2-amine;
5-(2,4-difluorophenyl)-4-methyl-N-(1-(1-methylpyrrolidin-3-yl)ethyl)pyrimidin-2-amine;
5-(2,4-difluorophenyl)-2-((1-isopropylpiperidin-4-yl)amino)pyrimidine-4-carbonitrile;
5-(2,4-difluorophenyl)-2-((1-methylpiperidin-4-yl)amino)pyrimidine-4-carbonitrile;
5-(2,4-difluorophenyl)-N-(1-isopropylpiperidin-4-yl)-4-methyl-pyrimidin-2-amine;
N¹-(5-(2,4-difluorophenyl)-4-methyl-pyrimidin-2-yl)-N³,N³-dimethylcyclobutane-1,3-diamine;
6-(2-fluoro-4-methoxyphenyl)-5-methyl-N-(1-methylpiperidin-3-yl)-1,2,4-triazin-3-amine;
5-(2-fluoro-4-methoxyphenyl)-4,6-dimethyl-N-((1-methylpyrrolidin-2-yl)methyl)pyrimidin-2-amine;
5-(2,4-difluorophenyl)-4-methyl-N-(1-methylpiperidin-4-yl)pyrimidin-2-amine;
5-(2,4-difluorophenyl)-N-(1-isopropylpiperidin-3-yl)-4-methyl-pyrimidin-2-amine;
5-(2-fluoro-4-(trifluoromethoxy)phenyl)-4-methyl-N-(pyrrolidin-2-ylmethyl)pyrimidin-2-amine;
5-(2-fluoro-4-methoxyphenyl)-4-methoxy-6-methyl-N-((1-methylpyrrolidin-2-yl)methyl)pyrimidin-2-amine;
6-(2-fluoro-4-methoxyphenyl)-N-(1-isopropylpiperidin-3-yl)-5-methyl-1,2,4-triazin-3-amine;
5-(2,4-difluorophenyl)-4-methyl-N-(1-methylpiperidin-3-yl)pyrimidin-2-amine;
5-(2,4-difluorophenyl)-N-(1-ethylpiperidin-3-yl)-4-methyl-pyrimidin-2-amine;
5-(2,4-difluorophenyl)-4-methyl-N-((4-methylmorpholin-3-yl)methyl)pyrimidin-2-amine;
N-((4,4-difluoro-1-methylpyrrolidin-2-yl)methyl)-5-(2,4-difluorophenyl)-4-methyl-pyrimidin-2-amine;
5-(2-fluoro-4-methoxyphenyl)-2-((1-isopropylpiperidin-4-yl)amino)pyrimidine-4-carbonitrile;
5-(2,4-difluorophenyl)-4-methyl-N-(pyrrolidin-2-ylmethyl)pyrimidin-2-amine;
5-(2,4-difluorophenyl)-N-((3,3-difluoropyrrolidin-2-yl)methyl)-4-methyl-pyrimidin-2-amine;
N-((3,3-difluoro-1-methylpyrrolidin-2-yl)methyl)-5-(2,4-difluorophenyl)-4-methyl-pyrimidin-2-amine;
5-(2,4-difluorophenyl)-N-((1-ethylpyrrolidin-2-yl)methyl)-4-methyl-pyrimidin-2-amine;
N-(5-(2,4-difluorophenyl)-4-methyl-pyrimidin-2-yl)hexahydro-1H-pyrrolizin-1-amine;
5-(2,4-difluorophenyl)-4-methyl-N-((1-(trideuteriomethyl)pyrrolidin-2-yl)methyl)pyrimidin-2-amine;
5-(2-fluoro-4-(trifluoromethoxy)phenyl)-N-(1-isopropylpiperidin-4-yl)-4-methyl-pyrimidin-2-amine;
5-(2-fluoro-4-(trifluoromethoxy)phenyl)-4-methyl-N-(1-methylpiperidin-4-yl)pyrimidin-2-amine;
5-(2-fluoro-4-(trifluoromethyl)phenyl)-4-methyl-N-((1-methylpyrrolidin-2-yl)methyl)pyrimidin-2-amine;
5-(4-fluoro-2-(trifluoromethyl)phenyl)-4-methyl-N-((1-methylpyrrolidin-2-yl)methyl)pyrimidin-2-amine;
5-(2-fluoro-4-(trifluoromethoxy)phenyl)-4-methyl-N-((1-methylpyrrolidin-2-yl)methyl)pyrimidin-2-amine;
5-(2,4-difluorophenyl)-4-methyl-N-((1-(2,2,2-trifluoroethyl)pyrrolidin-2-yl)methyl)pyrimidin-2-amine;
5-(2,4-difluorophenyl)-N-((3-fluoro-1-methylpyrrolidin-2-yl)methyl)-4-methyl-pyrimidin-2-amine;
N-(5-(2-fluoro-4-(trifluoromethoxy)phenyl)-4-methyl-pyrimidin-2-yl)hexahydro-1H-pyrrolizin-1-amine;
N-((1-ethylpyrrolidin-2-yl)methyl)-5-(2-fluoro-4-(trifluoromethyl)phenyl)-4-methyl-pyrimidin-2-amine;
5-(2-fluoro-4-(trifluoromethyl)phenyl)-4-methyl-N-((1-methylpyrrolidin-3-yl)methyl)pyrimidin-2-amine;
5-(2-fluoro-4-(trifluoromethyl)phenyl)-N-(1-isopropylpyrrolidin-3-yl)-4-methyl-pyrimidin-2-amine;
5-(2-fluoro-4-(trifluoromethyl)phenyl)-4-methyl-N-((1-methyl-4-piperidinyl)methyl)pyrimidin-2-amine;
5-(2-fluoro-4-(trifluoromethyl)phenyl)-4-methyl-N-(pyrrolidin-2-ylmethyl)pyrimidin-2-amine;
5-(2-fluoro-4-(trifluoromethyl)phenyl)-4-methyl-N-(2-piperidinylmethyl)pyrimidin-2-amine;
5-(2-fluoro-4-(trifluoromethyl)phenyl)-4-methyl-N-(2-(1-methylpyrrolidin-2-yl)ethyl)pyrimidin-2-amine;
5-(2-fluoro-4-(trifluoromethyl)phenyl)-4-methyl-N-((1-methyl-3-piperidinyl)methyl)pyrimidin-2-amine;
5-(2-fluoro-4-(trifluoromethyl)phenyl)-4-methyl-N-((4-methylmorpholin-2-yl)methyl)pyrimidin-2-amine;
N¹-(5-(2-fluoro-4-(trifluoromethyl)phenyl)-4-methyl-pyrimidin-2-yl)-N²,N²-dimethyl-cyclohexane-1,2-diamine;
5-(2-fluoro-4-(trifluoromethyl)phenyl)-4-methyl-N-(pyrrolidin-3-yl)pyrimidin-2-amine;
N³-(5-(2-fluoro-4-(trifluoromethyl)phenyl)-4-methyl-pyrimidin-2-yl)cyclopentane-1,3-diamine;
N-(4-fluoropyrrolidin-3-yl)-5-(2-fluoro-4-(trifluoromethyl)phenyl)-4-methyl-pyrimidin-2-amine;
5-(2-fluoro-4-(trifluoromethyl)phenyl)-4-methyl-N-((3-piperidinyl)methyl)pyrimidin-2-amine;
4-((5-(2-fluoro-4-(trifluoromethyl)phenyl)-4-methyl-pyrimidin-2-yl)amino)pyrrolidin-3-ol;
5-(2-fluoro-4-(trifluoromethyl)phenyl)-4-methyl-N-(3-piperidinyl)pyrimidin-2-amine;
5-(2-fluoro-4-(trifluoromethyl)phenyl)-4-methyl-N-((1-(trideuteriomethyl)pyrrolidin-2-yl)methyl)pyrimidin-2-amine;
N-(5-(2-fluoro-4-(trifluoromethyl)phenyl)-4-methyl-pyrimidin-2-yl)quinuclidin-3-amine;
N-(5-(2-fluoro-4-(trifluoromethyl)phenyl)-4-methyl-pyrimidin-2-yl)-8-azabicyclo[3.2.1]octan-3-amine;
N-(5-(2-fluoro-4-(trifluoromethyl)phenyl)-4-methyl-pyrimidin-2-yl)-2-azaspiro[3.3]heptan-6-amine;
N-(5-(2-fluoro-4-(trifluoromethyl)phenyl)-4-methyl-pyrimidin-2-yl)-6-azaspiro[2.5]octan-1-amine;
5-(2-fluoro-4-(trifluoromethyl)phenyl)-N-(3-fluoropiperidin-4-yl)-4-methyl-pyrimidin-2-amine;
N¹-(5-(2-fluoro-4-(trifluoromethyl)phenyl)-4-methyl-pyrimidin-2-yl)cyclohexane-1,3-diamine;
5-(2-fluoro-4-(trifluoromethyl)phenyl)-4-methyl-N-(2,2,6,6-tetramethylpiperidin-4-yl)pyrimidin-2-amine;
5-(2-fluoro-4-(trifluoromethyl)phenyl)-4-methyl-N-(piperidin-3-ylmethyl)pyrimidin-2-amine;
5-(2-fluoro-4-(trifluoromethyl)phenyl)-4-methyl-N-(piperidin-3-yl)pyrimidin-2-amine;
5-(2-fluoro-4-(trifluoromethyl)phenyl)-4-methyl-N-(1-(pyrrolidin-2-yl)ethyl)pyrimidin-2-amine;
5-(2-fluoro-4-(trifluoromethyl)phenyl)-4-methyl-N-(1-(1-methylpyrrolidin-2-yl)ethyl)pyrimidin-2-amine;
5-(2-fluoro-4-(trifluoromethyl)phenyl)-N-((3-fluoropyrrolidin-2-yl)methyl)-4-methyl-pyrimidin-2-amine;
5-(2-fluoro-4-(trifluoromethyl)phenyl)-4-methyl-N-((2-methyl-pyrrolidin-2-yl)methyl)pyrimidin-2-amine;
N-((1,2-dimethylpyrrolidin-2-yl)methyl)-5-(2-fluoro-4-(trifluoromethyl)phenyl)-4-methyl-pyrimidin-2-amine;
N-((1,5-dimethylpyrrolidin-2-yl)methyl)-5-(2-fluoro-4-(trifluoromethyl)phenyl)-4-methyl-pyrimidin-2-amine;
N-(5-(2-fluoro-4-(trifluoromethyl)phenyl)-4-methyl-pyrimidin-2-yl)-1-methylpyrrolidine-2-carboxamide;
   or an enantiomer of any of the foregoing;
   or a pharmaceutically acceptable salt, solvate or prodrug of any of the foregoing.

In one embodiment of the second aspect of the present invention, there is provided a compound selected from:
(R)-5-(2-fluoro-4-(trifluoromethyl)phenyl)-4-methyl-N-((1-methylpyrrolidin-2-yl)methyl)pyrimidin-2-amine;
(R)-5-(2-fluoro-4-(trifluoromethyl)phenyl)-4-methyl-N-(pyrrolidin-2-ylmethyl)pyrimidin-2-amine (such as a semi-fumarate salt);
(S)-5-(2-fluoro-4-(trifluoromethyl)phenyl)-4-methyl-N-(pyrrolidin-2-ylmethyl)pyrimidin-2-amine (such as a semi-fumarate salt);
N-(5-(2-fluoro-4-(trifluoromethyl)phenyl)-4-methyl-pyrimidin-2-yl)-2-azaspiro[3.3]heptan-6-amine (such as a formate salt);
5-(2-fluoro-4-(trifluoromethyl)phenyl)-4-methyl-N-((1*S)-1-((2S)-pyrrolidin-2-yl)ethyl)pyrimidin-2-amine (such as a semi-fumarate salt);
N-(((2*S,5*R)-1,5-dimethylpyrrolidin-2-yl)methyl)-5-(2-fluoro-4-(trifluoromethyl)phenyl)-4-methyl-pyrimidin-2-amine (such as a fumarate salt);
   or a pharmaceutically acceptable salt, solvate or prodrug of any of the foregoing.

In one embodiment of the second aspect of the present invention, there is provided a compound selected from:
(S)-5-(2,4-difluorophenyl)-4-methyl-N-((1-methylpyrrolidin-2-yl)methyl)pyrimidin-2-amine;
(R)-5-(2,4-difluorophenyl)-4-methyl-N-((1-methylpyrrolidin-2-yl)methyl)pyrimidin-2-amine;
5-(2,4-difluorophenyl)-N-(1-isopropylpiperidin-4-yl)-4-methyl-pyrimidin-2-amine;
(R)-5-(2,4-difluorophenyl)-N-(1-ethylpiperidin-3-yl)-4-methyl-pyrimidin-2-amine (such as a fumarate salt);
(S)-5-(2,4-difluorophenyl)-N-(1-ethylpiperidin-3-yl)-4-methyl-pyrimidin-2-amine (such as a fumarate salt);
5-(2,4-difluorophenyl)-4-methyl-N-(pyrrolidin-2-ylmethyl)pyrimidin-2-amine;
5-(2,4-difluorophenyl)-4-methyl-N-((1-(trideuteriomethyl)pyrrolidin-2-yl)methyl)pyrimidin-2-amine;
(S)-5-(2,4-difluorophenyl)-4-methyl-N-((1-(trideuteriomethyl)pyrrolidin-2-yl)methyl)pyrimidin-2-amine;
(R)-5-(2,4-difluorophenyl)-4-methyl-N-((1-(trideuteriomethyl)pyrrolidin-2-yl)methyl)pyrimidin-2-amine;
(R)-5-(2,4-difluorophenyl)-4-methyl-N-(pyrrolidin-2-ylmethyl)pyrimidin-2-amine;
(S)-5-(2,4-difluorophenyl)-4-methyl-N-(pyrrolidin-2-ylmethyl)pyrimidin-2-amine;
5-(2-fluoro-4-(trifluoromethyl)phenyl)-4-methyl-N-((3S)-3-piperidinyl)pyrimidin-2-amine (such as a hydrochloride salt);
   or a pharmaceutically acceptable salt, solvate or prodrug of any of the foregoing.

A third aspect of the present invention provides a process for the preparation of a compound of formula (I) or a pharmaceutically acceptable salt, solvate or prodrug thereof, according to the first aspect of the present invention, wherein the process comprises:
(a) reacting a compound of formula (II) or a salt thereof, with a compound of formula (III) or a salt thereof, to provide a compound of formula (IV) or a salt thereof:

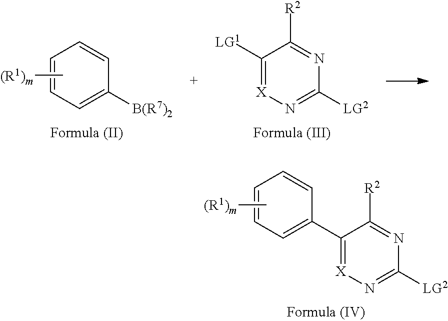

wherein m, R¹, R² and X are as defined in the first aspect of the present invention; R⁷ is independently selected from hydroxy, C₁-C₅ alkyl, or C₁-C₅ alkoxy, or two R⁷ together with the boron to which they are attached form an optionally substituted 3- to 12-membered heterocyclic group; and LG¹ and LG² are leaving groups such as a halo (such as fluoro, chloro, bromo, or iodo), a sulfate group (such as methyl sulfate), or a sulfonate group (such as mesylate, triflate, or tosylate):
(b) reacting a compound of formula (IV) or a salt thereof, with a compound of formula (V) or a salt thereof, to provide a compound of formula (I) or a salt thereof:

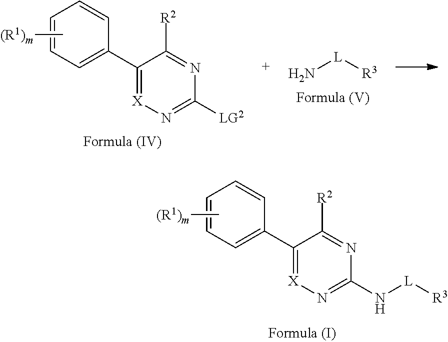

wherein R³ and L are as defined in the first aspect of the present invention;
and optionally thereafter carrying out one or more of the following procedures:
   converting a compound of formula (I) into another compound of formula (I);
   removing any protecting groups;
   forming a pharmaceutically acceptable salt, solvate or prodrug.

Step (a) of the above process may conveniently be carried out by a Suzuki reaction. R⁷ may be selected such that the compound of formula (II) is selected from:

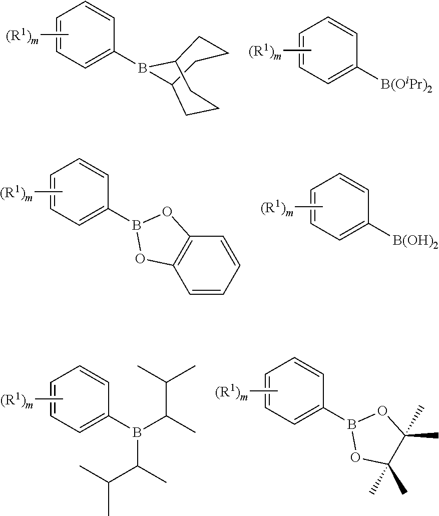

Step (a) may be carried out by combining the compound of formula (II) or a salt thereof with the compound of formula (III) or a salt thereof in the presence of a palladium catalyst (such as Pd(dppf)Cl₂; Pd(PPh₃)₄; Pd₂(dba)₃ optionally with PPh₃, P^{t}Bu₃ or PCy₃; or Pd(OAc)₂ optionally with PPh₃, P^{t}Bu₃ or PCy₃) and preferably a base (such as Na₂CO₃, K₂CO₃, Cs₂CO₃, NaOEt, NaOH, KOH, CsOH, or K₃PO₄) in a solvent (such as dioxane, water, methanol, ethanol, THF, toluene, benzene, or a mixture thereof) preferably at a temperature of from room temperature to 100° C. Typically the reaction takes from 0.5 to 8 hours.

Step (b) of the above process may conveniently be carried out by combining the compound of formula (IV) or a salt thereof with the compound of formula (V) or a salt thereof in the presence of a base (such as DIPEA, triethylamine, NaOH, or KOH) in a solvent (such as dioxane, methanol, ethanol, DMSO, NMP, DMF, or a mixture thereof) preferably at a temperature of from room temperature to 100° C. Typically the reaction takes from 4 to 20 hours.

It will be appreciated by those skilled in the art that in the processes of the present invention certain functional groups such as phenol, hydroxy or amino groups in the reagents may need to be protected by protecting groups. Thus, the preparation of the compounds, salts, solvates and prodrugs of the present invention may involve, at an appropriate stage, the introduction and/or removal of one or more protecting groups.

The protection and deprotection of functional groups are described, for example, in ‘Protective Groups in Organic Chemistry’, edited by J. W. F. McOmie, Plenum Press (1973); ‘Greene's Protective Groups in Organic Synthesis’, 4th edition, T. W. Greene and P. G. M. Wuts, Wiley-Interscience (2007); and ‘Protecting Groups’, 3rd edition, P. J. Kocienski, Thieme (2005).

The compounds of formula (I) may be converted into a pharmaceutically acceptable salt thereof, preferably an acid addition salt such as a formate, hemi-formate, hydrochloride, hydrobromide, benzenesulfonate (besylate), saccharin (e.g. monosaccharin), trifluoroacetate, sulfate, nitrate, phosphate, acetate, fumarate, semi-fumarate, maleate, tartrate, lactate, citrate, pyruvate, succinate, valerate, propanoate, butanoate, malonate, oxalate, 1-hydroxy-2-naphthoate (xinafoate), methanesulfonate or p-toluenesulfonate salt. In one embodiment of the invention, the compounds of formula (I) are in the form of a hydrochloride, formate or fumarate salt.

A salt of a compound of formula (I) may also be formed between a protic acid functionality of a compound of formula (I) and a suitable cation. Suitable cations include, but are not limited to lithium, sodium, potassium, magnesium, calcium and ammonium. In one embodiment of the invention, the salt is a mono- or di-sodium salt or a mono- or di-potassium salt.

Compounds of formula (I) and their salts may be in the form of hydrates or solvates which form another embodiment of the present invention. Such solvates may be formed with common organic solvents, including but not limited to, alcoholic solvents e.g. methanol, ethanol or isopropanol.

In one embodiment of the present invention, therapeutically inactive prodrugs are provided. Prodrugs are compounds which, when administered to a subject such as a human, are converted in whole or in part to a compound of formula (I). Generally, the prodrugs are pharmacologically inert chemical derivatives that can be converted in vivo to the active drug molecules to exert a therapeutic effect. Any of the compounds of formula (I) can be administered as a prodrug to increase the activity, bioavailability, or stability of the compound of formula (I) or to otherwise alter the properties of the compound of formula (I). Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

Where the compounds, salts, solvates and prodrugs of the present invention are capable of existing in stereoisomeric forms, it will be understood that the invention encompasses the use of all geometric and optical isomers (including atropisomers) and mixtures thereof.

The use of tautomers and mixtures thereof also forms an embodiment of the present invention. The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight. Enantiomerically pure isomers are particularly desired.

The compounds, salts, solvates and prodrugs of the present invention may contain any stable isotope including, but not limited to ¹²C, ³C, ¹H, ²H (D), ¹⁴N, ¹⁵N, ¹⁶O, ⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴, ¹²⁵I and ¹³¹I. Therefore, the term “hydrogen”, for example, encompasses ¹H, ²H (D) and ³H (T). Similarly, carbon atoms are to be understood to include ¹¹C, ¹²C, ¹³C and ¹⁴C, nitrogen atoms are to be understood to include ¹³N, ¹⁴N and ¹⁵N, oxygen atoms are to be understood to include ¹⁵O, ¹⁶O, ¹⁷O and ¹⁸O, fluorine atoms are to be understood to include ¹⁸F and ¹⁹F, and iodine atoms are to be understood to include ¹²³I, ¹²⁴I, ¹²⁵I, ¹²⁷I and ¹³¹I.

In one embodiment, the compounds, salts, solvates and prodrugs of the present invention may be isotopically labelled. As used herein, an “isotopically labelled” compound is one in which the abundance of a particular nuclide at a particular atomic position within the molecule is increased above the level at which it occurs in nature. Any of the compounds, salts, solvates and prodrugs of the present invention can be isotopically labelled, for example, any of examples 1 to 140.

In one embodiment, the compounds, salts, solvates and prodrugs of the present invention may bear one or more radiolabels. Such radiolabels may be introduced by using radiolabel-containing reagents in the synthesis of the compounds, salts, solvates or prodrugs, or may be introduced by coupling the compounds, salts, solvates or prodrugs to chelating moieties capable of binding to a radioactive metal atom. Such radiolabelled versions of compounds, salts, solvates and prodrugs may be used, for example, in diagnostic imaging studies.

In one embodiment, the compounds, salts, solvates and prodrugs of the present invention may be tritiated, i.e. they contain one or more ³H (T) atoms. Any of the compounds, salts, solvates and prodrugs of the present invention can be tritiated, for example, any of examples 1 to 140.

The compounds, salts, solvates and prodrugs of the present invention may be amorphous or in a polymorphic form or a mixture of any of these, each of which is an embodiment of the present invention.

The compounds, salts, solvates and prodrugs of the present invention have activity as pharmaceuticals and may be used in treating or preventing a disease, disorder or condition that has dysregulation of dopamine, noradrenaline or serotonin as a key pathological mechanism.

Therefore, a fourth aspect of the present invention provides a compound of formula (I) or a pharmaceutically acceptable salt, solvate or prodrug thereof, according to the first aspect of the present invention, for use in therapy, in particular for use in treating or preventing (i) a movement disorder such as tremor (including resting tremor in Parkinson's disease; and essential tremor including essential tremor in isolation, essential tremor in Parkinson's disease, essential tremor in Alzheimer's disease, and essential tremor in other neurodegenerative diseases), dystonia, dyskinesia (including L-DOPA induced dyskinesia in Parkinson's disease), Parkinson's disease, or Huntington's disease; (ii) a psychiatric disorder such as schizophrenia, psychotic disorder, psychosis, schizoaffective disorder, bipolar disorder (including bipolar I, bipolar II, bipolar mania, and bipolar depression), attention deficit hyperactivity disorder (ADHD), autism spectrum disorder (ASD) including Fragile X syndrome, Tourettes syndrome, or an addiction disorder (including substance or drug dependence, alcohol dependence, nicotine dependence, binge eating, and gambling disorder); or (iii) a non-motor symptom of Parkinson's disease such as apathy, anhedonia, or depression. In one embodiment, the present invention provides a compound of formula (I) or a pharmaceutically acceptable salt, solvate or prodrug thereof, according to the first aspect of the present invention, for use in treating or preventing a movement disorder such as tremor, dystonia, dyskinesia, Parkinson's disease, or Huntington's disease; or a psychiatric disorder such as schizophrenia, psychotic disorder, psychosis, schizoaffective disorder, bipolar disorder (including bipolar I, bipolar IL, bipolar mania, and bipolar depression), attention deficit hyperactivity disorder (ADHD), autism spectrum disorder (ASD) including Fragile X syndrome, Tourettes syndrome, or an addiction disorder (including substance or drug dependence, alcohol dependence, nicotine dependence, binge eating, and gambling disorder). In one embodiment, the present invention provides a compound of formula (I) or a pharmaceutically acceptable salt, solvate or prodrug thereof, according to the first aspect of the present invention, for use in treating or preventing Parkinson's disease, tremor, Tourettes syndrome, or an addiction.

A fifth aspect of the present invention provides a use of a compound of formula (I) or a pharmaceutically acceptable salt, solvate or prodrug thereof, according to the first aspect of the present invention, for the manufacture of a medicament for treating or preventing (i) a movement disorder such as tremor (including resting tremor in Parkinson's disease; and essential tremor including essential tremor in isolation, essential tremor in Parkinson's disease, essential tremor in Alzheimer's disease, and essential tremor in other neurodegenerative diseases), dystonia, dyskinesia (including L-DOPA induced dyskinesia in Parkinson's disease), Parkinson's disease, or Huntington's disease; (ii) a psychiatric disorder such as schizophrenia, psychotic disorder, psychosis, schizoaffective disorder, bipolar disorder (including bipolar I, bipolar II, bipolar mania, and bipolar depression), attention deficit hyperactivity disorder (ADHD), autism spectrum disorder (ASD) including Fragile X syndrome, Tourettes syndrome, or an addiction disorder (including substance or drug dependence, alcohol dependence, nicotine dependence, binge eating, and gambling disorder); or (iii) a non-motor symptom of Parkinson's disease such as apathy, anhedonia, or depression. In one embodiment, the present invention provides a use of a compound of formula (I) or a pharmaceutically acceptable salt, solvate or prodrug thereof, according to the first aspect of the present invention, for the manufacture of a medicament for treating or preventing a movement disorder such as tremor, dystonia, dyskinesia, Parkinson's disease, or Huntington's disease; or a psychiatric disorder such as schizophrenia, psychotic disorder, psychosis, schizoaffective disorder, bipolar disorder (including bipolar I, bipolar II, bipolar mania, and bipolar depression), attention deficit hyperactivity disorder (ADHD), autism spectrum disorder (ASD) including Fragile X syndrome, Tourettes syndrome, or an addiction disorder (including substance or drug dependence, alcohol dependence, nicotine dependence, binge eating, and gambling disorder). In one embodiment, the present invention provides a use of a compound of formula (I) or a pharmaceutically acceptable salt, solvate or prodrug thereof, according to the first aspect of the present invention, for the manufacture of a medicament for treating or preventing Parkinson's disease, tremor, Tourettes syndrome, or an addiction.

A sixth aspect of the present invention provides a method of treating or preventing (i) a movement disorder such as tremor (including resting tremor in Parkinson's disease; and essential tremor including essential tremor in isolation, essential tremor in Parkinson's disease, essential tremor in Alzheimer's disease, and essential tremor in other neurodegenerative diseases), dystonia, dyskinesia (including L-DOPA induced dyskinesia in Parkinson's disease), Parkinson's disease, or Huntington's disease; (ii) a psychiatric disorder such as schizophrenia, psychotic disorder, psychosis, schizoaffective disorder, bipolar disorder (including bipolar I, bipolar II, bipolar mania, and bipolar depression), attention deficit hyperactivity disorder (ADHD), autism spectrum disorder (ASD) including Fragile X syndrome, Tourettes syndrome, or an addiction disorder (including substance or drug dependence, alcohol dependence, nicotine dependence, binge eating, and gambling disorder); or (iii) a non-motor symptom of Parkinson's disease such as apathy, anhedonia, or depression; the method comprising administering a therapeutically or prophylactically effective amount of a compound of formula (I) or a pharmaceutically acceptable salt, solvate or prodrug thereof, according to the first aspect of the present invention, to a patient in need thereof. In one embodiment, the present invention provides a method of treating or preventing a movement disorder such as tremor, dystonia, dyskinesia, Parkinson's disease, or Huntington's disease; or a psychiatric disorder such as schizophrenia, psychotic disorder, psychosis, schizoaffective disorder, bipolar disorder (including bipolar I, bipolar IL, bipolar mania, and bipolar depression), attention deficit hyperactivity disorder (ADHD), autism spectrum disorder (ASD) including Fragile X syndrome, Tourettes syndrome, or an addiction disorder (including substance or drug dependence, alcohol dependence, nicotine dependence, binge eating, and gambling disorder); the method comprising administering a therapeutically or prophylactically effective amount of a compound of formula (I) or a pharmaceutically acceptable salt, solvate or prodrug thereof, according to the first aspect of the present invention, to a patient in need thereof. In one embodiment, the present invention provides a method of treating or preventing Parkinson's disease, tremor, Tourettes syndrome, or an addiction, the method comprising administering a therapeutically or prophylactically effective amount of a compound of formula (I) or a pharmaceutically acceptable salt, solvate or prodrug thereof, according to the first aspect of the present invention, to a patient in need thereof.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disorder or condition in question. Persons at risk of developing a particular disorder or condition generally include those having a family history of the disorder or condition, or those who have been identified by genetic testing or screening to be particularly susceptible to developing the disorder or condition or those in the prodromal phase of a disorder.

The terms “treat”, “treatment” and “treating” include improvement of the conditions described herein. The terms “treat”, “treatment” and “treating” include all processes providing slowing, interrupting, arresting, controlling, or stopping of the state or progression of the conditions described herein, but does not necessarily indicate a total elimination of all symptoms or a cure of the condition. The terms “treat”, “treatment” and “treating” are intended to include therapeutic as well as prophylactic treatment of such conditions.

As used herein the terms “disease”, “disorder” and “condition” relate to any unhealthy or abnormal state. The terms “disease, disorder or condition that has dysregulation of dopamine as a key pathological mechanism”, “disease, disorder or condition that has dysregulation of noradrenaline as a key pathological mechanism” and “disease, disorder or condition that has dysregulation of serotonin as a key pathological mechanism” include diseases, disorders and conditions in which the modulation of the receptor α6 (nAChRα6) may provide a therapeutic benefit, examples of which include:
(1) Movement disorders: such as tremor (including resting tremor in Parkinson's disease; and essential tremor including essential tremor in isolation, essential tremor in Parkinson's disease, essential tremor in Alzheimer's disease, and essential tremor in other neurodegenerative diseases), dystonia, dyskinesia (including L-DOPA induced dyskinesia in Parkinson's disease), Parkinson's disease, and Huntington's disease; and
(2) Psychiatric disorders: such as schizophrenia, psychotic disorder, psychosis, schizoaffective disorder, bipolar disorder (including bipolar I, bipolar IL, bipolar mania, and bipolar depression), attention deficit hyperactivity disorder (ADHD), autism spectrum disorder (ASD) including Fragile X syndrome, Tourettes syndrome, and an addiction disorder (including substance or drug dependence, alcohol dependence, nicotine dependence, binge eating, and gambling disorder); and
(3) Non-motor symptoms of Parkinson's disease: such as apathy, anhedonia, and depression.

Schizophrenia is a debilitating psychiatric disorder characterised by a combination of negative symptoms (such as social withdrawal, anhedonia, avolition and apathy) and positive symptoms (including hallucinations, delusions and paranoia) as well as marked cognitive deficits (such as impairment of executive function). The executive function (EF) has been defined as “a set of abilities, which allows us to invoke voluntary control of our behavioural responses. These functions enable human beings to develop and carry out plans, make up analogies, obey social rules, solve problems, adapt to unexpected circumstances, do many tasks simultaneously, and locate episodes in time and place. EF includes divided attention and sustained attention, working memory (WM), set-shifting, flexibility, planning, and the regulation of goal directed behaviour and can be defined as a brain function underlying the human faculty to act or think not only in reaction to external events but also in relation with internal goals and states” (Orellana and Slachevsky, Front Psychiatry, 2013, vol 4, article 35).

Accordingly, the present invention also provides a method of treating or preventing a negative symptom, a positive symptom and/or a cognitive deficit associated with a psychiatric disorder, especially schizophrenia, which comprises administering to a patient in need thereof a therapeutically or prophylactically effective amount of a compound of formula (I) or a pharmaceutically acceptable salt, solvate or prodrug thereof as hereinbefore defined.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts, solvates and prodrugs thereof may be used on their own, but will generally be administered in the form of a pharmaceutical composition in which the active ingredient is in association with a pharmaceutically acceptable adjuvant, diluent or carrier.

Therefore, a seventh aspect of the present invention provides a pharmaceutical composition comprising a compound of formula (I) or a pharmaceutically acceptable salt, solvate or prodrug thereof, according to the first aspect of the present invention, in association with a pharmaceutically acceptable adjuvant, diluent or carrier, and optionally one or more other therapeutic agents.

The invention still further provides a process for the preparation of a pharmaceutical composition of the invention which comprises mixing a compound of formula (I) or a pharmaceutically acceptable salt, solvate or prodrug thereof, according to the first aspect of the present invention, with a pharmaceutically acceptable adjuvant, diluent or carrier.

Conventional procedures for the selection and preparation of suitable pharmaceutical formulations are described in, for example, “Pharmaceutics—The Science of Dosage Form Design”, M. E. Aulton, Churchill Livingstone, 1988.

Pharmaceutically acceptable adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, rectally, nasally, buccally, vaginally or via an implanted reservoir. Oral administration is preferred. The pharmaceutical compositions of the invention may contain any conventional non-toxic pharmaceutically acceptable adjuvants, diluents or carriers. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. The suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable diluents and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to, capsules, tablets, powders, granules, and aqueous suspensions and solutions. These dosage forms are prepared according to techniques well-known in the art of pharmaceutical formulation. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient may be combined with emulsifying and suspending agents. If desired, certain sweetening and/or flavouring and/or colouring agents may be added.

The pharmaceutical compositions of the invention may also be administered in the form of suppositories for rectal administration. These compositions can be prepared by mixing the active ingredient with a suitable non-irritating excipient which is solid at room temperature but liquid at the rectal temperature and therefore will melt in the rectum to release the active ingredient. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

Depending on the mode of administration, the pharmaceutical composition will preferably comprise from 0.05 to 99% by weight, more preferably from 0.05 to 80% by weight, still more preferably from 0.10 to 70% by weight, and even more preferably from 0.10 to 50% by weight of active ingredient, all percentages by weight being based on total composition.

The compounds of the invention (that is, compounds of formula (I) and pharmaceutically acceptable salts, solvates and prodrugs thereof) may also be administered in conjunction with other compounds used for the treatment of the above conditions.

The invention therefore further relates to combination therapies wherein a compound of the invention or a pharmaceutical composition or formulation comprising a compound of the invention is administered with another therapeutic agent or agents for the treatment of one or more of the conditions previously indicated. The compound of the invention or the pharmaceutical composition or formulation comprising the compound of the invention may be administered simultaneously with, separately from or sequentially to the one or more other therapeutic agents. The compound of the invention and the one or more other therapeutic agents may be comprised in the same pharmaceutical composition or formulation, or in separate pharmaceutical compositions or formulations. The compound of the invention and the one or more other therapeutic agents may be provided in the form of a kit comprising: (i) a compound of the invention or a pharmaceutical composition or formulation comprising a compound of the invention; and (ii) one or more other therapeutic agents or a pharmaceutical composition or formulation comprising one or more other therapeutic agents. The one or more other therapeutic agents may be selected from the following:
(i) anti-addiction drugs including, for example, acamprosate, disulfiram, naltrexone and nalmefene for alcohol dependency, and venlafaxine, gabapentin, modafinil, topiramate, vigabatrin and baclofen for drug, particularly cocaine, addiction, and equivalents and pharmaceutically active isomer(s) and/or metabolite(s) thereof;
(ii) antidepressants including, for example, amitriptyline, amoxapine, bupropion, citalopram, clomipramine, desipramine, doxepin, duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robaizotan, sertraline, sibutramine, tianeptine, thionisoxetine, tranylcypromaine, trazodone, trimipramine, venlafaxine, and vortioxetine, and equivalents and pharmaceutically active isomer(s) and/or metabolite(s) thereof;
(iii) antipsychotics including, for example, amisulpride, aripiprazole, asenapine, benzisoxidil, bifeprunox, brexpiprazole, carbamazepine, cariprazine, clozapine, chlorpromazine, debenzapine, divalproex, duloxetine, eszopiclone, fluphenazine, haloperidol, iloperidone, lamotrigine, loxapine, lurasidone, mesoridazine, olanzapine, paliperidone, perlapine, perphenazine, phenothiazine, phenylbutlypiperidine, pimozide, prochlorperazine, quetiapine, risperidone, sertindole, sulpiride, suproclone, suriclone, thioridazine, trifluoperazine, trimetozine, valproate, valproic acid, zopiclone, zotepine, zicronapine, and ziprasidone, and equivalents and pharmaceutically active isomer(s) and/or metabolite(s) thereof;
(iv) anxiolytics including, for example, azapirones, benzodiazepines, barbiturates, and equivalents and pharmaceutically active isomer(s) and/or metabolite(s) thereof. Example anxiolytics include adinazolam, alnespirone, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, prazosin, quazepam, reclazepam, tracazolate, trepipam, temazepam, triazolam, uldazepam, and zolazepam, and equivalents and pharmaceutically active isomer(s) and/or metabolite(s) thereof;
(v) anticonvulsants including, for example, carbamazepine, valproate, lamotrigine, levetiracetam, and gabapentin, and equivalents and pharmaceutically active isomer(s) and/or metabolite(s) thereof;
(vi) Parkinson's therapies including, for example, L-dopa, ropinirole, pramipexole, apomorphine, monoamine oxidase type B (MAO-B) inhibitors such as deprenyl, selegiline and rasagiline, catechol-O-methyl transferase (COMT) inhibitors such as entacapone and tolcapone, adenosine A-2 inhibitors, dopamine re-uptake inhibitors, NMDA antagonists, nicotine agonists, dopamine agonists, and inhibitors of neuronal nitric oxide synthase, and equivalents and pharmaceutically active isomer(s) and/or metabolite(s) thereof;
(vii) mood stabilizers including, for example, carbamazepine, divalproex, gabapentin, lamotrigine, lithium, olanzapine, quetiapine, valproate, valproic acid, and verapamil, and equivalents and pharmaceutically active isomer(s) and/or metabolite(s) thereof;
(viii) stimulants including, for example, methylphenidate, amphetamine, and modafinil, and equivalents and pharmaceutically active isomer(s) and/or metabolite(s) thereof;
(ix) mGluR5 modulators including, for example, basimglurant, and equivalents and pharmaceutically active isomer(s) and/or metabolite(s) thereof; and
(x) non-stimulant behaviour modifiers including, for example, atomoxetine, clonidine, and guanfacine, and equivalents and pharmaceutically active isomer(s) and/or metabolite(s) thereof; and
(xi) muscle relaxants including, for example, Botulinum toxin, baclofen and anti-muscarinic compounds such as trihexyphenidyl, benztropine, biperiden, atropine, procyclidine, orphenadrine, and scopolamine, and equivalents and pharmaceutically active isomer(s) and/or metabolite(s) thereof.

Such combination products employ the compounds of this invention within the dosage range described herein and the other pharmaceutically active agent within approved dosage ranges.

### EXAMPLES

The present invention will now be further explained by reference to the following illustrative examples, in which the starting materials and reagents used are available from commercial suppliers or prepared via literature procedures.

Nuclear magnetic resonance (NMR) spectra were recorded at 400 MHz or 300 MHz as stated and at 300.3K, 298.2K or 293K unless otherwise stated; the chemical shifts (8) are reported in parts per million. Spectra were recorded using a Bruker (trade mark) 400 AVANCE instrument fitted with a 5 mm BBFO probe with instrument controlled by Bruker TopSpin 2.1 software, or a Bruker 400 AVANCE-III HD instrument fitted with a 5 mm BBO smart probe or a 5 mm BBFO probe with instrument controlled by Bruker TopSpin 3.5 software, or a Bruker 400 AVANCE-III instrument fitted with a 5 mm BBFO probe with instrument controlled by Bruker Topspin 3.0 software, or a Bruker 300 MHz AVANCE II instrument fitted with a 5 mm DUL probe with instrument controlled by Bruker TopSpin 1.3 software, or 5 mm BBFO probe controlled by Bruker Topspin 3.2 software.

Reactions were monitored using one or more of the following.

Agilent 1290 infinity II UPLC coupled with 6130 quadrupole LCMS; chromatographic conditions: mobile phase A: 0.1% HCOOH in H₂O; mobile phase B: 0.1% HCOOH in ACN; column: Acquity UPLC BEH C18 (50 mm×2.1 mm, 1.7 μm); column T: 40° C.; Sample T: RT; detection at 220 nm; flow rate: 0.7 mL/min; analysis time: 4.2 min; mass range: 100 to 1500.

Dionex Ultima 3000 UHPLC Coupled with Thermo Scientific LCQ Fleet Ion Trap. Chromatographic conditions: mobile phase A: 10 mM ammonium formate in H₂O:ACN (95:5); mobile phase B: 10 mM ammonium formate in H₂O:ACN (5:95); column: XBridge BEH C18 (50 mm×3.0 mm, 2.5 μm); column T: 40° C.; sample T: RT; detection at 220 nm; flow rate: 0.7 mL/min; analysis time: 4.2 min; mass range: 100 to 1500.

ACQUITY UPLC H-Class with single quadrupole LCMS; chromatographic conditions: mobile phase A: 0.1% HCOOH in H₂O; mobile phase B: 0.1% HCOOH in ACN; column: Acquity UPLC BEH C18 (50 mm×2.1 mm, 1.7 μm); column T: 40° C.; sample T: RT; detection at 220 nm; flow rate: 0.7 mL/min; analysis time: 4.2 min; mass range: 100 to 1500.

Purity was assessed using one or more of the following:

Ultra Performance Liquid Chromatography (UPLC) with UV (photodiode array) detection over a wide range of wavelengths, normally 220-450 nm, using a H₂O s (trade mark) Acquity UPLC system equipped with Acquity UPLC BEH, HSS or HSS T3 C18 columns (2.1 mm id×50 mm) operated at 50 or 60° C. Mobile phases typically consisted of ACN mixed with H₂O containing either 0.1% formic acid, 0.1% TFA or 0.025% ammonia. Mass spectra were recorded with a H₂O s SQD single quadrupole mass spectrometer using atmospheric pressure ionisation.

UPLC with UV (photodiode array) detection over a wide range of wavelengths, normally 220-450 nm, using Shimadzu (trade mark) Nexera X2 UPLC controlled by Lab Solution software equipped with Acquity UPLC BEH, HSS or HSS T3 C18 columns (2.1 mm id×50 mm), operated at 50° C. Mobile phases typically consisted of ACN mixed with H₂O containing either 0.1% formic acid, 0.1% TFA or 0.025% ammonia. Mass spectra were recorded with a Shimadzu single quadrupole mass spectrometer using DUIS ionisation.

Unless stated otherwise, compounds were purified using Grace purifier, Buchi Reveleris X2 flash purification system or Biotage using normal phase chromatography on silica, using Reveleris SRC flash cartridges, Interchim (trade mark) PuriFlash cartridges or Kinesis (trade mark) Telos silica cartridges, or on basic silica using Biotage KP-NH cartridges, or by reverse phase chromatographic methods using Reveleris RP flash cartridges or by Biotage Isolute SCX-2 or Phenomenex (trade mark) Strata ABW catch-release cartridges, or by preparative high performance liquid chromatography (HPLC).

Preparative HPLC was performed using Agilent Technologies, 1100 Series system or a H₂O autopurification LC/MS system typically using H₂O (19 mm id×250 mm C18 columns such as XBridge or SunFire 5 μm) at RT. Mobile phases typically consisted of ACN mixed with H₂O containing either 0.1% formic acid or 0.1% ammonia, unless otherwise stated.

Unless stated otherwise, super Critical Fluid Chromatography (SFC) chiral separations were performed on a H₂O s SFC investigator system, using a flow rate of 60-120 g/min, T=RT to 40° C. and a pressure of 100 bar. Mobile phases typically consisted of supercritical CO₂ and a polar solvent such as ACN, MeOH, EtOH or isopropanol, respectively. Column type and eluent are detailed for individual examples. Columns: Chiralcel OJ-H (250 mm×21 mm, 5 μm), Chiralpak-IG (250 mm×30 mm, 5 μm), Chiralpak-IE (250 mm×30 mm, 5 μm), Chiralpak-AD-H (250 mm×30 mm, 5 μm), Chiralcel OX-H (250 mm×21 mm, 5 μm), YMC SC (250 mm×30 mm, 5 μm), Chiralpak AS-H (250 mm×30 mm, 5 μm), R,R Whelk-01 (250 mm×30 mm, 5 μm), Chromega chiral CCO (250 mm×30 mm, 5 μm), Chromega Chiral CCA (250 mm×30 mm, 5 μm), Chiralpak IA (250 mm×30 mm, 5 μm), Lux Cellulose-02 (250 mm×30 mm, 5 μm), Chiralpak OD-H (250 mm×30 mm, 5 μm); UV detection at 200-400 nm; sample diluent: ACN, MeOH; injection: 0.1 mL to 5 mL; isocratic ratio: 5-50% of mobile phase.

‘Room temperature’, as used in the present specification, means a temperature in the range from about 18° C. to about 25° C.

### ABBREVIATIONS

ACN acetonitrile
aq. aqueous
DAST N,N-diethylaminosulfur trifluoride
DCM dichloromethane
DIAD diisopropyl azodicarboxylate
DIBAL-H diisobutylaluminium hydride
DIPEA N,N-diisopropylethylamine
DMF dimethylformamide
DMP Dess-Martin Periodinane
EDC HCl 1-ethyl-3-(3-dimethylaminopropyl)carbodiimide hydrochloride
EtOAc ethyl acetate
h hour
HATU 1-[bis(dimethylamino)methylene]-1H-1,2,3-triazolo[4,5-b]pyridinium 3-oxid hexafluorophosphate
HOBt 1-hydroxybenzotriazole
HPLC High-Performance Liquid Chromatography
MeOH methanol
min minute
NaH sodium hydride
NMP N-methyl-2-pyrrolidone
Pd₂(dba)₃ tris(dibenzylideneacetone)dipalladium(0)
RT room temperature
sat. saturated
T3P propylphosphonic anhydride
TEA triethylamine
TFA trifluoroacetic acid
TFAA trifluoroacetic anhydride
THF tetrahydrofuran

Synthetic Approaches:

The generic schemes below describe the routes used to synthesize the examples herein.

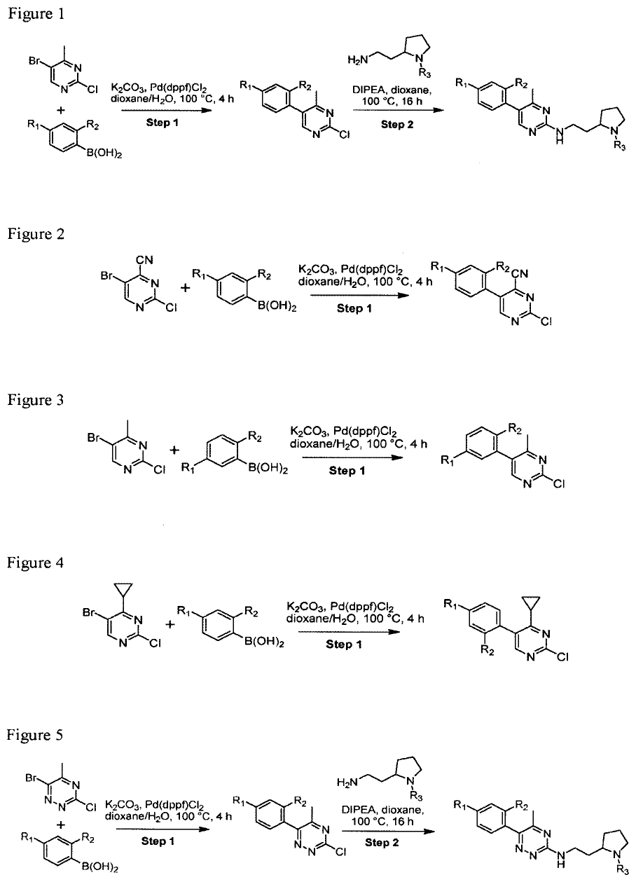

FIG. 1

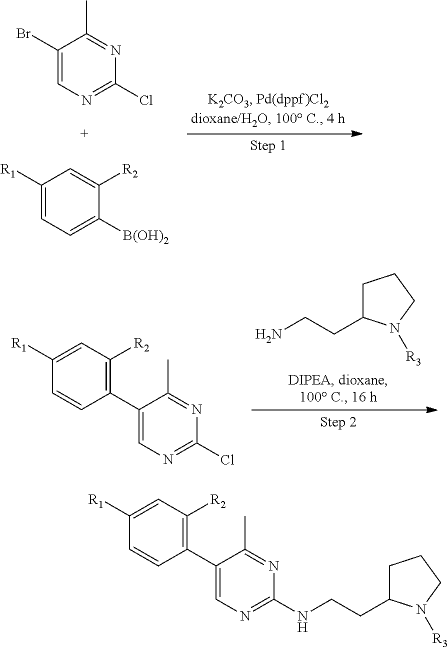

FIG. 2

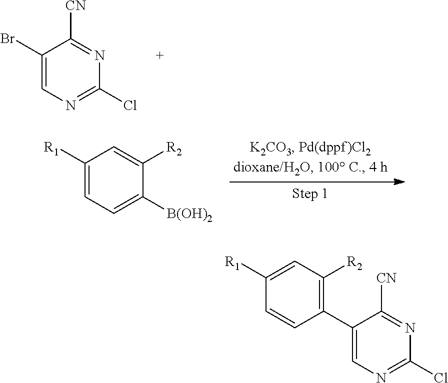

FIG. 3

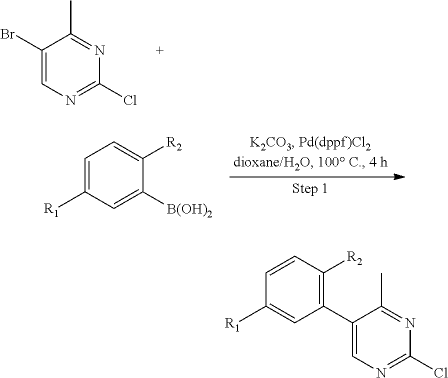

FIG. 4

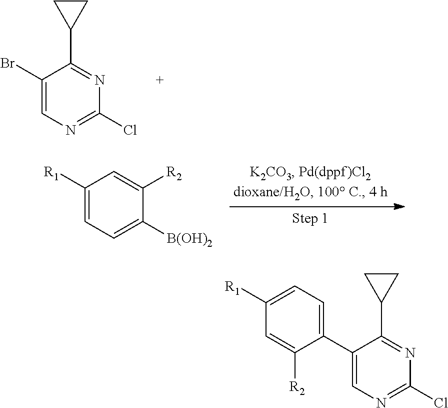

FIG. 5

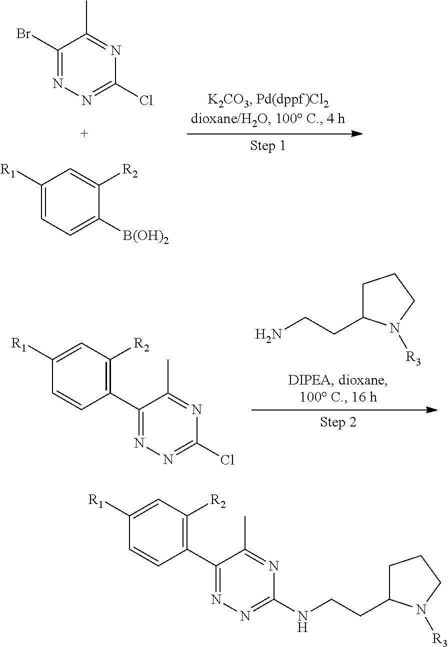

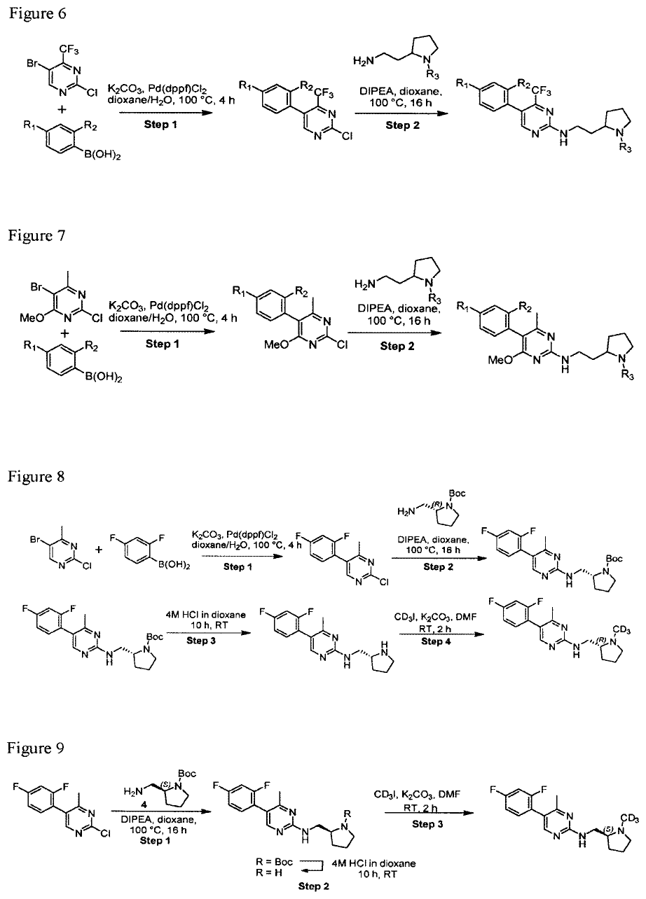

FIG. 6

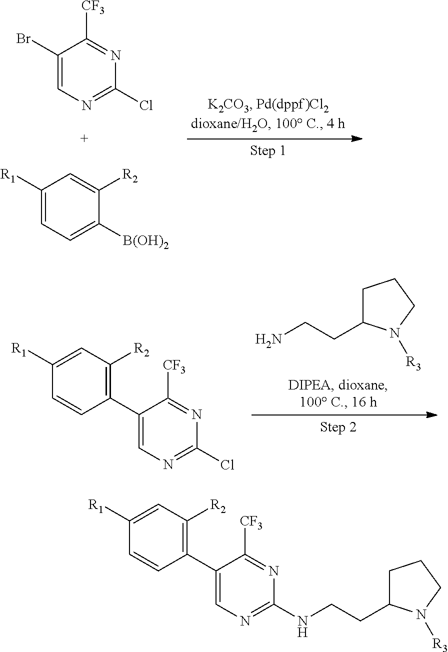

FIG. 7

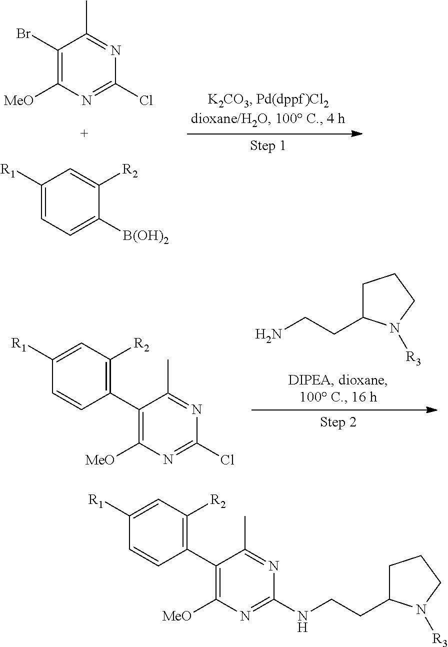

FIG. 8

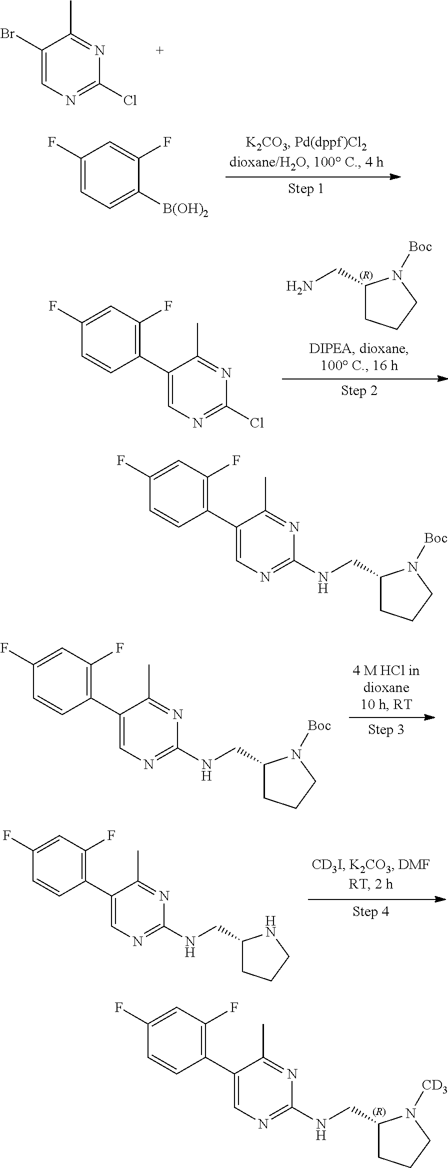

FIG. 9

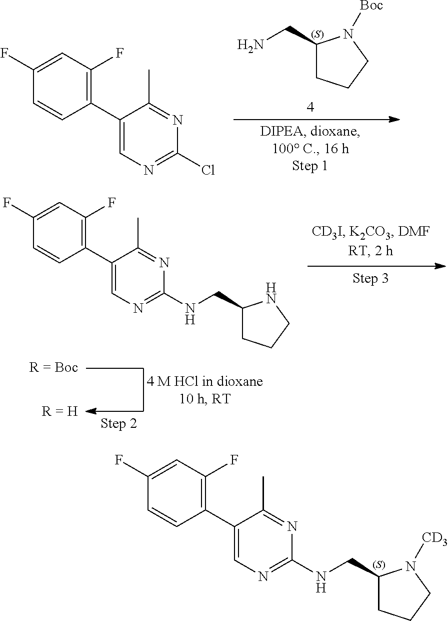

For FIGS. 1 to 9 above, where there are reaction conditions described, such as reagents, solvents, temperatures and reaction durations, it is to be understood that these reaction conditions, in particular solvents, temperatures and reaction durations, are not essential to the reaction being carried out and may be varied.

### 1. INTERMEDIATES

Commercial intermediates were used in preparation of the examples except for the following intermediates which were prepared using standard procedures as outlined below.

### Intermediate 1: 2-chloro-5-(2,4-difluorophenyl)-4-methyl-pyrimidine

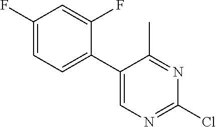 [SMILES: Cc1nc(Cl)ncc1-c1ccc(F)cc1F]

K₂CO₃ (1.01 g, 7.35 mmol) was added to a solution of 5-bromo-2-chloro-4-methyl-pyrimidine (1.00 g, 4.82 mmol) and 2,4-difluorophenylboronic acid (0.76 g, 4.82 mmol) in dioxane (10 mL) and H₂O (2 mL) in a glass tube and purged with argon for 10 min. The mixture was treated with a PdCl₂-dppf DCM complex (0.40 g, 0.49 mmol), purged with argon for 10 min, sealed and heated to 110° C. for 5 h. The solvent was evaporated from the mixture and the residue diluted with ice cold H₂O (10 mL) and extracted with EtOAc (3×25 mL). The combined organic layers were dried with Na₂SO₄ and evaporated under reduced pressure. The residue was purified by column chromatography in an automated purification system (Grace, 8-10% EtOAc/petrol ether) to obtain the title compound (400 mg, 35%) as an off-white solid. ¹H NMR (400 MHz, CDCl₃): δ 8.39 (s, 1H), 7.21-7.23 (m, 1H), 6.96-6.97 (m, 2H), 2.44 (s, 3H). MS ES⁺: 241.1.

### Intermediate 2: 2-chloro-5-(2-fluoro-4-methoxyphenyl)-4-methyl-pyrimidine

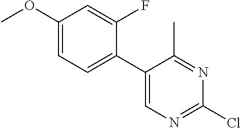 [SMILES: COc1ccc(-c2cnc(Cl)nc2C)c(F)c1]

Prepared as described for Intermediate 1 using 5-bromo-2-chloro-4-methyl-pyrimidine (0.6 g, 2.89 mmol) and (2-fluoro-4-methoxyphenyl)boronic acid (0.55 g, 3.23 mmol) to afford the title compound (0.65 g, 89%). ¹H NMR (400 MHz, CDCl₃): δ 8.39 (s, 1H), 7.14 (t, J=8.4 Hz, 1H), 6.83 (d, J=6.4 Hz, 1H), 6.76 (d, J=11.6 Hz, 1H), 3.87 (s, 3H), 2.50 (s, 3H). MS ES⁺: 253.0.

### Intermediate 3: 5-(2,4-difluorophenyl)-2-(methylsulfonyl)pyrimidine-4-carbonitrile

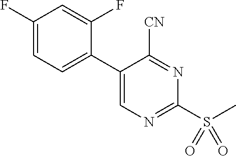 [SMILES: CS(=O)(=O)c1ncc(-c2ccc(F)cc2F)c(C#N)n1]

Step 1: To a stirred solution of 5-bromo-2-(methylthio)pyrimidine-4-carboxylic acid (2.0 g, 8.0 mmol) and ammonium chloride (2.56 g, 40 mmol) in DMF (20 mL) was added EDC HCl (2.3 g, 12 mmol), morpholine (0.002 mL, 0.023 mmol) and HOBt (1.62 g, 12 mmol) at RT. The mixture was stirred for 16 h at RT, quenched with ice cold H₂O and extracted with EtOAc (2×25 mL). The combined organic layers were washed with brine, dried over anhydrous Na₂SO₄ and concentrated under reduced pressure. The residue was triturated with pentane to obtain 5-bromo-2-(methylthio)pyrimidine-4-carboxamide (1.50 g, 75%) as a yellow solid. ¹H NMR (400 MHz, DMSO-d₆): δ 8.91 (s, 1H), 8.18 (s, 1H), 7.96 (s, 1H), 2.54 (s, 3H). MS ES⁺: 247.8.

Step 2: A stirred solution of 5-bromo-2-(methylthio)pyrimidine-4-carboxamide (0.75 g, 3.0 mmol), 2,4-difluorophenylboronic acid (0.52 g, 3.3 mmol) and K₂CO₃ (1.24 g, 9 mmol) in dioxane/H₂O (4:1, 15 mL) in a sealed tube was purged with N₂ for 10 min, treated with Pd(dppf)Cl₂ (0.21 g, 0.3 mmol), sealed and heated at 100° C. for 1 h (microwave). The mixture was treated with H₂O and extracted with EtOAc (2×30 mL). The combined organic layers were dried over Na₂SO₄ and concentrated. The crude compound was purified by column chromatography (on Grace instrument, 0-50% EtOAc/petrol ether) to afford 5-(2,4-difluorophenyl)-2-(methylthio)pyrimidine-4-carboxamide (0.6 g, 71%) as an off-white solid. ¹H NMR (400 MHz, DMSO-d₆): δ 8.72 (s, 1H), 8.15 (s, 1H), 7.76 (s, 1H), 7.60-7.50 (m, 1H), 7.45-7.35 (m, 1H), 7.25-7.2 (m, 1H), 2.62 (s, 3H). MS ES⁺: 282.14.

Step 3: To a stirred solution of 5-(2,4-difluorophenyl)-2-(methylthio)pyrimidine-4-carboxamide (0.60 g, 2.13 mmol) and triethylamine (0.75 mL) in DCM (15 mL) was added trichloroacetyl chloride (0.80 mL, 4.26 mmol) at 0° C. The mixture was warmed to RT, stirred for 4 h, diluted with H₂O and extracted with EtOAc (2×50 mL). The combined organic layers were dried over Na₂SO₄ and concentrated to obtain 5-(2,4-difluorophenyl)-2-(methylthio)pyrimidine-4-carbonitrile (0.5 g), which was used in the next step without further purification.

Step 4: To a stirred solution of 5-(2,4-difluorophenyl)-2-(methylthio)pyrimidine-4-carbonitrile (0.5 g, 1.9 mmol) in acetone/H₂O (1:1, 12 mL) was added oxone (0.60 g, 3.8 mmol) at RT. The mixture was stirred at RT for 4 h and extracted with EtOAc (2×20 mL). The combined organic layers were dried over Na₂SO₄ and concentrated. The residue was purified by column chromatography (in Grace instrument, 0-50% EtOAc/petrol ether) to obtain the title compound (0.32 g, 58%) as an off-white solid. ¹H NMR (400 MHz, DMSO-d₆): δ 9.2 (s, 1H), 7.58-7.53 (m, 1H), 7.20-7.00 (m, 2H), 3.45 (s, 3H). MS ES⁺: 296.14.

### Intermediate 4: 5-(2-fluoro-4-methoxyphenyl)-2-(methylsulfonyl)pyrimidine-4-carbonitrile

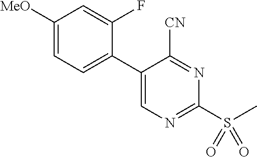 [SMILES: COc1ccc(-c2cnc(S(C)(=O)=O)nc2C#N)c(F)c1]

Prepared as described for Intermediate 3 using 5-bromo-2-(methylthio)pyrimidine-4-carboxamide (2 g, 8 mmol) and (2-fluoro-4-methoxyphenyl)boronic acid (1.36 g, 8 mmol) in step 2 to afford the title compound (0.6 g, 24%) as an off-white solid. ¹H NMR (400 MHz, DMSO-d₆): δ 9.18 (s, 1H), 6.98-6.92 (m, 1H), 6.87 (d, J=12.4 Hz, 2H), 3.91 (s, 3H), 3.45 (s, 3H). MS ES⁺: 308.20.

### Intermediate 5: 2-chloro-5-(2-fluoro-5-methoxyphenyl)-4-methyl-pyrimidine

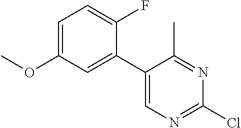 [SMILES: COc1ccc(F)c(-c2cnc(Cl)nc2C)c1]

Prepared as described for Intermediate 1 using 5-bromo-2-chloro-4-methyl-pyrimidine (0.2 g, 1.0 mmol) and (2-fluoro-5-methoxyphenyl)boronic acid (0.17 g, 1.0 mmol) to afford the title compound (0.1 g, 41%) as an off-white solid. ¹H NMR (400 MHz, CDCl₃): δ 8.42 (s, 1H), 7.15-7.10 (t, J=9.2 Hz, 1H), 6.98-6.95 (m, 1H), 6.74-6.72 (m, 1H), 3.83 (s, 3H), 2.47 (s, 3H). MS ES⁺: 253.47.

### Intermediate 6: 2-chloro-5-(2-chloro-4-methoxyphenyl)-4-methyl-pyrimidine

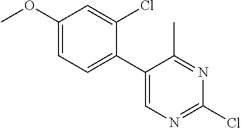 [SMILES: COc1ccc(-c2cnc(Cl)nc2C)c(Cl)c1]

Prepared as described for Intermediate 1 using 5-bromo-2-chloro-4-methyl-pyrimidine (0.45 g, 2.2 mmol) and (2-chloro-4-methoxyphenyl)boronic acid (0.409 g, 2.0 mmol) to afford the title compound (0.23 g, 40%) as an off-white solid. ¹H NMR (400 MHz, CDCl₃): δ 8.34 (s, 1H), 7.12 (d, J=8.4 Hz, 1H), 7.07 (d, J=2.4 Hz, 1H), 6.94-6.91 (dd, J=2.4 Hz and 8.8 Hz, 1H), 3.87 (s, 3H), 2.38 (s, 3H). MS ES⁺: 269.18.

### Intermediate 7: 2-chloro-4-cyclopropyl-5-(2,4-difluorophenyl)pyrimidine

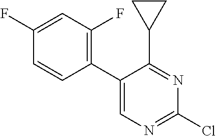 [SMILES: Fc1ccc(-c2cnc(Cl)nc2C2CC2)c(F)c1]

Step 1: A solution of 5-bromo-2-chloropyrimidine (5 g, 25.9 mmol) in a mixture of ACN/H₂O (1:1, 100 mL) was treated with cyclopropanecarboxylic acid (2.22 g, 25.9 mmol), ammonium persulfate (37.2 g, 167.17 mmol) and silver nitrate (1.32 g, 7.77 mmol) under N₂ at RT, stirred for 16 h and evaporated. The residue was extracted with EtOAc (2×50 mL) and the combined organic layers were washed with H₂O followed by brine, dried over Na₂SO₄ and concentrated. The residue was purified by column chromatography (on Grace instrument, 2% EtOAc in petrol ether) to afford 5-bromo-2-chloro-4-cyclopropylpyrimidine (2.5 g, 48%) as a colourless oil. ¹H NMR (400 MHz, CDCl₃): δ 8.49 (s, 1H), 2.47-2.44 (m, 1H), 1.31-1.24 (m, 4H). MS ES⁺: 233.06.

Step 2: To a solution of 5-bromo-2-chloro-4-cyclopropylpyrimidine (0.90 g, 3.9 mmol) in dioxane/H₂O (3:1, 20 mL) was added (2,4-difluorophenyl)boronic acid (0.677 g, 4.29 mmol) and K₂CO₃ (1.61 g, 11.7 mmol). The mixture was purged with N₂, treated with Pd(dppf)Cl₂ (0.16 g, 0.19 mmol), sealed and stirred at 100° C. for 2 h. The solvent was evaporated and the mixture extracted with EtOAc (2×50 mL). The combined organic layers were washed with H₂O followed by brine, dried over Na₂SO₄ and concentrated to obtain crude product. The crude product was purified by column chromatography (on Grace instrument, 5% EtOAc in petrol ether) to afford the title compound (0.45 g, 38%) as a colourless gum. ¹H NMR (400 MHz, CDCl₃): δ 8.28 (s, 1H), 7.35-7.31 (m, 1H), 7.06-6.96 (m, 2H), 1.82-1.79 (m, 1H), 1.34-1.30 (m, 2H), 1.10-1.07 (m, 2H). MS ES⁺: 267.0.

### Intermediate 8: 2-chloro-4-cyclopropyl-5-(2-fluoro-4-methoxyphenyl)pyrimidine

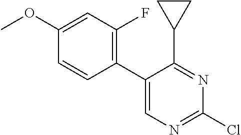 [SMILES: COc1ccc(-c2cnc(Cl)nc2C2CC2)c(F)c1]

Step 1: A solution of 5-bromo-2-chloropyrimidine (5 g, 25.9 mmol) in a mixture of ACN/H₂O (1:1, 100 mL) was treated with cyclopropanecarboxylic acid (2.22 g, 25.9 mmol), ammonium persulfate (37.2 g, 167.17 mmol) and silver nitrate (1.32 g, 7.77 mmol) under N₂ at RT, stirred for 16 h and evaporated. The residue was extracted with EtOAc (2×50 mL) and the combined organic layers were washed with H₂O followed by brine, dried over Na₂SO₄ and concentrated. The residue was purified by column chromatography (on Grace instrument, 2% EtOAc in petrol ether) to afford 5-bromo-2-chloro-4-cyclopropylpyrimidine (2.5 g, 48%) as a colourless oil. ¹H NMR (400 MHz, CDCl₃): δ 8.49 (s, 1H), 2.47-2.44 (m, 1H), 1.31-1.24 (m, 4H). MS ES⁺: 233.06.

Step 2: A solution of 5-bromo-2-chloro-4-cyclopropylpyrimidine (2.5 g, 10.8 mmol) in dioxane/H₂O (3:1, 100 mL) was treated with (2-fluoro-4-methoxyphenyl)boronic acid (2.01 g, 11.88 mmol) and K₂CO₃ (4.47 g, 32.4 mmol), purged with N₂, treated with Pd(dppf)Cl₂ (0.44 g, 0.54 mmol), sealed and stirred at 100° C. for 2 h. The solvent was evaporated and the residue extracted with EtOAc (2×50 mL). The combined organic layers were washed with H₂O followed by brine, dried over Na₂SO₄ and concentrated. The residue was purified by column chromatography (on Grace instrument, 5% EtOAc in petrol ether) to afford the title compound (1.60 g, 42%) as a colourless gum. ¹H NMR (400 MHz, CDCl₃): δ 8.30 (s, 1H), 7.28-7.23 (m, 1H), 6.86-6.84 (m, 1H), 6.80-6.76 (m, 1H), 3.88 (s, 3H), 1.92-1.88 (m, 1H), 1.35-1.30 (m, 2H), 1.10-1.07 (m, 2H). MS ES⁺: 279.22.

### Intermediate 9: 2-chloro-5-(2-fluoro-4-trifluoromethoxy)phenyl)-4-methyl-pyrimidine

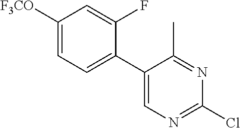 [SMILES: Cc1nc(Cl)ncc1-c1ccc(OC(F)(F)F)cc1F]

Prepared as described for Intermediate 1 using 5-bromo-2-chloro-4-methyl-pyrimidine (0.1 g, 0.5 mmol) and (2-fluoro-4-(trifluoromethoxy)phenyl)boronic acid (0.112 g, 0.5 mmol) to afford the title compound (0.045 g, 30%) as an off-white solid. ¹H NMR (400 MHz, CDCl₃): δ 8.41 (s, 1H), 7.30 (t, J=8.0 Hz, 1H), 7.20-7.18 (m, 1H), 7.17-7.12 (m, 1H), 2.46 (s, 3H). MS ES⁺: 307.09.

### Intermediate 10: 2-chloro-5-(2-fluoro-4-(trifluoromethyl)phenyl)-4-methyl-pyrimidine

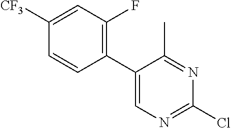 [SMILES: Cc1nc(Cl)ncc1-c1ccc(C(F)(F)F)cc1F]

A solution of 5-bromo-2-chloro-4-methyl-pyrimidine (0.6 g, 2.9 mmol), (2-fluoro-4-(trifluoromethyl)phenyl)boronic acid (0.7 g, 3.2 mmol) and K₂CO₃ (0.8 g, 5.8 mmol) in H₂O and dioxane in a glass tube was purged with N₂ for 10 min, treated with Pd(dppf)Cl₂·DCM (0.2 g, 0.2 mmol) and purged with N₂. The tube was sealed and the mixture heated at 90-100° C. for 14 h, cooled, diluted with EtOAc and washed with H₂O and brine. The organic layer was dried over Na₂SO₄ and concentrated under reduced pressure. The residue was purified by reverse phase column chromatography (on Grace instrument, 60% MeOH in H₂O) to afford the title compound (0.36 g, 43%) as an off-white solid. ¹H NMR (400 MHz, CDCl₃): δ 8.34 (s, 1H), 7.13 (d, J=8.4 Hz, 1H), 7.22-7.16 (m, 1H), 7.14-7.10 (m, 1H), 2.40 (s, 3H). MS ES⁺: 291.09.

### Intermediate 11: 2-chloro-5-(4-fluoro-2-(trifluoromethyl)phenyl)-4-methyl-pyrimidine

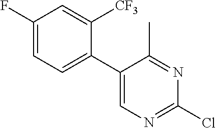 [SMILES: Cc1nc(Cl)ncc1-c1ccc(F)cc1C(F)(F)F]

Prepared as described for Intermediate 1 using 5-bromo-2-chloro-4-methyl-pyrimidine (0.3 g, 1.5 mmol) and (4-fluoro-2-(trifluoromethyl)phenyl)boronic acid (0.4 g, 1.8 mmol) to afford the title compound (0.21 g, 49%) as an off-white solid. ¹H NMR (400 MHz, CDCl₃): δ 8.42 (s, 1H), 7.21 (m 1H), 7.29-7.10 (m, 1H), 7.10-7.01 (m, 1H), 2.42 (s, 3H). MS ES⁺: 291.01.

### Intermediate 12: 6-(2,4-difluorophenyl)-5-methyl-3-(methylsulfonyl)-1,2,4-triazine

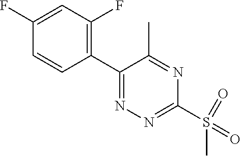 [SMILES: Cc1nc(S(C)(=O)=O)nnc1-c1ccc(F)cc1F]

Step 1: A stirred solution of 1-(2,4-difluorophenyl)propan-1-one (1.5 g, 8.8 mmol) in DCM was treated with TMSCl (1.91, 17.6 mmol) followed by isoamyl nitrite (2.06 g, 17.6 mmol) at −20° C., allowed to warm to RT and stirred at RT for 16 h. The mixture was quenched with ice cold H₂O (100 mL) and extracted with DCM (2×50 mL). The combined organic layers were dried over Na₂SO₄ and concentrated under reduced pressure. The residue was purified by column chromatography (2-3% EtOAc in petrol ether) to obtain (E)-1-(2,4-difluorophenyl)-2-(hydroxyimino)propan-1-one (900 mg, 51.4%) as an off-white solid. ¹H NMR (400 MHz, CDCl₃): δ 8.07 (s, 1H), 7.56-7.50 (m, 1H), 6.96-6.91 (m, 1H), 6.87-6.81 (m, 1H), 2.14 (s, 3H). MS ES⁺: 200.06.

Step 2: Carbon disulfide (12.5 mL, 0.2 mol) was added dropwise to an ice cooled mixture of hydrazine hydrate (85%, 10 mL, 0.2 mol) in aq. KOH (13.4 g, 0.24 mol) and 2-propanol (50 mL). The mixture was allowed to stir at −10° C. for 1.5 h, dropwise treated with methyl iodide (12.5 mL, 0.20 mol) and continued to stir at 0° C. for 1 h. The precipitated solid was filtered and washed with ice H₂O. The crude material was purified by column chromatography (20-45% EtOAc in petrol ether) to give methyl hydrazinecarbodithioate (4.0 g, 65%) as an off-white solid. ¹H NMR (400 MHz, CDCl₃): δ 8.86-8.31 (m, 1H), 4.70 (s, 1H), 4.17 (s, 1H), 2.66 (s, 3H). MS ES⁺: 123.0.

Step 3: A stirred solution of (E)-1-(2,4-difluorophenyl)-2-(hydroxyimino)propan-1-one (2.5 g, 12.6 mmol) and methyl hydrazinecarbodithioate (1.69 g, 13.7 mmol) in EtOH (10 mL) at 0° C. was treated with conc. HCl (1.2 mL), allowed to warm to RT and stirred for 16 h. The precipitated solid was filtered, washed with petrol ether and dried in vacuo to obtain methyl (Z)-2-((E)-1-(2,4-difluorophenyl)-2-(hydroxyimino)propylidene)hydrazine-1-carbodithioate (2.1 g, 55%) as a light yellow solid. ¹H NMR (400 MHz, CDCl₃): δ 9.63 (s, 1H), 7.64 (s, 1H), 7.21-7.15 (m, 1H), 7.08-7.03 (m, 1H), 7.01-6.95 (m, 1H), 2.65 (s, 3H), 2.28 (s, 3H). MS ES⁺: 304.0.

Step 4: A solution of methyl (Z)-2-((E)-1-(2,4-difluorophenyl)-2-(hydroxyimino) propylidene)hydrazine-1-carbodithioate (2.5 g, 8.3 mmol) in aq. 20% potassium carbonate solution (30 mL) was refluxed at 110° C. for 1 h and allowed to cool to RT. The precipitated solid was filtered, washed with petrol ether and Et₂O, and dried in vacuo to obtain 6-(2,4-difluorophenyl)-3-mercapto-5-methyl-1,2,4-triazine-4-oxide (1.8 g, 85%) as a light yellow solid. ¹H NMR (400 MHz, CDCl₃): δ 7.61-7.55 (m, 1H), 7.42-7.36 (m, 1H), 7.25-7.20 (m, 1H), 2.06 (s, 3H). MS ES⁺: 256.0.

Step 5: Methyl iodide (2.08 g, 14.7 mmol) was added to a stirred solution of 6-(2,4-difluorophenyl)-3-mercapto-5-methyl-1,2,4-triazine-4-oxide (2.5 g, 9.8 mmol) and potassium carbonate (2.7 g, 19.6 mmol) in DMF (20 mL) at 0° C. The mixture was allowed to stir at RT for 2 h, quenched with ice cold H₂O (100 mL) and extracted with EtOAc (2×70 mL). The combined organic layers were dried over Na₂SO₄ and concentrated under reduced pressure. The residue was purified by column chromatography (10-12% EtOAc in petrol ether) to give 6-(2,4-difluorophenyl)-5-methyl-3-(methylthio)-1,2,4-triazine-4-oxide (1.0 g, 38.2%) as an off-white solid. ¹H NMR (400 MHz, CDCl₃): δ 7.64-7.57 (m, 1H), 7.12-7.09 (m, 1H), 7.08-6.97 (m, 1H), 2.73 (s, 3H), 2.44 (s, 3H). MS ES⁺: 270.0.

Step 6: A stirred solution of 6-(2,4-difluorophenyl)-5-methyl-3-(methylthio)-1,2,4-triazine-4-oxide (0.05 g, 0.2 mmol) in triethyl phosphite (1 mL) was stirred at 160° C. for 4 h, cooled to RT, loaded onto a silica gel column and purified by chromatography (2-3% EtOAc in petrol ether) to give 6-(2,4-difluorophenyl)-5-methyl-3-(methylthio)-1,2,4-triazine (0.03 g, 63%) as a colourless liquid. ¹H NMR (400 MHz, CDCl₃): δ 7.59-7.54 (m, 1H), 7.10-7.05 (m, 1H), 7.00-6.95 (m, 1H), 2.71 (s, 3H), 2.45 (s, 3H). MS ES⁺: 254.17.

Step 7: Oxone (0.491 mg, 1.6 mmol) was added to a stirred solution of 6-(2,4-difluorophenyl)-5-methyl-3-(methylthio)-1,2,4-triazine (0.2 g, 0.8 mmol) in acetone/H₂O (1:1, 10 mL) at 0° C. The mixture was allowed to stir at RT for 3 h, quenched with ice H₂O (20 mL) and extracted with EtOAc (2×20 mL). The combined organic layers were dried over Na₂SO₄ and concentrated. The residue was purified by column chromatography (50-60% EtOAc in petrol ether) to give the title compound (0.2 g, 90°/%) as a pale yellow solid. ¹H NMR (400 MHz, CDCl₃): δ 7.61-7.52 (m, 1H), 7.09-7.01 (m, 1H), 7.00-6.92 (m, 1H), 4.01 (s, 3H), 2.47 (s, 3H). MS ES⁺: 286.89.

### Intermediate 13: N¹-(5-bromo-4-(trifluoromethyl)pyrimidin-2-yl)-N³,N³-dimethylcyclopentane-1,3-diamine

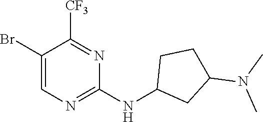 [SMILES: CN(C)C1CCC(Nc2ncc(Br)c(C(F)(F)F)n2)C1]

A stirred solution of 5-bromo-2-chloro-4-(trifluoromethyl)pyrimidine (0.3 g, 1.15 mmol) in dioxane (10 mL) was treated with DIPEA (0.28 g, 2.3 mmol) and N¹,N¹-dimethylcyclopentane-1,3-diamine (0.14 g, 1.15 mmol) and stirred at 105° C. for 16 h. The solvent was evaporated under reduced pressure to yield crude title compound (0.4 g), which was used in the next step without further purification. ¹H NMR (400 MHz, DMSO-d₆): δ 8.73 (m, 1H), 8.26 (br s, 1H), 4.20-4.12 (m, 2H), 3.16-3.02 (m, 2H), 2.40-2.25 (m, 2H), 2.00-1.62 (m, 6H), 1.30-1.22 (m, 2H). MS ES⁺: 353.20.

### Intermediate 14: 2-chloro-5-(2-fluoro-4-methoxyphenyl)-4,6-dimethylpyrimidine

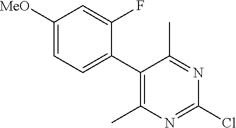 [SMILES: COc1ccc(-c2c(C)nc(Cl)nc2C)c(F)c1]

A solution of 5-bromo-2-chloro-4,6-dimethylpyrimidine (0.5 g, 2.25 mmol) in dioxane/H₂O (3:1, 20 mL) was treated with K₂CO₃ (0.624 g, 4.5 mmol) and (2-fluoro-4-methoxyphenyl)boronic acid (0.382 g, 2.21 mmol), purged with N₂ for 10 min, treated with Pd(dppf)Cl₂ (0.054 g, 0.03 mmol), sealed and stirred at 100° C. for 16 h. The solvent was evaporated under reduced pressure and the residue dissolved in H₂O and extracted with EtOAc (3×30 mL). The combined organic layers were dried over Na₂SO₄ and evaporated. The residue was purified by column chromatography (on Grace instrument, 12% EtOAc in petrol ether) to give the title compound (0.10 g, 17%) as a colourless gum. MS ES⁺: 267.10

### Intermediate 15: (S)-5-bromo-4-methoxy-6-methyl-N-((1-methylpyrrolidin-2-yl)methyl)pyrimidin-2-amine

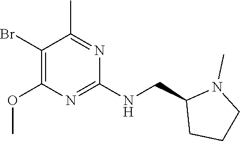 [SMILES: COc1nc(NC[C@@H]2CCCN2C)nc(C)c1Br]

Step 1: A solution of 5-bromo-2,4-dichloro-6-methyl pyrimidine (1.00 g, 4.13 mmol) in THF (10 mL) at 0° C. was treated with NaOMe (0.21 g, 3.72 mmol) and stirred for 30 min at 0° C. The mixture was diluted with EtOAc and washed with H₂O followed by brine. The organic layer was dried over Na₂SO₄ and concentrated under reduced pressure to give 5-bromo-2-chloro-4-methoxy-6-methylpyrimidine (0.90 g, 92%) as a yellow solid, which was used in the next step without further purification.

Step 2: A stirred solution of 5-bromo-2-chloro-4-methoxy-6-methylpyrimidine (0.60 g, 2.55 mmol) in 1,4-dioxane (5 mL) was treated with (S)-(1-methylpyrrolidin-2-yl)methanamine (0.35 g, 3.06 mmol) under N₂ at RT, then treated with DIPEA (1.8 ml, 10.2 mmol), heated to 100° C. and stirred for 16 h. The mixture was cooled to RT, treated with H₂O and extracted with EtOAc (3×10 mL). The combined organic layers were washed with brine, dried over Na₂SO₄ and concentrated under reduced pressure. The residue was purified by reverse phase column chromatography (60% MeOH/H₂O) to give the title compound (0.35 g, 50%) as an off-white gummy solid. MS ES⁺: 317.23.

### Intermediate 16: 5-bromo-4-methyl-N-(tetrahydro-2H-pyran-4-yl)pyrimidin-2-amine

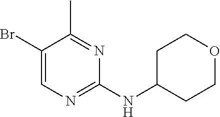

A mixture of 5-bromo-2-chloro-4-methyl-pyrimidine (0.40 g, 1.9 mmol), tetrahydro-2H-pyran-4-amine (0.19 g, 1.92 mmol) and DIPEA (0.367 g, 2.85 mmol) in dioxane (5 mL) was stirred for 16 h at 105° C. in a sealed tube. The mixture was cooled to RT, treated with H₂O and extracted with EtOAc. The organic layer was evaporated under reduced pressure. The residue was purified by column chromatography (0-50% EtOAc in petrol ether) to afford the title compound (0.30 g, 58%) as a white solid. ¹H NMR (400 MHz, DMSO-d₆): δ 8.26 (s, 1H), 7.34 (br s, 1H), 3.86-3.84 (m, 3H), 3.38-3.32 (m, 2H), 2.33 (s, 3H), 1.80-1.76 (m, 2H), 1.49-1.45 (m, 2H). MS ES⁺: 274.16.

### Intermediate 17: 5-bromo-4-methyl-N-tetrahydro-2H-pyran-3-yl)pyrimidin-2-amine

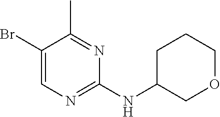

Prepared as described for Intermediate 16 using 5-bromo-2-chloro-4-methyl-pyrimidine (0.40 g, 1.9 mmol) and tetrahydro-2H-pyran-3-amine (0.19 g, 1.92 mmol) to afford the title compound (0.30 g, 58%) as a white solid. ¹H NMR (400 MHz, DMSO-d₆): δ 8.26 (s, 1H), 7.34 (br s, 1H), 3.86-3.84 (m, 3H), 3.38-3.32 (m, 1H), 3.10-3.00 (m, 1H), 2.34 (s, 3H), 2.00-1.95 (m, 1H), 1.80-1.76 (m, 1H), 1.49-1.45 (m, 2H). MS ES⁺: 274.03.

### Intermediate 18: 5-bromo-4-methyl-N-((tetrahydrofuran-2-yl)methyl)pyrimidin-2-amine

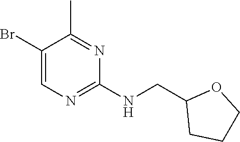

Prepared as described for Intermediate 16 using 5-bromo-2-chloro-4-methyl-pyrimidine (0.40 g, 1.9 mmol) and (tetrahydrofuran-2-yl)methanamine (0.19 g, 1.92 mmol) to afford the title compound (0.25 g, 48%) as an off-white solid. ¹H NMR (400 MHz, DMSO-d₆): δ 8.24 (s, 1H), 7.3 (br s, 1H), 4.00-3.90 (m, 1H), 3.85-3.75 (m, 1H), 3.70-3.60 (m, 1H), 3.40-3.30 (m, 1H), 3.25-3.15 (m, 1H), 2.35 (s, 3H), 1.95-1.70 (m, 3H), 1.60-1.50 (m, 1H). MS ES⁺: 272.10.

### Intermediate 19: 5-bromo-4-methyl-N-((4-methylmorpholin-3-yl)methyl)pyrimidin-2-amine

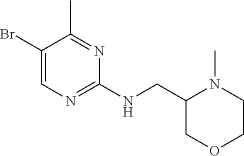 [SMILES: Cc1nc(NCC2COCCN2C)ncc1Br]

A solution of 5-bromo-2-chloro-4-methyl-pyrimidine (0.3 g, 1.5 mmol), (4-methylmorpholin-3-yl)methanamine (0.214 g, 1.65 mmol) and DIPEA (0.58 g, 6 mmol) in dioxane (10 mL) was allowed to stir for 24 h at 110° C. The mixture was diluted with H₂O and extracted with EtOAc (3×30 mL). The combined organic layers were dried over Na₂SO₄ and concentrated under reduced pressure to afford the title compound (0.39 g, 90%) as sticky solid. MS ES⁺: 301.0.

### Intermediate 20: (R)-1-(4,4-difluoro-1-methylpyrrolidin-2-yl)-N-(4 methoxybenzyl) methanamine

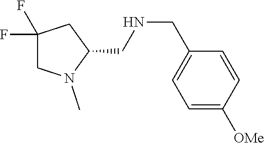 [SMILES: COc1ccc(CNC[C@H]2CC(F)(F)CN2C)cc1]

Step 1: A solution of (R)-1-(tert-butoxycarbonyl)-4,4-difluoropyrrolidine-2-carboxylic acid (1 g, 4 mmol) and (4-methoxyphenyl)methanamine (0.66 g, 4.8 mmol) in THF (20 mL) was treated with HATU (2.28 g, 6.0 mmol) and DIPEA (1.54 g, 12 mmol) and allowed to stir for 24 h at RT. The mixture was quenched with ice cold H₂O and extracted with 10% MeOH/DCM (2×40 mL). The combined organic layers were dried over Na₂SO₄ and concentrated under reduced pressure. The residue was purified by flash column chromatography (60-70% EtOAc in petrol ether) to afford tert-butyl (R)-4,4-difluoro-2-((4-methoxybenzyl)carbamoyl)pyrrolidine-1-carboxylate (1.3 g, 88%) as thick liquid.

Step 2: A solution of tert-butyl (R)-4,4-difluoro-2-((4-methoxybenzyl)carbamoyl) pyrrolidine-1-carboxylate (0.50 g, 1.4 mmol) in THF was treated at 0° C. with an LiAlH₄ solution (6.5 mL, 2M in THF) and stirred for 30 min. The mixture was warmed to 60° C. and continued to stir for 2 h, cooled to 0° C., quenched with a paste of Na₂SO₄ in H₂O (0.5 mL) and allowed to stir for 5 min. Excess EtOAc was added to the mixture and filtered through Celite®. The filtrate was dried over Na₂SO₄ and concentrated under reduced pressure. The residue was purified by flash column chromatography (amine-silica Davisil, 20-25% EtOAc in petrol ether) to afford the title compound (0.09 g, 25%) as thick liquid.

### Intermediate 21: tert-butyl 2-(((5-bromo-4-methyl-pyrimidin-2-yl)amino)methyl)-3,3-difluoropyrrolidine-1-carboxylate

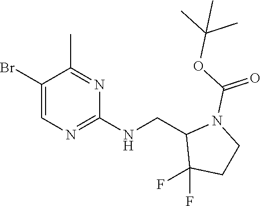 [SMILES: Cc1nc(NCC2N(C(=O)OC(C)(C)C)CCC2(F)F)ncc1Br]

Step 1: A solution of 1-(tert-butyl) 2-ethyl 3-oxopyrrolidine-1,2-dicarboxylate (8.00 g, 31.1 mmol) in DCM (160 mL) was treated with DAST (15.04 g, 93.28 mmol, 12.32 mL) at 0° C. under N₂ and stirred at 25° C. for 16 h. The mixture was diluted with DCM (300 mL), treated with sat. aq. NaHCO₃ (800 mL) and extracted with DCM (500 mL). The combined organic layers were washed with H₂O (400 mL) and brine (400 mL), dried over MgSO₄, filtered and concentrated under reduced pressure. The residue was purified by flash silica gel chromatography to afford 1-(tert-butyl) 2-ethyl 3,3-difluoropyrrolidine-1,2-dicarboxylate (4.9 g, 56%) as a yellow oil. ¹H NMR (400 MHz, CDCl₃): δ 4.40 (m, 1H), 4.25 (m, 2H), 3.75 (m, 1H), 3.55 (m, 1H), 2.41 (m, 2H), 1.45 (s, 9H), 1.3 (m, 3H).

Step 2: A solution of 1-(tert-butyl) 2-ethyl 3,3-difluoropyrrolidine-1,2-dicarboxylate (5.1 g, 18.3 mmol) in anhydrous THF (50 mL) at 0° C. was treated with LiAlH₄ (762 mg, 20.1 mmol) in portions and stirred for 30 min at 0° C. The mixture was quenched with H₂O (75 μL), NaOH (15% 75 μL) and H₂O (150 μL), filtered, and the filtrate concentrated in vacuo. The residue was purified by flash silica gel chromatography to afford tert-butyl 3,3-difluoro-2-(hydroxymethyl)pyrrolidine-1-carboxylate (3.42 g, 78.9%) as a yellow oil. ¹H NMR (400 MHz, CDCl₃): δ 4.0-3.6 (m, 4H), 3.5 (m, 2H), 2.45 (m, 2H), 1.5 (s, 9H).

Step 3: A solution of tert-butyl 3,3-difluoro-2-(hydroxymethyl)pyrrolidine-1-carboxylate (3.50 g, 14.8 mmol) in EtOAc (50 mL) (ice-bath) was treated with Et₃N (2.24 g, 22.1 mmol, 3.08 mL) and MsCl (2.21 g, 19.29 mmol, 1.49 mL) and stirred at 25° C. for 1 h. The mixture was filtered and washed with EtOAc (20 mL) and the filtrate concentrated under reduced pressure, resulting in crude tert-butyl 3,3-difluoro-2-(((methylsulfonyl)oxy) methyl)pyrrolidine-1-carboxylate (5.5 g, 99%) as yellow oil, which was used in the next step without further purification.

Step 4: A solution of tert-butyl 3,3-difluoro-2-(((methylsulfonyl)oxy)methyl)pyrrolidine-1-carboxylate (5.50 g, 14.7 mmol) in DMSO (50 mL) was treated with NaN₃ (1.49 g, 22.9 mmol) and stirred at 70° C. for 16 h. The mixture was added into ice H₂O (200 mL) and extracted with MTBE (3×50 mL). The combined organic layers were washed with H₂O (2×5 mL) and dried over MgSO₄. The resulting solution was treated with Pd/C (300 mg, 14.7 mmol, 10%/6), purged with H₂ (3×) and stirred at 25° C. for 20 h under H₂. The mixture was filtered and concentrated under reduced pressure. The residue tert-butyl 2-(aminomethyl)-3,3-difluoropyrrolidine-1-carboxylate (3.1 g, 88.9%) was used in the next step without further purification.

Step 5: A solution of tert-butyl 2-(aminomethyl)-3,3-difluoropyrrolidine-1-carboxylate (3.00 g, 12.7 mmol) in DMF (60 mL) was treated with Cs₂CO₃ (8.27 g, 25.40 mmol) and 5-bromo-2-chloro-4-methyl-pyrimidine (3.42 g, 16.51 mmol) and stirred at 70° C. for 8 h. The mixture was quenched with cold H₂O (200 mL) and extracted with EtOAc (2×100 mL). The combined organic layers were washed with H₂O (2×100 mL) and brine (200 mL), dried over MgSO₄ and evaporated. The residue was purified by flash silica gel chromatography to afford the title compound (2.2 g, 42.5%) as a yellow oil. ¹H NMR (400 MHz, CDCl₃): δ 8.19 (s, 1H), 5.5 (br s, 1H), 4.1 (m, 1H), 3.7-3.40 (m, 4H), 2.4 (s, 3H), 2.4-2.3 (m, 2H), 1.4 (s, 9H).

### Intermediate 22: tert-butyl 2-(aminomethyl)-3-fluoropyrrolidine-1-carboxylate

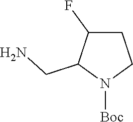 [SMILES: CC(C)(C)OC(=O)N1CCC(F)C1CN]

Step 1: A stirred solution of 1-(tert-butyl) 2-ethyl 3-oxopyrrolidine-1,2-dicarboxylate (1.50 g, 5.83 mmol) in MeOH (50 mL) was treated with NaBH₄ (0.44 g 11.7 mmol) at 0° C., stirred at RT for 2 h and evaporated under reduced pressure. The residue was diluted with H₂O and extracted with EtOAc (2×30 mL). The combined organic layers were dried over Na₂SO₄ and evaporated under reduced pressure to give 1-(tert-butyl) 2-ethyl 3-hydroxypyrrolidine-1,2-dicarboxylate (1.46 g) as an off-white solid, which was used in the next step without further purification.

Step 2: A stirred solution of 1-(tert-butyl) 2-ethyl 3-hydroxypyrrolidine-1,2-dicarboxylate (1.46 g, 5.6 mmol) in DCM (60 mL) was treated with DAST (1.80 g, 11.2 mmol) at 0° C., allowed to warm to RT and stirred for 16 h. The solvent was evaporated under reduced pressure and the residue diluted with H₂O and extracted with EtOAc (2×30 ml). The combined organic layers were dried over Na₂SO₄ and evaporated. Purification of the residue on amine silica (Davisil, 30% EtOAc in petrol ether) gave 1-(tert-butyl) 2-ethyl 3-fluoropyrrolidine-1,2-dicarboxylate (1.30 g, 90%) as an off-white gummy solid.

Step 3: A stirred solution of 1-(tert-butyl) 2-ethyl 3-fluoropyrrolidine-1,2-dicarboxylate (1.1 g, 4.2 mmol) in THF (50 mL) was treated with lithium borohydride (0.183 g, 8.4 mmol) at 0° C. and then stirred at RT for 3 h. The solvent was evaporated from the mixture, and the residue diluted with H₂O and extracted with EtOAc (2×30 ml). The combined organic layers were dried over Na₂SO₄ and evaporated under reduced pressure to give crude tert-butyl 3-fluoro-2-(hydroxymethyl)pyrrolidine-1-carboxylate (0.55 g) as an off-white solid, which was used in the next step without further purification.

Step 4: A stirred solution of tert-butyl 3-fluoro-2-(hydroxymethyl)pyrrolidine-1-carboxylate (0.25 g, 1.14 mmol) in toluene (50 mL) was treated with triphenyl phosphine (0.449 g, 1.71 mmol) and phthalimide (0.251 g, 1.71 mmol) at 0° C., stirred for 5 min, treated with DIAD (0.345 g, 1.71 mmol) and stirred at RT for 2.5 h. The mixture was then treated with hydrazine hydrate in EtOH (1:1, 5 mL) at 0° C. and stirred for 3 h at 85° C. The solvent was evaporated under reduced pressure and the residue diluted with H₂O and extracted with EtOAc (2×30 ml). The combined organic layers were dried over Na₂SO₄ and evaporated. The residue was purified by reverse phase column chromatography (30% MeOH/H₂O) to give the title compound (0.12 g, 50%) as a pale yellow solid. ¹H NMR (400 MHz, CDCl₃): δ 6.36 (s, 2H), 5.01 (m, 3H), 3.50 (s, 2H), 2.85-2.70 (m, 1H), 2.20-2.00 (m, 2H), 1.48 (s, 9H). ELSD MS: 219.1.

### 2. EXAMPLES

### Example 1: 4-methyl-N-(2-(1-methylpyrrolidin-2-yl)ethyl)-5-phenylpyrimidin-2-amine

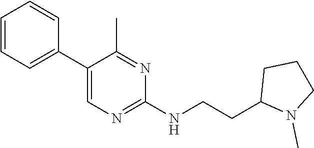 [SMILES: Cc1nc(NCCC2CCCN2C)ncc1-c1ccccc1]

A solution of 2-chloro-4-methyl-5-phenylpyrimidine (0.20 g, 1.00 mmol) in dry DMF was treated with 2-(1-methylpyrrolidin-2-yl)ethan-1-amine (0.16 g, 1.25 mmol), stirred at 150° C. for 15 h, cooled to RT, quenched with ice H₂O and extracted with EtOAc (2×20 mL). The combined organic layers were washed with H₂O and brine, dried over Na₂SO₄ and concentrated under reduced pressure. Purification of the residue by column chromatography on amine silica (Davisil) gave the title compound (0.053 g, 18%) as an off-white solid. ¹H NMR (400 MHz, CDCl₃): δ 8.52 (s, 2H), 7.49-7.42 (m, 3H), 7.36-7.33 (m, 1H), 5.83 (s, 1H), 3.59-3.48 (m, 2H), 3.10 (t, J=7.6 Hz, 1H), 2.94 (s, 3H), 2.36 (s, 3H), 2.36-2.20 (m, 1H), 2.20-2.13 (q, J=9.2 Hz, 1H), 2.05-1.90 (m, 2H), 1.85-1.55 (m, 4H). MS ES⁺: 296.42.

### Example 2: N-(2-(1-methylpyrrolidin-2-yl)ethyl)-5-phenylpyrimidin-2-amine

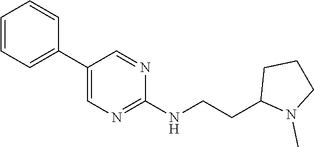 [SMILES: CN1CCCC1CCNc1ncc(-c2ccccc2)cn1]

A solution of 2-chloro-5-phenylpyrimidine (0.20 g, 1.05 mmol) in dry DMF was treated with 2-(1-methylpyrrolidin-2-yl)ethan-1-amine (0.16 g, 1.248 mmol), stirred at 150° C. for 15 h, cooled to RT, quenched with ice H₂O and extracted with EtOAc (2×20 mL). The combined organic layers were washed with H₂O and brine, dried over Na₂SO₄ and concentrated under reduced pressure. Purification of the residue by column chromatography on amine silica (Davisil) gave the title compound (0.053 g, 18%) as an off-white solid. ¹H NMR (400 MHz, CDCl₃): δ 8.52 (s, 2H), 7.49-7.42 (m, 4H), 7.36-7.33 (m, 1H), 5.83 (s, 1H), 3.59-3.48 (m, 2H), 3.10 (t, J=7.6 Hz, 1H), 2.36 (s, 3H), 2.36-2.20 (m, 1H), 2.20-2.13 (q, J=9.2 Hz, 1H), 2.05-1.90 (m, 2H), 1.85-1.55 (m, 4H). MS ES⁺: 283.32.

### Example 3: 5-(4-fluorophenyl)-N-(2-(1-methylpyrrolidin-2-yl)ethyl)pyrimidin-2-amine

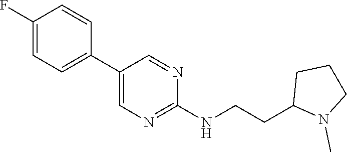 [SMILES: CN1CCCC1CCNc1ncc(-c2ccc(F)cc2)cn1]

Prepared as described for Example 2 using 2-chloro-5-(4-fluorophenyl)pyrimidine (0.20 g, 0.96 mmol) and 2-(1-methylpyrrolidin-2-yl)ethan-1-amine (0.16 g, 1.25 mmol) to afford the title compound (0.072 g, 25%) as an off-white solid. ¹H NMR (400 MHz, DMSO-d₆): δ 8.60 (s, 2H), 7.69-7.63 (m, 2H), 7.37 (br t, 1H), 7.29-7.24 (m, 2H), 3.40 (m, 2H), 3.25-3.10 (m, 1H), 2.50-2.28 (m, 5H), 2.10-1.90 (m, 2H), 1.80-1.68 (m, 2H), 1.65-1.50 (m, 2H). MS ES⁺: 301.31.

### Example 4: 5-(4-fluorophenyl)-N-((1-methylpiperidin-3-yl)methyl)pyrimidin-2-amine

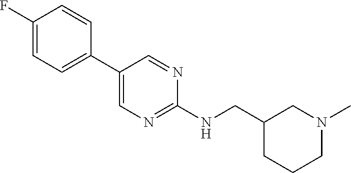 [SMILES: CN1CCCC(CNc2ncc(-c3ccc(F)cc3)cn2)C1]

Prepared as described for Example 2 using 2-chloro-5-(4-fluorophenyl)pyrimidine (0.20 g, 0.96 mmol) and (1-methylpiperidin-3-yl)methanamine (0.16 g, 1.13 mmol) to afford the title compound (0.070 g, 24%) as an off-white solid. ¹H NMR (400 MHz, DMSO-d₆): δ 8.58 (s, 2H), 7.68-7.62 (m, 2H), 7.41 (t, J=6.0 Hz, 1H), 7.29-7.24 (m, 2H), 3.30-3.15 (m, 2H), 2.90-2.65 (m, 2H), 2.25 (br s, 3H), 2.05-1.75 (m, 3H), 1.72-1.60 (m, 2H), 1.55-1.40 (m, 1H), 1.00-0.90 (m, 1H). MS ES⁺: 301.32.

### Example 5: 5-(4-fluorophenyl)-N-(1-methylpiperidin-3-yl)pyrimidin-2-amine

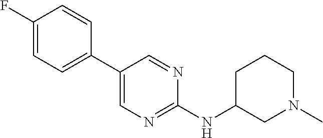 [SMILES: CN1CCCC(Nc2ncc(-c3ccc(F)cc3)cn2)C1]

Prepared as described for Example 2 using 2-chloro-5-(4-fluorophenyl)pyrimidine (0.22 g, 1.08 mmol) and 3-amino-1-methylpiperidine (0.135 g, 1.19 mmol) to afford the title compound (0.033 g, 12%) as an off-white solid. ¹H NMR (400 MHz, DMSO-d₆): δ 8.60 (s, 2H), 7.68-7.64 (m, 2H), 7.30-7.25 (m, 2H), 7.18-7.12 (m, 1H), 3.95-3.88 (m, 1H), 2.90-2.80 (m, 1H), 2.65-2.55 (m, 1H), 2.19 (br s, 3H), 1.90-1.78 (m, 3H), 1.72-1.65 (m, 1H), 1.58-1.48 (m, 1H), 1.35-1.20 (m, 1H). MS ES⁺: 287.24.

### Example 6: 5-(4-fluorophenyl)-N-(1-isopropylpiperidin-3-yl)pyrimidin-2-amine

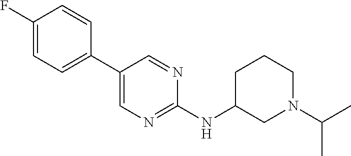 [SMILES: CC(C)N1CCCC(Nc2ncc(-c3ccc(F)cc3)cn2)C1]

Prepared as described for Example 2 using 2-chloro-5-(4-fluorophenyl)pyrimidine (0.30 g, 1.44 mmol) and (1-isopropylpiperidin-3-yl)amine (0.340 g, 1.58 mmol) to afford the title compound (0.090 g, 20%) as a brown solid. ¹H NMR (400 MHz, DMSO-d₆): δ 8.60 (s, 2H), 7.68-7.64 (m, 2H), 7.30-7.25 (t, J=6.6 Hz, 2H), 7.12-7.02 (br s, 1H), 3.95-3.85 (br s, 1H), 2.95-2.85 (m, 1H), 2.70-2.55 (m, 2H), 2.20-2.00 (m, 2H), 1.85-1.78 (m, 1H), 1.75-1.60 (m, 1H), 1.55-1.40 (m, 1H), 1.40-1.30 (m, 1H), 1.10-0.90 (br s, 6H). MS ES⁺: 315.31.

### Example 7: N¹-(5-(4-fluorophenyl)pyrimidin-2-yl)-N³,N³-dimethylcyclohexane-1,3-diamine

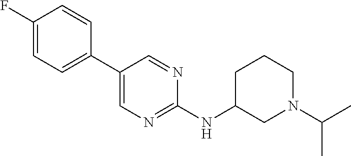 [SMILES: CC(C)N1CCCC(Nc2ncc(-c3ccc(F)cc3)cn2)C1]

Prepared as described for Example 2 using 2-chloro-5-(4-fluorophenyl)pyrimidine (0.20 g, 0.96 mmol) and 3-(amino)-N,N-dimethylcyclohexanamine (0.16 g, 1.05 mmol) to afford the title compound (0.065 g, 21%) as an off-white solid. ¹H NMR (400 MHz, DMSO-d₆): δ 8.65 (s, 1H), 8.62 (s, 1H), 7.69-7.65 (m, 2H), 7.50 (s, 1H), 7.30-7.25 (m, 2H), 4.30-3.90 (m, 1H), 3.40-3.20 (m, 1H), 2.75 (s, 6H), 2.20-2.10 (m, 1H), 1.95-1.85 (m, 1H), 1.80-1.70 (m, 2H), 1.60 (s, 1H), 1.50-1.20 (m, 1H), 1.10-0.90 (br s, 2H). MS ES⁺: 315.39.

### Example 8: 5-(4-fluorophenyl)-N-((1-methylpiperidin-2-yl)methyl)pyrimidin-2-amine

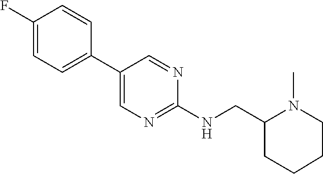 [SMILES: CN1CCCCC1CNc1ncc(-c2ccc(F)cc2)cn1]

Prepared as described for Example 2 using 2-chloro-5-(4-fluorophenyl)pyrimidine (0.22 g, 1.1 mmol) and (1-methylpiperidin-2-yl)methanamine (0.162 g, 1.3 mmol) to afford the title compound (0.072 g, 25%) as an off-white solid. ¹H NMR (400 MHz, DMSO-d₆): δ 8.60 (s, 2H), 7.70-7.65 (m, 2H), 7.27 (t, J=8.8 Hz, 2H), 7.10-6.90 (br s, 1H), 3.65-3.58 (m, 1H), 3.25 (br s, 1H), 2.85-2.70 (m, 1H), 2.40-1.90 (m, 5H), 1.75-1.60 (m, 2H), 1.55-1.15 (m, 4H). MS ES⁺: 301.29.

### Example 9: 5-(4-fluorophenyl)-N-(1-isopropylpiperidin-3-yl)-4-methyl-pyrimidin-2-amine

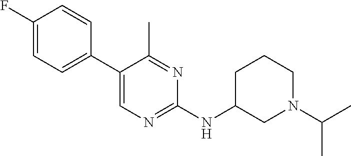 [SMILES: Cc1nc(NC2CCCN(C(C)C)C2)ncc1-c1ccc(F)cc1]

Prepared as described for Example 2 using 2-chloro-5-(4-fluorophenyl)-4-methyl-pyrimidine (0.22 g, 1.1 mmol) and 1-isopropylpiperidin-3-amine (0.222 g, 1 mmol) to afford the title compound (0.07 g, 25%) as an off-white solid. ¹H NMR (400 MHz, DMSO-d₆): δ 8.07 (s, 1H), 7.40-7.37 (m, 2H), 7.25 (t, J=8.4 Hz, 2H), 6.92-6.82 (br s, 1H), 3.95-3.85 (br s, 1H), 2.90-2.80 (m, 1H), 2.75-2.65 (m, 1H), 2.65-2.60 (m, 1H), 2.25 (s, 3H), 2.15-2.08 (m, 1H), 2.02 (t, J=9.2 Hz, 1H), 1.85-1.75 (m, 1H), 1.70-1.62 (m, 1H), 1.55-1.40 (m, 1H), 1.35-1.25 (m, 1H), 0.95 (d, J=6.4 Hz, 6H). MS ES⁺: 329.10.

### Example 10: 5-(2,4-difluorophenyl)-N-((1-ethylpiperidin-2-yl)methyl)-4-methyl-pyrimidin-2-amine, hydrochloride salt

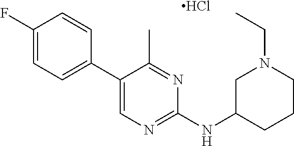 [SMILES: Cl ; CCN1CCCC(Nc2ncc(-c3ccc(F)cc3)c(C)n2)C1]

Prepared as described for Example 2 using 2-chloro-5-(2,4-difluorophenyl)-4-methyl-pyrimidine (0.1 g, 0.4 mmol) and (1-ethylpiperidin-2-yl)methanamine (0.056 g, 0.40 mmol) to afford 5-(2,4-difluorophenyl)-N-((1-ethylpiperidin-2-yl)methyl)-4-methyl-pyrimidin-2-amine (0.12 g, 57%) as a pale brown liquid. A solution of 5-(2,4-difluorophenyl)-N-((1-ethylpiperidin-2-yl)methyl)-4-methyl-pyrimidin-2-amine (0.12 g, 0.35 mmol) in dioxane (2 mL) at 0° C. was treated with 4M HCl in dioxane (2 mL) and stirred at RT for 2 h. Solvent was evaporated from the mixture under reduced pressure and the remaining solid was triturated with diethyl ether and decanted to afford the title compound as a pale yellow sticky solid (0.12 g, 99%). ¹H NMR (400 MHz, DMSO-d₆): δ 9.85 (s, 1H), 8.14 (s, 1H), 7.64 (t, J=5.6 Hz, 1H), 7.45-7.35 (m, 2H), 7.22-7.17 (dt, J=2.4 Hz and 8.4 Hz, 1H), 3.65-3.10 (m, 6H), 3.05-2.95 (m, 1H), 2.17 (s, 3H), 2.05-1.95 (m, 1H), 1.85-1.40 (m, 5H), 1.35-1.20 (m, 3H). MS ES⁺: 347.28.

### Example 11: 5-(2,4-difluorophenyl)-N-((1-(dimethylamino)cyclopentyl)methyl)-4-methyl-pyrimidin-2-amine, hydrochloride salt

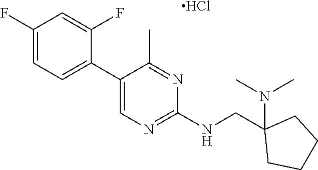 [SMILES: Cl ; Cc1nc(NCC2(N(C)C)CCCC2)ncc1-c1ccc(F)cc1F]

Prepared as described for Example 10 using 2-chloro-5-(2,4-difluorophenyl)-4-methyl-pyrimidine (0.20 g, 0.80 mmol) and 1-(aminomethyl)-N,N-dimethyl-cyclopentanamine (0.102 g, 0.8 mmol) to afford the title compound (0.065 g, 30%) as an off-white solid. ¹H NMR (400 MHz, DMSO-d₆): δ 10.28 (s, 1H), 8.17 (s, 1H), 7.68 (t, J=6.4 Hz, 1H), 7.48-7.35 (m, 2H), 7.22-7.17 (dt, J=2.0 Hz and 8.0 Hz, 1H), 3.72 (d, J=6.8 Hz, 2H), 2.82 (d, J=4.8 Hz, 6H), 2.18 (s, 3H), 1.95-1.85 (m, 4H), 1.80-1.70 (m, 4H). MS ES⁺: 347.10.

### Example 12: N-(5-(2,4-difluorophenyl)-4-methyl-pyrimidin-2-yl)-8-methyl-8-azabicyclo[3.2.1]octan-3-amine, hydrochloride salt

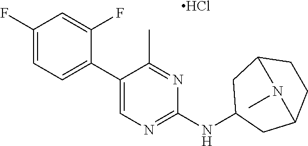 [SMILES: Cl ; Cc1nc(NC2CC3CCC(C2)N3C)ncc1-c1ccc(F)cc1F]

Prepared as described for Example 10 using 2-chloro-5-(2,4-difluorophenyl)-4-methyl-pyrimidine (0.2 g, 0.8 mmol) and 8-methyl-8-azabicyclo[3.2.1]octan-3-amine (0.112 g, 0.8 mmol) to afford the title compound (0.077 g, 30%) as an off-white solid. ¹H NMR (400 MHz, DMSO-d₆): δ 10.14 (s, 1H), 8.16 (s, 1H), 7.76 (br s, 1H), 7.48-7.35 (m, 2H), 7.25-7.17 (dt, J=2.4 Hz and 6.0 Hz, 1H), 4.12 (br s, 1H), 3.95-3.80 (m, 2H), 2.90-2.60 (m, 1H), 2.65 (br d, 2H), 2.30-2.10 (m, 7H), 2.05-1.95 (m, 4H). MS ES⁺: 345.29.

### Example 13: 5-(2,4-difluorophenyl)-4-methyl-N-((1-methylpyrrolidin-3-yl)methyl) pyrimidin-2-amine, hydrochloride salt

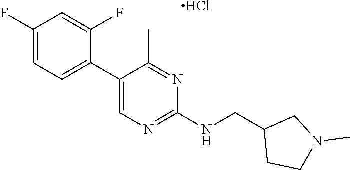 [SMILES: Cl ; Cc1nc(NCC2CCN(C)C2)ncc1-c1ccc(F)cc1F]

Prepared as described for Example 10 using 2-chloro-5-(2,4-difluorophenyl)-4-methyl-pyrimidine (0.2 g, 0.8 mmol) and (1-methylpyrrolidin-3-yl)methanamine (0.091 g, 0.8 mmol) to afford the title compound (0.068 g, 24%) as an off-white solid. ¹H NMR (400 MHz, DMSO-d₆): δ 10.50 (s, 1H), 8.10 (s, 1H), 7.65 (br s, 1H), 7.46-7.35 (m, 2H), 7.22-7.17 (dt, J=2.4 Hz and 8.4 Hz, 1H), 3.60-3.40 (m, 3H), 3.15-2.98 (m, 2H), 2.85-2.75 (m, 4H), 2.70-2.60 (m, 1H), 2.25-2.00 (m, 1H), 2.15 (s, 3H), 1.90-1.60 (m, 1H). MS ES⁺: 319.10.

### Example 14: 5-(2,4-difluorophenyl)-4-methyl-N-(2-(1-methylpyrrolidin-2-yl)ethyl) pyrimidin-2-amine, hydrochloride salt

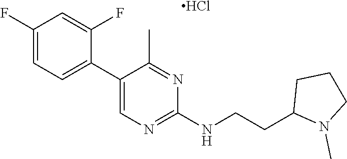 [SMILES: Cl ; Cc1nc(NCCC2CCCN2C)ncc1-c1ccc(F)cc1F]

Prepared as described for Example 10 using 2-chloro-5-(2,4-difluorophenyl)-4-methyl-pyrimidine (0.2 g, 0.8 mmol) and 2-(1-methylpyrrolidin-2-yl)ethan-1-amine (0.102 g, 0.8 mmol) to afford the title compound (0.17 g, 68%) as an off-white solid. ¹H NMR (400 MHz, DMSO-d₆): δ 10.35 (s, 1H), 8.10 (s, 1H), 7.65 (br s, 1H), 7.46-7.35 (m, 2H), 7.20-7.10 (m, 1H), 3.60-3.50 (m, 3H), 3.30 (m, 1H), 3.10-2.95 (m, 1H), 2.80 (s, 3H), 2.30-2.20 (m, 1H), 2.10 (m, 4H), 2.00-1.80 (m, 3H), 1.80-1.70 (m, 1H). MS ES⁺: 333.31.

### Example 15: 6-(2,4-difluorophenyl)-5-methyl-N-((1-methylpyrrolidin-2-yl)methyl)-1,2,4-triazin-3-amine

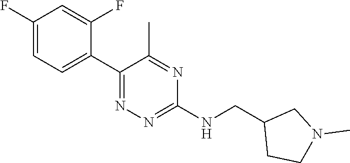 [SMILES: Cc1nc(NCC2CCN(C)C2)nnc1-c1ccc(F)cc1F]

A stirred solution of 6-(2,4-difluorophenyl)-5-methyl-3-(methylsulfonyl)-1,2,4-triazine (0.3 g, 1.1 mol) and (1-methylpyrrolidin-2-yl)methanamine (0.15 g, 1.32 mol) in 1,4-dioxane was refluxed at 110° C. and evaporated under reduced pressure. Purification of the residue by prep-HPLC gave the title compound (150 mg, 44%) as a pale brown liquid. ¹H NMR (400 MHz, DMSO-d₆): δ 8.00-7.70 (br s, 1H), 7.60 (q, J=6.80 Hz, 1H), 7.42 (dt, J=2.40 Hz and 9.2 Hz, 1H), 7.25 (t, J=8.4 Hz, 1H), 3.60 (br s, 1H), 3.30-3.15 (m, 1H), 3.00-2.90 (m, 1H), 2.40 (br s, 1H), 2.32 (s, 3H), 2.20-2.10 (m, 4H), 1.95-1.80 (m, 1H), 1.70-1.55 (m, 3H). MS ES⁺: 320.32.

### Example 16: 5-(2-fluoro-4-methoxyphenyl)-4-methyl-N-(2-(1-methylpyrrolidin-2-yl) ethyl)pyrimidin-2-amine, hydrochloride salt

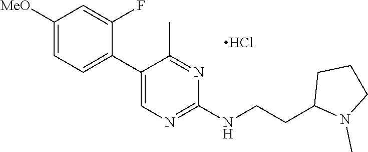 [SMILES: COc1ccc(-c2cnc(NCCC3CCCN3C)nc2C)c(F)c1 ; Cl]

Prepared as described for Example 10 using 2-chloro-5-(2-fluoro-4-methoxyphenyl)-4-methyl-pyrimidine (0.20 g, 0.80 mmol) and 2-(1-methylpyrrolidin-2-yl)ethan-1-amine (0.12 g, 0.9 mmol) to afford the title compound (0.09 g, 33%) as a pale yellow sticky solid. ¹H NMR (400 MHz, DMSO-d₆): δ 10.00 (s, 1H), 8.10 (s, 1H), 7.55 (s, 1H), 7.26 (t, J=8.8 Hz, 1H), 6.94 (dd, J=2.4 Hz and 12.0 Hz, 1H), 6.87 (dd, J=2.4 Hz and 12.0 Hz, 1H), 3.81 (s, 3H), 3.60-3.50 (m, 11H), 3.45-3.35 (m, 2H), 3.35-3.25 (m, 1H), 3.05-2.98 (m, 1H), 2.79 (d, J=5.2 Hz, 3H), 2.35-2.28 (m, 1H), 2.25-2.15 (m, 4H), 2.00-1.85 (m, 2H), 185-1.75 (m, 1H), 1.72-1.60 (m, 1H). MS ES⁺: 345.10.

### Example 17: 5-(2-fluoro-4-methoxyphenyl)-4-methyl-N-((1-methylpiperidin-2-yl)methyl) pyrimidin-2-amine

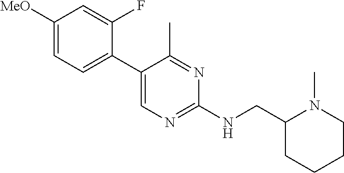 [SMILES: COc1ccc(-c2cnc(NCC3CCCCN3C)nc2C)c(F)c1]

Prepared as described for Example 2 using 2-chloro-5-(2-fluoro-4-methoxyphenyl)-4-methyl-pyrimidine (0.2 g, 0.8 mmol) and (1-methylpiperidin-2-yl)methanamine (0.12 g, 0.90 mmol) to afford the title compound (0.094 g, 35%) as an off-white solid. ¹H NMR (400 MHz, DMSO-d₆): δ 9.92 (s, 1H), 8.13 (s, 1H), 7.60 (t, J=6.1 Hz, 1H), 7.26 (t, J=8.8 Hz, 1H), 6.95 (dd, J=2.4 Hz and 12.0 Hz, 1H), 6.89 (dd, J=2.4 Hz and 8.4 Hz, 1H), 3.81 (s, 3H), 3.60-3.45 (m, 2H), 3.40-3.32 (m, 1H), 3.25-3.12 (m, 1H), 3.05-2.95 (m, 1H), 2.92-2.70 (m, 3H), 2.18 (s, 3H), 1.97 (d, J=13.2 Hz, 1H), 1.80-1.60 (m, 3H), 1.50-1.35 (m, 1H). MS ES⁺: 345.10.

### Example 18a/b: 6-(2-fluoro-4-methoxyphenyl)-5-methyl-N-((1-methylpyrrolidin-2-yl) methyl)-1,2,4-triazin-3-amine

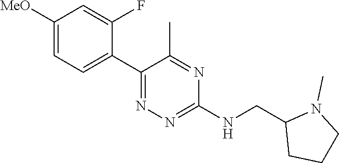 [SMILES: COc1ccc(-c2nnc(NCC3CCCN3C)nc2C)c(F)c1]

Prepared as described for Example 15 using 6-(2-fluoro-4-methoxyphenyl)-5-methyl-3-(methylsulfonyl)-1,2,4-triazine (0.70 g, 2.4 mmol) and (1-methylpyrrolidin-2-yl) methanamine (0.301 g, 2.64 mmol) to afford the title compound as racemate which was subjected to separation of enantiomers by SFC (YMC-SC (30 mm×250 mm, 5 μm); CO₂: 60%, co-solvent: 40% of 0.2% 7M NH₃ in MeOH; total flow: 110.0 g/min; T=30° C.; UV detection at: 250 nm) to obtain isomer 1 (0.175 g, 23%) and isomer 2 (0.17 g, 23%) as pale brown sticky solids.

Example 18a (isomer 1): ¹H NMR (400 MHz, DMSO-d₆): δ 7.75 (br s, 1H), 7.43 (t, J=8.80 Hz, 1H), 6.97 (dd, J=2.40 Hz, 12.0 Hz, 1H), 6.93 (dd, J=2.4 Hz and 8.4 Hz, 1H), 3.85 (s, 3H), 3.61 (br s, 1H), 3.25-3.15 (m, 1H), 2.98-2.92 (m, 1H), 2.45-2.35 (br s, 1H), 2.32 (s, 3H), 2.20 (s, 3H), 2.15-2.08 (m, 1H), 1.90-1.82 (m, 1H), 1.70-1.55 (m, 3H). MS ES⁺: 332.29. Chiral HPLC: 99.8, 3.49 min. SOR: −54.3 (c 0.1, MeOH).

Example 18b (isomer 2): ¹H NMR (400 MHz, DMSO-d₆): δ 7.70 (br s, 1H), 7.43 (t, J=8.40 Hz, 1H), 6.97 (dd, J=2.40 Hz, 12.0 Hz, 1H), 6.93 (dd, J=2.4 Hz and 8.4 Hz, 1H), 3.85 (s, 3H), 3.61 (br s, 1H), 3.25-3.15 (m, 1H), 2.98-2.92 (m, 1H), 2.45-2.35 (br s, 1H), 2.32 (s, 3H), 2.20 (s, 3H), 2.15-2.08 (m, 1H), 1.90-1.82 (m, 1H), 1.70-1.55 (m, 3H). MS ES⁺: 332.29. Chiral HPLC: 99.7, 6.4 min. SOR: +54.7 (c 0.1, MeOH).

### Example 19: 5-(2,4-difluorophenyl)-2-(((1-methylpyrrolidin-2-yl)methyl)amino) pyrimidine-4-carbonitrile

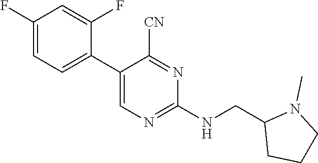 [SMILES: CN1CCCC1CNc1ncc(-c2ccc(F)cc2F)c(C#N)n1]

A mixture of 5-(2,4-difluorophenyl)-2-(methylsulfonyl)pyrimidine-4-carbonitrile (0.20 g, 0.7 mmol) and (1-methylpyrrolidin-2-yl)methanamine (0.087 g, 0.21 mmol) were heated at 100° C. for 2 h (microwave), cooled to RT, treated with H₂O and extracted with EtOAc (2×50 mL). The combined organic layers were dried over Na₂SO₄ and concentrated. Purification of the residue by prep-HPLC gave the title compound (0.12 g, 52%) as a pale yellow gummy solid. ¹H NMR (400 MHz, DMSO-d₆): δ 8.61 (d, J=18.00 Hz, 1H), 7.97 (br s, 1H), 7.68-7.62 (q, J=8.4 Hz, 1H), 7.51-7.45 (dt, J=2.4 Hz and 9.4 Hz, 1H), 7.30-7.25 (dt, J=2.0 Hz and 8.4 Hz, 1H), 3.55-3.45 (m, 1H), 3.25-3.15 (m, 1H), 3.00-2.90 (m, 1H), 2.40-2.30 (m, 3H), 2.32 (s, 1H), 2.20-2.10 (m, 1H), 1.95-1.80 (m, 1H), 1.70-1.55 (m, 3H). MS ES⁺: 330.30.

### Example 20: 6-(2,4-difluorophenyl)-5-methyl-N-((1-methylpiperidin-2-yl)methyl)-1,2,4-triazin-3-amine

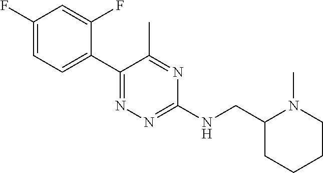 [SMILES: Cc1nc(NCC2CCCCN2C)nnc1-c1ccc(F)cc1F]

Prepared as described for Example 15 using 6-(2,4-difluorophenyl)-5-methyl-3-(methylsulfonyl)-1,2,4-triazine (0.25 g, 0.87 mmol) and (1-methylpiperidin-2-yl)methanamine (0.15 g, 1.2 mmol) to afford the title compound (0.12 g, 41%) as a pale brown sticky solid. ¹H NMR (400 MHz, DMSO-d₆): δ 7.76 (br s, 1H), 7.64-7.57 (q, J=7.6 Hz, 1H), 7.45-7.39 (dt, J=2.4 Hz and 9.8 Hz, 1H), 7.30-7.25 (dt, J=2.0 Hz and 8.8 Hz, 1H), 3.66 (br s, 1H), 3.22 (s, 3H), 2.76 (d, J=8.00 Hz, 1H), 2.25 (s, 3H), 2.15-2.05 (s, 1H), 2.05-1.95 (m, 2H), 1.72-1.62 (m, 2H), 1.55-1.38 (m, 2H), 1.25-1.15 (m, 2H). MS ES⁺: 334.10.

### Example 21: 6-(2,4-difluorophenyl)-5-methyl-N-(2-(1-methylpyrrolidin-2-yl)ethyl)-1,2,4-triazin-3-amine

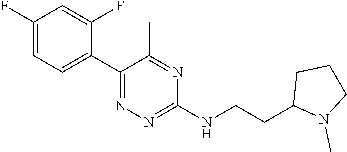 [SMILES: Cc1nc(NCCC2CCCN2C)nnc1-c1ccc(F)cc1F]

Prepared as described for Example 15 using 6-(2,4-difluorophenyl)-5-methyl-3-(methylsulfonyl)-1,2,4-triazine (0.25 g, 0.87 mmol) and 2-(1-methylpyrrolidin-2-yl)ethan-1-amine (0.126 g, 1.2 mmol) to afford the title compound (0.13 g, 44%) as an off-white solid. ¹H NMR (400 MHz, DMSO-d₆): δ 7.85 (br s, 1H), 7.64-7.57 (q, J=7.6 Hz, 1H), 7.45-7.39 (dt, J=2.4 Hz and 9.6 Hz, 1H), 7.28-7.22 (dt, J=2.0 Hz and 8.8 Hz, 1H), 3.45 (br s, 2H), 2.95-2.90 (m, 1H), 2.25 (s, 3H), 2.20 (s, 3H), 2.15-2.00 (m, 2H), 1.95-1.85 (m, 2H), 1.68-1.58 (m, 2H), 1.55-1.40 (m, 2H). MS ES⁺: 334.33.

### Example 22: 5-(2,4-difluorophenyl)-N-(1-isopropylpyrrolidin-3-yl)-4-methyl-pyrimidin-2-amine

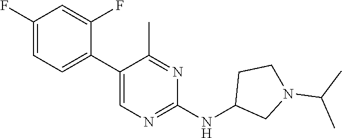 [SMILES: Cc1nc(NC2CCN(C(C)C)C2)ncc1-c1ccc(F)cc1F]

Prepared as described for Example 2 using 2-chloro-5-(2,4-difluorophenyl)-4-methyl-pyrimidine (0.13 g, 0.54 mmol) and 1-isopropylpyrrolidin-3-amine (0.083 g, 0.65 mmol) to afford the title compound (0.067 g, 42%) as an off-white solid. ¹H NMR (400 MHz, DMSO-d₆): δ 8.06 (s, 1H), 7.45-7.33 (m, 3H), 7.25-7.16 (m, 1H), 4.32 (s, 1H), 2.95-2.85 (m, 1H), 2.65-2.50 (m, 2H), 2.40-2.30 (m, 2H), 2.15-2.05 (m, 4H), 1.75-1.65 (m, 1H), 1.01 (d, J=12.80 Hz, 6H). MS ES⁺: 333.39.

### Example 23: 6-(2-fluoro-4-methoxyphenyl)-5-methyl-N-(2-(1-methylpyrrolidin-2-yl) ethyl)-1,2,4-triazin-3-amine

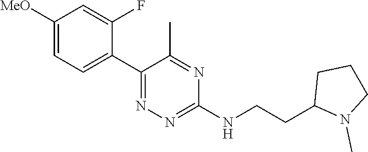 [SMILES: COc1ccc(-c2nnc(NCCC3CCCN3C)nc2C)c(F)c1]

Prepared as described for Example 15 using 6-(2-fluoro-4-methoxyphenyl)-5-methyl-3-(methylsulfonyl)-1,2,4-triazine (0.27 g, 0.9 mmol) and 2-(1-methylpyrrolidin-2-yl)ethan-1-amine (0.126 g, 1.2 mmol) to afford the title compound (0.2 g, 68%) as an off-white solid. ¹H NMR (400 MHz, DMSO-d₆): δ 7.693 (br, 1H), 7.43 (t, J=8.80 Hz, 1H), 6.95 (dd, J=2.00 Hz and 8.0 Hz, 1H), 6.93 (dd, J=2.40 Hz and 8.4 Hz, 1H), 3.84 (s, 3H), 3.40 (s, 2H), 2.95-2.90 (t, J=3.20 Hz, 1H), 2.21 (s, 3H), 2.20 (s, 3H), 2.15-2.20 (m, 2H), 1.95-1.85 (m, 2H), 1.68-1.58 (m, 2H), 1.55-1.40 (m, 2H). MS ES⁺: 346.42.

### Example 24a: (S)-5-(2,4-difluorophenyl)-4-methyl-N-((1-methylpyrrolidin-2-yl)methyl) pyrimidin-2-amine

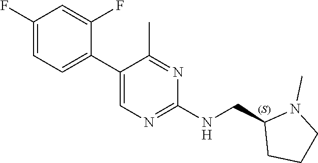

A stirred solution of 2-chloro-5-(2,4-difluorophenyl)-4-methyl-pyrimidine (0.15 g, 0.63 mmol) and (S)-(1-methylpyrrolidin-2-yl)methanamine (0.107 g, 0.93 mmol) in dioxane (9 mL) and DIPEA (1 mL) was stirred at 90° C. for 16 h in a sealed tube, cooled to RT and evaporated. The residue was diluted with DCM and washed with H₂O. The organic layer was dried over Na₂SO₄ and concentrated under reduced pressure. Purification of the residue by reverse phase column chromatography gave the title compound (90 mg, 46%) as an off-white semi-solid. ¹H NMR (400 MHz, DMSO-d₆): δ 8.05 (s, 1H), 7.45-7.32 (m, 2H), 7.18 (m, 1H), 7.03 (s, 1H), 3.55-3.50 (m, 1H), 3.15-3.10 (m, 1H), 2.95-2.90 (m, 1H), 2.40-2.32 (m, 1H), 2.30 (s, 3H), 2.15-2.10 (m, 4H), 1.84 (m, 1H), 1.60 (m, 3H). MS ES⁺: 319.23. Chiral HPLC: 99.43%, 4.70 min. SOR: −51.5 (c 0.1, MeOH).

### Example 24b: (R)-5-(2,4-difluorophenyl)-4-methyl-N-((1-methylpyrrolidin-2-yl)methyl) pyrimidin-2-amine

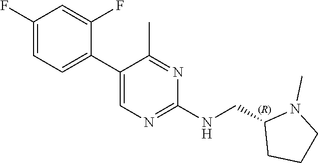

Prepared as described for Example 24a using 2-chloro-5-(2,4-difluorophenyl)-4-methyl-pyrimidine (0.15 g, 0.63 mmol) and (R)-(1-methylpyrrolidin-2-yl)methanamine (0.107 g, 0.93 mmol) to afford the title compound (88 mg, 45%) as an off-white semi-solid. ¹H NMR (400 MHz, DMSO-d₆): δ 8.05 (s, 1H), 7.45-7.32 (m, 2H), 7.22-7.15 (dt, J=2.4 Hz and 8.0 Hz, 1H), 7.05-6.98 (br s, 1H), 3.58-3.50 (m, 1H), 3.18-3.08 (m, 1H), 2.98-2.90 (m, 1H), 2.40-2.32 (m, 1H), 2.30 (s, 3H), 2.15-2.08 (m, 4H), 1.90-1.80 (m, 1H), 1.65-1.52 (m, 3H). MS ES⁺: 319.23. Chiral HPLC: 96.75%, 5.65 min. SOR: +48.9 (c 0.1, MeOH).

### Example 25: 5-(2-fluoro-4-methoxyphenyl)-2-(((1-methylpiperidin-2-yl)methyl)amino) pyrimidine-4-carbonitrile

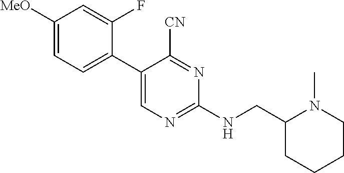 [SMILES: COc1ccc(-c2cnc(NCC3CCCCN3C)nc2C#N)c(F)c1]

A solution of 5-(2-fluoro-4-methoxyphenyl)-2-(methylsulfonyl)pyrimidine-4-carbonitrile (0.15 g, 0.5 mmol) and (1-methylpiperidin-2-yl)methanamine (0.064 g, 0.5 mmol) in dioxane (3 ml) was heated at 100° C. (microwave) for 1 h and evaporated under reduced pressure. Purification of the residue by column chorography (Grace instrument, 0-5% MeOH in DCM) gave the title compound (0.06 g, 33%) as a pale yellow sticky solid. ¹H NMR (400 MHz, DMSO-d₆): δ 8.58 (d, J=13.60 Hz, 1H), 7.78 (d, J=21.60 Hz, 1H), 7.48 (t, J=8.80 Hz, 1H), 7.03 (dd, J=2.40 Hz and 12.40 Hz, 1H), 6.94 (dd, J=2.40 Hz and 8.40 Hz, 1H), 3.83 (s, 3H), 3.65-3.50 (m, 1H), 3.30-3.20 (m, 1H), 2.80-2.70 (d, J=11.20 Hz, 1H), 2.25 (s, 3H), 2.10-2.02 (m, 2H), 1.70-1.60 (m, 2H), 1.55-1.40 (m, 2H), 1.30-1.10 (m, 2H). MS ES⁺: 356.10.

### Example 26: 6-(2-fluoro-4-methoxyphenyl)-5-methyl-N-((1-methylpiperidin-2-yl) methyl)-1,2,4-triazin-3-amine, hydrochloride salt

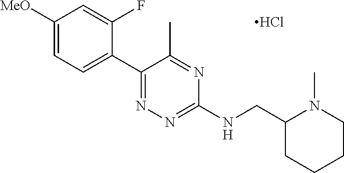 [SMILES: COc1ccc(-c2nnc(NCC3CCCCN3C)nc2C)c(F)c1 ; Cl]

Prepared as described for Example 15 using 6-(2-fluoro-4-methoxyphenyl)-5-methyl-3-(methylsulfonyl)-1,2,4-triazine (0.27 g, 0.9 mmol) and (1-methylpiperidin-2-yl)methanamine (0.127 g, 1.0 mmol) to afford 6-(2-fluoro-4-methoxyphenyl)-5-methyl-N-((1-methylpiperidin-2-yl)methyl)-1,2,4-triazin-3-amine (0.15 g, 48%) as a pale brown sticky solid. ¹H NMR (400 MHz, DMSO-de): δ 7.70-7.40 (br s, 1H), 7.44 (t, J=8.80 Hz, 1H), 7.00-6.96 (dd, J=2.4 Hz and 12.4 Hz, 1H), 6.94-6.91 (dd, J=2.4 Hz and 8.4 Hz, 1H), 3.84 (s, 3H), 3.70-3.60 (br s, 1H), 3.35-3.22 (m, 1H), 2.76 (d, J=11.60 Hz, 1H), 2.27 (s, 3H), 2.19 (s, 3H), 2.10 (s, 1H), 2.05-1.95 (t, J=3.20 Hz, 1H), 1.75-1.60 (m, 2H), 1.55-1.40 (m, 2H), 1.35-1.10 (m, 2H). MS ES⁺: 346.41.

4M HCl in dioxane was added to a solution of 6-(2-fluoro-4-methoxyphenyl)-5-methyl-N-((1-methylpiperidin-2-yl)methyl)-1,2,4-triazin-3-amine (0.1 g) in dioxane at 0° C. and stirred for 2 h. The solvent was evaporated and the residual solids triturated with Et₂O and concentrated under vacuum to obtain the title compound (90 mg, 82%) as a pale brown solid. ¹H NMR (400 MHz, DMSO-d₆): δ 8.05 (s, 1H), 7.43 (t, J=8.80 Hz, 1H), 6.97 (m, 2H), 3.84 (s, 3H), 3.80 (s, 1H), 3.568 (s, 0.5H), 3.39 (d, J=7.60 Hz, 2H), 3.28 (s, 0.5H), 3.10-3.09 (m, 1H), 2.96 (d, J=4.80 Hz, 3H), 2.243 (m, 3H), 1.99 (d, J=14.40 Hz, 1H), 1.70-1.30 (m, 6H). MS ES⁺: 346.39.

### Example 27: 5-(2-fluoro-4-methoxyphenyl)-2-((2-(1-methylpyrrolidin-2-yl)ethyl)amino) pyrimidine-4-carbonitrile

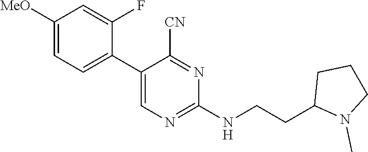 [SMILES: COc1ccc(-c2cnc(NCCC3CCCN3C)nc2C#N)c(F)c1]

Prepared as described for Example 15 using 5-(2-fluoro-4-methoxyphenyl)-2-(methylsulfonyl)pyrimidine-4-carbonitrile (0.2 g, 0.7 mmol) and 2-(1-methylpyrrolidin-2-yl)ethan-1-amine (0.127 g, 1.0 mmol) to afford the title compound (0.10 g, 56%) as a pale brown sticky solid. ¹H NMR (400 MHz, DMSO-d₆): δ 8.59 (d, J=12.8 Hz, 1H), 8.07 (t, J=5.60 Hz, 1H), 7.47 (t, J=8.80 Hz, 1H), 7.03 (dd, J=2.40 Hz and 12.2 Hz, 1H), 6.94 (dd, J=2.0 Hz and 8.6 Hz, 1H), 3.83 (s, 3H), 3.15 (s, 3H), 2.43-2.35 (m, 5H), 2.10-1.95 (m, 2H), 1.80-1.70 (m, 2H), 1.54-1.50 (m, 2H). MS ES⁺: 356.41.

### Example 28a/b: 5-(2-fluoro-4-methoxyphenyl)-4-methyl-N-((1-methylpyrrolidin-2-yl) methyl)pyrimidin-2-amine

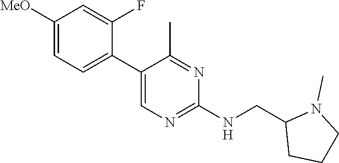 [SMILES: COc1ccc(-c2cnc(NCC3CCCN3C)nc2C)c(F)c1]

A solution of 2-chloro-5-(2-fluoro-4-methoxyphenyl)-4-methyl-pyrimidine (3.00 g, 11.9 mmol) and (1-methylpyrrolidin-2-yl)methanamine (1.83 g, 14.28 mmol) in dioxane (10 mL) was treated with DIPEA (6.0 mL, 36 mmol), stirred at 100° C. for 24 h, cooled to RT and treated with H₂O. The mixture was extracted with EtOAc (2×50 mL). The combined organic layers were dried over Na₂SO₄ and concentrated. Purification of the residue by column chromatography (0-20% MeOH/DCM) gave the title compound (1.25 g, 40%) as a thick gummy liquid. The racemic compound was subjected to separation of enantiomers by prep-SFC. Each enantiomer was subjected to HCl salt formation. A mixture of compound in 4M HCl in dioxane was stirred for 2 h at RT and evaporated under reduced pressure, then lyophilized to give isomer 1 (0.6 g, 91%) and isomer 2 (0.55 g, 84%) as brown sticky solids.

Example 28a (isomer 1): ¹H NMR (400 MHz, DMSO-d₆): δ 10.60 (s, 1H), 8.18 (s, 1H), 7.78 (s, 1H), 7.28 (t, J=8.80 Hz, 1H), 6.96 (dd, J=2.40, 12.00 Hz, 1H), 6.89 (dd, J=2.40, 8.40 Hz, 1H), 3.85-3.70 (m, 5H), 3.60-3.50 (m, 2H), 3.08-3.02 (m, 1H), 2.88 (d, J=4.80 Hz, 3H), 2.26-2.15 (m, 4H), 2.05-1.75 (m, 3H). MS ES⁺: 331.35. Chiral HPLC: 99.9%, 4.73 min.

Example 28b (isomer 2): ¹H NMR (400 MHz, DMSO-d₆): δ 10.60 (s, 1H), 8.18 (s, 1H), 7.78 (s, 1H), 7.28 (t, J=8.80 Hz, 1H), 6.96 (dd, J=2.40, 12.00 Hz, 1H), 6.89 (dd, J=2.40, 8.40 Hz, 1H), 3.85-3.70 (m, 5H), 3.60-3.50 (m, 2H), 3.08-3.02 (m, 1H), 2.88 (d, J=4.80 Hz, 3H), 2.26-2.15 (m, 4H), 2.05-1.75 (m, 3H). MS ES⁺: 331.35. Chiral HPLC: 99.7%, 5.63 min.

### Example 29: 5-(2,4-difluorophenyl)-4-methyl-N-(1-methylpyrrolidin-3-yl)pyrimidin-2-amine

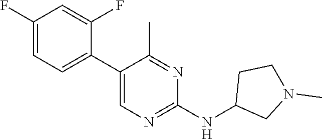 [SMILES: Cc1nc(NC2CCN(C)C2)ncc1-c1ccc(F)cc1F]

Prepared as described for Example 2 using 2-chloro-5-(2,4-difluorophenyl)-4-methyl-pyrimidine (0.3 g, 1.25 mmol) and 1-methylpyrrolidin-3-amine (0.137 g, 1.37 mmol) to afford the title compound (0.05 g, 13%) as an off-white solid. ¹H NMR (400 MHz, DMSO-d₆): δ 8.06 (s, 1H), 7.45-7.33 (m, 3H), 7.18 (t, J=2.00 Hz, 1H), 4.34 (br s, 1H), 2.78 (t, J=6.80 Hz, 1H), 2.55 (s, 1H), 2.45-2.45 (m, 1H), 2.41-2.43 (m, 1H), 2.31 (s, 3H), 2.18-2.10 (m, 4H), 1.75-1.675 (m, 1H). MS ES⁺: 305.30.

### Example 30: 5-(2-fluoro-4-methoxyphenyl)-4-methyl-N-((1-methylpiperidin-3-yl)methyl) pyrimidin-2-amine, hydrochloride salt

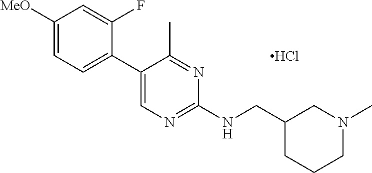 [SMILES: COc1ccc(-c2cnc(NCC3CCCN(C)C3)nc2C)c(F)c1 ; Cl]

Prepared as described for Example 10 using 2-chloro-5-(2-fluoro-4-methoxyphenyl)-4-methyl-pyrimidine (0.25 g, 1.0 mmol) and (1-methylpiperidin-3-yl)methanamine (0.128 g, 1.00 mmol) to afford the title compound (0.06 g, 18%) as an off-white solid. ¹H NMR (400 MHz, DMSO-d₆): δ 9.99 (s, 1H), 8.09 (s, 1H), 7.56 (s, 1H), 7.26 (t, J=8.80 Hz, 1H), 6.94 (dd, J=2.40, 12.00 Hz, 1H), 6.88 (dd, J=2.40, 8.60 Hz, 1H), 3.81 (s, 3H), 3.40-3.23 (m, 4H), 2.80-2.67 (m, 5H), 2.20 (m, 4H), 1.90-1.65 (m, 3H), 1.15-1.05 (m, 1H). MS ES⁺: 345.33.

### Example 31: 5-(2-fluoro-4-methoxyphenyl)-2-(((1-methylpyrrolidin-3-yl)methyl)amino) pyrimidine-4-carbonitrile

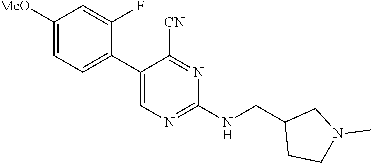 [SMILES: COc1ccc(-c2cnc(NCC3CCN(C)C3)nc2C#N)c(F)c1]

Prepared as described for Example 15 using 5-(2-fluoro-4-methoxyphenyl)-2-(methylsulfonyl)pyrimidine-4-carbonitrile (0.1 g, 0.3 mmol) and (1-methylpyrrolidin-3-yl)methanamine (0.034 g, 0.3 mmol) to afford the title compound (0.05 g, 49%) as a pale yellow sticky solid. ¹H NMR (400 MHz, DMSO-d₆): δ 8.57 (d, J=15.20 Hz, 1H), 8.13 (s, 1H), 7.47 (t, J=8.80 Hz, 1H), 7.03 (dd, J=2.40, 12.40 Hz, 1H), 6.94 (dd, J=2.80, 8.60 Hz, 1H), 3.85 (s, 3H), 3.26-3.20 (m, 2H), 2.50-2.44 (m, 3H), 2.35-2.30 (m, 2H), 2.22 (s, 3H), 1.90-1.82 (m, 1H), 1.50-1.40 (m, 1H). MS ES⁺: 342.31.

### Example 32: 5-(2-fluoro-4-methoxyphenyl)-4-methyl-N-((4-methylmorpholin-3-yl) methyl)pyrimidin-2-amine, hydrochloride salt

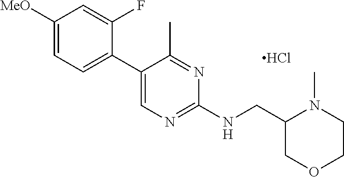 [SMILES: COc1ccc(-c2cnc(NCC3COCCN3C)nc2C)c(F)c1 ; Cl]

Prepared as described for Example 10 using 2-chloro-5-(2-fluoro-4-methoxyphenyl)-4-methyl-pyrimidine (0.30 g, 1.2 mmol) and (4-methylmorpholin-3-yl)methanamine (0.125 g, 0.96 mmol) to afford the title compound (0.1 g, 23%) as an off-white sticky solid. ¹H NMR (400 MHz, DMSO-d₆): δ 10.70 (s, 1H), 8.13 (s, 1H), 7.59 (m, 1H), 7.26 (t, J=17.60 Hz, 1H), 6.95 (d, J=14.80 Hz, 1H), 6.88 (d, J=11.20 Hz, 1H), 4.06 (m, 1H), 3.93-3.70 (m, 6H), 3.66-3.50 (m, 2H), 3.50-3.39 (m, 2H), 3.20-3.10 (m, 1H), 2.95 (s, 3H), 2.19 (s, 3H). MS ES⁺: 347.32.

### Example 33: 5-(2-fluoro-5-methoxyphenyl)-4-methyl-N-((1-methylpiperidin-2-yl)methyl) pyrimidin-2-amine, hydrochloride salt

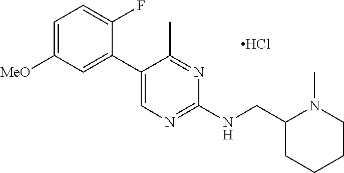 [SMILES: COc1ccc(F)c(-c2cnc(NCC3CCCCN3C)nc2C)c1 ; Cl]

Prepared as described for Example 10 using 2-chloro-5-(2-fluoro-5-methoxyphenyl)-4-methyl-pyrimidine (0.30 g, 1.2 mmol) and (1-methylpiperidin-2-yl)methanamine (0.184 g, 1.44 mmol) to afford the title compound (0.19 g, 42%) as a pale brown solid. ¹H NMR (400 MHz, DMSO-d₆): δ 9.87 (s, 1H), 8.17 (s, 1H), 7.61 (t, J=11.60 Hz, 1H), 7.24 (t, J=9.20 Hz, 1H), 6.98-6.90 (m, 1H), 6.88-6.82 (m, 1H), 3.77 (m, 5H), 3.37 (d, J=13.60 Hz, 1H), 3.28-3.20 (m, 1H), 3.05-3.02 (m, 1H), 2.82 (d, J=5.20 Hz, 3H), 2.20 (s, 3H), 1.97 (d, J=13.60 Hz, 1H), 1.65-1.55 (m, 4H), 1.44 (s, 1H). MS ES⁺: 345.34.

### Example 34: 5-(2-fluoro-5-methoxyphenyl)-4-methyl-N-((1-methylpiperidin-3-yl)methyl) pyrimidin-2-amine, hydrochloride salt

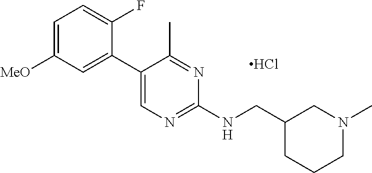 [SMILES: COc1ccc(F)c(-c2cnc(NCC3CCCN(C)C3)nc2C)c1 ; Cl]

Prepared as described for Example 10 using 2-chloro-5-(2-fluoro-5-methoxyphenyl)-4-methyl-pyrimidine (0.27 g, 1.1 mmol) and (1-methylpiperidin-3-yl)methanamine (0.169 g, 1.32 mmol) to afford the title compound (0.19 g, 42%) as a pale brown solid. ¹H NMR (400 MHz, DMSO-d₆): δ 9.89 (s, 1H), 8.14 (s, 1H), 7.57 (s, 1H), 7.23 (t, J=9.20 Hz, 1H), 7.00-6.96 (m, 1H), 6.90-6.88 (m, 1H), 3.77 (s, 3H), 3.45-3.31 (m, 4H), 2.85-2.60 (m, 5H), 2.25-2.10 (m, 4H), 1.90-1.60 (m, 3H), 1.20-1.05 (d, J=3.20 Hz, 1H). MS ES⁺: 345.1.

### Example 35: 5-(2-chloro-4-methoxyphenyl)-4-methyl-N-((1-methylpiperidin-2-yl)methyl) pyrimidin-2-amine, hydrochloride salt

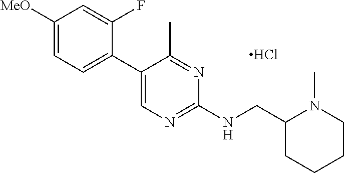 [SMILES: COc1ccc(-c2cnc(NCC3CCCCN3C)nc2C)c(F)c1 ; Cl]

Prepared as described for Example 10 using 2-chloro-5-(2-chloro-4-methoxyphenyl)-4-methyl-pyrimidine (0.27 g, 1.0 mmol) and (1-methylpiperidin-2-yl)methanamine (0.14 g, 1.1 mmol) to afford the title compound (0.13 g, 30%) as a pale brown solid. ¹H NMR (400 MHz, DMSO-d₆): δ 9.99 (s, 1H), 8.06 (d, J=4.00 Hz, 1H), 7.60 (t, J=6.00 Hz, 1H), 7.26 (d, J=8.80 Hz, 1H), 7.18 (d, J=2.40 Hz, 1H), 7.01 (dd, J=1.20, 4.80 Hz, 1H), 3.80-3.70 (m, 3H), 3.68-3.65 (m, 2H), 3.37 (d, J=14.80 Hz, 1H), 3.25-3.21 (m, 1H), 3.18 (d, J=6.80 Hz, 1H), 2.82 (d, J=5.20 Hz, 3H), 2.11 (s, 3H), 1.97 (d, J=14.00 Hz, 1H), 1.63-1.66 (m, 4H), 1.42-1.45 (m, 1H). MS ES⁺: 361.1.

### Example 36: 5-(2-fluoro-4-methoxyphenyl)-4-methyl-N-((4-methylmorpholin-2-yl) methyl)pyrimidin-2-amine, hydrochloride salt

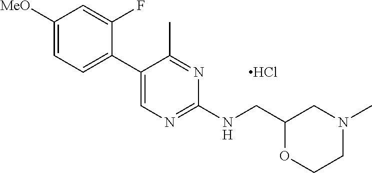 [SMILES: COc1ccc(-c2cnc(NCC3CN(C)CCO3)nc2C)c(F)c1 ; Cl]

Prepared as described for Example 10 using 2-chloro-5-(2-fluoro-4-methoxyphenyl)-4-methyl-pyrimidine (0.3 g, 1.2 mmol) and (4-methylmorpholin-2-yl)methanamine (0.187 g, 1.44 mmol) to afford the title compound (0.1 g, 26%) as an off-white solid. ¹H NMR (400 MHz, DMSO-d&): δ 10.81 (s, 1H), 8.11 (s, 1H), 7.50 (s, 1H), 7.27 (t, J=8.80 Hz, 1H), 6.95 (dd, J=2.80, 12.00 Hz, 1H), 6.88 (dd, J=2.80, 8.60 Hz, 1H), 4.03-4.00 (m, 2H), 3.79-3.72 (m, 4H), 3.50-3.35 (m, 4H), 3.02-3.00 (m, 1H), 2.80-2.75 (m, 4H), 2.17 (s, 3H). MS ES⁺: 347.32.

### Example 37: 4-cyclopropyl-5-(2-fluoro-4-methoxyphenyl)-N-((1-methylpyrrolidin-3-yl) methyl)pyrimidin-2-amine

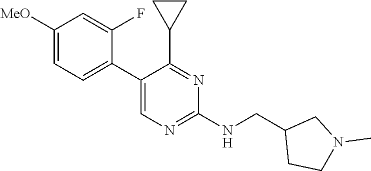 [SMILES: COc1ccc(-c2cnc(NCC3CCN(C)C3)nc2C2CC2)c(F)c1]

A stirred solution of 2-chloro-4-cyclopropyl-5-(2-fluoro-4-methoxyphenyl)pyrimidine (0.15 g, 0.50 mmol) in dioxane (5 mL) was treated with (1-methylpyrrolidin-3-yl)methanamine (0.06 g, 0.55 mmol) and DIPEA (0.193 g, 1.5 mmol), stirred at 100° C. in a sealed tube for 16 h and evaporated. The residue was diluted with EtOAc, washed with H₂O followed by brine. The organic layer was dried over Na₂SO₄ and concentrated. Purification of the residue in automated instrument (GRACE, in reverse phase) gave the title compound (0.06 g, 36%) as a pale yellow gummy mass. ¹H NMR (400 MHz, DMSO-d₆): δ 7.94 (s, 1H), 7.28 (t, J=8.40 Hz, 1H), 7.14 (s, 1H), 6.94 (dd, J=2.40, 12.00 Hz, 1H), 6.88 (dd, J=2.40, 8.40 Hz, 1H), 3.81 (s, 3H), 3.19 (m, 2H), 2.50-2.21 (m, 8H), 1.85-1.81 (m, 1H), 1.66 (t, J=4.40 Hz, 1H), 1.42-1.44 (m, 1H), 1.01 (s, 2H), 0.98-0.85 (m, 2H). MS ES⁺: 357.36.

### Example 38a/b: 2-((3-(dimethylamino)cyclopentyl)amino)-5-(2-fluoro-4-methoxyphenyl) pyrimidine-4-carbonitrile

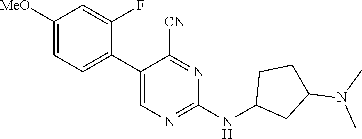 [SMILES: COc1ccc(-c2cnc(NC3CCC(N(C)C)C3)nc2C#N)c(F)c1]

Prepared as described for Example 19 using 5-(2-fluoro-4-methoxyphenyl)-2-(methylsulfonyl)pyrimidine-4-carbonitrile (0.31 g, 1.0 mmol) and N¹,N¹-dimethylcyclopentane-1,3-diamine (0.13 g, 1.0 mmol) to afford the racemic title compound which was subjected to separation of isomers by chiral SFC to give isomer 1 (0.07 g, 48%) and isomer 2 (0.05 g, 42%) as off-white solids.

Example 38a (isomer 1): ¹H NMR (400 MHz, DMSO-d₆): δ 8.57 (d, J=12.40 Hz, 1H), 8.10 (s, 1H), 7.46 (t, J=8.80 Hz, 1H), 7.03 (dd, J=2.40, 12.00 Hz, 1H), 6.94 (dd, J=2.40, 8.60 Hz, 1H), 4.17 (br s, 1H), 3.83 (s, 3H), 2.45-2.40 (m, 1H), 2.18-2.12 (m, 7H), 2.00-1.95 (m, 1H), 1.76 (d, J=7.60 Hz, 1H), 1.54-1.56 (m, 2H), 1.43-1.40 (m, 1H). MS ES⁺: 356.1.

Example 38b (isomer 2): ¹H NMR (400 MHz, DMSO-d₆): δ 8.57 (d, J=12.00 Hz, 1H), 8.10 (s, 1H), 7.46 (t, J=8.80 Hz, 1H), 7.03 (dd, J=2.40 Hz and 12.00 Hz, 1H), 6.94 (dd, J=2.40 Hz and 8.60 Hz, 1H), 4.17 (br s, 1H), 3.83 (s, 3H), 2.50-2.45 (m, 1H), 2.20-2.10 (m, 7H), 2.00-1.90 (m, 1H), 1.80-1.70 (m, 1H), 1.65-1.50 (m, 2H), 1.45-1.35 (m, 1H). MS ES⁺: 356.1.

### Example 39a/b: N¹-(6-(2,4-difluorophenyl)-5-methyl-1,2,4-triazin-3-yl)-N³,N³-dimethylcyclohexane-1,3-diamine

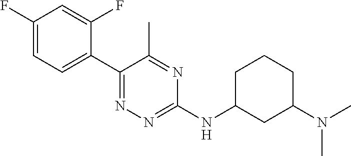 [SMILES: Cc1nc(NC2CCCC(N(C)C)C2)nnc1-c1ccc(F)cc1F]

Prepared as described for Example 15 using 6-(2,4-difluorophenyl)-5-methyl-3-(methylsulfonyl)-1,2,4-triazine (0.50 g, 1.8 mmol) and N³,N³-dimethylcyclohexane-1,3-diamine (0.28 g, 1.98 mol) to afford the title compound, which was subjected to separation of isomers by SFC to obtain isomer 1 (0.045 g, 12%) and isomer 2 (0.041 g, 11%) as off-white solids.

Example 39a (isomer 1): ¹H NMR (400 MHz, DMSO-d₆): δ 7.80 (br m, 1H), 7.64 (m, 1H), 7.42 (t, J=2.40 Hz, 1H), 7.25 (t, J=2.00 Hz, 1H), 4.25 (s, 1H), 2.33 (s, 1H), 2.20-2.17 (m, 9H), 1.96-1.94 (m, 1H), 1.80-1.45 (m, 7H). MS ES⁺: 348.15.

Example 39b (isomer 2): ¹H NMR (400 MHz, DMSO-d₆): δ 7.77 (br m, 1H), 7.60 (m, 1H), 7.42 (t, J=2.40 Hz, 1H), 7.25 (t, J=8.40 Hz, 1H), 3.87 (s, 1H), 2.30 (t, J=11.60 Hz, 1H), 2.20-2.00 (m, 10H), 1.95-1.85 (m, 1H), 1.70-1.60 (t, J=12.80 Hz, 2H), 1.35-1.05 (m, 4H). MS ES⁺: 348.29.

### Example 40: 5-(2-fluoro-4-methoxyphenyl)-4-methyl-N-(1-methylpiperidin-4-yl) pyrimidin-2-amine

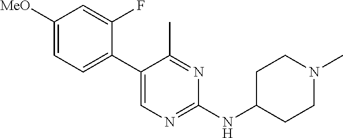 [SMILES: COc1ccc(-c2cnc(NC3CCN(C)CC3)nc2C)c(F)c1]

Prepared as described for Example 2 using 2-chloro-5-(2-fluoro-4-methoxyphenyl)-4-methyl-pyrimidine (0.45 g, 1.70 mmol) and 1-methylpiperidin-4-amine (0.2 g, 1.36 mmol) to afford the title compound (0.05 g, 9%) as an off-white solid. ¹H NMR (400 MHz, DMSO-d₆): δ 8.01 (s, 1H), 7.24 (t, J=8.80 Hz, 1H), 7.09 (d, J=7.20 Hz, 1H), 6.92 (dd, J=2.40 Hz and 11.80 Hz, 1H), 6.86 (dd, J=2.40 Hz and 8.40 Hz, 1H), 3.81 (s, 3H), 3.75-3.65 (br s, 1H), 2.74 (d, J=11.20 Hz, 2H), 2.15 (s, 3H), 2.10 (s, 3H), 1.95 (t, J=11.20 Hz, 2H), 1.80 (d, J=3.20 Hz, 2H), and 1.55-1.40 (m, 2H). MS ES⁺: 331.25.

### Example 41: 5-(2-fluoro-4-methoxyphenyl)-N-(1-isopropylpiperidin-4-yl)-4-methyl-pyrimidin-2-amine, hydrochloride salt

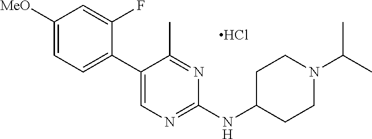 [SMILES: COc1ccc(-c2cnc(NC3CCN(C(C)C)CC3)nc2C)c(F)c1 ; Cl]

Prepared as described for Example 10 using 2-chloro-5-(2-fluoro-4-methoxyphenyl)-4-methyl-pyrimidine (0.35 g, 1.38 mmol) and 1-isopropylpiperidin-4-amine (0.16 g, 1.38 mmol) to afford the title compound (0.10 g, 40%) as a pale yellow solid. ¹H NMR (400 MHz, DMSO-d₆): δ 10.01 (m, 1H), 8.17 (s, 1H), 7.82 (d, J=13.20 Hz, 1H), 7.30-7.25 (m, 1H), 6.96 (dd, J=2.40 Hz and 12.00 Hz, 1H), 6.89 (dd, J=2.40 Hz and 8.40 Hz, 1H), 4.00 (s, 1H), 3.82 (s, 3H), 3.50-3.35 (m, 3H), 3.30-3.20 (m, 1H), 3.15-3.00 (m, 1H), 2.20-2.10 (m, 5H), 2.05-1.85 (m, 2H), and 1.35-1.30 (m, 6H). MS ES⁺: 359.31.

### Example 42: 5-(2,4-difluorophenyl)-4-methyl-N-(pyridin-3-ylmethyl)pyrimidin-2-amine

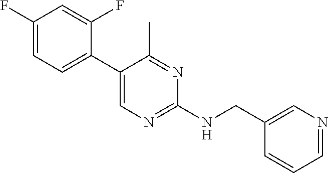 [SMILES: Cc1nc(NCc2cccnc2)ncc1-c1ccc(F)cc1F]

Prepared as described for Example 2 using 2-chloro-5-(2,4-difluorophenyl)-4-methyl-pyrimidine (0.1 g, 0.42 mmol) and pyridin-3-ylmethanamine (0.058 g, 0.55 mmol) to afford the title compound (0.08 g, 61%) as an off-white solid. ¹H NMR (400 MHz, DMSO-d₆): δ 8.56 (s, 1H), 8.43 (dd, J=1.60 Hz and 4.80 Hz, 1H), 8.07 (s, 1H), 7.95-7.85 (br s, 1H), 7.78 (d, J=8.00 Hz, 1H), 7.44-7.29 (m, 3H), 7.17 (t, J=2.00 Hz, 1H), 4.54 (d, J=6.40 Hz, 2H), 2.14 (s, 3H). MS ES⁺: 313.0.

### Example 43: N¹-(5-(2-fluoro-4-methoxyphenyl)-4-(trifluoromethyl)pyrimidin-2-yl)-N³,N³-dimethylcyclopentane-1,3-diamine

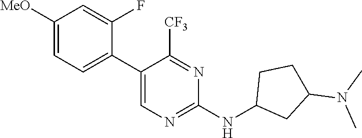 [SMILES: COc1ccc(-c2cnc(NC3CCC(N(C)C)C3)nc2C(F)(F)F)c(F)c1]

A stirred solution of N¹-(5-bromo-4-(trifluoromethyl)pyrimidin-2-yl)-N³,N³-dimethylcyclopentane-1,3-diamine (0.40 g, 1.13 mmol) in dioxane/H₂O (12 mL) was treated with K₂CO₃ (0.455 g, 3.39 mmol) and (2-fluoro-4-methoxyphenyl)boronic acid (0.192 g, 1.13 mmol), degassed with N₂ for 10 min, treated with Pd(dppf)Cl₂-DCM adduct (0.044 g, 0.056 mmol) degassed with N₂ for 10 min, sealed and stirred at 105° C. for 3 h. The mixture was cooled to RT, filtered through Celite®, washed with EtOAc, and the filtrate was concentrated under reduced pressure. Purification of the residue by prep-HPLC gave the title compound (0.06 g, 13%) as a pale brown solid. ¹H NMR (400 MHz, DMSO-d₆): δ 8.41 (s, 1H), 8.06 (m, 1H), 7.27 (t, J=8.8 Hz, 1H), 6.94 (dd, J=2.4 Hz and 11.60 Hz, 1H), 6.86 (dd, J=2.4 Hz and 8.40 Hz, 1H), 4.21 (s, 1H), 3.81 (s, 3H), 2.42 (s, 1H), 2.21-2.12 (m, 7H), 2.00-1.90 (s, 1H), 1.80-1.70 (m, 1H), 1.65-1.52 (m, 2H), 1.50-1.35 (m, 1H). MS ES⁺: 399.38.

### Example 44a/b: N¹-(5-(2-fluoro-4-methoxyphenyl)-4-methyl-pyrimidin-2-yl)-N³,N³-dimethylcyclopentane-1,3-diamine

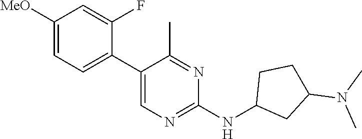 [SMILES: COc1ccc(-c2cnc(NC3CCC(N(C)C)C3)nc2C)c(F)c1]

Prepared as described for Example 2 using 2-chloro-5-(2-fluoro-4-methoxyphenyl)-4-'s methyl-pyrimidine (0.45 g, 1.8 mmol) and N¹,N¹-dimethylcyclopentane-1,3-diamine (0.414 g, 1.8 mmol) to afford the title compound as mixture of diastereomers. The diastereomers were separated by SFC (column/dimensions: Lux Amylose-2 (21 mm×250 mm, 5 μm); CO₂ (75%) and co-solvent (25%, 0.2% 7M methanolic NH₃ in ACN/MeOH 7:3); total flow: 65.0 g/min; T=30° C.; UV detection at 250 nm) to obtain isomer 1 (0.157 g, 22%) and isomer 2 (0.073 g, 11%) as off-white solids.

Example 44a (isomer 1): ¹H NMR (400 MHz, DMSO-d₆): δ 8.01 (s, 1H), 7.21-7.22 (m, 2H), 6.92 (d, J=2.40 Hz, 1H), 6.86 (d, J=2.40 Hz, 1H), 4.19-4.21 (m, 1H), 3.81 (s, 3H), 2.38-2.30 (m, 1H), 2.12 (m, 10H), 1.95-1.92 (m, 1H), 1.78-1.73 (m, 1H), 1.59-1.52 (m, 2H), 1.57-1.53 (m, 1H). MS ES⁺: 345.38.

Example 44b (isomer 2): ¹H NMR (400 MHz, DMSO-d₆): δ 8.01 (s, 1H), 7.29-7.22 (m, 2H), 6.92 (d, J=2.40 Hz, 1H), 6.86 (d, J=2.80 Hz, 1H), 4.19-4.10 (m, 1H), 3.81 (s, 3H), 2.38-2.30 (m, 1H), 2.16 (m, 10H), 1.99-1.93 (m, 1H), 1.83-1.73 (m, 1H), 1.60-1.52 (m, 2H), 1.37-1.32 (m, 1H). MS ES⁺: 345.38.

### Example 45: 5-(2-fluoro-4-methoxyphenyl)-4-methyl-N-(tetrahydro-2H-pyran-4-yl) pyrimidin-2-amine

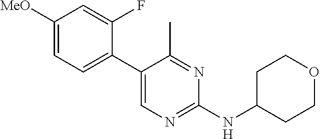 [SMILES: COc1ccc(-c2cnc(NC3CCOCC3)nc2C)c(F)c1]

A solution of 5-bromo-4-methyl-N-(tetrahydro-2H-pyran-4-yl)pyrimidin-2-amine (0.25 g, 0.9 mmol), (2-fluoro-4-methoxyphenyl)boronic acid (0.15 g, 0.9 mmol) and K₂CO₃ (0.372 g, 2.7 mmol) in dioxane/H₂O (8:2, 10 mL) was purged with N₂ for 5 min, treated with Pd(dppf)Cl₂ (0.036 mg, 0.045 mmol) and heated for 3 h at 105° C. The mixture was cooled to RT, filtered through Celite® and the filtrate concentrated under reduced pressure. Purification of the residue by column chromatography (0-50% EtOAc in petrol ether) gave the title compound (0.12 g, 42%) as an off-white solid. ¹H NMR (400 MHz, DMSO-d₆): δ 8.03 (s, 1H), 7.23-7.20 (m, 2H), 6.93 (dd, J=2.40 Hz and 12.00 Hz, 1H), 6.86 (dd, J=2.40 Hz and 8.80 Hz, 1H), 3.96-3.80 (m, 6H), 3.45-3.36 (m, 2H), 2.12 (s, 3H), 1.83 (d, J=10.80 Hz, 2H), 1.49-1.50 (m, 2H). MS ES⁺: 318.1.

### Example 46: 5-(2-fluoro-4-methoxyphenyl)-4-methyl-N-(tetrahydro-2H-pyran-3-yl) pyrimidin-2-amine

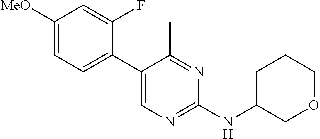 [SMILES: COc1ccc(-c2cnc(NC3CCCOC3)nc2C)c(F)c1]

Prepared as described for Example 45 using 5-bromo-4-methyl-N-(tetrahydro-2H-pyran-3-yl)pyrimidin-2-amine (0.250 g, 0.9 mmol) and (2-fluoro-4-methoxyphenyl)boronic acid (0.153 g, 0.9 mmol) to afford the title compound (0.16 g, 56%) as an off-white solid. ¹H NMR (400 MHz, DMSO-d₆): δ 8.03 (s, 1H), 7.25 (t, J=8.80 Hz, 1H), 7.10 (d, J=5.20 Hz, 1H), 6.93 (dd, J=2.80, 12.00 Hz, 1H), 6.86 (dd, J=2.80, 8.60 Hz, 1H), 3.87-3.73 (m, 6H), 3.27-3.25 (m, 1H), 3.08 (t, J=11.20 Hz, 1H), 2.13 (s, 3H), 1.98-1.90 (m, 1H), 1.72-1.5 (m, 3H). MS ES⁺: 318.28.

### Example 47: 5-(2-chloro-4-methoxyphenyl)-4-methyl-N-((tetrahydrofuran-2-yl)methyl) pyrimidin-2-amine

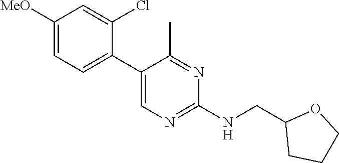 [SMILES: COc1ccc(-c2cnc(NCC3CCCO3)nc2C)c(Cl)c1]

Prepared as described for Example 45 using 5-bromo-4-methyl-N-((tetrahydrofuran-2-yl)methyl)pyrimidin-2-amine (0.250 g, 0.9 mmol) and (2-chloro-4-methoxyphenyl)boronic acid (0.153 g, 0.9 mmol) to afford the title compound (0.16 g, 56%) as an off-white solid. ¹H NMR (400 MHz, DMSO-d₆): δ 7.95 (s, 1H), 7.26 (d, J=8.80 Hz, 1H), 7.16 (d, J=2.80 Hz, 2H), 6.99 (dd, J=2.40 Hz and 8.60 Hz, 1H), 4.05-3.95 (m, 1H), 3.85-3.76 (m, 4H), 3.65-3.55 (m, 1H), 3.45-3.35 (m, 1H), 3.30-3.25 (m, 1H), 2.06 (s, 3H), 1.95-1.75 (m, 3H), 1.65-1.55 (m, 1H). MS ES⁺: 334.25.

### Example 48: 5-(2-fluoro-4-methoxyphenyl)-N-(1-isopropylpiperidin-3-yl)-4-methyl-pyrimidin-2-amine, hydrochloride salt

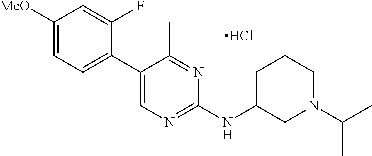 [SMILES: COc1ccc(-c2cnc(NC3CCCN(C(C)C)C3)nc2C)c(F)c1 ; Cl]

A solution of 2-chloro-5-(2-fluoro-4-methoxyphenyl)-4-methyl-pyrimidine (0.27 g, 1.07 mmol) in NMP (5 mL), 1-isopropylpiperidin-3-amine (0.23 g, 1.68 mmol) and triethylamine (0.8 ml, 5.3 mmol) was stirred at 180° C. for 4 h (microwave). The mixture was cooled to RT, treated with H₂O and extracted with EtOAc. The combined organic layers were washed with brine, dried over Na₂SO₄ and concentrated. Purification of the residue by prep-HPLC gave 5-(2-fluoro-4-methoxyphenyl)-N-(1-isopropylpiperidin-3-yl)-4-methyl-pyrimidin-2-amine as a sticky solid. The compound was subjected to HCl salt formation by stirring a solution of the material in dioxane (4 mL) with 2 mL of 4M HCl in dioxane at RT for 3 h. The solution was evaporated in vacuo and lyophilized to obtain the title compound as hydrochloride (0.20 g, 93%) as a brown solid. ¹H-NMR (400 MHz, DMSO-d₆): δ 10.1 (s, 1H), 8.12 (s, 1H), 7.79-7.49 (m, 1H), 7.29-7.24 (m, 1H), 6.95 (dd, J=2.80 Hz and 12.00 Hz, 1H), 6.88 (dd, J=2.40 Hz and 8.40 Hz, 1H), 4.25 (s, 1H), 3.85 (s, 3H), 3.51-3.46 (m, 2H), 3.39-3.31 (m, 1H), 2.95-2.83 (m, 1H), 2.73-2.65 (m, 11H), 2.17 (s, 3H), 2.01-1.81 (m, 3H), 1.61-1.55 (m, 1H), and 1.31-1.20 (m, 6H). MS ES⁺: 359.38.

### Example 49: 5-(2-fluoro-4-methoxyphenyl)-N-((1-isopropylpiperidin-3-yl)methyl)-4-methyl-pyrimidin-2-amine, hydrochloride salt

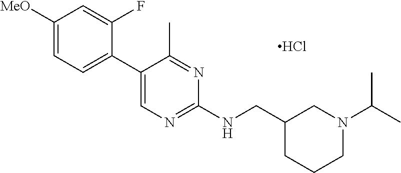 [SMILES: COc1ccc(-c2cnc(NCC3CCCN(C(C)C)C3)nc2C)c(F)c1 ; Cl]

Prepared as described for Example 10 using 2-chloro-5-(2-fluoro-4-methoxyphenyl)-4-methyl-pyrimidine (0.25 g, 0.992 mmol) and (1-isopropylpiperidin-3-yl)methanamine (0.24 g, 1.48 mmol) to afford the title compound (0.21 g, 51%) as an off-white solid. ¹H-NMR (400 MHz, DMSO-d₆): δ 9.85 (s, 1H), 8.13 (s, 1H), 7.72 (s, 1H), 7.28 (t, J=8.80 Hz, 1H), 6.95 (dd, J=2.40 Hz and 12.00 Hz, 1H), 6.88 (dd, J=2.80 Hz and 9.60 Hz, 1H), 3.81 (s, 3H), 3.45-3.28 (m, 5H), 2.85-2.75 (m, 1H), 2.75-2.65 (m, 1H), 2.30-2.15 (m, 4H), 1.90-1.75 (m, 3H), and 1.30-1.15 (m, 7H). MS ES⁺: 373.41.

### Example 50: 5-(2-fluoro-4-methoxyphenyl)-N-((1-isopropylpiperidin-2-yl)methyl)-4-methyl-pyrimidin-2-amine, hydrochloride salt

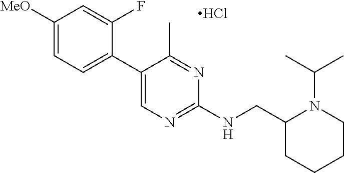 [SMILES: COc1ccc(-c2cnc(NCC3CCCCN3C(C)C)nc2C)c(F)c1 ; Cl]

Prepared as described for Example 10 using 2-chloro-5-(2-fluoro-4-methoxyphenyl)-4-methyl-pyrimidine (0.3 g, 1.19 mmol) and (1-isopropylpiperidin-2-yl)methanamine (0.28 g, 1.78 mmol) to afford the title compound (0.2 g, 49%) as a brown solid. ¹H-NMR (400 MHz, DMSO-d₆): δ 9.75 (s, 1H), 8.10 (d, J=3.20 Hz, 1H), 7.62 (t, J=6.00 Hz, 1H), 7.28-7.24 (m, 1H), 6.95 (dd, J=2.40 Hz and 12.00 Hz, 1H), 6.88 (dd, J=2.80 Hz and 8.60 Hz, 1H), 4.10 (m, 1H), 3.80 (s, 4H), 3.50-3.30 (m, 3H), 2.84 (t, J=2.80 Hz, 1H), 2.15 (s, 3H), 2.02 (d, J=12.40 Hz, 1H), 1.75-1.60 (m, 4H), 1.55-1.45 (m, 1H), 1.35-1.20 (m, 6H). MS ES⁺: 373.41.

### Example 51: 5-(2-fluoro-4-methoxyphenyl)-4-methyl-N-((tetrahydro-2H-pyran-3-yl) methyl)pyrimidin-2-amine

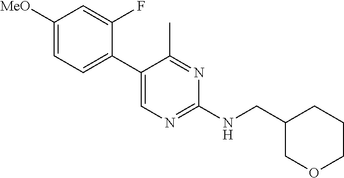 [SMILES: COc1ccc(-c2cnc(NCC3CCCOC3)nc2C)c(F)c1]

Prepared as described for Example 2 using 2-chloro-5-(2-fluoro-4-methoxyphenyl)-4-methyl-pyrimidine (0.18 g, 0.7 mmol) and (tetrahydro-2H-pyran-3-yl)methanamine (0.088 g, 0.77 mmol) to afford the title compound (0.055 g, 21%) as a brown solid. ¹H NMR (400 MHz, DMSO-d₆): δ 8.10 (s, 1H), 7.60 (s, 1H), 7.27 (t, J=8.80 Hz, 1H), 6.95 (dd, J=2.80 Hz and 12.00 Hz, 1H), 6.88 (dd, J=2.80 Hz and 8.60 Hz, 1H), 3.79 (m, 4H), 3.75-3.65 (m, 1H), 3.34 (t, J=8.00 Hz, 1H), 3.23 (s, 2H), 3.12 (m, 1H), 2.17 (s, 3H), 1.90-1.75 (m, 2H), 1.65-1.55 (m, 1H), 1.50-1.40 (m, 1H), 1.30-1.20 (m, 1H). MS ES⁺: 332.10.

### Example 52: 5-(2,4-difluorophenyl)-4-methyl-N-(3-(methylsulfonyl)cyclopentyl) pyrimidin-2-amine

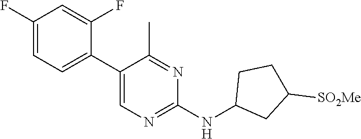 [SMILES: Cc1nc(NC2CCC(S(C)(=O)=O)C2)ncc1-c1ccc(F)cc1F]

A solution of 2-chloro-5-(2,4-difluorophenyl)-4-methyl-pyrimidine (0.10 g, 0.40 mmol) in toluene (3 mL) was treated with 3-(methylsulfonyl)cyclopentan-1-amine (0.74 g, 0.4 mmol) and Cs₂CO₃ (0.19 g, 0.6 mmol), purged with N₂ for 5-10 min, treated with Pd(OAc)₂ (0.002 g, 0.012 mmol) and BINAP (0.007 g, 0.012 mmol) and stirred for 16 h at 100° C. in a sealed tube. The solvent was evaporated and the residue was treated with H₂O and extracted with EtOAc. The organic layer was dried over Na₂SO₄ and concentrated. Purification of the residue by reverse phase column chromatography (60-65% of MeOH in a 0.1% solution of ammonium bicarbonate in H₂O) gave the title compound (0.75 g, 51%) as an off-white solid. ¹H NMR (400 MHz, DMSO-d₆): δ 8.06 (d, J=3.4 Hz, 1H), 7.48-7.30 (m, 3H), 7.25-7.15 (m, 11H), 4.35 (m, 1H), 3.70-3.65 (m, 1H), 2.95 (s, 3H), 2.45-1.60 (m, 9H). MS ES⁺: 368.27.

### Example 53a/b: 5-(2-fluoro-4-methoxyphenyl)-N-(3-methoxycyclohexyl)-4-methyl-pyrimidin-2-amine, hydrochloride salt

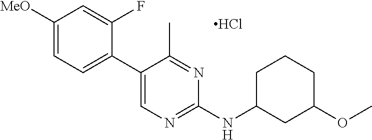 [SMILES: COc1ccc(-c2cnc(NC3CCCC(OC)C3)nc2C)c(F)c1 ; Cl]

Prepared as described for Example 2 using 2-chloro-5-(2-fluoro-4-methoxyphenyl)-4-methyl-pyrimidine (0.30 g, 1.2 mmol) and 3-methoxycyclohexan-1-amine (0.17 g, 1.32 mmol). The crude product was purified by column chromatography on amine silica (Davisil) to afford the individual diastereomers of the title compound, which were subjected to hydrochloride salt formation to afford isomer 1 (0.03 g, 10%) and isomer 2 (0.035 g, 12%) as off-white sticky solids.

Example 53a (isomer 1): ¹H NMR (400 MHz, DMSO-d₆): δ 8.09 (s, 1H), 7.43 (s, 1H), 7.27 (t, J=8.80 Hz, 1H), 6.95 (dd, J=2.40, 12.00 Hz, 1H), 6.88 (dd, J=2.40, 8.40 Hz, 1H), 4.07 (s, 1H), 3.81 (s, 3H), 3.57 (s, 1H), 3.24 (s, 3H), 2.16 (s, 3H), 2.01 (s, 1H), 1.81 (s, 1H), 1.68 (s, 1H), 1.55-1.49 (m, 4H), 1.40-1.25 (m, 2H). MS ES⁺: 346.31.

Example 53b (isomer 2): ¹H NMR (400 MHz, DMSO-d₆): δ 8.07 (s, 1H), 7.38 (s, 1H), 7.25 (t, J=3.60 Hz, 1H), 6.94 (dd, J=2.40, 12.00 Hz, 1H), 6.87 (dd, J=2.40, 8.40 Hz, 1H), 3.81 (s, 4H), 3.25 (s, 4H), 2.22 (s, 1H), 2.15 (s, 3H), 1.97 (s, 1H), 1.86 (s, 1H), 1.71-1.72 (m, 1H), 1.17-1.19 (m, 5H). MS ES⁺: 346.31.

### Example 54a/b: 6-(2-fluoro-4-methoxyphenyl)-5-methyl-N-((1-methylpyrrolidin-2-yl) methyl)-1,2,4-triazin-3-amine

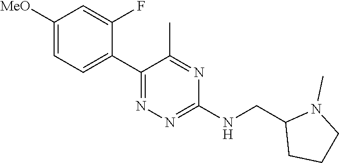 [SMILES: COc1ccc(-c2nnc(NCC3CCCN3C)nc2C)c(F)c1]

A stirred solution of 6-(2-fluoro-4-methoxyphenyl)-5-methyl-3-(methylsulfonyl)-1,2,4-triazine (0.70 g, 2.4 mmol) and (1-methylpyrrolidin-2-yl)methanamine (0.30 g, 2.64 mmol) in dioxane was heated in a sealed tube at 110° C. for 1.5 h and evaporated. Purification of the residue by prep-HPLC followed by SFC (YMC-SC (30 mm×250 mm, 5 μm); CO₂ (60%), co-solvent (40%, 0.2% 7M NH₃ in MeOH); total flow=110.0 g/min, T=30° C.; UV detection at 250 nm) to obtain both enantiomers as pale brown sticky solids: isomer 1 (0.175 g, 23%) and isomer 2 (0.171 g, 23.1%).

Example 54a (isomer 1): ¹H NMR (400 MHz, DMSO-d₆): δ 7.64 (br s, 1H), 7.43 (t, J=8.80 Hz, 1H), 6.97 (dd, J=2.40 Hz and 8.40 Hz, 1H), 6.91 (dd, J=2.4 Hz and 8.4 Hz, 1H), 3.81 (s, 3H), 3.61 (s, 1H), 3.25-3.15 (m, 1H), 3.00-2.95 (m, 1H), 2.49 (s, 1H), 2.32 (s, 3H), 2.20-2.10 (m, 4H), 1.90-1.85 (m, 1H), 1.70-1.55 (m, 3H). MS ES⁺: 332.29. Chiral HPLC: 99.8%, 3.49 min. SOR: −54.3 (c 0.1, MeOH).

Example 54b (isomer 2): ¹H NMR (400 MHz, DMSO-d₆): δ 7.65 (br s, 1H), 7.43 (t, J=8.80 Hz, 1H), 6.95 (dd, J=2.40 Hz and 8.40 Hz, 1H), 6.92 (dd, J=2.40 Hz and 8.4 Hz, 1H), 3.81 (s, 3H), 3.61 (s, 1H), 3.26-3.22 (m, 1H), 3.00-2.90 (m, 1H), 2.40 (br s, 1H), 2.32 (s, 3H), 2.20-2.10 (m, 4H), 1.92-1.86 (m, 1H), 1.68-1.53 (m, 3H). MS ES⁺: 332.29. Chiral HPLC: 99.7%, 6.4 min. SOR: +54.7 (c 0.1, MeOH).

### Example 55a/b: N¹-(5-(2,4-difluorophenyl)-4-methyl-pyrimidin-2-yl)-N³,N³-dimethylcyclohexane-1,3-diamine

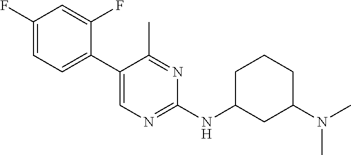 [SMILES: Cc1nc(NC2CCCC(N(C)C)C2)ncc1-c1ccc(F)cc1F]

Prepared as described for Example 2 using 2-chloro-5-(2,4-difluorophenyl)-4-methyl-pyrimidine (0.21 g, 0.875 mmol) and N¹,N¹-dimethylcyclohexane-1,3-diamine (0.150 g 1.05 mmol) to afford the title compound, which was subjected to separation of isomers by SFC to afford isomer 1 (0.05 mg, 17%) and isomer 2 (0.05 mg, 16%) as off-white solids.

Example 55a (isomer 1): ¹H NMR (400 MHz, DMSO-d₆): δ 8.047 (s, 1H), 7.45-7.32 (m, 2H), 7.19-7.11 (m, 2H), 4.16 (t, d, J=8.00 Hz, 1H), 2.37-2.32 (m, 1H), 2.21 (s, 6H), 2.11 (s, 3H), 1.90-1.88 (m, 1H), 1.68-1.46 (m, 7H). MS ES⁺: 347.36.

Example 55b (isomer 2): ¹H NMR (400 MHz, DMSO-d₆): δ 8.057 (s, 1H), 7.44-7.27 (m, 3H), 7.19-7.16 (m, 1H), 3.81 (s, 1H), 2.69 (s, 1H), 2.41 (s, 6H), 2.12 (m, 4H), 1.90-1.75 (m, 1H), 1.40-1.10, (m, 6H). MS ES⁺: 347.28.

### Example 56a/b: 2-((3-(dimethylamino)cyclohexyl)amino)-5-(2-fluoro-4-methoxyphenyl) pyrimidine-4-carbonitrile

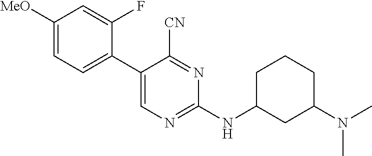 [SMILES: COc1ccc(-c2cnc(NC3CCCC(N(C)C)C3)nc2C#N)c(F)c1]

Prepared as described for Example 19 using 5-(2-fluoro-4-methoxyphenyl)-2-(methylsulfonyl)pyrimidine-4-carbonitrile (0.20 g, 0.70 mmol) and N¹,N¹-dimethylcyclohexane-1,3-diamine (0.10 g, 0.70 mmol) to afford the title compound, which was subjected to separation of isomers by SFC to afford isomer 1 (0.07 g, 48%) and isomer 2 (0.05 g, 42%) as off-white solids.

Example 56a (isomer 1): ¹H NMR (400 MHz, DMSO-d₆): δ 8.56 (d, J=13.20 Hz, 1H), 7.99 (s, 1H), 7.46 (t, J=8.80 Hz, 1H), 7.03 (dd, J=2.40, 12.40 Hz, 1H), 6.94 (dd, J=2.40, 8.80 Hz, 1H), 3.83-3.77 (m, 4H), 2.28-2.17 (m, 7H), 2.02 (d, J=11.20 Hz, 1H), 1.89-1.70 (m, 3H), 1.30-1.05 (m, 4H). MS ES⁺: 370.28.

Example 56b (isomer 2): ¹H NMR (400 MHz, DMSO-d₆): δ 8.56 (s, 1H), 7.93 (s, 1H), 7.47 (t, J=8.80 Hz, 1H), 7.03 (dd, J=2.40, 12.40 Hz, 1H), 6.94 (dd, J=2.40, 8.40 Hz, 1H), 4.19-4.09 (m, 1H), 3.83 (s, 3H), 2.32 (m, 1H), 2.19-2.15 (m, 6H), 2.00-1.40 (m, 8H). MS ES⁺: 370.21.

### Example 57a/b: 6-(2-fluoro-4-methoxyphenyl)-5-methyl-N-((1-methylpiperidin-2-yl) methyl)-1,2,4-triazin-3-amine

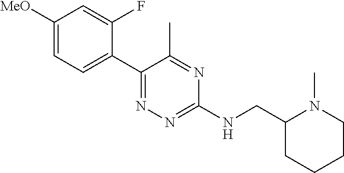 [SMILES: COc1ccc(-c2nnc(NCC3CCCCN3C)nc2C)c(F)c1]

Prepared as described for Example 15 using 6-(2-fluoro-4-methoxyphenyl)-5-methyl-3-(methylsulfonyl)-1,2,4-triazine (0.54 g, 1.82 mmol) and (1-methylpiperidin-2-yl)methanamine (0.254 g, 2.00 mmol) to afford the title compound as a light brown gummy solid, which was subjected to separation of enantiomers by SFC (Chiralpak AD-H (30 mm×250 mm, 5 μm); CO₂ (70%), co-solvent (30%, 0.2% 7M ammonia in MeOH/ACN 1:1); total flow=110.0 g/min; T=30° C.; UV detection at 230 nm) to give isomer 1 (0.255 g, 33%) and isomer 2 (0.265 g, 33%) as light brown sticky solids.

Example 57a (isomer U: ¹H NMR (400 MHz, DMSO-d₆): δ 7.61 (br s, 1H), 7.41 (t, J=8.40 Hz, 1H), 6.95 (dd, J=2.40, 8.40 Hz, 2H), 3.81 (s, 3H), 3.61 (s, 1H), 3.35 (s, 1H), 2.76 (d, J=11.20 Hz, 1H), 2.27 (s, 3H), 2.20 (s, 3H), 2.15-2.00 (m, 2H), 1.75-1.60 (m, 2H), 1.55-1.40 (m, 2H), 1.35-1.15 (m, 2H). MS ES⁺: 346.35. Chiral HPLC: 99.51%, 5.93 min.

Example 57b (isomer 2): ¹H NMR (400 MHz, DMSO-d₆): δ 7.60 (br s, 1H), 7.41 (t, J=8.8 Hz, 1H), 6.95 (dd, J=2.40, 8.40 Hz, 2H), 3.84 (s, 3H), 3.65 (s, 1H), 3.32 (s, 1H), 2.77 (d, J=11.20 Hz, 1H), 2.30 (s, 3H), 2.20 (s, 3H), 2.15-1.99 (m, 2H), 1.72-1.65 (m, 2H), 1.55-1.40 (m, 2H), 1.35-1.15 (m, 2H). MS ES⁺: 346.35. Chiral HPLC: 99.3%, 9.44 min.

### Example 58a/b: 5-(2,4-difluorophenyl)-2-((3-(dimethylamino)cyclopentyl)amino) pyrimidine-4-carbonitrile

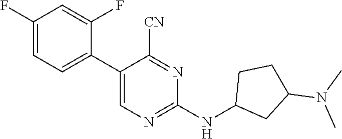 [SMILES: CN(C)C1CCC(Nc2ncc(-c3ccc(F)cc3F)c(C#N)n2)C1]

Prepared as described for Example 19 using 5-(2,4-difluorophenyl)-2-(methylsulfonyl) pyrimidine-4-carbonitrile (1.2 g, 4.0 mmol) and N¹,N¹-dimethylcyclopentane-1,3-diamine (0.70 g, 5.4 mmol) to afford the title compound, which was subjected to separation of isomers by SFC to afford isomer 1 (0.337 g, 24%) and isomer 2 (0.244 g, 18%) as off-white solids.

Example 58a (isomer 1): ¹H NMR (400 MHz, DMSO-d₆): δ 8.04 (s, 1H), 7.45-7.25 (m, 3H), 7.18 (t, J=2.00 Hz, 1H), 4.21 (d, J=6.80 Hz, 1H), 2.45 (t, J=6.80 Hz, 1H), 2.12 (s, 7H), 1.89-1.91 (m, 1H), 1.74 (q, J=10.40 Hz, 1H), 1.54-1.55 (m, 2H), 1.42-1.30 (m, 1H). MS ES⁺: 333.32.

Example 58b (isomer 2): ¹H NMR (400 MHz, DMSO-d₆): δ 8.04 (s, 1H), 7.45-7.28 (m, 3H), 7.18 (t, J=2.00 Hz, 1H), 4.20 (s, 1H), 2.42-2.38 (m, 1H), 2.16 (s, 7H), 1.90-1.91 (m, 1H), 1.72 (m, 1H), 1.60-1.50 (m, 2H), 1.42-1.30 (m, 1H). MS ES⁺: 333.10.

### Example 59a/b: 5-(2,4-difluorophenyl)-2-((3-(dimethylamino)cyclohexyl)amino) pyrimidine-4-carbonitrile

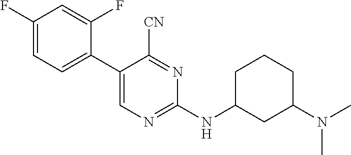 [SMILES: CN(C)C1CCCC(Nc2ncc(-c3ccc(F)cc3F)c(C#N)n2)C1]

Prepared as described for Example 19 using 5-(2,4-difluorophenyl)-2-(methylsulfonyl) pyrimidine-4-carbonitrile (0.3 g, 1 mmol) and N¹,N¹-dimethylcyclohexane-1,3-diamine (0.17 g, 1.35 mmol) to afford the title compound, which was subjected to separation of isomers by SFC (Lux Amylose-2 (21 mm×250 mm, 5 μm); CO₂ (82%), co-solvent (18% of 0.2% DIPEA in ACN/ⁱPrOH 1:1); total flow=60.0 g/min; T=30° C.; UV detection at 260 nm) to afford isomer 1 (0.023 g, 24%) and isomer 2 (0.010 g, 18%) as off-white solids.

Example 59a (isomer 1): ¹H NMR (400 MHz, DMSO-d₆): δ 8.60 (s, 1H), 8.04 (s, 1H), 7.68-7.60 (m, 1H), 7.50-7.42 (m, 1H), 7.30-7.22 (m, 1H), 4.16 (m, 1H), 2.40-2.20 (m, 7H), 2.00-1.40 (m, 8H). MS ES⁺: 358.28.

Example 59b (isomer 2): ¹H NMR (400 MHz, DMSO-d₆): δ 8.62 (d, J=11.60 Hz, 1H), 8.14 (d, J=6.80 Hz, 1H), 7.65-7.61 (m, 1H), 7.52-7.45 (m, 1H), 7.32-7.25 (m, 1H), 3.81 (d, J=14.40 Hz, 1H), 2.55 (m, 1H), 2.40-2.25 (m, 6H), 2.15-2.05 (m, 1H), 1.90-1.75 (m, 3H), 1.40-1.10 (m, 4H). MS ES⁺: 358.28.

### Example 60: 5-(2,4-difluorophenyl)-4-methyl-N-(1-(2,2,2-trifluoroethyl)piperidin-3-yl) pyrimidin-2-amine

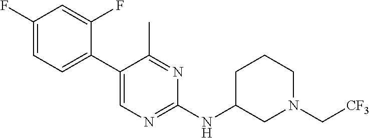 [SMILES: Cc1nc(NC2CCCN(CC(F)(F)F)C2)ncc1-c1ccc(F)cc1F]

Prepared as described for Example 2 using 2-chloro-5-(2,4-difluorophenyl)-4-methyl-pyrimidine (0.150 g, 0.6 mmol) and 1-(2,2,2-trifluoroethyl)piperidin-3-amine (0.091 g, 0.60 mmol) to afford the title compound (0.04 g, 17%) as a white solid. ¹H NMR (400 MHz, DMSO-d₆): δ 8.07 (s, 1H), 7.45-7.32 (m, 2H), 7.20-7.08 (m, 2H), 3.99 (s, 1H), 3.25-3.17 (m, 2H), 3.06 (d, J=8.00 Hz, 1H), 2.82 (d, J=11.20 Hz, 1H), 2.33 (t, J=5.20 Hz, 1H), 2.24 (q, J=10.00 Hz, 1H), 2.12 (s, 3H), 1.85-1.75 (m, 1H), 1.72-1.62 (m, 1H), 1.62-1.45 (m, 1H), 1.35-1.20 (m, 1H). MS ES⁺: 387.32.

### Example 61: 5-(2-fluoro-5-methoxyphenyl)-4-methyl-N-((4-methylmorpholin-3-yl) methyl)pyrimidin-2-amine, hydrochloride salt

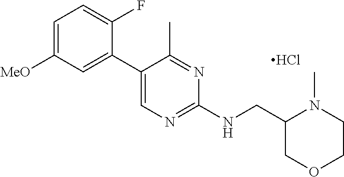 [SMILES: COc1ccc(F)c(-c2cnc(NCC3COCCN3C)nc2C)c1 ; Cl]

Prepared as described for Example 45 using 5-bromo-4-methyl-N-((4-methylmorpholin-3-yl)methyl)pyrimidin-2-amine (0.20 g, 0.70 mmol) and (2-fluoro-5-methoxyphenyl)boronic acid (0.138 g, 0.77 mmol) to afford the title compound (0.07 g, 30%) as an off-white solid. ¹H NMR (400 MHz, DMSO-d₆): δ 10.78 (s, 1H), 8.18 (s, 1H), 7.62 (t, J=5.9 Hz, 1H), 7.24 (t, J=9.2 Hz, 1H), 6.99 (m, J=3.2 Hz, 1H), 6.89 (q, J=3.1 Hz, 1H), 4.07 (t, J=6.4 Hz, 1H), 3.94 (d, J=10.3 Hz, 1H), 3.77 (s, 5H), 3.60 (m, J=6.1 Hz, 2H), 3.39 (t, J=14.0 Hz, 2H), 3.22 (m, J=7.3 Hz, 1H), 2.98 (d, J=4.0 Hz, 3H), 2.21 (s, 3H). MS ES⁺: 347.32.

### Example 62: 5-(2-chloro-4-methoxyphenyl)-4-methyl-N-((4-methylmorpholin-3-yl) methyl)pyrimidin-2-amine

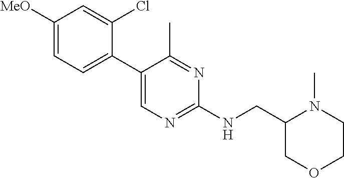 [SMILES: COc1ccc(-c2cnc(NCC3COCCN3C)nc2C)c(Cl)c1]

Prepared as described for Example 45 using 5-bromo-4-methyl-N-((4-methylmorpholin-3-yl)methyl)pyrimidin-2-amine (0.2 g, 0.7 mmol) and (2-chloro-4-methoxyphenyl)boronic acid (0.128 g, 0.77 mmol) to afford the title compound (0.05 g, 20%) as a brown sticky solid. ¹H NMR (400 MHz, DMSO-d₆): δ 7.96 (s, 1H), 7.27 (d, J=8.5 Hz, 1H), 7.16 (d, J=2.6 Hz, 1H), 7.01 (m, J=4.4 Hz, 2H), 3.82 (s, 3H), 3.76 (q, J=4.6 Hz, 1H), 3.66 (d, J=11.0 Hz, 1H), 3.60 (t, J=13.9 Hz, 1H), 3.47 (m, J=4.8 Hz, 1H), 3.26 (t, J=10.4 Hz, 1H), 3.16 (q, J=6.5 Hz, 1H), 2.64 (d, J=11.8 Hz, 1H), 2.30 (s, 3H), 2.20 (m, J=5.7 Hz, 2H), 2.06 (s, 3H). MS ES⁺: 363.32.

### Example 63: 5-(2-chloro-4-methoxyphenyl)-4-methyl-N-((1-methyl-1H-imidazol-5-yl) methyl)pyrimidin-2-amine

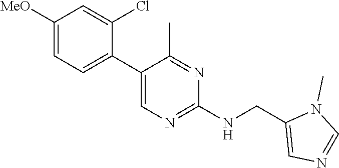 [SMILES: COc1ccc(-c2cnc(NCc3cncn3C)nc2C)c(Cl)c1]

Prepared as described for Example 2 using 2-chloro-5-(2-chloro-4-methoxyphenyl)-4-methyl-pyrimidine (0.15 g, 0.6 mmol) and (1-methyl-1H-imidazol-5-yl)methanamine (0.086 g, 0.78 mmol) to afford the title compound (0.019 g, 10%) as a brown sticky solid. ¹H NMR (400 MHz, DMSO-d₆): δ 7.99 (s, 1H), 7.59 (t, J=5.5 Hz, 1H), 7.52 (s, 1H), 7.27 (d, J=8.5 Hz, 1H), 7.16 (d, J=2.5 Hz, 1H), 6.99 (q, J=3.7 Hz, 1H), 6.82 (s, 1H), 4.47 (d, J=5.6 Hz, 2H), 3.82 (s, 3H), 3.65 (s, 3H), 2.08 (s, 3H). MS ES⁺: 344.0.

### Example 64a/b: 5-(2,4-difluorophenyl)-4-methyl-N-(1-(1-methylpyrrolidin-3-yl)ethyl) pyrimidin-2-amine

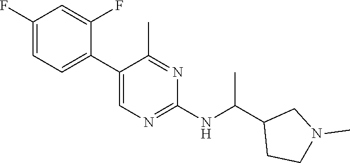 [SMILES: Cc1nc(NC(C)C2CCN(C)C2)ncc1-c1ccc(F)cc1F]

Prepared as described for Example 2 using 2-chloro-5-(2,4-difluorophenyl)-4-methyl-pyrimidine (0.8 g, 3.33 mmol) and 1-(1-methylpyrrolidin-3-yl)ethan-1-amine (0.64 g, 4.99 mmol) to afford the title compound. The racemic compound was subjected to separation of isomers by SFC to obtain isomer 1 (0.05 g, 10%) and isomer 2 (0.05 g, 10%) as off-white solids.

Example 64a (isomer U: ¹H NMR (400 MHz, DMSO-d₆): δ 8.03 (s, 1H), 7.45-7.32 (m, 2H), 7.20-7.10 (m, 2H), 3.91 (m, 1H), 2.59 (t, J=8.3 Hz, 1H), 2.43 (d, J=28.8 Hz, 1H), 2.30 (q, J=7.2 Hz, 2H), 2.20 (d, J=6.4 Hz, 3H), 2.11 (s, 4H), 1.84 (m, J=3.2 Hz, 1H), 1.51 (m, J=4.4 Hz, 1H), 1.09 (q, J=5.2 Hz, 3H). MS ES⁺: 333.28.

Example 64b (isomer 2): ¹H NMR (400 MHz, DMSO-d₆): δ 8.03 (s, 1H), 7.45-7.32 (m, 2H), 7.21-7.10 (m, 2H), 3.91 (m, 1H), 2.60 (t, J=8.3 Hz, 1H), 2.43 (m, J=6.6 Hz, 1H), 2.29 (d, J=5.9 Hz, 2H), 2.20 (d, J=6.3 Hz, 3H), 2.11 (s, 4H), 1.90-1.80 (m, 1H), 1.55-1.45 (m, 1H), 1.09 (m, 3H). MS ES⁺: 333.32.

### Example 65: 5-(2,4-difluorophenyl)-2-((1-isopropylpiperidin-4-yl)amino)pyrimidine-4-carbonitrile

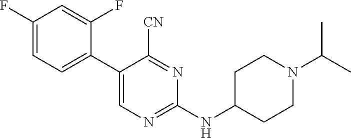 [SMILES: CC(C)N1CCC(Nc2ncc(-c3ccc(F)cc3F)c(C#N)n2)CC1]

A solution of 5-(2,4-difluorophenyl)-2-(methylsulfonyl)pyrimidine-4-carbonitrile (0.60 g, 2.0 mmol) and 1-isopropylpiperidin-4-amine (0.28 g, 0.21 mmol) in dioxane (10 ml) was stirred at 100° C. for 1 h (microwave) and evaporated. Purification of the residue by reverse phase column chromatography in automated instrument (Grace, 0.1% ammonium bicarbonate solution in H₂O and MeOH) gave the title compound (0.36 g, 52%) as an off-white solid. ¹H NMR (400 MHz, DMSO-d₆): δ 8.60 (d, J=10.6 Hz, 1H), 8.08 (s, 1H), 7.64 (q, J=8.0 Hz, 1H), 7.48 (m, J=4.4 Hz, 1H), 7.28 (m, J=3.8 Hz, 1H), 3.69 (d, J=6.8 Hz, 1H), 2.78 (d, J=11.7 Hz, 2H), 2.75-2.65 (m, 1H), 2.19 (d, J=5.5 Hz, 2H), 1.85 (d, J=10.2 Hz, 2H), 1.49 (d, J=9.8 Hz, 2H), 0.97 (d, J=6.2 Hz, 6H). MS ES⁺: 358.33.

### Example 66: 5-(2,4-difluorophenyl)-2-((1-methylpiperidin-4-yl)amino)pyrimidine-4-carbonitrile

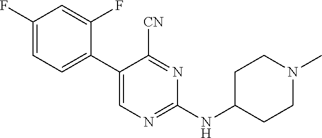 [SMILES: CN1CCC(Nc2ncc(-c3ccc(F)cc3F)c(C#N)n2)CC1]

Prepared as described for Example 10 using 5-(2,4-difluorophenyl)-2-(methylsulfonyl)pyrimidine-4-carbonitrile (0.30 g, 1.19 mmol) and 1-methylpiperidin-4-amine (0.15 g, 1.31 mmol) to afford the title compound (0.17 g, 58%) as an off-white solid. ¹H NMR (400 MHz, DMSO-d₆): δ 8.61 (d, J=9.60 Hz, 1H), 8.08 (s, 1H), 7.68-7.61 (m, 1H), 7.52-7.45 (m, 1H), 7.32-7.25 (m, 1H), 3.68 (s, 1H), 2.75 (d, J=11.60 Hz, 2H), 2.16 (s, 3H), 1.96 (d, J=9.60 Hz, 2H), 1.83 (d, J=11.20, Hz, 2H), 1.61-1.51 (m, 2H). MS ES⁺: 330.26.

### Example 67: 5-(2,4-difluorophenyl)-N-(1-isopropylpiperidin-4-yl)-4-methyl-pyrimidin-2-amine

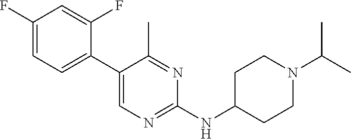 [SMILES: Cc1nc(NC2CCN(C(C)C)CC2)ncc1-c1ccc(F)cc1F]

Triethylamine (0.72 ml, 5.2 mmol) was added to a solution of 2-chloro-5-(2,4-difluorophenyl)-4-methyl-pyrimidine (0.50 g, 2.1 mmol) and 1-isopropylpiperidin-4-amine (0.357 g, 2.52 mmol) in EtOH (10 mL). The mixture was stirred for 4 h at 140° C. (microwave) and evaporated. Purification of the residue by column chromatography in automated instrument (Grace, 2-5% DCM in MeOH) gave the title compound (0.3 g), which was further purified by prep-HPLC to obtain the title compound (0.23 g, 32%) as an off-white solid. ¹H NMR (400 MHz, DMSO-d₆): δ 8.05 (s, 1H), 7.45-7.32 (m, 2H), 7.25-7.15 (m, 2H), 3.71 (s, 1H), 2.77 (s, 3H), 2.12 (s, 5H), 1.85 (s, 2H), 1.47 (s, 2H), 0.97 (s, 6H). MS ES⁺: 347.32.

### Example 68a/b: N¹-(5-(2,4-difluorophenyl)-4-methyl-pyrimidin-2-yl)-N³,N³-dimethylcyclobutane-1,3-diamine, hydrochloride salt

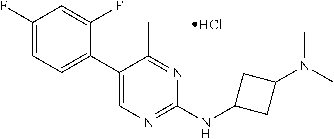 [SMILES: Cc1nc(NC2CC(N(C)C)C2)ncc1-c1ccc(F)cc1F ; Cl]

Prepared as described for Example 2 using 2-chloro-5-(2,4-difluorophenyl)-4-methyl-pyrimidine (0.30 g, 1.30 mmol) and N¹,N¹-dimethylcyclobutane-1,3-diamine hydrochloride (0.248 g, 1.60 mmol) to afford the title compound, which was subjected to separation of isomers by prep-HPLC in HCl medium to obtain isomer 1 (0.026 g, 8%) and isomer 2 (0.034 g, 10%) as off-white solids as hydrochloride salts.

Example 68a (isomer 1): ¹H NMR (400 MHz, DMSO-d₆): δ 10.72 (s, 1H), 8.12 (s, 1H), 7.93 (s, 1H), 7.45-7.35 (m, 2H), 7.22-7.15 (m, 1H), 4.33 (s, 1H), 3.81 (t, J=7.7 Hz, 1H), 2.68 (d, J=4.9 Hz, 8H), 2.35 (m, J=3.3 Hz, 2H), 2.16 (s, 3H). MS ES⁺: 319.33.

Example 68b (isomer 2): ¹H NMR (400 MHz, DMSO-d₆): δ 10.40 (s, 1H), 8.11 (s, 1H), 7.75 (s, 1H), 7.47-7.33 (m, 2H), 7.22-7.16 (m, 1H), 4.06 (s, 1H), 3.46 (d, J=8.2 Hz, 1H), 2.67 (d, J=4.9 Hz, 8H), 2.15 (m, 5H). MS ES⁺: 319.33.

### Example 69a/b: 6-(2-fluoro-4-methoxyphenyl)-5-methyl-N-(1-methylpiperidin-3-yl)-1,2,4-triazin-3-amine

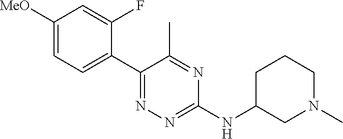 [SMILES: COc1ccc(-c2nnc(NC3CCCN(C)C3)nc2C)c(F)c1]

Prepared as described for Example 15 using 6-(2-fluoro-4-methoxyphenyl)-5-methyl-3-(methylsulfonyl)-1,2,4-triazine (0.80 g, 2.7 mmol) and 1-methylpiperidin-3-amine (0.380 g, 2.97 mmol) to afford the title compound, which was subjected to separation of isomers by SFC (Chiralpak IC (30 mm×250 mm, 5 μm); CO₂ (70%), co-solvent (30% of 0.2% 7M methanolic NH₃ in EtOH); total flow=100.0 g/min; T=30° C.; UV detection at 254) to obtain isomer 1 (0.125 g, 15%) and isomer 2 (0.07 g, 9%) as off-white solids.

Example 69a (isomer 1): ¹H NMR (400 MHz, DMSO-d₆): δ 7.43 (t, J=8.7 Hz, 2H), 6.95 (m, J=4.7 Hz, 2H), 4.00 (d, J=12.8 Hz, 1H), 3.84 (s, 3H), 2.87 (d, J=5.6 Hz, 1H), 2.62 (d, J=10.6 Hz, 1H), 2.18 (t, J=4.3 Hz, 6H), 1.86 (q, J=10.8 Hz, 3H), 1.69 (m, J=4.0 Hz, 1H), 1.53 (m, J=7.2 Hz, 1H), 1.31 (m, J=5.8 Hz, 1H). MS ES⁺: 332.32. Chiral HPLC: 99.98%, 7 min.

Example 69b (isomer 2): ¹H NMR (400 MHz, DMSO-d₆): δ 7.43 (t, J=8.7 Hz, 2H), 6.95 (m, J=4.7 Hz, 2H), 4.02 (s, 1H), 3.84 (s, 3H), 2.87 (s, 1H), 2.63 (d, J=10.6 Hz, 1H), 2.19 (t, J=2.9 Hz, 6H), 1.88 (t, J=11.1 Hz, 3H), 1.70 (m, J=4.0 Hz, 1H), 1.53 (d, J=12.8 Hz, 1H), 1.31 (q, J=5.0 Hz, 1H). MS ES⁺: 332.32. Chiral HPLC: 99.32%, 10.01 min.

### Example 70: 5-(2-fluoro-4-methoxyphenyl)-4,6-dimethyl-N-((1-methylpyrrolidin-2-yl) methyl)pyrimidin-2-amine, hydrochloride salt

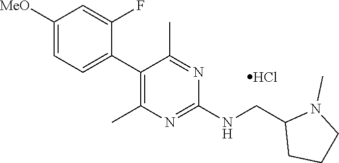 [SMILES: COc1ccc(-c2c(C)nc(NCC3CCCN3C)nc2C)c(F)c1 ; Cl]

Prepared as described for Example 10 using 2-chloro-5-(2-fluoro-4-methoxyphenyl)-4,6-dimethylpyrimidine (0.216 g, 0.81 mmol) and (1-methylpyrrolidin-2-yl)methanamine (0.109 g, 0.97 mmol) to obtain the title compound (0.040 g, 17%) as a gummy solid. ¹H NMR (400 MHz, DMSO-d₆): δ 10.38 (s, 1H), 7.67 (s, 1H), 7.21 (t, J=8.80 Hz, 1H), 6.95 (m, 1H), 6.85 (m, 1H), 3.85 (s, 3H), 3.80 (s, 2H), 3.60 (m, 2H), 3.15-3.05 (m, 1H), 2.95 (s, 3H), 2.15-2.10 (m, 1H), 2.05 (s, 6H), 2.00-1.95 (s, 1H), 1.90-1.80 (m, 2H). MS ES⁺: 345.34.

### Example 71: 5-(2,4-difluorophenyl)-4-methyl-N-(1-methylpiperidin-4-yl)pyrimidin-2-amine

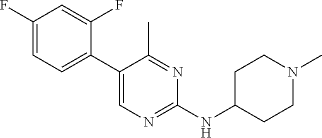 [SMILES: Cc1nc(NC2CCN(C)CC2)ncc1-c1ccc(F)cc1F]

Prepared as described for Example 2 using 2-chloro-5-(2,4-difluorophenyl)-4-methyl-pyrimidine (0.05 g, 0.2 mmol) and 1-methylpiperidin-4-amine (0.26 g, 0.3 mmol) to obtain the title compound (0.02 g, 40%) as an off-white solid. ¹H NMR (400 MHz, DMSO-d₆): δ 8.05 (s, 1H), 7.45-7.32 (m, 2H), 7.21-7.15 (m, 2H), 3.70 (br s, 1H), 2.74 (d, J=11.6 Hz, 2H), 2.15 (s, 3H), 2.11 (s, 3H), 1.94 (t, J=10.8 Hz, 2H), 1.82 (d, J=10.0 Hz, 2H), 1.52 (m, J=5.9 Hz, 2H). MS ES⁺: 319.33.

### Example 72: 5-(2,4-difluorophenyl)-N-(1-isopropylpiperidin-3-yl)-4-methyl-pyrimidin-2-amine

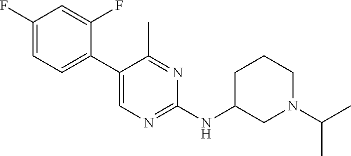 [SMILES: Cc1nc(NC2CCCN(C(C)C)C2)ncc1-c1ccc(F)cc1F]

Prepared as described for Example 2 using 2-chloro-5-(2,4-difluorophenyl)-4-methyl-pyrimidine (0.10 g, 0.40 mmol) and 1-isopropylpiperidin-3-amine hydrochloride (0.106 g, 0.60 mmol) to obtain the title compound (0.05 g, 35%) as an off-white solid. ¹H NMR (400 MHz, DMSO-d₆): δ 8.05 (s, 1H), 7.46-7.32 (m, 2H), 7.18 (m, J=3.3 Hz, 1H), 7.00 (s, 1H), 3.89 (s, 1H), 2.86 (d, J=7.6 Hz, 1H), 2.70 (q, J=6.7 Hz, 1H), 2.62 (d, J=10.9 Hz, 1H), 2.12 (s, 4H), 2.02 (t, J=9.8 Hz, 1H), 1.79 (m, J=4.0 Hz, 1H), 1.66 (m, J=4.0 Hz, 1H), 1.45 (q, J=11.6 Hz, 1H), 1.31 (m, J=5.4 Hz, 1H), 0.95 (q, J=2.7 Hz, 6H). MS ES⁺: 347.35.

### Example 73: (R)-5-(2-fluoro-4-(trifluoromethoxy)phenyl)-4-methyl-N-(pyrrolidin-2-ylmethyl)pyrimidin-2-amine, fumarate salt

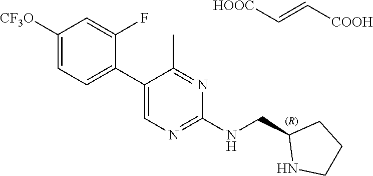

Step 1: A stirred solution of 2-chloro-5-(2-fluoro-4-(trifluoromethoxy)phenyl)-4-methylpyrimidine (300 mg, 0.98 mmol), tert-butyl (R)-2-(aminomethyl)pyrrolidine-1-carboxylate (294 mg, 1.47 mmol) in 1,4-dioxane (10 mL) was treated with DIPEA (190 mg, 1.47 mmol), stirred at 100° C. for 16 h and concentrated under reduced pressure. Purification of the residue by column chromatography (0 to 40% EtOAc/petrol ether) gave tert-butyl (R)-2-(((5-(2-fluoro-4-(trifluoromethoxy)phenyl)-4-methyl-pyrimidin-2-yl)amino)methyl)pyrrolidine-1-carboxylate (320 mg, 82%) as a thick liquid. ¹H NMR (400 MHz, DMSO-d₆): δ 8.07 (s, 1H), 7.53-7.49 (m, 2H), 7.34-7.32 (m, 2H), 3.96 (s, 1H), 3.55 (s, 1H), 3.25 (s, 3H), 2.14 (s, 3H), 1.85-1.76 (m, 4H), 1.39 (s, 9H).

Step 2: A stirred solution of tert-butyl (R)-2-(((5-(2-fluoro-4-(trifluoromethoxy)phenyl)-4-methyl-pyrimidin-2-yl)amino)methyl)pyrrolidine-1-carboxylate (320 mg, 0.68 mmol) in 1,4-dioxane (10 mL) was treated with 4M HCl in 1,4-dioxane (10 mL), stirred at RT for 3 h, and concentrated under reduced pressure. The residue was neutralized with an aq. NaOH solution and extracted with EtOAc (3×10 mL). The combined organic layers were concentrated under reduced pressure and the resulting residue was purified by prep-HPLC to afford (R)-5-(2-fluoro-4-(trifluoromethoxy)phenyl)-4-methyl-N-(pyrrolidin-2-ylmethyl)pyrimidin-2-amine as a thick liquid.

Step 3: A stirred solution of (R)-5-(2-fluoro-4-(trifluoromethoxy)phenyl)-4-methyl-N-(pyrrolidin-2-ylmethyl)pyrimidin-2-amine (140 mg, 0.378 mmol) in Et₂O (5 mL) was treated with fumaric acid (44 mg, 0.378 mmol, as a solution in MeOH, 2 mL) slowly and stirred at RT for 5 h. The mixture was concentrated under reduced pressure and the residue was triturated with n-pentane, filtered and dried under vacuum to give the title compound (180 mg, 54%) as an off-white solid. ¹H NMR (401 MHz, DMSO-d₆): δ 8.16 (s, 1H), 7.68 (br s, 11H), 7.55-7.50 (m, 2H), 7.35 (d, J=8.4 Hz, 1H), 6.48 (s, 2H), 3.70-3.50 (m, 3H), 3.20-3.10 (m, 2H), 2.17 (s, 3H), 2.08-2.00 (m, 1H), 1.95-1.75 (m, 2H), 1.70-1.60 (m, 1H). MS ES⁺: 372.3. SFC: 98.9%, 1.57 min.

### Example 74a/b: 6-(2,4-difluorophenyl)-5-methyl-N-((1-methylpyrrolidin-2-yl)methyl)-1,2,4-triazin-3-amine

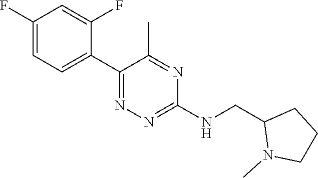 [SMILES: Cc1nc(NCC2CCCN2C)nnc1-c1ccc(F)cc1F]

Prepared as described for Example 15 using 6-(2,4-difluorophenyl)-5-methyl-3-(methylsulfonyl)-1,2,4-triazine (1.0 g, 3.5 mmol) and (1-methylpyrrolidin-2-yl)methanamine (0.48 g, 4.2 mmol) to obtain the title compound, which was subjected to separation of enantiomers by SFC (Chiralpak IC (30 mm×250 mm, 5 μm); CO₂ (55%), co-solvent (45%, 0.2% 7M NH₃ in MeOH); total flow=120.0 g/min; T=30° C.; UV detection at 250 nm) to obtain isomer 1 (0.35, 31%) as a pale brown gummy liquid and isomer 2 (0.35 g, 31%) as a pale brown gum.

Example 74a (isomer 1): ¹H NMR (400 MHz, DMSO-d₆): δ 7.84 (br s, 1H), 7.65-7.57 (m, 1H), 7.45-7.38 (m, 1H), 7.28-7.22 (m, 1H), 3.61 (br s, 1H), 3.28-3.18 (m, 1H), 2.98-2.90 (m, 1H), 2.41 (br s, 1H), 2.32 (s, 3H), 2.20 (s, 3H), 2.18-2.10 (m, 1H), 1.92-1.82 (m, 1H), 1.68-1.58 (m, 3H). MS ES⁺: 320.21. Chiral HPLC: 99.93%, 2.33 min.

Example 74b (isomer 2): ¹H NMR (400 MHz, DMSO-d₆): δ 7.84 (br s, 1H), 7.63-7.57 (m, 1H), 7.42 (t, J=2.40 Hz, 1H), 7.25 (t, J=2.40 Hz, 1H), 3.60 (br s, 1H), 3.30-3.18 (m, 1H), 2.98-2.90 (m, 1H), 2.40 (br s, 1H), 2.30 (s, 3H), 2.20 (s, 3H), 2.18-2.10 (m, 1H), 1.92-1.82 (m, 1H), 1.70-1.55 (m, 3H). MS ES⁺: 320.17. Chiral HPLC: 99.56%, 3.97 min.

### Example 75: 5-(2-fluoro-4-methoxyphenyl)-4-methoxy-6-methyl-N-((1-methylpyrrolidin-2-yl)methyl)pyrimidin-2-amine

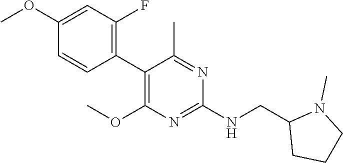 [SMILES: COc1ccc(-c2c(C)nc(NCC3CCCN3C)nc2OC)c(F)c1]

A solution of 5-bromo-4-methoxy-6-methyl-N-((1-methylpyrrolidin-2-yl)methyl) pyrimidin-2-amine (0.40 g, 1.27 mmol), (2-fluoro-4-methoxyphenyl)boronic acid (0.26 g, 1.52 mmol) and K₂CO₃ (0.53 g, 3.82 mmol) in H₂O and dioxane (4:1, 20 ml) in a glass tube was purged with N₂ for 10 min, treated with Pd(PPh₃)₄ (0.15 g, 0.1278 mmol), purged with N₂, sealed, and heated at 90-100° C. for 1.5 h. The mixture was cooled to RT, diluted with EtOAc and washed with H₂O and brine. The organic layer was dried over Na₂SO₄ and concentrated, and the residue purified by prep-HPLC to afford the title compound (0.10 g, 36%) as an off-white gummy solid. ¹H NMR (400 MHz, DMSO-d₆): δ 7.14 (t, J=8.7 Hz, 1H), 7.05 (br s, 1H), 6.85 (m, 1H), 6.80 (m, 1H), 3.74 (s, 3H), 3.71 (s, 3H), 3.60-3.52 (m, 1H), 3.15-3.00 (br s, 1H), 2.98-2.90 (m, 1H), 2.40-2.30 (m, 1H), 2.32 (s, 3H), 2.12 (q, J=8.4 Hz, 1H), 1.98 (s, 3H), 1.90-1.80 (m, 1H), 1.68-1.55 (m, 3H). MS ES⁺: 361.33. Chiral HPLC: 99.26%, 4.11 min. SOR: −56.1 (c 0.1, MeOH).

### Example 76a/b: 6-(2-fluoro-4-methoxyphenyl)-N-(1-isopropylpiperidin-3-yl)-5-methyl-1,2,4-triazin-3-amine

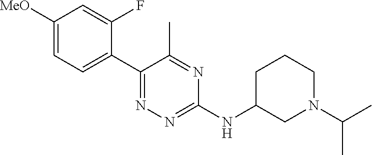 [SMILES: COc1ccc(-c2nnc(NC3CCCN(C(C)C)C3)nc2C)c(F)c1]

Prepared as described for Example 15 using 6-(2-fluoro-4-methoxyphenyl)-5-methyl-3-(methylsulfonyl)-1,2,4-triazine (1.0 g, 3.4 mmol) and 1-isopropylpiperidin-3-amine dihydrochloride (0.48 g, 3.74 mmol) to afford the title compound as the racemate, which was subjected to separation of enantiomers by SFC (Chiralpak IC (30 mm×250 mm, 5 μm); CO₂ (65%), co-solvent (35%, 0.2% 7M methanolic NH₃ in EtOH); total flow=110.0 g/min; T=35° C.; UV detection at 220 nm) to obtain isomer 1 (0.14 g, 12%) and isomer 2 (0.14 g, 12%) as off-white solids.

Example 76a (isomer 1): ¹H NMR (400 MHz, DMSO-d₆): δ 7.60 (br s, 1H), 7.43 (t, J=8.7 Hz, 1H), 6.95 (m, J=4.7 Hz, 2H), 3.97 (br s, 1H), 3.84 (s, 3H), 2.92 (d, J=7.4 Hz, 1H), 2.73 (q, J=6.6 Hz, 1H), 2.65 (d, J=10.9 Hz, 1H), 2.19 (s, 3H), 2.18-2.00 (m, 2H), 1.85 (d, J=8.8 Hz, 1H), 1.69 (m, J=3.9 Hz, 1H), 1.48 (d, J=10.9 Hz, 1H), 1.35 (q, J=4.8 Hz, 1H), 0.96 (d, J=6.5 Hz, 6H). MS ES⁺: 360.33. Chiral HPLC: 99.94%, 5.05 min. SOR: +33.86 (c 0.1, MeOH).

Example 76b (isomer 2): ¹H NMR (400 MHz, DMSO-d₆): δ 7.60 (br s, 1H), 7.43 (t, J=8.7 Hz, 1H), 6.95 (m, J=4.7 Hz, 2H), 3.99 (br s, 1H), 3.84 (s, 3H), 2.92 (d, J=8.2 Hz, 1H), 2.72 (t, J=6.6 Hz, 1H), 2.65 (t, J=5.4 Hz, 1H), 2.19 (s, 3H), 2.20-2.00 (m, 2H), 1.85 (d, J=8.8 Hz, 1H), 1.69 (q, J=5.4 Hz, 1H), 1.48 (d, J=11.1 Hz, 1H), 1.35 (q, J=4.9 Hz, 1H), 0.96 (d, J=6.5 Hz, 6H). MS ES⁺: 360.34. Chiral HPLC: 99.20%, 7.52 min. SOR: −34.00 (c 0.1, MeOH).

### Example 77a/b: 5-(2,4-difluorophenyl)-4-methyl-N-(1-methylpiperidin-3-yl)pyrimidin-2-amine

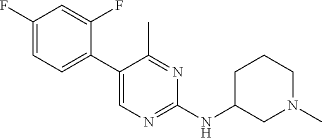 [SMILES: Cc1nc(NC2CCCN(C)C2)ncc1-c1ccc(F)cc1F]

Prepared as described for Example 2 using 2-chloro-5-(2,4-difluorophenyl)-4-methyl-pyrimidine (1.0 g, 4.2 mmol) and 1-methylpiperidin-3-amine (0.62 g, 5.46 mmol) to afford the title compound, which was subjected to separation of enantiomers by SFC to obtain isomer 1 (0.27 g, 20%) and isomer 2 (0.295 g, 22%) as off-white solids.

Example 77a (isomer 1): ¹H NMR (400 MHz, DMSO-d₆): δ 8.06 (s, 1H), 7.39 (m, J=4.5 Hz, 2H), 7.18 (m, J=3.2 Hz, 1H), 7.08 (br s, 1H), 3.92 (br s, 1H), 2.82 (d, J=9.0 Hz, 1H), 2.59 (d, J=10.4 Hz, 1H), 2.16 (s, 3H), 2.12 (s, 3H), 1.83 (m, J=11.2 Hz, 3H), 1.66 (m, J=4.1 Hz, 1H), 1.51 (m, J=6.6 Hz, 1H), 1.27 (t, J=11.2 Hz, 1H). MS ES⁺: 319.24. Chiral HPLC: 99.86%, 4.18 min. SOR: +31.00 (c 0.1, MeOH).

Example 77b (isomer 2): ¹H NMR (400 MHz, DMSO-d₆): δ 8.06 (s, 1H), 7.39 (m, J=4.5 Hz, 2H), 7.18 (m, J=2.5 Hz, 1H), 7.08 (br s, 1H), 3.92 (br s, 1H), 2.82 (d, J=8.0 Hz, 1H), 2.59 (d, J=10.8 Hz, 1H), 2.14 (s, 3H), 2.12 (s, 3H), 1.83 (m, J=9.1 Hz, 3H), 1.66 (m, J=4.0 Hz, 1H), 1.52 (t, J=11.4 Hz, 1H), 1.27 (t, J=11.4 Hz, 1H). MS ES⁺: 319.24. Chiral HPLC: 99.91%, 6.34 min. SOR: −31.00 (c 0.1, MeOH).

### Example 78a/b: 5-(2,4-difluorophenyl)-N-(1-ethylpiperidin-3-yl)-4-methyl-pyrimidin-2-amine

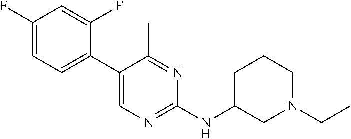 [SMILES: CCN1CCCC(Nc2ncc(-c3ccc(F)cc3F)c(C)n2)C1]

Prepared as described for Example 2 using 2-chloro-5-(2,4-difluorophenyl)-4-methyl-pyrimidine (0.50 g, 2.1 mmol) and 1-ethylpiperidin-3-amine dihydrochloride (0.63 g, 3.1 mmol) to afford the title compound (0.7 g, 65%), which was subjected to separation of isomers by SFC to obtain isomer 1 (0.2 g, 29%) and isomer 2 (0.2 g, 29%) as off-white solids.

Example 78a (isomer 1 (R)): ¹H NMR (400 MHz, DMSO-d₆): δ 8.06 (s, 1H), 7.39 (m, J=4.5 Hz, 2H), 7.18 (m, J=3.9 Hz, 1H), 7.05 (br s, 1H), 3.91 (br s, 1H), 2.90 (d, J=10.0 Hz, 1H), 2.69 (d, J=11.5 Hz, 1H), 2.33 (q, J=7.1 Hz, 2H), 2.12 (s, 3H), 1.86 (m, 3H), 1.67 (m, 1H), 1.49 (q, J=11.9 Hz, 1H), 1.30 (m, J=6.0 Hz, 1H), 0.98 (t, J=7.1 Hz, 3H). MS ES⁺: 333.36. Chiral HPLC: 99.95%, 3.75 min. SOR: +21.12 (c 0.1, MeOH).

Example 78b (isomer 2 (S)): ¹H NMR (400 MHz, DMSO-d₆): δ 8.06 (s, 1H), 7.39 (m, J=4.5 Hz, 2H), 7.18 (m, J=3.9 Hz, 1H), 7.05 (br s, 1H), 3.91 (br s, 1H), 2.90 (d, J=8.9 Hz, 1H), 2.69 (d, J=11.6 Hz, 1H), 2.33 (q, J=7.1 Hz, 2H), 2.12 (s, 3H), 1.86 (m, J=10.6 Hz, 3H), 1.67 (q, J=4.4 Hz, 1H), 1.49 (d, J=11.8 Hz, 1H), 1.31 (q, J=7.4 Hz, 1H), 0.98 (t, J=7.1 Hz, 3H). MS ES⁺: 333.32. Chiral HPLC: 99.28%, 4.87 min. SOR: −21.12 (c 0.1, MeOH).

### Example 78a: (R)-5-(2,4-difluorophenyl)-N-(1-ethylpiperidin-3-yl)-4-methyl-pyrimidin-2-amine, fumarate salt

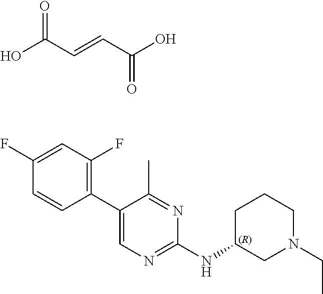

Step 1: A stirred solution of 2-chloro-5-(2,4-difluorophenyl)-4-methyl-pyrimidine (500 mg, 2.07 mmol) and (R)-1-ethylpiperidin-3-amine (399 mg, 3.11 mmol) in NMP (2.5 ml) was treated with DIPEA (2.14 g, 16.6 mmol) and allowed to stir for 1 h at 180° C. under microwave irradiation. The mixture was diluted with ice cold H₂O and extracted with EtOAc (3×15 ml). The combined organic layers were dried (Na₂SO₄) and concentrated under reduced pressure. Purification of the residue by prep-HPLC (XBridge C18, 19 mm×250 mm, 5 μm; mobile phase A: 10 mM ammonium bicarbonate in H₂O, mobile phase B: CH₃CN, 10-85% B) gave (R)-5-(2,4-difluorophenyl)-N-(1-ethylpiperidin-3-yl)-4-methyl-pyrimidin-2-amine (400 mg, 58%) as an off-white solid. MS ES⁺: 333.38.

Step 2: A stirred solution of (R)-5-(2,4-difluorophenyl)-N-(1-ethylpiperidin-3-yl)-4-methyl-pyrimidin-2-amine (400 mg, 1.20 mmol) in diethyl ether (5 ml) was treated with a solution of fumaric acid (139 mg, 1.20 mmol) in MeOH (0.1 ml) at RT, allowed to stir for 12 h and concentrated under reduced pressure. The residue was triturated with n-pentane and the remaining solids dried under vacuum to obtain the title compound (475 mg, 88%) as an off-white solid. ¹H NMR (400 MHz, DMSO-d₆): δ 12.97 (br s, 1H), 8.07 (s, 1H), 7.45-7.33 (m, 2H), 7.20-7.10 (m, 2H), 6.58 (s, 2H), 3.97 (br s, 1H), 3.05-2.95 (m, 1H), 2.85-2.75 (m, 1H), 2.50-2.42 (m, 2H), 2.12 (s, 3H), 2.10-1.95 (m, 2H), 1.85-180 (m, 1H), 1.75-1.65 (m, 1H), 1.60-1.45 (m, 1H), 1.40-1.30 (m, 1H), 1.02 (t, J=7.2 Hz, 3H). MS ES⁺: 333.21. HPLC: 99.7%, 4.83 min. Chiral SFC: 99.9%, 1.89 min.

### Example 78b: (S)-5-(2,4-difluorophenyl)-N-(1-ethylpiperidin-3-yl)-4-methyl-pyrimidin-2-amine, fumarate salt

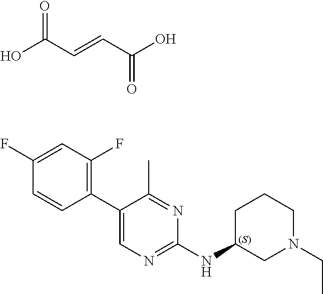

Step 1: A stirred solution of (S)-1-ethylpiperidin-3-amine dihydrochloride (2.00 g, 9.94 mmol) in absolute EtOH (10 ml) was treated with K₂CO₃ (1.65 g, 11.9 mmol) and stirred at RT for 1 h. The suspension was filtered and the filtrate concentrated in vacuo. The resulting residue was dissolved in NMP (10 ml) and treated with 2-chloro-5-(2,4-difluorophenyl)-4-methyl-pyrimidine (2.00 g, 8.40 mmol) and DIPEA (8.56 g, 66.5 mmol). The mixture was stirred for 1 h at 180° C. under microwave irradiation, cooled to RT, diluted with ice cold H₂O and extracted with EtOAc (3×15 ml). The combined organic layers were dried (Na₂SO₄) and concentrated under reduced pressure. Purification of the residue by prep-HPLC (XBridge C18, 19 mm×250 mm, 5 μm; mobile phase A: 10 mM ammonium bicarbonate in H₂O, mobile phase B: CH₃CN, 10-85% B) gave (S)-5-(2,4-difluorophenyl)-N-(1-ethylpiperidin-3-yl)-4-methyl-pyrimidin-2-amine (2.01 g, 75%) as an off-white solid. MS ES⁺: 333.17.

Step 2: A stirred solution of (S)-5-(2,4-difluorophenyl)-N-(1-ethylpiperidin-3-yl)-4-methyl-pyrimidin-2-amine (2.01 g, 6.33 mmol) in diethyl ether (20 ml) was treated with a solution of fumaric acid (0.70 g, 6.01 mmol) in MeOH (1 ml) at RT and stirred for 12 h at RT. Solvents were evaporated from the mixture under reduced pressure. The residue was triturated with n-pentane, the solids were collected and dried in vacuo to obtain the title compound (2.57 g, 91%) as an off-white solid. ¹H NMR (400 MHz, DMSO-d₆): δ 12.97 (br s, 1H), 8.07 (s, 1H), 7.45-7.32 (m, 2H), 7.20-7.10 (m, 2H), 6.58 (s, 2H) 3.96 (br s, 1H), 3.05-2.95 (m, 1H), 2.85-2.75 (m, 1H), 2.50-2.30 (m, 2H), 2.12 (s, 3H), 2.10-1.95 (m, 2H), 1.87-1.77 (m, 1H), 1.75-1.65 (m, 1H), 1.60-1.45 (m, 1H), 1.40-1.30 (m, 1H), 1.02 (t, J=7.2 Hz, 3H). MS ES⁺: 333.34. HPLC: 98.5%, 3.15 min. Chiral SFC: 99.7%, 1.08 min.

### Example 79a/b: 5-(2,4-difluorophenyl)-4-methyl-N-((4-methylmorpholin-3-yl)methyl) pyrimidin-2-amine

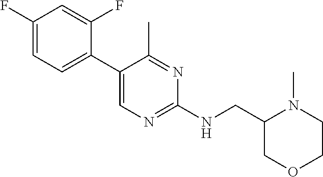 [SMILES: Cc1nc(NCC2COCCN2C)ncc1-c1ccc(F)cc1F]

Prepared as described for Example 2 using 2-chloro-5-(2,4-difluorophenyl)-4-methyl-pyrimidine (0.70 g, 2.91 mmol) and (4-methylmorpholin-3-yl)methanamine (0.60 g, 4.37 mmol) to afford the title compound, which was subjected to separation of enantiomers by SFC (Chiralpak IG (30 mm×250 mm, 5 μm); CO₂ (60/6), co-solvent (40%, 0.2% 7M methanolic NH₃ in MeOH); total flow=100.0 g/min; T=35° C.; UV detection at 250 nm) to afford isomer 1 (0.05 g, 5%) and isomer 2 (0.05 g, 5%) as off-white solids.

Example 79a (isomer 1): ¹H NMR (400 MHz, DMSO-d₆): δ 8.06 (s, 1H), 7.39 (m, 2H), 7.18 (m, 1H), 7.05 (br s, 1H), 3.75 (m, 1H), 3.68 (m, 1H), 3.58 (m, 1H), 3.45 (m, 1H), 3.30-3.15 (m, 2H), 2.65 (m, 1H), 2.30 (s, 3H), 2.25-2.10 (m, 5H). MS ES⁺: 335.29. Chiral HPLC: 99.97%, 2.34 min. SOR: −51.48 (c 0.1, MeOH).

Example 79b (isomer 2): ¹H NMR (400 MHz, DMSO-d₆): δ 8.07 (s, 1H), 7.39 (m, 2H), 7.18 (m, 11H), 7.05 (br s, 1H), 3.75 (m, 1H), 3.68 (m, 1H), 3.58 (m, 1H), 3.45 (m, 1H), 3.30-3.15 (m, 2H), 2.65 (m, 1H), 2.30 (s, 3H), 2.25-2.10 (m, 5H). MS ES⁺: 335.29. Chiral HPLC: 99.63%, 2.94 min. SOR: +48.40 (c 0.1, MeOH).

### Example 80: (R)—N-((4,4-difluoro-1-methylpyrrolidin-2-yl)methyl)-5-(2,4-difluorophenyl)-4-methyl-pyrimidin-2-amine

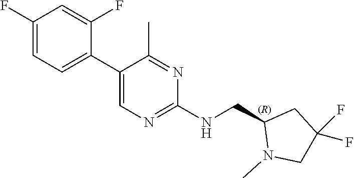

Step 1: A solution of (R)-1-(4,4-difluoro-1-methylpyrrolidin-2-yl)-N-(4-methoxybenzyl)methanamine (0.07 g, 0.3 mmol) and 2-chloro-5-(2,4-difluorophenyl)₄-methyl-pyrimidine (0.065 g, 0.27 mmol) in THF (1.5 mL) and toluene (1.5 mL) was treated with DIPEA (0.116 g, 0.90 mmol) and stirred for 16 h at 125° C. in a sealed tube. The mixture was cooled to RT and purified by flash column chromatography (amine silica Davisil, 12-15% EtOAc in petrol ether) to afford (R)—N-((4,4-difluoro-1-methylpyrrolidin-2-yl)methyl)-5-(2,4-difluorophenyl)-N-(4-methoxybenzyl)-4-methyl-pyrimidin-2-amine, the protected precursor of the title compound (0.07 g, 58%) as a gummy solid.

Step 2: A solution of (R)—N-((4,4-difluoro-1-methylpyrrolidin-2-yl)methyl)-5-(2,4-difluorophenyl)-N-(4-methoxybenzyl)-4-methyl-pyrimidin-2-amine (0.07 g, 0.10 mmol) in EtOAc was treated with Pd—C (0.15 g 10%) and degassed with N₂, followed by H₂ (60 psi). The mixture was stirred for 2 days at RT and filtered through Celite®. The filtrate was dried over Na₂SO₄, concentrated under reduced pressure and purified by flash column chromatography (amine silica Davisil, 5-7% MeOH/DCM) to afford the title compound (10 mg, 20%) as a brown solid. ¹H NMR (400 MHz, DMSO-d₆): δ 8.23 (s, 1H), 7.45-7.37 (m, 2H), 7.23-7.18 (m, 1H), 6.37 (s, 1H), 4.55 (m, 1H), 4.20-4.10 (m, 1H), 3.95-3.80 (m, 1H), 3.20-3.00 (m, 11H), 2.90-2.80 (m, 1H), 2.75-2.50 (m, 2H), 2.50 (s, 3H), 2.20 (s, 3H). MS ES⁺: 355.10. Chiral HPLC: 96.83%, 2.47 min.

### Example 81: 5-(2-fluoro-4-methoxyphenyl)-2-((1-isopropylpiperidin-4-yl)amino) pyrimidine-4-carbonitrile

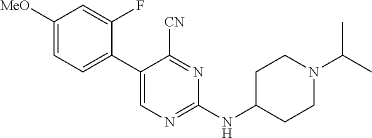 [SMILES: COc1ccc(-c2cnc(NC3CCN(C(C)C)CC3)nc2C#N)c(F)c1]

Prepared as described for Example 19 using 5-(2-fluoro-4-methoxyphenyl)-2-(methylsulfonyl)pyrimidine-4-carbonitrile (0.10 g, 0.33 mmol) and 1-isopropylpiperidin-4-amine (0.46 g, 0.33 mmol) to afford the title compound (0.041 g, 34%) as an off-white solid. ¹H NMR (401 MHz, DMSO-d₆): δ 8.57 (d, J=5.9 Hz, 1H), 7.99 (s, 1H), 7.46 (t, J=8.8 Hz, 1H), 7.03 (q, J=4.9 Hz, 1H), 6.94 (q, J=3.7 Hz, 1H), 3.83 (s, 3H), 3.68 (s, 1H), 2.73 (m, J=12.1 Hz, 3H), 2.18 (d, J=9.2 Hz, 2H), 1.85 (d, J=11.4 Hz, 2H), 1.48 (q, J=10.5 Hz, 2H), 0.96 (d, J=6.5 Hz, 6H). MS ES⁺: 370.1.

### Example 82: 5-(2,4-difluorophenyl)-4-methyl-N-(pyrrolidin-2-ylmethyl)pyrimidin-2-amine

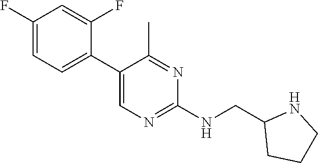 [SMILES: Cc1nc(NCC2CCCN2)ncc1-c1ccc(F)cc1F]

Step 1: Prepared as described for Example 2 using 2-chloro-5-(2,4-difluorophenyl)-4-methyl-pyrimidine (0.50 g, 2.1 mmol) and tert-butyl 2-(aminomethyl)pyrrolidine-1-carboxylate (0.92 g, 4.5 mmol) to afford tert-butyl 2-(((5-(2,4-difluorophenyl)-4-methyl-pyrimidin-2-yl)amino)methyl)pyrrolidine-1-carboxylate, the protected precursor of the title compound (0.3 g) as a yellowish gummy intermediate.

Step 2: A solution of tert-butyl 2-(((5-(2,4-difluorophenyl)-4-methyl-pyrimidin-2-yl) amino)methyl)pyrrolidine-1-carboxylate (0.3 g, 0.7 mmol) in dioxane (5 ml) at 0° C. was treated with 4M HCl in dioxane (0.12 mL) and stirred for 12 h at RT. The mixture was basified with sodium bicarbonate and extracted with EtOAc. The organic layer was dried over Na₂SO₄ and evaporated under reduced pressure. Purification of the residue by reverse phase flash column chromatography (60-65% MeOH in H₂O) gave the title compound (0.27 g, 91%) as a solid. ¹H NMR (401 MHz, DMSO-d₆): δ 8.05 (s, 1H), 7.39 (m, J=4.5 Hz, 2H), 7.18 (m, J=3.8 Hz, 2H), 3.25 (d, J=10.5 Hz, 4H), 2.82 (m, J=4.6 Hz, 1H), 2.74 (m, J=6.0 Hz, 1H), 2.12 (s, 3H), 1.68 (m, J=5.0 Hz, 3H), 1.39 (m, J=5.2 Hz, 1H). MS ES⁺: 305.27.

### Example 83a/b: 5-(2,4-difluorophenyl)-N-((3,3-difluoropyrrolidin-2-yl)methyl)-4-methyl-pyrimidin-2-amine

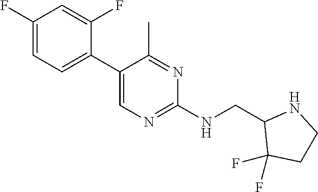 [SMILES: Cc1nc(NCC2NCCC2(F)F)ncc1-c1ccc(F)cc1F]

Step 1: A solution of tert-butyl 2-(((5-bromo-4-methyl-pyrimidin-2-yl)amino)methyl)-3,3-difluoropyrrolidine-1-carboxylate (1.6 g, 3.93 mmol), (2,4-difluorophenyl)boronic acid (0.931 g, 5.89 mmol), K₂CO₃ (1.09 g, 7.86 mmol) in dioxane (15 mL) and H₂O (3 mL) was degassed with N₂ (3×), treated with Pd(dppf)Cl₂ (0.158 g, 0.216 mmol) and stirred at 100° C. for 6 h under N₂. The mixture was cooled to RT, diluted with H₂O (50 mL) and extracted with EtOAc (2×40 mL). The combined organic layers were washed with brine, dried over MgSO₄, filtered and concentrated. Purification of the residue by flash silica gel chromatography (5 to 30% EtOAc/petroleum ether) gave tert-butyl 2-(((5-(2,4-difluorophenyl)-4-methyl-pyrimidin-2-yl)amino)methyl)-3,3-difluoropyrrolidine-1-carboxylate (1.65 g, 95%) as a yellow oil.

Step 2: A solution of 0.25 g of this oil in dioxane (0.5 mL) was treated with HCl/dioxane (4M, 0.5 mL), stirred at 25° C. for 16 h and evaporated. The residue was dissolved in H₂O, and adjusted pH=10 with aq. NaHCO₃. The aqueous layer was extracted with DCM (2×5 mL). The combined organic layers were washed with brine (10 mL), dried over MgSO₄, filtered and concentrated. Purification of the residue by SFC (DAICEL CHIRALPAK AD-H (250 mm×30 mm, 5 μm); 0.1% NH₃·H₂O in EtOH) gave isomer 1 (0.037 g, 29%) and isomer 2 (0.037 g, 29%) as yellow oils.

Example 83a (isomer 1): ¹H NMR (401 MHz, DMSO-d₆): δ 8.1 (s, 1H), 7.39 (m, 1H), 7.01 (m, 2H), 5.5 (s, 1H), 3.70 (m, 1H), 3.6 (m, 1H), 3.5 (m, 1H), 3.25 (m, 2H), 2.3 (m, 5H). MS ES⁺: 341.0. Chiral HPLC: 99.00%, 3.83 min.

Example 83b (isomer 2): ¹H NMR (401 MHz, CDCl₃): δ 8.1 (s, 1H), 7.3 (m, 1H), 6.9 (m, 2H), 5.5 (s, 1H), 3.60 (m, 1H), 3.5 (m, 1H), 3.4 (m, 1H), 3.25 (m, 2H), 2.25 (m, 5H). MS ES⁺: 341.1. Chiral HPLC: 96.3%, 4.07 min.

### Example 84a/b: N-((3,3-difluoro-1-methylpyrrolidin-2-yl)methyl)-5-(2,4-difluorophenyl)-4-methyl-pyrimidin-2-amine

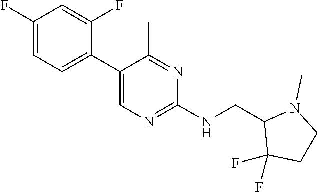 [SMILES: Cc1nc(NCC2N(C)CCC2(F)F)ncc1-c1ccc(F)cc1F]

A solution of tert-butyl 2-(((5-(2,4-difluorophenyl)-4-methyl-pyrimidin-2-yl)amino) methyl)-3,3-difluoropyrrolidine-1-carboxylate (see Example 83, 0.65 g, 1.48 mmol) in THF (15 mL) was treated with LiAlH₄ (0.168 g, 4.43 mmol) at 25° C. and stirred at 25° C. for 0.5 h. The mixture was filtered and concentrated under reduced pressure to remove solvent. The residue was purified by flash silica gel chromatography and the product was separated into enantiomers by SFC (DAICEL CHIRALPAK AD-H, 250 mm×30 mm, 5 μm; 25% of 0.1% aqueous NH₃ solution in ⁱPrOH), followed by SFC (DAICEL CHIRALPAK IG, 250 mm×30 mm, 10 μm; 20% of 0.1% aqueous NH₃ solution in MEOH) to give isomers 1 and 2.

Example 84a (isomer 1): ¹H NMR (401 MHz, CDCl₃): δ 8.06 (s, 1H), 7.25 (m, 1H), 6.95 (m, 2H), 5.53 (s, 1H), 3.95 (m, 1H), 3.5 (m, 1H), 3.1 (m, 1H), 2.6 (m, 1H), 2.5 (m, 4H), 2.25 (m, 5H). MS ES⁺: 355.1. Chiral HPLC: 100%, 2.94 min.

Example 84b (isomer 2): ¹H NMR (401 MHz, DMSO-d₆): δ 8.06 (s, 1H), 7.2 (m, 1H), 6.95 (m, 2H), 5.53 (s, 1H), 3.95 (m, 1H), 3.5 (m, 1H), 3.1 (m, 1H), 2.6 (m, 1H), 2.45 (m, 4H), 2.30 (m, 5H). MS ES⁺: 355.1. Chiral HPLC: 99.66%, 3.42 min.

### Example 85: 5-(2,4-difluorophenyl)-N-((1-ethylpyrrolidin-2-yl)methyl)-4-methyl-pyrimidin-2-amine

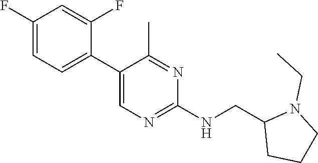 [SMILES: CCN1CCCC1CNc1ncc(-c2ccc(F)cc2F)c(C)n1]

Prepared as described for Example 2 using 2-chloro-5-(2,4-difluorophenyl)-4-methyl-pyrimidine (0.20 g, 0.83 mmol) and (1-ethylpyrrolidin-2-yl)methanamine (0.213 g, 1.66 mmol) to afford the title compound (0.170 g, 60%) as a yellow gummy solid. ¹H NMR (401 MHz, DMSO-d₆): δ 8.06 (s, 1H), 7.45-7.32 (m, 2H), 7.22-7.07 (m, 2H), 3.55-3.45 (m, 1H), 3.20-2.60 (m, 4H), 2.40-2.10 (m, 5H), 1.85 (br s, 1H), 1.70-1.55 (m, 3H), 1.15-1.00 (m, 3H). MS ES⁺: 333.29.

### Example 86a/b: N-(5-(2,4-difluorophenyl)-4-methyl-pyrimidin-2-yl)hexahydro-1H-pyrrolizin-1-amine

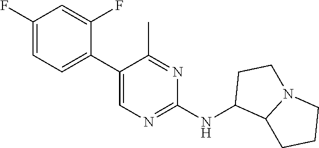 [SMILES: Cc1nc(NC2CCN3CCCC23)ncc1-c1ccc(F)cc1F]

Prepared as described for Example 2 using 2-chloro-5-(2,4-difluorophenyl)-4-methyl-pyrimidine (0.72 g, 3.0 mmol) and hexahydro-1H-pyrrolizin-1-amine (0.45 g, 3.6 mmol) to afford the racemic title compound (0.20 g, 25%) as an off-white solid. This racemate was subjected to separation of diastereomers by SFC (Chiralpak AD-3 (4.6 mm×150 mm, 5 μm); CO₂ (80%), co-solvent (20%, 0.2% DIPEA in MeOH); total flow=3 g/min; T=30° C.; UV detection at 220 nm) to afford isomer 1 (0.043 g) and isomer 2 (˜0.04 g) as yellow solids.

Example 86a (isomer 1): ¹H NMR (400 MHz, DMSO-d₆): δ 8.12 (s, 1H), 7.39 (m, J=4.6 Hz, 2H), 7.19 (m, J=3.8 Hz, 1H), 3.73 (q, J=6.7 Hz, 1H), 3.46 (m, J=5.8 Hz, 1H), 3.15 (t, J=4.7 Hz, 1H), 3.00 (m, J=5.7 Hz, 2H), 2.71 (m, J=3.7 Hz, 1H), 2.62 (q, J=5.1 Hz, 1H), 2.15 (s, 3H), 2.01 (m, J=4.6 Hz, 1H), 1.64 (m, J=11.6 Hz, 2H), 1.49 (m, J=4.4 Hz, 1H), 1.24 (m, J=5.7 Hz, 1H). MS ES⁺: 331.36. Chiral HPLC: 50%, 2.45 min; 49.5%, 8.30 min.

Example 86b (isomer 2): ¹H NMR (400 MHz, DMSO-d₆): δ 8.09 (s, 1H), 7.38 (m, J=4.6 Hz, 2H), 7.18 (m, J=3.8 Hz, 1H), 3.92 (m, J=4.2 Hz, 1H), 3.61 (m, J=3.9 Hz, 1H), 3.48 (m, J=6.2 Hz, 2H), 2.69 (m, J=4.8 Hz, 2H), 2.20-2.05 (m, 2H), 2.14 (s, 3H), 1.78 (m, J=3.7 Hz, 1H), 1.60 (m, J=7.4 Hz, 1H), 1.47 (q, J=4.4 Hz, 1H), 1.30 (m, J=8.0 Hz, 1H). MS ES⁺: 331.32. Chiral HPLC: 50.97%, 3.50 min; 48.30%, 5.03 min.

### Example 87: 5-(2,4-difluorophenyl)-4-methyl-N-((1-(trideuteriomethyl)pyrrolidin-2-yl))methyl)pyrimidin-2-amine

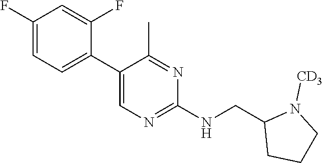 [SMILES: [2H]C([2H])([2H])N1CCCC1CNc1ncc(-c2ccc(F)cc2F)c(C)n1]

A stirred solution of 5-(2,4-difluorophenyl)-4-methyl-N-(pyrrolidin-2-ylmethyl)pyrimidin-2-amine (0.27 g, 0.90 mmol) in DMF (5 mL) was slowly treated at 0° C. with K₂CO₃ (0.42 g, 1.35 mmol) and CD₃I (0.16 ml, 1.17 mmol), warmed to RT and stirred for 2 h. The mixture was diluted with ice cold H₂O and extracted with EtOAc. The organic layer was dried over Na₂SO₄ and evaporated. Purification of the residue by column chromatography followed by prep-HPLC gave the title compound (0.10 g, 31%) as an off-white semi-solid. ¹H NMR (400 MHz, DMSO-d₆): δ 8.15 (s, 1H), 7.81 (s, 1H), 7.41 (m, J=4.1 Hz, 2H), 7.20 (m, J=3.8 Hz, 1H), 3.83 (t, J=6.7 Hz, 2H), 3.58 (m, J=6.2 Hz, 3H), 2.30 (m, J=4.9 Hz, 1H), 2.17 (s, 3H), 1.96 (m, J=8.4 Hz, 3H). MS ES⁺: 322.34.

### Example 88: 5-(2-fluoro-4-(trifluoromethoxy)phenyl)-N-(1-isopropylpiperidin-4-yl)-4-methyl-pyrimidin-2-amine

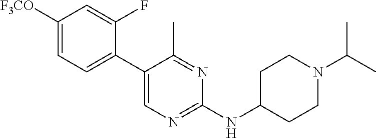 [SMILES: Cc1nc(NC2CCN(C(C)C)CC2)ncc1-c1ccc(OC(F)(F)F)cc1F]

Prepared as described for Example 2 using 2-chloro-5-(2-fluoro-4-(trifluoromethoxy)phenyl)-4-methyl-pyrimidine (0.15 g, 0.50 mmol) and 1-isopropylpiperidin-4-amine (0.085 g, 0.60 mmol) to afford the title compound (0.10 g, 49%) as an off-white solid. ¹H NMR (401 MHz, DMSO-d₆): δ 8.09 (s, 1H), 7.52 (t, J=8.6 Hz, 2H), 7.33 (d, J=8.6 Hz, 1H), 7.23 (s, 1H), 3.71 (d, J=6.8 Hz, 1H), 2.77 (d, J=11.7 Hz, 2H), 2.68 (m, J=6.6 Hz, 1H), 2.14 (d, J=14.2 Hz, 5H), 1.84 (d, J=10.1 Hz, 2H), 1.46 (m, J=5.5 Hz, 2H), 0.96 (d, J=6.6 Hz, 6H). MS ES⁺: 413.50.

### Example 89: 5-(2-fluoro-4-(trifluoromethoxy)phenyl)-4-methyl-N-(1-methylpiperidin-4-yl)pyrimidin-2-amine

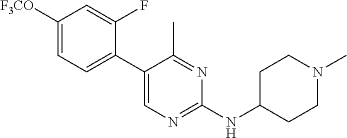 [SMILES: Cc1nc(NC2CCN(C)CC2)ncc1-c1ccc(OC(F)(F)F)cc1F]

Prepared as described for Example 2 using 2-chloro-5-(2-fluoro-4-(trifluoromethoxy)phenyl)-4-methyl-pyrimidine (0.2 g, 1.0 mmol) and 1-methylpiperidin-4-amine (0.125 g, 1.1 mmol) to afford the title compound (0.040 g, 16.1%) as a white solid. ¹H NMR (400 MHz, DMSO-d₆): δ 8.09 (s, 1H), 7.52 (t, J=8.6 Hz, 2H), 7.33 (d, J=8.5 Hz, 1H), 7.23 (s, 1H), 3.70 (t, J=3.4 Hz, 1H), 2.74 (d, J=11.7 Hz, 2H), 2.16 (s, 3H), 2.13 (s, 3H), 1.94 (t, J=10.9 Hz, 2H), 1.82 (d, J=9.9 Hz, 2H), 1.53 (m, J=5.9 Hz, 2H). MS ES⁺: 385.26.

### Example 90a: (S)-5-(2,4-difluorophenyl)-4-methyl-N-((1-(trideuteriomethyl)pyrrolidin-2-yl)methyl)pyrimidin-2-amine

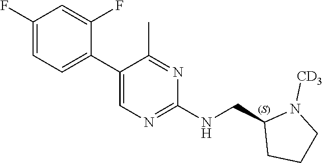

Step 1: A solution of 2-chloro-5-(2,4-difluorophenyl)-4-methyl-pyrimidine (0.50 g, 2.1 mmol) and tert-butyl (S)-2-(aminomethyl)pyrrolidine-1-carboxylate (0.50 g, 2.52 mmol) in dioxane (10 mL) was treated at RT with DIPEA (0.812 g, 6.30 mmol), sealed and stirred for 16 h at 100° C. The mixture was concentrated and the residue purified by column chromatography (15-20% EtOAc in petrol ether) to afford tert-butyl (S)-2-(((5-(2,4-difluorophenyl)-4-methyl-pyrimidin-2-yl)amino)methyl)pyrrolidine-1-carboxylate, the BOC-protected intermediate (0.3 g) as a yellowish gum.

Step 2: A stirred solution of tert-butyl (S)-2-(((5-(2,4-difluorophenyl)-4-methyl-pyrimidin-2-yl)amino)methyl)pyrrolidine-1-carboxylate (0.30 g, 0.7 mmol) in dioxane (5 mL) was slowly treated with 4M HCl in dioxane (0.12 mL) at 0° C. and stirred for 12 h at RT. The mixture was basified with sodium bicarbonate and extracted with EtOAc. The organic layer was dried over Na₂SO₄ and evaporated. Purification of the residue by reverse phase flash column chromatography (60-65% of MeOH in H₂O) gave (S)-5-(2,4-difluorophenyl)-4-methyl-N-(pyrrolidin-2-ylmethyl)pyrimidin-2-amine (0.270 g, 91%) as pale yellow gum.

Step 3: A stirred solution of (S)-5-(2,4-difluorophenyl)-4-methyl-N-(pyrrolidin-2-ylmethyl)pyrimidin-2-amine (0.27 g, 0.90 mmol) in DMF (5 mL) was slowly treated at 0° C. with K₂CO₃ (0.42 g, 1.35 mmol) followed by CD₃I (0.16 ml, 1.17 mmol) and stirred at RT for 2 h. The mixture was diluted with ice cold H₂O and extracted with EtOAc. The organic layer was dried over Na₂SO₄ and evaporated. The residue was purified by column chromatography followed by prep-HPLC to give the title compound (0.10 g, 31%) as an off-white solid. ¹H NMR (400 MHz, DMSO-d₆): δ 8.15 (s, 1H), 7.81 (s, 1H), 7.41 (m, J=4.1 Hz, 2H), 7.20 (m, J=3.8 Hz, 1H), 3.83 (t, J=6.7 Hz, 2H), 3.58 (m, J=6.2 Hz, 3H), 2.30 (m, J=4.9 Hz, 1H), 2.17 (s, 3H), 1.96 (m, J=8.4 Hz, 3H). MS ES⁺: 339.27 as NH₃ adduct.

### Example 90b: (R)-5-(2,4-difluorophenyl)-4-methyl-N-((1-(trideuteriomethyl)pyrrolidin-2-yl)methyl)pyrimidin-2-amine

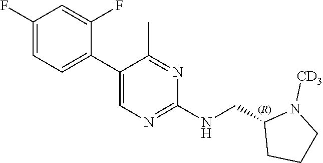

Prepared as described for Example 90a using 2-chloro-5-(2,4-difluorophenyl)-4-methyl-pyrimidine (0.50 g, 2.1 mmol) and tert-butyl (R)-2-(aminomethyl)pyrrolidine-1-carboxylate (0.50 g, 2.52 mmol) to afford in 3 steps the title compound (0.10 g, 31%) as an off-white semi-solid. ¹H NMR (400 MHz, DMSO-d₆): δ 8.15 (s, 1H), 7.84 (s, 1H), 7.41 (m, J=3.7 Hz, 2H), 7.20 (m, J=3.8 Hz, 1H), 3.83 (t, J=7.1 Hz, 2H), 3.58 (m, J=6.3 Hz, 3H), 2.30 (q, J=6.3 Hz, 1H), 2.17 (s, 3H), 1.98 (m, J=6.9 Hz, 3H). MS ES⁺: 339.31 as NH₃ adduct.

### Example 91a: (R)-5-(2-fluoro-4-(trifluoromethyl)phenyl)-4-methyl-N-((1-methylpyrrolidin-2-yl)methyl)pyrimidin-2-amine

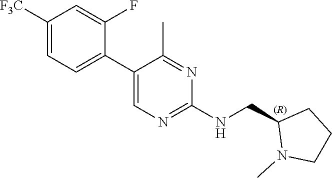

A solution of 2-chloro-5-(2-fluoro-4-(trifluoromethyl)phenyl)-4-methyl-pyrimidine (0.10 g, 0.30 mmol) in dioxane (20 mL) was treated with (R)-(1-methylpyrrolidin-2-yl)methanamine (0.04 g, 0.40 mmol) and DIPEA (0.08 g, 0.60 mmol), stirred at 100° C. for 16 h and cooled to RT. The mixture was diluted with H₂O and extracted with EtOAc. The organic layer was dried over Na₂SO₄ and concentrated. Purification of the crude material by prep-HPLC gave the title compound (0.10 g, 83%) as a pale yellow solid. ¹H NMR (DMSO-d₆, 400 MHz, 90° C.): δ 8.13 (s, 1H), 7.79 (d, J=10.1 Hz, 1H), 7.65 (m, J=5.5 Hz, 2H), 7.15 (s, 1H), 3.55 (m, J=3.3 Hz, 1H), 3.15 (m, J=6.4 Hz, 1H), 2.95 (m, J=3.0 Hz, 1H), 2.31 (m, 4H), 2.13 (t, J=8.6 Hz, 4H), 1.85 (m, J=3.8 Hz, 1H), 1.61 (m, J=4.3 Hz, 3H). MS ES⁺: 369.26. SOR: +52.7 (c 0.1, CHCl₃).

### Example 91b: (S)-5-(2-fluoro-4-(trifluoromethyl)phenyl)-4-methyl-N-((1-methylpyrrolidin-2-yl)methyl)pyrimidin-2-amine

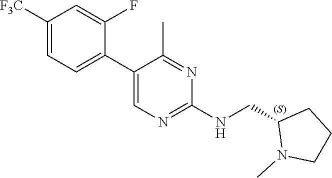

Prepared as described for Example 91a using 2-chloro-5-(2-fluoro-4-(trifluoromethyl)phenyl)-4-methyl-pyrimidine (0.20 g, 0.60 mmol) and (S)-(1-methylpyrrolidin-2-yl)methanamine (0.08 g, 0.80 mmol) to afford the title compound (120 mg, 52%) as a pale yellow solid. ¹H NMR (DMSO-d₆, 400 MHz, 90° C.): δ 8.13 (s, 1H), 7.79 (d, J=10.1 Hz, 1H), 7.65 (m, J=5.5 Hz, 2H), 7.15 (s, 1H), 3.55 (m, J=3.3 Hz, 1H), 3.15 (m, J=6.4 Hz, 1H), 2.95 (m, J=3.0 Hz, 1H), 2.31 (m, 4H), 2.13 (t, J=8.6 Hz, 4H), 1.85 (m, J=3.8 Hz, 1H), 1.61 (m, J=4.3 Hz, 3H). MS ES⁺: 369.26. Chiral HPLC: 99.20%, 1.39 min. SOR: −49.68 (c 0.1, CHCl₃).

### Example 92a: (R)-5-(4-fluoro-2-(trifluoromethyl)phenyl)-4-methyl-N-((1-methylpyrrolidin-2-yl)methyl)pyrimidin-2-amine

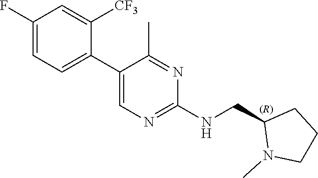

Prepared as described for Example 91 using 2-chloro-5-(4-fluoro-2-(trifluoromethyl)phenyl)-4-methyl-pyrimidine (0.10 g, 0.30 mmol) and (R)-(1-methylpyrrolidin-2-yl)methanamine (0.04 g, 0.4 mmol) to afford the title compound (0.060 g, 50%) as a pale yellow solid. ¹H NMR (DMSO-d₆, 400 MHz, 90° C.): δ 7.94 (s, 1H), 7.75 (q, J=4.0 Hz, 1H), 7.61 (m, J=3.9 Hz, 1H), 7.49 (q, J=4.7 Hz, 1H), 6.99 (s, 1H), 3.54 (s, 1H), 3.13 (s, 1H), 2.95 (m, J=4.4 Hz, 1H), 2.30 (s, 4H), 2.12 (q, J=8.5 Hz, 1H), 1.96 (s, 3H), 1.87 (q, J=5.5 Hz, 1H), 1.61 (m, J=4.5 Hz, 3H). MS ES⁺: 369.29. Chiral HPLC: 97.5%, 4.99 min. SOR: +52.72 (c 0.1, CHCl₃).

### Example 92b: (S)-5-(4-fluoro-2-(trifluoromethyl)phenyl)-4-methyl-N-((1-methylpyrrolidin-2-yl)methyl)pyrimidin-2-amine

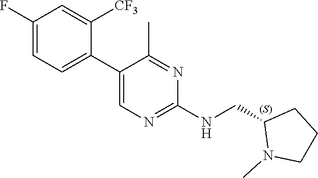

Prepared as described for Example 91 using 2-chloro-5-(4-fluoro-2-(trifluoromethyl)phenyl)-4-methyl-pyrimidine (0.10 g, 0.30 mmol) and (S)-(1-methylpyrrolidin-2-yl)methanamine (0.04 g, 0.4 mmol) to afford the title compound (0.05 g, 45%) as a pale yellow solid. ¹H NMR (DMSO-d₆, 400 MHz, 90° C.): δ 7.94 (s, 1H), 7.75 (q, J=4.0 Hz, 1H), 7.61 (m, J=3.9 Hz, 1H), 7.49 (q, J=4.7 Hz, 1H), 6.99 (s, 1H), 3.54 (s, 1H), 3.13 (d, J=5.5 Hz, 1H), 2.95 (m, J=3.5 Hz, 1H), 2.30 (s, 4H), 2.12 (q, J=8.5 Hz, 1H), 1.96 (s, 3H), 1.86 (m, J=6.6 Hz, 1H), 1.60 (m, J=4.3 Hz, 3H). MS ES⁺: 369.28. Chiral HPLC: 99.41%, 6.09 min. SOR: −49.04 (c 0.1, CHCl₃).

### Example 93a: (R)-5-(2-fluoro-4-(trifluoromethoxy)phenyl)-4-methyl-N-((1-methylpyrrolidin-2-yl)methyl)pyrimidin-2-amine

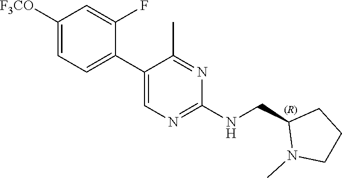

Prepared as described for Example 91 using 2-chloro-5-(2-fluoro-4-(trifluoromethoxy) phenyl)-4-methyl-pyrimidine (0.06 g, 0.20 mmol) and (R)-(1-methylpyrrolidin-2-yl) methanamine (0.034 g, 0.30 mmol) to afford the title compound (0.30 g, 30%) as an off-white solid. ¹H NMR (400 MHz, DMSO-d₆): δ 8.09 (s, 1H), 7.52 (m, J=4.6 Hz, 2H), 7.33 (t, J=4.2 Hz, 1H), 7.09 (s, 1H), 3.54 (m, J=3.3 Hz, 1H), 3.14 (m, J=5.2 Hz, 1H), 2.95 (m, J=3.0 Hz, 1H), 2.31 (m 4H), 2.12 (d, J=10.4 Hz, 4H), 1.85 (m, J=4.5 Hz, 1H), 1.60 (m, J=4.5 Hz, 3H). MS ES⁺: 385.39.

### Example 93b: (S)-5-(2-fluoro-4-(trifluoromethoxy)phenyl)-4-methyl-N-((1-methylpyrrolidin-2-yl)methyl)pyrimidin-2-amine

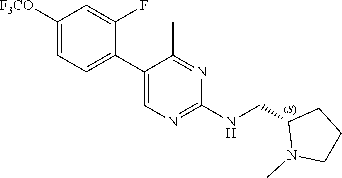

Prepared as described for Example 91 using 2-chloro-5-(2-fluoro-4-(trifluoromethoxy) phenyl)-4-methyl-pyrimidine (0.06 g, 0.20 mmol) and (S)-(1-methylpyrrolidin-2-yl) methanamine (0.034 g, 0.30 mmol) to afford the title compound (0.40 g, 42%) as an off-white solid. ¹H NMR (400 MHz, DMSO-d₆): δ 8.09 (s, 1H), 7.52 (m, J=3.9 Hz, 2H), 7.33 (d, J=8.5 Hz, 1H), 7.08 (s, 1H), 3.54 (m, J=3.3 Hz, 1H), 3.14 (m, J=6.4 Hz, 1H), 2.95 (t, J=6.4 Hz, 1H), 2.31 (m, 4H), 2.14 (m, 4H), 1.86 (m, J=5.2 Hz, 1H), 1.60 (m, J=4.6 Hz, 3H). MS ES⁺: 385.38.

### Example 94a: (R)-5-(2,4-difluorophenyl)-4-methyl-N-(pyrrolidin-2-ylmethyl)pyrimidin-2-amine

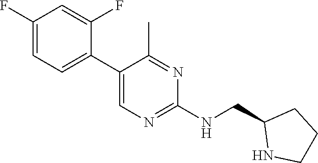 [SMILES: Cc1nc(NC[C@H]2CCCN2)ncc1-c1ccc(F)cc1F]

Step 1: A solution of 2-chloro-5-(2,4-difluorophenyl)-4-methyl-pyrimidine (0.50 g, 2.1 mmol) and ((R)-1-BOC-pyrrolidin-2-yl)methanamine (0.215 g, 1.90 mmol) in dioxane (10 mL) at RT was treated with DIPEA (0.82 g, 6.3 mmol) and stirred for 2 h at 120° C. (microwave). The solvent was evaporated and the residue purified by reverse phase flash column chromatography (50-55% MeOH in H₂O) to afford the intermediate BOC compound.

Step 2: The intermediate from step 1 was stirred in 4M HCl in dioxane (10 mL) at RT for 6 h and evaporated. The residue was purified by reverse phase column chromatography to obtain the title compound (0.101 g, 17%) as a pale brown solid. ¹H NMR (400 MHz, CDCl₃): δ 8.05 (s, 1H), 7.45 (m, J=4.0 Hz, 1H), 7.35 (m, J=2.2 Hz, 11H), 7.25-7.10 (m, 2H), 3.24 (s, 3H), 2.85-2.70 (m, 2H), 2.15-2.05 (m, 4H), 1.80-1.58 (m, 3H), 1.4 (br s, 1H). MS ES⁺: 305.16. SOR: −5.03 (c 0.5, DMSO-d₆).

### Example 94b: (S)-5-(2,4-difluorophenyl)-4-methyl-N-(pyrrolidin-2-ylmethyl)pyrimidin-2-amine

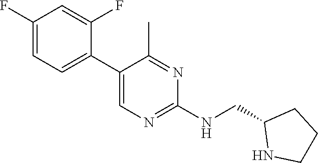 [SMILES: Cc1nc(NC[C@@H]2CCCN2)ncc1-c1ccc(F)cc1F]

Prepared as described for Example 94a using 2-chloro-5-(2,4-difluorophenyl)-4-methyl-pyrimidine (0.50 g, 2.1 mmol), ((S)-1-BOC-pyrrolidin-2-yl)methanamine (0.215 g, 1.90 mmol) to afford the title compound (0.088 g, 15%) as a pale brown solid. ¹H NMR (401 MHz, DMSO-d₆): δ 8.05 (s, 1H), 7.39 (m, 2H), 7.18 (m, 2H), 3.31 (s, 3H), 2.85-2.70 (m, 2H), 2.12 (m, 4H), 1.80-1.55 (m, 3H), 1.40-1.30 (m, 1H). MS ES⁺: 305.16. SOR: −4.54 (c 0.5, DMSO-d₆).

### Example 95a: (R)-5-(2,4-difluorophenyl)-4-methyl-N-((1-(2,2,2-trifluoroethyl)pyrrolidin-2-yl)methyl)pyrimidin-2-amine

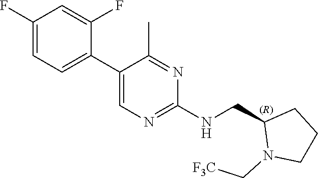

A solution of (R)-5-(2,4-difluorophenyl)-4-methyl-N-(pyrrolidin-2-ylmethyl)pyrimidin-2-amine (0.10 g, 0.33 mmol) in DCM (10 mL) was treated with DIPEA (0.13 g, 0.99 mmol) followed by 2,2,2-trifluoroethyl trifluoromethanesulfonate (0.15 g, 0.66 mmol) and stirred at RT for 4 h. The solvent was evaporated and the residue purified by prep-HPLC to obtain the title compound (0.035 g, 27%) as a brown solid. ¹H NMR (400 MHz, DMSO-d₆): δ 8.06 (s, 1H), 7.39 (m, 2H), 7.18 (m, J=2.8 Hz, 1H), 7.11 (t, J=5.9 Hz, 1H), 3.65-3.45 (m, 2H), 3.25-3.05 (m, 3H), 2.95-2.85 (m, 1H), 2.45 (m, 1H), 2.21 (s, 3H), 1.90-1.80 (m, 1H), 1.75-1.65 (m, 2H), 1.65-1.55 (m, 1H). MS ES⁺: 387.28.

### Example 95b: (S)-5-(2,4-difluorophenyl)-4-methyl-N-((1-(2,2,2-trifluoroethyl)pyrrolidin-2-yl)methyl)pyrimidin-2-amine

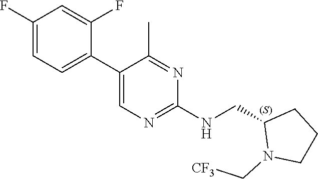

Prepared as described for Example 95a using (S)-5-(2,4-difluorophenyl)-4-methyl-N-(pyrrolidin-2-ylmethyl)pyrimidin-2-amine (0.10 g, 0.33 mmol) and 2,2,2-trifluoroethyl trifluoromethanesulfonate (0.15 g, 0.66 mmol) to obtain the title compound (0.025 g, 20%) as a brown solid. ¹H NMR (400 MHz, DMSO-d₆): δ 8.06 (s, 1H), 7.40 (m, 2H), 7.18 (m, J=2.8 Hz, 1H), 7.11 (t, J=5.9 Hz, 1H), 3.65-3.45 (m, 2H), 3.25-3.05 (m, 3H), 2.95-2.85 (m, 1H), 2.45 (m, 1H), 2.21 (s, 3H), 1.90-1.80 (m, 1H), 1.75-1.65 (m, 2H), 1.65-1.55 (m, 1H). MS ES⁺: 387.28.

### Example 96a/b: 5-(2,4-difluorophenyl)-N-((3-fluoro-1-methylpyrrolidin-2-yl)methyl)-4-methyl-pyrimidin-2-amine

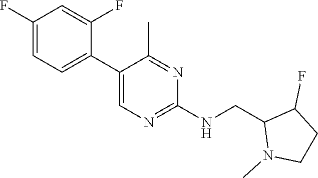 [SMILES: Cc1nc(NCC2C(F)CCN2C)ncc1-c1ccc(F)cc1F]

Step 1: A solution of 2-chloro-5-(2,4-difluorophenyl)-4-methyl-pyrimidine (0.24 g, 1.0 mmol) and tert-butyl 2-(aminomethyl)-3-fluoropyrrolidine-1-carboxylate (0.22 g, 1.0 mmol) in dioxane (10 mL) at RT under N₂ was treated with DIPEA (0.17 g, 1.33 mmol) and stirred at 100° C. for 16 h. The mixture was cooled to RT, treated with H₂O and extracted with EtOAc (2×20 mL). The combined organic layers were washed with brine, dried over Na₂SO₄ and concentrated in vacuo to give crude tert-butyl 2-(((5-(2,4-difluorophenyl)-4-methyl-pyrimidin-2-yl)amino)methyl)-3-fluoropyrrolidine-1-carboxylate (0.40 g) as an off-white solid, which was used in the next step without further purification.

Step 2: A solution of tert-butyl 2-(((5-(2,4-difluorophenyl)-4-methyl-pyrimidin-2-yl)amino)methyl)-3-fluoropyrrolidine-1-carboxylate (0.40 g, 0.94 mmol) in dioxane was treated at 0° C. with 4M HCl in dioxane (3 mL) and stirred at RT for 2 h. The solvent was evaporated under reduced pressure to obtain the crude product, which was dissolved in EtOAc (25 mL). The pH of the solution was adjusted to pH=9 using aq. NaOH solution and the aqueous layer was extracted with EtOAc. The combined organic layers were washed with brine, dried over Na₂SO₄ and concentrated under reduced pressure to obtain 5-(2,4-difluorophenyl)-N-((3-fluoropyrrolidin-2-yl)methyl)-4-methyl-pyrimidin-2-amine (0.38 g) as a brown solid, which was used in the next step without further purification. ¹H NMR (400 MHz, DMSO-d₆): δ 8.08 (s, 1H), 7.39 (m, J=5.1 Hz, 3H), 7.18 (m, J=3.8 Hz, 1H), 5.05 (d, J=28.2 Hz, 1H), 3.45 (m, J=3.3 Hz, 1H), 3.35 (m, 1H), 3.15 (m, J=4.8 Hz, 1H), 2.93 (t, J=7.4 Hz, 2H), 2.15 (m, 4H), 1.95-1.80 (m, 2H). MS ES⁺: 323.28.

Step 3: A stirred solution of 5-(2,4-difluorophenyl)-N-((3-fluoropyrrolidin-2-yl)methyl)-4-methyl-pyrimidin-2-amine (0.38 g, 1.17 mmol) in DCM (20 mL) was treated at 0° C. with DIPEA (0.30 mg, 2.35 mmol), stirred at 0° C. for 10 min, treated with CH₃I (0.167 mg, 1.17 mmol) and stirred at RT for 16 h. The mixture was diluted with DCM and washed with H₂O (20 mL). The organic layer was dried over Na₂SO₄ and evaporated. Purification of the residue by reverse phase column chromatography (30% MeOH/H₂O) gave the title compound as diastereomeric mixture. This material was subjected to separation of isomers by SFC (Chiralpak-IG (30 mm×250 mm, 5 μm); CO₂ (75%), co-solvent (25%, 0.2% 7M methanolic ammonia in MEOH); total flow=100.0 g/min; T=30° C.; UV detection at 248 nm) to obtain isomer 1 (0.015 g, 4.5%) and isomer 2 (0.016 g, 5%) as pale yellow semi-solids.

Example 96a (isomer 1): ¹H NMR (400 MHz, DMSO-d₆): δ 8.08 (s, 1H), 7.39 (m, J=5.1 Hz, 3H), 7.18 (m, J=3.8 Hz, 1H), 5.05 (t, J=28.2 Hz, 1H), 3.54 (m, J=3.3 Hz, 1H), 3.13 (t, J=4.3 Hz, 1H), 2.93 (t, J=7.5 Hz, 1H), 2.60 (m, J=9.3 Hz, 1H), 2.40 (m, 4H), 2.14 (s, 3H), 1.89 (m. J=5.6 Hz, 2H). MS ES⁺: 337.31. Chiral HPLC: 99.74%, 1.97 min.

Example 96b (isomer 2): ¹H NMR (400 MHz, DMSO-d₆): δ 8.08 (s, 1H), 7.39 (m, J=5.1 Hz, 3H), 7.18 (m, J=3.8 Hz, 1H), 5.05 (t, J=28.2 Hz, 1H), 3.54 (m, J=3.3 Hz, 1H), 3.10 (m, J=4.8 Hz, 1H), 2.93 (t, J=7.4 Hz, 1H), 2.61 (m, J=9.5 Hz, 1H), 2.40 (m, 4H), 2.14 (s, 3H), 1.90 (m, J=4.9 Hz, 2H). MS ES⁺: 337.28. Chiral HPLC: 95.15%, 2.49 min.

### Example 97a/b/c/d: N-(5-(2-fluoro-4-(trifluoromethoxy)phenyl)-4-methyl-pyrimidin-2-yl)hexahydro-1H-pyrrolizin-1-amine

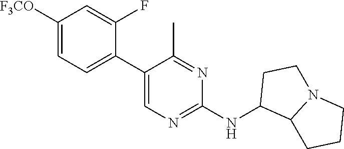 [SMILES: Cc1nc(NC2CCN3CCCC23)ncc1-c1ccc(OC(F)(F)F)cc1F]

Prepared as described for Example 2 using 2-chloro-5-(2-fluoro-4-(trifluoromethoxy) phenyl)-4-methyl-pyrimidine (0.2 g, 0.65 mmol) and hexahydro-1H-pyrrolizin-1-amine (0.13 g, 1.05 mmol) to afford the title compound as a mixture of diastereomers (0.20 g, 77%) as an off-white solid. This material was subjected to separation of isomers by SFC (Lux Cellulose 2 (30 mm×250 mm, 5 μm); CO₂ (80%), co-solvent (20%, 0.2% DIPEA in EtOH); total flow=100.0 g/min; T=30° C.; UV detection at 263 nm) to obtain two diastereomeric pairs of enantiomers: isomer 1a (0.010 g), isomer 1b (0.015 g), isomer 2a (0.016 g) and isomer 2b (0.008 g, 35%), respectively, as off-white solids.

Example 97a (isomer 1a): ¹H NMR (400 MHz, DMSO-d₆): δ 8.13 (s, 1H), 7.51 (t, J=8.6 Hz, 2H), 7.33 (d, J=8.7 Hz, 1H), 3.91 (m, J=5.1 Hz, 1H), 3.61 (m, J=3.9 Hz, 1H), 3.49 (m, J=5.9 Hz, 2H), 3.20 (m, 1H), 2.67 (m, J=9.7 Hz, 2H), 2.15 (d, J=0.9 Hz, 5H), 1.78 (t, J=8.8 Hz, 1H), 1.60 (q, J=10.2 Hz, 1H), 1.46 (q, J=4.4 Hz, 1H), 1.29 (m, J=11.1 Hz, 1H). MS ES⁺: 397.41.

Example 97b (isomer 1b): ¹H NMR (400 MHz, DMSO-d₆): δ 8.13 (s, 1H), 7.51 (t, J=8.6 Hz, 2H), 7.33 (d, J=8.6 Hz, 1H), 3.89 (m, J=4.1 Hz, 1H), 3.61 (m, J=3.9 Hz, 1H), 3.48 (m, J=5.9 Hz, 2H), 3.21 (m, 1H), 2.65 (m, J=10.0 Hz, 2H), 2.15 (s, 5H), 1.76 (m, J=5.5 Hz, 1H), 1.59 (t, J=10.4 Hz, 1H), 1.46 (q, J=4.4 Hz, 1H), 1.28 (q, J=13.0 Hz, 1H). MS ES⁺: 397.41.

Example 97c (isomer 2a): ¹H NMR (400 MHz, DMSO-d₆): δ 8.17 (s, 1H), 7.52 (t, J=8.6 Hz, 2H), 7.34 (d, J=8.5 Hz, 1H), 3.74 (q, J=6.7 Hz, 1H), 3.47 (m, J=5.8 Hz, 1H), 3.15 (d, J=8.9 Hz, 1H), 3.04 (d, J=9.8 Hz, 2H), 2.75 (m, J=6.7 Hz, 2H), 2.64 (m, J=5.0 Hz, 1H), 2.17 (d, J=0.9 Hz, 3H), 2.03 (m, J=5.6 Hz, 1H), 1.66 (m, J=11.5 Hz, 2H), 1.52 (m, J=5.7 Hz, 1H), 1.28 (m, J=6.3 Hz, 1H). MS ES⁺: 397.40.

Example 97d (isomer 2b): ¹H NMR (400 MHz, DMSO-d₆): δ 8.16 (s, 1H), 7.52 (t, J=8.6 Hz, 2H), 7.34 (d, J=8.6 Hz, 1H), 3.72 (q, J=6.7 Hz, 1H), 3.46 (m, J=5.8 Hz, 1H), 3.15 (d, J=11.8 Hz, 1H), 2.95 (m, J=5.8 Hz, 2H), 2.61 (m, J=7.1 Hz, 2H), 2.41 (s, 1H), 2.16 (d, J=0.8 Hz, 3H), 1.98 (q, J=5.6 Hz, 1H), 1.61 (m, J=11.1 Hz, 2H), 1.47 (m, J=4.9 Hz, 1H), 1.25 (m, J=6.5 Hz, 1H). MS ES⁺: 397.40.

### Example 98: N-((1-ethylpyrrolidin-2-yl)methyl)-5-(2-fluoro-4-(trifluoromethyl)phenyl)-4-methyl-pyrimidin-2-amine, formate salt

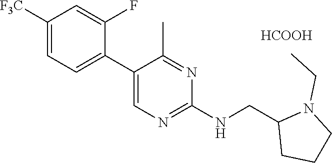 [SMILES: CCN1CCCC1CNc1ncc(-c2ccc(C(F)(F)F)cc2F)c(C)n1 ; "HCOOH"]

A solution of 2-chloro-5-(2-fluoro-4-(trifluoromethyl)phenyl)-4-methyl-pyrimidine (50 mg, 0.172 mmol), (1-ethylpyrrolidin-2-yl)methanamine (33 mg, 0.26 mmol) and DIPEA (44 mg, 0.34 mmol, 60 μL) in dioxane (0.5 mL) was stirred at 110° C. for 3 h, cooled to RT and extracted with DCM (3×5 mL). The combined organic layers were dried over Na₂SO₄, filtered and evaporated. Purification of the residue by prep-HPLC (Phenomenex Luna C18 (80 mm×40 mm, 3 μm); mobile phase A: 0.2% aq. HCOOH, mobile phase B: ACN; flow rate: 25 mL/min; gradient from 17% to 57% B). The desired material was dissolved in ACN (2 mL) and H₂O (10 mL) and lyophilized to dryness to give the title compound (30 mg, 40%, 98.6% purity by HPLC) as light yellow gummy solid. MS ES⁺: 383.1.

### Example 99: 5-(2-fluoro-4-(trifluoromethyl)phenyl)-4-methyl-N-((1-methylpyrrolidin-3-yl)methyl)pyrimidin-2-amine, formate salt

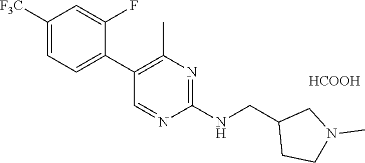 [SMILES: Cc1nc(NCC2CCN(C)C2)ncc1-c1ccc(C(F)(F)F)cc1F ; "HCOOH"]

Prepared as described for Example 98 using 2-chloro-5-(2-fluoro-4-(trifluoromethyl) phenyl)-4-methyl-pyrimidine (50 mg, 0.17 mmol) and (1-methylpyrrolidin-3-yl)methanamine (19 mg, 0.17 mmol) to afford the title compound (30 mg, 41%, 97.6% purity by HPLC) as a light yellow gummy solid. MS ES⁺: 369.1.

### Example 100: (S)-5-(2-fluoro-4-(trifluoromethyl)phenyl)-N-(1-isopropylpyrrolidin-3-yl)-4-methyl-pyrimidin-2-amine, formate salt

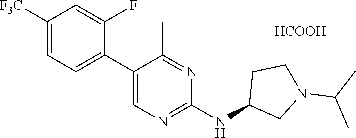 [SMILES: Cc1nc(N[C@H]2CCN(C(C)C)C2)ncc1-c1ccc(C(F)(F)F)cc1F ; "HCOOH"]

Prepared as described for Example 98 using 2-chloro-5-(2-fluoro-4-(trifluoromethyl)phenyl)-4-methyl-pyrimidine (50 mg, 0.17 mmol) and (3S)-1-isopropylpyrrolidin-3-amine (33 mg, 0.26 mmol) to afford the title compound (34 mg, 45%, 97.4% purity by HPLC) as a light yellow gummy solid. MS ES⁺: 383.1.

### Example 101: 5-(2-fluoro-4-(trifluoromethyl)phenyl)-4-methyl-N-((1-methyl-4-piperidinyl)methyl)pyrimidin-2-amine, formate salt

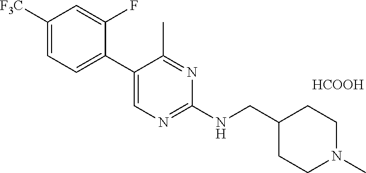 [SMILES: Cc1nc(NCC2CCN(C)CC2)ncc1-c1ccc(C(F)(F)F)cc1F ; "HCOOH"]

Prepared as described for Example 98 using 2-chloro-5-(2-fluoro-4-(trifluoromethyl)phenyl)-4-methyl-pyrimidine (50 mg, 0.17 mmol) and (1-methyl-4-piperidinyl)methanamine (33 mg, 0.26 mmol) to afford the title compound (26 mg, 35%, 98.7% purity by HPLC) as a light yellow gummy solid. MS ES⁺: 383.1.

### Example 102: (R)-5-(2-fluoro-4-(trifluoromethyl)phenyl)-4-methyl-N-(pyrrolidin-2-ylmethyl)pyrimidin-2-amine, semi-fumarate salt

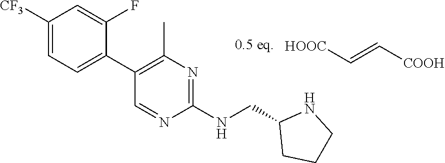 [SMILES: Cc1nc(NC[C@H]2CCCN2)ncc1-c1ccc(C(F)(F)F)cc1F ; O=C(O)/C=C/C(=O)O]

Prepared as described for Example 82 using 2-chloro-5-(2-fluoro-4-(trifluoromethyl)phenyl)-4-methyl-pyrimidine (200 mg, 0.69 mmol) and tert-butyl (2R)-2-(aminomethyl)pyrrolidine-1-carboxylate (138 mg, 0.69 mmol) to afford (R)-5-(2-fluoro-4-(trifluoromethyl)phenyl)-4-methyl-N-(pyrrolidin-2-ylmethyl)pyrimidin-2-amine (108 mg, 44%) as a yellow gummy solid. A solution of (R)-5-(2-fluoro-4-(trifluoromethyl)phenyl)-4-methyl-N-(pyrrolidin-2-ylmethyl)pyrimidin-2-amine (108 mg, 0.305 mmol) and fumaric acid (17.7 mg, 0.153 mmol) in EtOH (2 mL) was stirred at 25° C. for 1 h. The formed precipitate was collected by filtration and dried to give the title compound (80 mg, 63%) as a white solid. ¹H NMR (400 MHz, D₂O): δ 8.16 (s, 1H), 7.66-7.56 (m, 3H), 7.53-7.43 (m, 1H), 6.47 (d, J=1.0 Hz, 1H), 3.91 (dq, J=3.6, 7.9 Hz, 1H), 3.82-3.75 (m, 1H), 3.64-3.57 (m, 1H), 3.35-3.25 (m, 2H), 2.24 (s, 3H), 2.21-2.14 (m, 1H), 2.10-1.94 (m, 2H), 1.90-1.75 (m, 1H). MS ES⁺: 354.9. Analyt. SFC: 100%, 3.26 min.

### Example 103: (S)-5-(2-fluoro-4-(trifluoromethyl)phenyl)-4-methyl-N-(pyrrolidin-2-ylmethyl)pyrimidin-2-amine, semi-fumarate salt

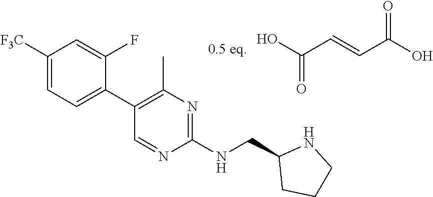 [SMILES: Cc1nc(NC[C@@H]2CCCN2)ncc1-c1ccc(C(F)(F)F)cc1F ; O=C(O)/C=C/C(=O)O]

Prepared as described for Example 102 using 2-chloro-5-(2-fluoro-4-(trifluoromethyl) phenyl)-4-methyl-pyrimidine (209 mg, 0.72 mmol) and tert-butyl (2S)-2-(aminomethyl)pyrrolidine-1-carboxylate (187 mg, 0.935 mmol) to afford the title compound (108 mg, 36%) as a white solid. ¹H NMR (400 MHz, D₂O): δ 8.09 (s, 1H), 7.59-7.49 (m, 3H), 7.47-7.36 (m, 1H), 6.41 (s, 1H), 3.92-3.79 (m, 1H), 3.76-3.66 (m, 1H), 3.63-3.52 (m, 1H), 3.34-3.17 (m, 2H), 2.17 (s, 3H), 2.15-2.07 (m, 1H), 2.06-1.88 (m, 2H), 1.84-1.70 (m, 1H). MS ES⁺: 355.1. Analyt. SFC: 99.5%, 3.48 min.

### Example 104: 5-(2-fluoro-4-(trifluoromethyl)phenyl)-4-methyl-N-(2-piperidinylmethyl) pyrimidin-2-amine, hydrochloride salt

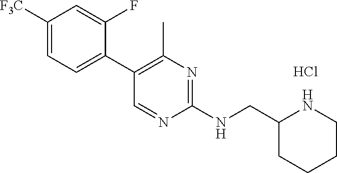 [SMILES: Cc1nc(NCC2CCCCN2)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (50 mg, 0.17 mmol) and tert-butyl 2-(aminomethyl) piperidine-1-carboxylate (37 mg, 0.17 mmol) to afford the title compound (25 mg, 34%, 95.2% purity by HPLC) as a yellow gummy solid. MS ES⁺: 369.1.

### Example 104a/b: (R)-5-(2-fluoro-4-(trifluoromethyl)phenyl)-4-methyl-N-(2-piperidinylmethyl)pyrimidin-2-amine semi-fumarate salt and (S)-5-(2-fluoro-4-(trifluoromethyl)phenyl)-4-methyl-N-(2-piperidinylmethyl)pyrimidin-2-amine semi-fumarate salt

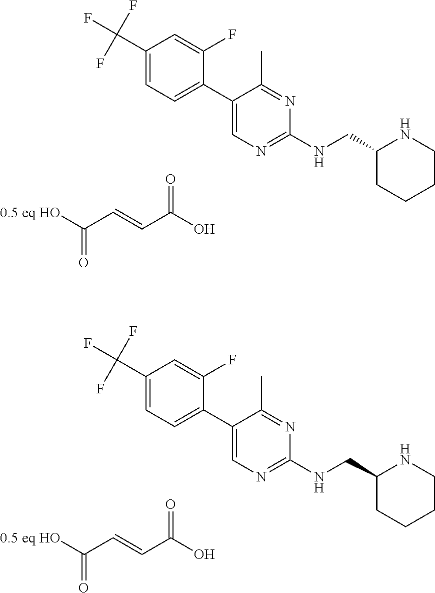

Step 1: To a solution of 2-chloro-5-(2-fluoro-4-(trifluoromethyl)phenyl)-4-methyl-pyrimidine (300 mg, 1.03 mmol) and tert-butyl 2-(aminomethyl)piperidine-1-carboxylate (441 mg, 2.06 mmol) in 1,4-dioxane (0.5 ml) was added DIPEA (266 mg, 2.06 mmol). The mixture was stirred at 110° C. for 16 h. The mixture cooled to RT, diluted with EtOAc (5 ml), washed with H₂O (10 ml) and extracted with EtOAc (3×5 ml). The combined organic layers were washed with brine (10 ml), dried (Na₂SO₄) and evaporated. The residue was purified by flash silica gel chromatography (ISCO®; 4 g SepaFlash® Silica Flash Column, 0˜50% EtOAc/petroleum ether) to give rac-tert-butyl 2-[[[5-[2-fluoro-4-(trifluoromethyl)phenyl]-4-methyl-pyrimidin-2-yl]amino]methyl]piperidine-1-carboxylate (370 mg, 0.755 mmol, 73%, 95.6% HPLC purity) as a colourless gummy solid. MS ES⁺: 469.4.

Step 2: The rac-tert-butyl 2-[[[5-[2-fluoro-4-(trifluoromethyl)phenyl]-4-methyl-pyrimidin-2-yl]amino]methyl]piperidine-1-carboxylate (370 mg 0.755 mmol) was purified by SFC (DAICEL CHIRALPAK AD, 250 mm×30 mm, 10 μm; mobile phase: A: supercritical CO₂, B: 0.1% NH₃. H₂O in EtOH, A:B=4:1) to give isomer 1 (170 mg, 0.361 mmol, 47%, 99.4% HPLC purity) as a white solid and isomer 2 (160 mg, 0.341 mmol, 44%, 99.7% HPLC purity) as a white solid.

Step 3a: A solution of (*R)-tert-butyl 2-[[[5-[2-fluoro-4-(trifluoromethyl)phenyl]-4-methyl-pyrimidin-2-yl]amino]methyl]piperidine-1-carboxylate (170 mg, 0.363 mmol) was dissolved into HCl/dioxane (4M, 3 mL), stirred at 25° C. for 1 h and concentrated to afford a residue which was dissolved into CH₂Cl₂ (5 ml) and H₂O (5 ml). The aqueous phase was basified to pH=8 using sat. aq. NaHCO₃ and the mixture was extracted with CH₂Cl₂ (5×5 ml). The combined organic layers were dried (Na₂SO₄) and concentrated under reduced pressure to give an off-white gummy solid. The residue was dissolved in EtOH (1 ml), treated with fumaric acid (21 mg, 0.18 mmol) and stirred at 25° C. for 0.5 h. White precipitates were formed, which were collected and dried to afford 104a semi-fumarate (R-enantiomer; 51.3 mg, 0.118 mmol, 32%, 98.1% HPLC purity) as a white solid. ¹H NMR (400 MHz, D₂O): δ 8.13 (s, 1H), 7.62-7.53 (m, 2H), 7.51-7.42 (m, 1H), 6.50 (s, 1H), 3.70-3.61 (m, 1H), 3.59-3.51 (m, 1H), 3.40-3.32 (m, 2H), 2.96-2.86 (m, 1H), 2.23-2.17 (m, 3H), 2.00-1.81 (m, 3H), 1.67-1.56 (m, 1H), 1.55-1.46 (m, 2H). MS ES⁺: 369.3. Chiral SFC: 100%, 2.40 min.

Step 3b: A solution of (*S)-tert-butyl 2-[[[5-[2-fluoro-4-(trifluoromethyl)phenyl]-4-methyl-pyrimidin-2-yl]amino]methyl]piperidine-1-carboxylate (130 mg, 0.353 mmol) in EtOH (1 ml) was treated with fumaric acid (20.5 mg, 0.176 mmol). The mixture was stirred at 25° C. for 0.5 h. White precipitates were formed, which were collected to afford 104b semi-fumarate (S-enantiomer; 53.9 mg, 34.7%, 96.9% HPLC purity) as a white solid. ¹H NMR (400 MHz, D₂O): δ 8.13 (s, 1H), 7.62-7.52 (m, 2H), 7.50-7.42 (m, 1H), 6.51 (s, 1H), 3.70-3.61 (m, 1H), 3.60-3.51 (m, 1H), 3.43-3.31 (m, 2H), 2.99-2.84 (m, 1H), 2.25-2.17 (m, 3H), 2.00-1.81 (m, 3H), 1.65-1.56 (m, 1H), 1.55-1.44 (m, 2H). MS ES⁺: 369.3. Chiral SFC: 100%, 1.99 min.

### Example 105: (S)—N-((1-ethylpyrrolidin-2-yl)methyl)-5-(2-fluoro-4-(trifluoromethyl) phenyl)-4-methyl-pyrimidin-2-amine, formate salt

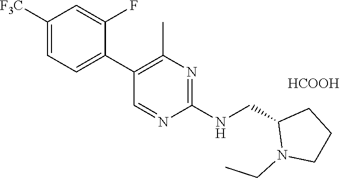 [SMILES: CCN1CCC[C@H]1CNc1ncc(-c2ccc(C(F)(F)F)cc2F)c(C)n1 ; "HCOOH"]

Prepared as described for Example 98 using 2-chloro-5-(2-fluoro-4-(trifluoromethyl) phenyl)-4-methyl-pyrimidine (50 mg, 0.17 mmol) and ((2S)-1-ethylpyrrolidin-2-yl) methanamine (33 mg, 0.26 mmol) to afford the title compound (20 mg, 27%, 99.6% purity by HPLC) as a light yellow gummy solid. MS ES⁺: 383.1.

### Example 106: 5-(2-fluoro-4-(trifluoromethyl)phenyl)-4-methyl-N-(2-(1-methylpyrrolidin-2-yl)ethyl)pyrimidin-2-amine, formate salt

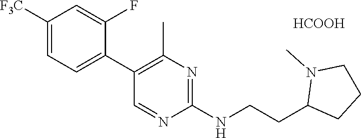 [SMILES: Cc1nc(NCCC2CCCN2C)ncc1-c1ccc(C(F)(F)F)cc1F ; "HCOOH"]

Prepared as described for Example 98 using 2-chloro-5-(2-fluoro-4-(trifluoromethyl) phenyl)-4-methyl-pyrimidine (50 mg, 0.17 mmol) and 2-(1-methylpyrrolidin-2-yl)ethan-1-amine (33 mg, 0.26 mmol) to afford the title compound (20 mg, 27%, 99% purity by HPLC) as a light yellow gummy solid. MS ES⁺: 383.1.

### Example 106a/b: (R)-5-(2-fluoro-4-(trifluoromethyl)phenyl)-4-methyl-N-(2-(1-methylpyrrolidin-2-yl)ethyl)pyrimidin-2-amine (fumarate, 2.5 equiv) and (S)-5-(2-fluoro-4-(trifluoromethyl)phenyl)-4-methyl-N-(2-(1-methylpyrrolidin-2-yl)ethyl)pyrimidin-2-amine (bis-fumarate)

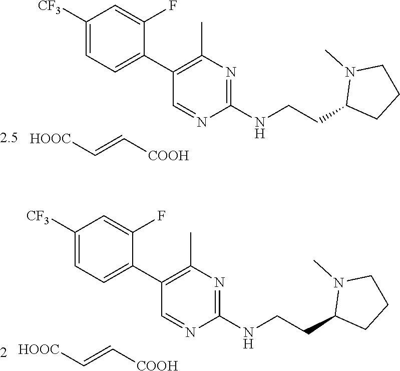 [SMILES: Cc1nc(NCC[C@H]2CCCN2C)ncc1-c1ccc(C(F)(F)F)cc1F ; O=C(O)/C=C/C(=O)O ; Cc1nc(NCC[C@@H]2CCCN2C)ncc1-c1ccc(C(F)(F)F)cc1F ; O=C(O)/C=C/C(=O)O]

Step 1: Racemic material of 106 free base (180 mg) was purified by SFC separation (DAICEL CHIRALPAK IC, 250 mm×30 mm, 10 μm; mobile phase: A: supercritical CO₂, B: 0.1% NH₃. H₂O in EtOH, A:B=1:1 at 60 mL/min) to give the (R)-isomer 106a (70 mg, 27%) as a light yellow oil and the (S)-isomer 106b (40 mg, 15%) as a light yellow oil.

Step 2a: To a solution of (R)-isomer 106a (85 mg, 0.22 mmol) in EtOH (1 ml) was added fumaric acid (51.6 mg, 0.445 mmol). The mixture was stirred at 25° C. for 0.25 h, concentrated to afford a solid which was partitioned between CH₃CN (2 ml) and H₂O (10 ml) and lyophilized to give the (R)-isomer 106a fumarate (2.5 equiv, 70 mg, 46%, 99.2% HPLC purity) as an off-white solid. ¹H NMR (400 MHz, D₂O): δ 8.19 (s, 1H), 7.57-7.50 (m, 2H), 7.48-7.37 (m, 1H), 6.65 (s, 5H), 3.62-3.55 (m, 1H), 3.54-3.47 (m, 2H), 3.37-3.25 (m, 1H), 3.09-2.99 (m, 1H), 2.81 (s, 3H), 2.40-2.30 (m, 1H), 2.24 (s, 3H), 2.12-1.85 (m, 3H), 1.84-1.68 (m, 2H). MS ES⁺: 383.3. Chiral SFC: 100%, 3.22 min.

Step 2b: To a solution of (S)-isomer 106b (70 mg, 0.18 mmol) in EtOH (1 ml) was added fumaric acid (42.5 mg, 0.366 mmol). The mixture was stirred at 25° C. for 0.25 h, concentrated to afford a solid which was partitioned between CH₃CN (2 ml) and H₂O (10 ml) and lyophilized to give the (S)-isomer 106b bis-fumarate (70 mg, 62%, 99.6% HPLC purity) as an off-white solid. ¹H NMR (400 MHz, D₂O): δ 8.18 (s, 1H), 7.60-7.51 (m, 2H), 7.49-7.42 (m, 1H), 6.67-6.63 (m, 4H), 3.67-3.58 (m, 1H), 3.57-3.49 (m, 2H), 3.40-3.30 (m, 1H), 3.12-3.03 (m, 11H), 2.85 (s, 3H), 2.43-2.33 (m, 1H), 2.24 (s, 3H), 2.13-1.86 (m, 3H), 1.86-1.71 (m, 2H). MS ES⁺: 383.3. Chiral SFC: 97.8%, 3.35 min.

### Example 107: 5-(2-fluoro-4-(trifluoromethyl)phenyl)-4-methyl-N-((1-methyl-3-piperidinyl)methyl)pyrimidin-2-amine, formate salt

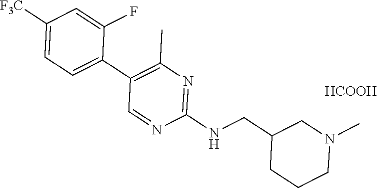 [SMILES: Cc1nc(NCC2CCCN(C)C2)ncc1-c1ccc(C(F)(F)F)cc1F ; "HCOOH"]

Prepared as described for Example 98 using 2-chloro-5-(2-fluoro-4-(trifluoromethyl) phenyl)-4-methyl-pyrimidine (50 mg, 0.17 mmol) and (1-methyl-3-piperidinyl)methanamine (33 mg, 0.26 mmol) to afford the title compound (20 mg, 27%, 99.4% purity by HPLC) as a light yellow gummy solid. MS ES⁺: 383.1.

### Example 108: 5-(2-fluoro-4-(trifluoromethyl)phenyl)-4-methyl-N-((4-methylmorpholin-2-yl)methyl)pyrimidin-2-amine, formate salt

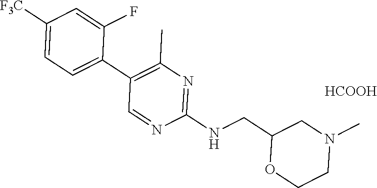 [SMILES: Cc1nc(NCC2CN(C)CCO2)ncc1-c1ccc(C(F)(F)F)cc1F ; "HCOOH"]

Prepared as described for Example 98 using 2-chloro-5-(2-fluoro-4-(trifluoromethyl) phenyl)-4-methyl-pyrimidine (50 mg, 0.17 mmol) and (4-methylmorpholin-2-yl)methanamine (22 mg, 0.17 mmol) to afford the title compound (20 mg, 27%, 94.4% purity by HPLC) as a light yellow gummy solid. MS ES⁺: 385.1.

### Example 108a/b: (R)-5-(2-fluoro-4-(trifluoromethyl)phenyl)-4-methyl-N-((4-methylmorpholin-2-yl)methyl)pyrimidin-2-amine (bis-fumarate) and (S)-5-(2-fluoro-4-(trifluoromethyl)phenyl)-4-methyl-N-((4-methylmorpholin-2-yl)methyl)pyrimidin-2-amine (bis-fumarate)

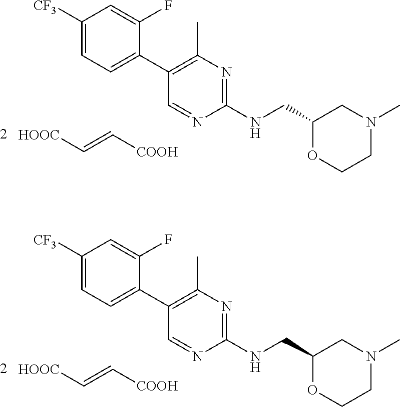 [SMILES: Cc1nc(NC[C@@H]2CN(C)CCO2)ncc1-c1ccc(C(F)(F)F)cc1F ; O=C(O)/C=C/C(=O)O ; Cc1nc(NC[C@H]2CN(C)CCO2)ncc1-c1ccc(C(F)(F)F)cc1F ; O=C(O)/C=C/C(=O)O]

Step 1: rac-5-(2-Fluoro-4-(trifluoromethyl)phenyl)-4-methyl-N-((4-methylmorpholin-2-yl)methyl)pyrimidin-2-amine (190 mg) was purified by SFC (DAICEL CHIRALPAK AD, 250 mm×30 mm, 10 μm; mobile phase: 15% of 0.1% aq. NH₃ in EtOH) to give isomer 108a (95 mg, 0.24 mmol, 48%, 97.5% HPLC purity) as a colourless oil and isomer 108b (96 mg, 0.25 mmol, 51%, 100% HPLC purity) as a colourless oil.

Step 2a: To a solution of isomer 108a (90 mg, 0.234 mmol) in EtOH (1 ml) was added fumaric acid (81.5 mg, 0.702 mmol). The mixture was stirred at 25° C. for 1 h and concentrated to afford a residue which was partitioned between CH₃CN (10 ml) and H₂O (20 ml). The solution was lyophilized to dryness to give the title compound as the bis-fumarate salt (enantiomerically pure with the absolute configuration not known; 70 mg, 46%, 95.9% HPLC purity) as an off-white solid. ¹H NMR (400 MHz, D₂O): δ 8.21 (s, 1H), 7.62-7.56 (m, 2H), 7.52-7.46 (m, 1H), 6.71 (s, 4H), 4.18-4.11 (m, 1H), 4.06-3.99 (m, 1H), 3.84-3.76 (m, 1H), 3.71-3.62 (m, 2H), 3.61-3.54 (m, 1H), 3.49-3.42 (m, 1H), 3.17-3.08 (m, 1H), 3.02-2.95 (m, 1H), 2.91-2.86 (m, 3H), 2.29-2.25 (m, 3H). MS ES⁺: 385.3. Chiral SFC: 100%, 1.95 min.

Step 2b: To a solution of isomer 108b (90 mg, 0.23 mmol) in EtOH (1 ml) was added fumaric acid (81.5 mg, 0.702 mmol). The mixture was stirred at 25° C. for 1 h and concentrated to afford a residue which was partitioned between CH₃CN (10 ml) and H₂O (20 ml). The solution was lyophilized to dryness to give the title compound as the bis-fumarate salt (enantiomerically pure with the absolute configuration not known; 80 mg, 55%, 99.2% HPLC purity) as an off-white solid. ¹H NMR (400 MHz, D₂O): δ 8.22 (s, 1H), 7.64-7.55 (m, 2H), 7.54-7.44 (m, 1H), 6.71 (s, 4H), 4.18-4.11 (m, 1H), 4.06-3.99 (m, 1H), 3.85-3.75 (m, 1H), 3.72-3.62 (m, 2H), 3.62-3.54 (m, 1H), 3.49-3.42 (m, 1H), 3.18-3.08 (m, 1H), 3.03-2.94 (m, 1H), 2.89 (s, 3H), 2.31-2.23 (m, 3H). MS ES⁺: 385.3. Chiral SFC: 100%, 2.12 min.

### Example 109: (1S,2S)—N¹-(5-(2-fluoro-4-(trifluoromethyl)phenyl)-4-methyl-pyrimidin-2-yl)-N²,N²-dimethyl-cyclohexane-1,2-diamine, formate salt

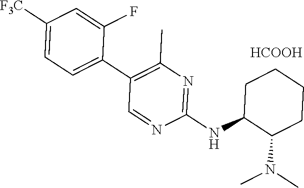 [SMILES: Cc1nc(N[C@H]2CCCC[C@@H]2N(C)C)ncc1-c1ccc(C(F)(F)F)cc1F ; "HCOOH"]

Prepared as described for Example 98 using 2-chloro-5-(2-fluoro-4-(trifluoromethyl) phenyl)-4-methyl-pyrimidine (30 mg, 0.10 mmol) and (1S,2S)—N²,N²-dimethylcyclohexane-1,2-diamine (29 mg, 0.21 mmol) to afford the title compound (15 mg, 32%, 97.5% purity by HPLC) as a light yellow gummy solid. MS ES⁺: 397.1.

### Example 110: (R)-5-(2-fluoro-4-(trifluoromethyl)phenyl)-4-methyl-N-(pyrrolidin-3-yl)pyrimidin-2-amine, hydrochloride salt

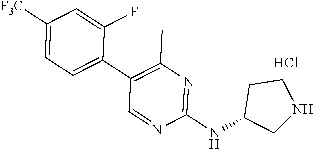 [SMILES: Cc1nc(N[C@@H]2CCNC2)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (50 mg, 0.17 mmol) and tert-butyl (3R)-3-aminopyrrolidine-1-carboxylate (48 mg, 0.26 mmol) to afford the title compound (5 mg, 8%, 98.6% purity by HPLC) as a light yellow gummy solid. MS ES⁺: 341.0.

### Example 111: (1S,3S)—N³-(5-(2-fluoro-4-(trifluoromethyl)phenyl)-4-methyl-pyrimidin-2-yl)cyclopentane-1,3-diamine, hydrochloride salt

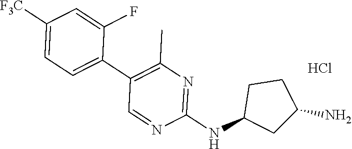 [SMILES: Cc1nc(N[C@H]2CC[C@H](N)C2)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (50 mg, 0.17 mmol) and tert-butyl N-((1S,3S)-3-aminocyclopentyl)carbamate (52 mg, 0.26 mmol) to afford the title compound (5.6 mg, 8.2%, 98.5% purity by HPLC) as a light yellow gummy solid. MS ES⁺: 355.1.

### Example 112: N-((cis)-4-fluoropyrrolidin-3-yl)-5-(2-fluoro-4-(trifluoromethyl)phenyl)-4-methyl-pyrimidin-2-amine, hydrochloride salt

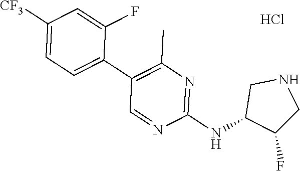 [SMILES: Cc1nc(N[C@@H]2CNC[C@@H]2F)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (50 mg, 0.17 mmol) and tert-butyl cis-3-amino-4-fluoro-pyrrolidine-1-carboxylate (35 mg, 0.17 mmol) to afford the title compound (6.7 mg, 9.7%, 98.3% purity by HPLC) as a light yellow gummy solid. MS ES⁺: 359.0.

### Example 113: (R)-5-(2-fluoro-4-(trifluoromethyl)phenyl)-4-methyl-N-((3-piperidinyl) methyl)pyrimidin-2-amine, hydrochloride salt

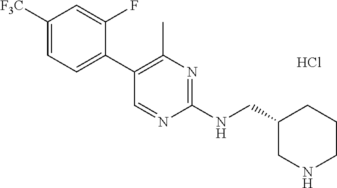 [SMILES: Cc1nc(NC[C@@H]2CCCNC2)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (30 mg, 0.10 mmol) and tert-butyl (3S)-3-(aminomethyl) piperidine-1-carboxylate (44 mg, 0.21 mmol) to afford the title compound (10 mg, 23%, 97.7% purity by HPLC) as a light yellow gummy solid. MS ES⁺: 369.1.

### Example 114: N-((trans)-4-fluoropyrrolidin-3-yl)-5-(2-fluoro-4-(trifluoromethyl)phenyl)-4-methyl-pyrimidin-2-amine, hydrochloride salt

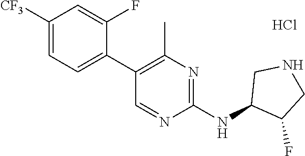 [SMILES: Cc1nc(N[C@H]2CNC[C@@H]2F)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (50 mg, 0.17 mmol) and tert-butyl (trans)-3-amino-4-fluoro-pyrrolidine-1-carboxylate (35 mg, 0.17 mmol) to afford the title compound (10 mg, 14%, 98.0% purity by HPLC) as a light yellow gummy solid. MS ES⁺: 359.0.

### Example 115: (3R,4R)-4-((5-(2-fluoro-4-(trifluoromethyl)phenyl)-4-methyl-pyrimidin-2-yl)amino)pyrrolidin-3-ol, hydrochloride salt

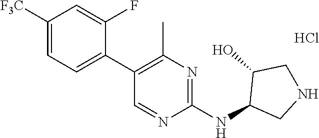 [SMILES: Cc1nc(N[C@@H]2CNC[C@H]2O)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (50 mg, 0.17 mmol) and tert-butyl (3R,4R)-3-amino-4-hydroxy-pyrrolidine-1-carboxylate (35 mg, 0.17 mmol) to afford the title compound (10 mg, 14%, 98.1% purity by HPLC) as a light yellow gummy solid. MS ES⁺: 357.0.

### Example 116: (S)-5-(2-fluoro-4-(trifluoromethyl)phenyl)-4-methyl-N-(3-piperidinyl) pyrimidin-2-amine, hydrochloride salt

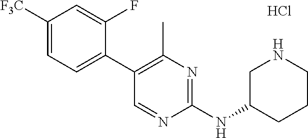

Prepared as described for Example 82 using 2-chloro-5-(2-fluoro-4-(trifluoromethyl) phenyl)-4-methyl-pyrimidine (30 mg, 0.10 mmol) and tert-butyl (3S)-3-aminopiperidine-1-carboxylate (41 mg, 0.21 mmol) to afford the title compound (10 mg, 25%, 98.9% purity by HPLC) as a light yellow gummy solid. MS ES⁺: 355.0.

### Example 117: (R)-5-(2-fluoro-4-(trifluoromethyl)phenyl)-4-methyl-N-((1-(trideuteriomethyl)pyrrolidin-2-yl)methyl)pyrimidin-2-amine, fumarate salt

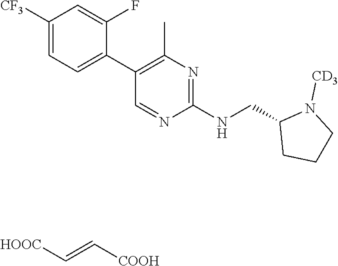 [SMILES: [2H]C([2H])([2H])N1CCC[C@@H]1CNc1ncc(-c2ccc(C(F)(F)F)cc2F)c(C)n1 ; O=C(O)/C=C/C(=O)O]

Step 1: A solution of tert-butyl N-(((2R)-pyrrolidin-2-yl)methyl)carbamate (1.00 g, 4.99 mmol) and DIPEA (1.29 g, 9.99 mmol, 1.74 mL) in DCM (10 mL) was dropwise treated with trideuterio(iodo)methane (724 mg, 4.99 mmol, 310 μL) at 0° C. and stirred at 25° C. for 1 h. The mixture was extracted with DCM (3×20 mL). The combined organic layers were washed with brine (50 mL), dried over Na₂SO₄ and filtered. The filtrate was evaporated and the residue purified by silica gel column chromatography (DCM/MeOH 10:1) to afford tert-butyl N-(((2R)-1-(trideuteriomethyl)pyrrolidin-2-yl)methyl)carbamate (0.80 g, 63%, 85% purity by HPLC) as a yellow solid.

Step 2: A solution of tert-butyl N-(((2R)-1-(trideuteriomethyl)pyrrolidin-2-yl)methyl)carbamate (0.80 g, 3.13 mmol, 85% purity by HPLC) in 4M HCl in dioxane (10 mL) was stirred at 25° C. for 0.5 h. The mixture was evaporated to dryness to give ((2R)-1-(trideuteriomethyl)pyrrolidin-2-yl)methanamine hydrochloride (0.56 g, crude) as a brown solid, which was used in the next step without further purification.

Step 3: A solution of ((2R)-1-(trideuteriomethyl)pyrrolidin-2-yl)methanamine hydrochloride (158 mg, 1.03 mmol), 2-chloro-5-(2-fluoro-4-(trifluoromethyl)phenyl)-4-methyl-pyrimidine (150 mg, 0.516 mmol) and DIPEA (200 mg, 1.55 mmol) in dioxane (1 mL) was stirred at 110° C. for 8 h, cooled to RT and extracted with DCM (3×50 mL). The combined organic layers were washed with brine (50 mL), dried over Na₂SO₄ and filtered.

The filtrate was evaporated to dryness. Purification of the residue by prep-TLC (DCM/MeOH 10:1) and prep-HPLC (Phenomenex Luna C18 (80 mm×40 mm, 3 μm); mobile phase A: 0.225% aq. HCOOH, mobile phase B: ACN; flow rate: 25 mL/min; gradient from 14% to 54% B) gave the product, which was dissolved in ACN (2 mL) and H₂O (10 mL) and lyophilized to dryness to give the product as a yellow gummy solid. This material was dissolved in H₂O (10 mL) and the pH was adjusted pH=8 using sat. aq. NaHCO₃. The mixture was extracted with DCM (3×10 mL). The combined organic layers were washed with brine (10 mL), dried over Na₂SO₄ and filtered. The filtrate was evaporated to dryness to give 5-(2-fluoro-4-(trifluoromethyl)phenyl)-4-methyl-N-(((2R)-1-(trideuteriomethyl) pyrrolidin-2-yl)methyl)pyrimidin-2-amine (20 mg, 10%) as a yellow gummy solid. MS ES⁺: 372.2.

Step 4: A solution of 5-(2-fluoro-4-(trifluoromethyl)phenyl)-4-methyl-N-(((2R)-1-(trideuteriomethyl)pyrrolidin-2-yl)methyl)pyrimidin-2-amine (20 mg, 0.53 mmol) and fumaric acid (6.25 mg, 0.53 mmol) in EtOH (1 mL) was stirred at 25° C. for 1 h. The mixture was evaporated to dryness and the residue suspended in H₂O (10 mL). The solution was lyophilized to give the title compound (17.7 mg, 64%, 95.4% purity by HPLC) as a white powder. ¹H NMR (400 MHz, D₂O): 7.85 (s, 1H), 7.38-7.29 (m, 1H), 7.28-7.21 (m, 1H), 7.19-7.11 (m, 1H), 6.44 (s, 2H), 3.76-3.43 (m, 3H), 3.18-2.95 (m, 1H), 2.20-2.05 (m, 1H), 2.01-1.68 (m, 6H). MS ES⁺: 372.0.

### Example 118: (S)-5-(2-fluoro-4-(trifluoromethyl)phenyl)-4-methyl-N-((1-(trideuteriomethyl)pyrrolidin-2-yl)methyl)pyrimidin-2-amine, fumarate salt

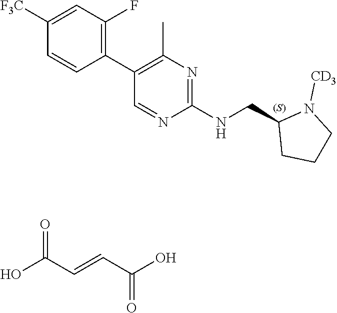

Prepared as described for Example 117 using 2-chloro-5-(2-fluoro-4-(trifluoromethyl) phenyl)-4-methyl-pyrimidine (200 mg, 0.69 mmol) and ((2S)-1-(trideuteriomethyl) pyrrolidin-2-yl)methanamine (81 mg, 0.69 mmol) to afford the title compound (80 mg, 23%, 98.8% purity by HPLC) as a light yellow powder. ¹H NMR (400 MHz, D₂O): 8.16 (s, 1H), 7.64-7.51 (m, 2H), 7.50-7.42 (m, 1H), 6.64 (s, 2H), 3.94-3.79 (m, 1H), 3.92-3.57 (m, 3H), 3.17-3.04 (m, 1H), 2.32-2.16 (m, 3H), 2.13-2.01 (m, 2H), 1.98-1.85 (m, 2H). MS ES⁺: 372.1. SFC: 100%, 2.18 min.

### Example 119: (R)—N-(5-(2-fluoro-4-(trifluoromethyl)phenyl)-4-methyl-pyrimidin-2-yl) quinuclidin-3-amine, formate salt

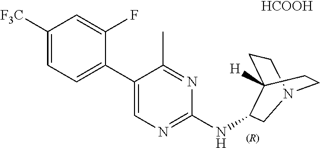

Prepared as described for Example 98 using 2-chloro-5-(2-fluoro-4-(trifluoromethyl) phenyl)-4-methyl-pyrimidine (300 mg, 1.03 mmol) and (3R)-quinuclidin-3-amine dihydrochloride (205 mg, 1.03 mmol) to afford the title compound (3 mg, 0.6%, 87% purity by HPLC) as a light yellow gummy solid. MS ES⁺: 381.1.

### Example 120: (S)—N-(5-(2-fluoro-4-(trifluoromethyl)phenyl)-4-methyl-pyrimidin-2-yl) quinuclidin-3-amine, formate salt

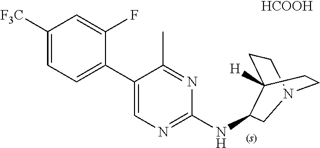

Prepared as described for Example 98 using 2-chloro-5-(2-fluoro-4-(trifluoromethyl) phenyl)-4-methyl-pyrimidine (30 mg, 0.10 mmol) and (3S)-quinuclidin-3-amine dihydrochloride (41 mg, 0.21 mmol) to give the title compound (5 mg, 10%, 89.7% purity by HPLC) as a light yellow gummy solid. MS ES⁺: 381.1.

### Example 121: (1R,3s,5S)—N-(5-(2-fluoro-4-(trifluoromethyl)phenyl)-4-methyl-pyrimidin-2-yl)-8-azabicyclo[3.2.1]octan-3-amine, hydrochloride salt

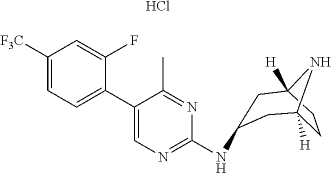 [SMILES: Cl ; Cc1nc(N[C@H]2C[C@H]3CC[C@@H](C2)N3)ncc1-c1ccc(C(F)(F)F)cc1F]

Prepared as described for Example 82 using 2-chloro-5-(2-fluoro-4-(trifluoromethyl) phenyl)-4-methyl-pyrimidine (30 mg, 0.10 mmol) and tert-butyl (1R,5S)-3-amino-8-azabicyclo[3.2.1]octane-8-carboxylate (47 mg, 0.21 mmol) to give the title compound (10 mg, 22%, 96.7% purity by HPLC) as a light yellow gummy solid. MS ES⁺: 381.1.

### Example 122: N-(5-(2-fluoro-4-(trifluoromethyl)phenyl)-4-methyl-pyrimidin-2-yl)-2-azaspiro[3.3]heptan-6-amine, formate salt

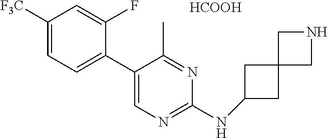 [SMILES: Cc1nc(NC2CC3(CNC3)C2)ncc1-c1ccc(C(F)(F)F)cc1F ; "HCOOH"]

Prepared as described for Example 98 using 2-chloro-5-(2-fluoro-4-(trifluoromethyl) phenyl)-4-methyl-pyrimidine (30 mg, 0.10 mmol) and tert-butyl 6-amino-2-azaspiro[3.3]heptane-2-carboxylate (44 mg, 0.20 mmol) to give the title compound (10 mg, 23%, 96.3% purity by HPLC) as a white powder. MS ES⁺: 367.1.

### Example 123: N-(5-(2-fluoro-4-(trifluoromethyl)phenyl)-4-methyl-pyrimidin-2-yl)-6-azaspiro[2.5]octan-1-amine, hydrochloride salt

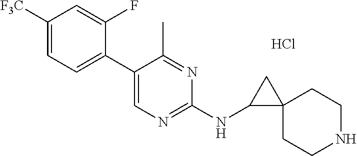 [SMILES: Cc1nc(NC2CC23CCNCC3)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (30 mg, 0.10 mmol) and tert-butyl 1-amino-6-azaspiro[2.5]octane-6-carboxylate hydrochloride (27 mg, 0.10 mmol) to give the title compound (10 mg, 22%, 97.4% purity by HPLC) as a light yellow gummy solid. MS ES⁺: 381.1.

### Example 124: 5-(2-fluoro-4-(trifluoromethyl)phenyl)-N-((3S,4S)-3-fluoropiperidin-4-yl)-4-methyl-pyrimidin-2-amine, hydrochloride salt

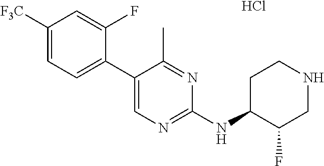 [SMILES: Cl ; Cc1nc(N[C@H]2CCNC[C@@H]2F)ncc1-c1ccc(C(F)(F)F)cc1F]

Prepared as described for Example 82 using 2-chloro-5-(2-fluoro-4-(trifluoromethyl) phenyl)-4-methyl-pyrimidine (30 mg, 0.10 mmol) and tert-butyl (3S,4S)-4-amino-3-fluoro-piperidine-1-carboxylate (45 mg, 0.20 mmol) to give the title compound (10 mg, 22%, 93% purity by HPLC) as a light yellow gummy solid. MS ES⁺: 373.0.

### Example 125: (1S,3S)—N¹-(5-(2-fluoro-4-(trifluoromethyl)phenyl)-4-methyl-pyrimidin-2-yl)cyclohexane-1,3-diamine, hydrochloride salt

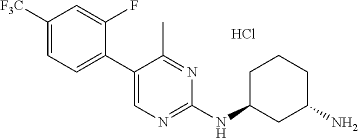 [SMILES: Cc1nc(N[C@H]2CCC[C@H](N)C2)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (30 mg, 0.10 mmol) and tert-butyl N-((1S,3S)-3-aminocyclohexyl)carbamate (44 mg, 0.20 mmol) to give the title compound (10 mg, 26%, 97% purity by HPLC) as a light yellow gummy solid. MS ES⁺: 369.1

### Example 126: 5-(2-fluoro-4-(trifluoromethyl)phenyl)-4-methyl-N-(2,2,6,6-tetramethylpiperidin-4-yl)pyrimidin-2-amine, formate salt

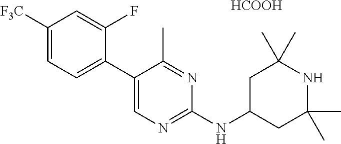 [SMILES: Cc1nc(NC2CC(C)(C)NC(C)(C)C2)ncc1-c1ccc(C(F)(F)F)cc1F ; "HCOOH"]

Prepared as described for Example 98 using 2-chloro-5-(2-fluoro-4-(trifluoromethyl) phenyl)-4-methyl-pyrimidine (30 mg, 0.10 mmol) and 2,2,6,6-tetramethylpiperidin-4-amine (32 mg, 0.20 mmol) to give the title compound (15 mg, 32%, 99.8% purity by HPLC) as a light yellow gummy solid. ¹H NMR (400 MHz, DMSO-d₆): 8.38 (s, 1H), 8.16 (s, 1H), 7.85-7.74 (m, 1H), 7.71-7.59 (m, 2H), 7.47 (br s, 1H), 4.30 (s, 1H), 2.16 (s, 3H), 1.89 (d, J=11.5 Hz, 2H), 1.48-1.18 (m, 14H). MS ES⁺: 411.1.

### Example 127: (S)-5-(2-fluoro-4-(trifluoromethyl)phenyl)-4-methyl-N-(piperidin-3-ylmethyl)pyrimidin-2-amine, hydrochloride salt

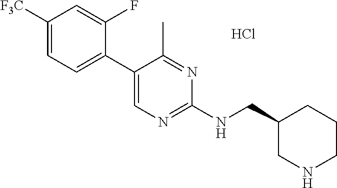 [SMILES: Cc1nc(NC[C@H]2CCCNC2)ncc1-c1ccc(C(F)(F)F)cc1F ; Cl]

Prepared as described for Example 82 using 2-chloro-5-(2-fluoro-4-(trifluoromethyl) phenyl)-4-methyl-pyrimidine (30 mg, 0.10 mmol) and tert-butyl (3R)-3-(aminomethyl)piperidine-1-carboxylate (44 mg, 0.20 mmol) to give the title compound (10 mg, 23%, 97% purity by HPLC) as a light yellow gummy solid. MS ES⁺: 369.0.

### Example 128: (R)-5-(2-fluoro-4-(trifluoromethyl)phenyl)-4-methyl-N-(piperidin-3-yl) pyrimidin-2-amine, hydrochloride salt

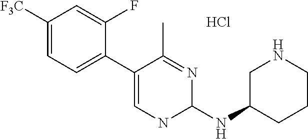

Prepared as described for Example 82 using 2-chloro-5-(2-fluoro-4-(trifluoromethyl) phenyl)-4-methyl-pyrimidine (30 mg, 0.10 μmol) and tert-butyl (3R)-3-aminopiperidine-1-carboxylate (41 mg, 0.206 mmol) to give the title compound (10 mg, 24%, 97.1% purity by HPLC) as a light yellow gummy solid. MS ES⁺: 355.0.

### Example 129a/b: 5-(2-fluoro-4-(trifluoromethyl)phenyl)-4-methyl-N-(1-((2R)-pyrrolidin-2-yl)ethyl)pyrimidin-2-amine

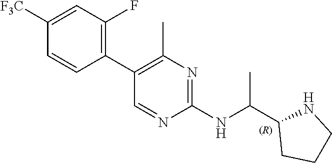

Step 1: A mixture of tert-butyl (2R)-2-acetylpyrrolidine-1-carboxylate (600 mg, 2.81 mmol), NH₄OAc (1.08 g, 14.05 mmol) and NaBH₃CN (441.45 mg, 7.03 mmol) in MeOH (7 mL) was sealed, heated at 80° C. for 2 h (microwave) and concentrated. The resulting yellow solid was dissolved in H₂O (10 mL) and extracted with EtOAc (3×10 mL). The combined organic layers were washed with brine (30 mL), dried over Na₂SO₄, filtered and concentrated under reduced pressure to give (2R)-tert-butyl 2-(1-aminoethyl)pyrrolidine-1-carboxylate (780 mg) as a yellow gummy solid, which was used in the next step without further purification.

Step 2: A solution of 2-chloro-5-(2-fluoro-4-(trifluoromethyl)phenyl)-4-methyl-pyrimidine (1.24 g, 4.26 mmol) and (2R)-tert-butyl 2-(1-aminoethyl)pyrrolidine-1-carboxylate (760 mg, 3.55 mmol) in dioxane (3.5 mL) was treated with DIPEA (916 mg, 7.10 mmol), stirred at 110° C. for 16 h, cooled to RT and extracted with EtOAc (3×10 mL). The combined organic layers were washed with brine (30 mL), dried over Na₂SO₄, filtered and concentrated under reduced pressure. The residue was purified by flash silica gel chromatography (ISCO, 25 g SepaFlash Silica Flash Column, 0˜40% EtOAc/petroleum ether) to give (2R)-tert-butyl 2-(1-((5-(2-fluoro-4-(trifluoromethyl)phenyl)-4-methyl-pyrimidin-2-yl)amino)ethyl)-pyrrolidine-1-carboxylate (520 mg, 24%, 78% purity by HPLC) as a brown gummy solid. MS ES⁺: 469.2.

Step 3: The material from step 2 was purified by SFC separation (DAICEL CHIRALPAK AD, 250 mm×30 mm×10 μm); mobile phase: A (supercritical CO₂), B (0.1% NH₃·H₂O in EtOH), A:B=85:15, flow=60 mL/min) to give diastereomer 1 (420 mg, 96% purity by HPLC) and diastereomer 2 (460 mg, 96% purity by HPLC) as brown gummy solids.

### Example 129a (isomer 1): 5-(2-fluoro-4-(trifluoromethyl)phenyl)-4-methyl-N-((1*R)-1-((2R)-pyrrolidin-2-yl)ethyl)pyrimidin-2-amine, semi-fumarate salt

Step 1: A solution of diastereomer 1, (R)-tert-butyl 2-((*R)-1-((5-(2-fluoro-4-(trifluoromethyl)phenyl)-4-methyl-pyrimidin-2-yl)amino)ethyl)pyrrolidine-1-carboxylate (420 mg, 0.897 mmol) was treated with HCl/dioxane (4M, 4 mL), stirred at 25° C. for 1 h and concentrated. The resulting gummy solid was dissolved in H₂O (5 mL) and EtOAc (5 mL), treated with sat. aq. NaHCO₃ (to adjust to pH=8) and the layers were separated. The aqueous phase was extracted with EtOAc (3×5 mL). The combined organic layers were washed with brine (10 mL), dried over Na₂SO₄, and concentrated under reduced pressure to give 5-(2-fluoro-4-(trifluoromethyl)phenyl)-4-methyl-N—((*R)-1-((R)-pyrrolidin-2-yl) ethyl)pyrimidin-2-amine (290 mg, 83%, 94.7% purity by HPLC) as a yellow gummy solid, which was used in the next step without further purification.

Step 2: A solution of 5-(2-fluoro-4-(trifluoromethyl)phenyl)-4-methyl-N—((*R)-1-((R)-pyrrolidin-2-yl)ethyl)pyrimidin-2-amine (40 mg, 0.109 mmol) in EtOH (0.5 mL) was treated with fumaric acid (6.30 mg, 0.054 mmol), stirred at 25° C. for 1 h and concentrated to afford a white solid, which was partitioned between ACN (2 mL) and H₂O (10 mL). The mixture was lyophilized to dryness to give isomer 1 (17 mg, 35%, 95.7% purity by HPLC) as a white solid. ¹H NMR (400 MHz, D₂O): 8.15 (s, 1H), 7.71-7.54 (m, 2H), 7.54-7.41 (m, 1H), 6.49 (s, 11H), 4.30-4.13 (m, 1H), 3.78-3.55 (m, 1H), 3.46-3.19 (m, 2H), 2.28-2.18 (m, 4H), 2.17-1.80 (m, 3H), 1.38-1.26 (m, 3H). MS ES⁺: 369.2. SFC: 90.8%, 2.32 min.

### Example 129b (isomer 2): 5-(2-fluoro-4-(trifluoromethyl)phenyl)-4-methyl-N-((1*S)-1-((2R)-pyrrolidin-2-yl)ethyl)pyrimidin-2-amine, 0.7 eq. fumarate salt

Prepared as described for Example 129a using diastereomer 2 (460 mg, 0.94 mmol) to afford the title compound (36 mg, 8%, 97.1% purity by HPLC) as a light yellow solid. ¹H NMR (400 MHz, D₂O): 8.18 (s, 1H), 7.67-7.56 (m, 2H), 7.55-7.47 (m, 1H), 6.58 (s, 1.4H), 4.57-4.43 (m, 1H), 3.91-3.75 (m, 1H), 3.37-3.07 (m, 2H), 2.25 (s, 3H), 2.21-1.89 (m, 4H), 1.35 (d, J=7.2 Hz, 3H). MS ES⁺: 369.3. SFC: 100%, 3.14 min.

### Example 130a/b: 5-(2-fluoro-4-(trifluoromethyl)phenyl)-4-methyl-N-(1-((2S)-pyrrolidin-2-yl)ethyl)pyrimidin-2-amine

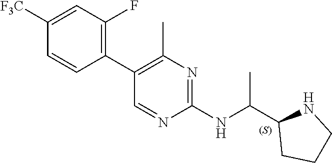

### Example 130a (isomer 1): 5-(2-fluoro-4-(trifluoromethyl)phenyl)-4-methyl-N-((1*R)-1-((2S)-pyrrolidin-2-yl)ethyl)pyrimidin-2-amine, fumarate salt

Prepared as described for Example 129a using tert-butyl (2S)-2-acetylpyrrolidine-1-carboxylate (500 mg, 2.34 mmol) to afford the title compound (25 mg, 2%, 96.8% purity by HPLC) as a white solid. ¹H NMR (400 MHz, D₂O): 8.00 (s, 1H), 7.49-7.39 (m, 2H), 7.36-7.29 (m, 1H), 6.39 (s, 2H), 4.16-4.00 (m, 1H), 3.59-3.43 (m, 1H), 3.29-3.07 (m, 2H), 2.16-2.09 (m, 1H), 2.09-2.04 (m, 3H), 2.01-1.79 (m, 2H), 1.72-1.57 (m, 1H), 1.15 (d, J=6.6 Hz, 3H). MS ES⁺: 369.2. SFC: 100%, 3.55 min.

### Example 130b (isomer 2): 5-(2-fluoro-4-(trifluoromethyl)phenyl)-4-methyl-N-((1*S)-1-((2S)-pyrrolidin-2-yl)ethyl)pyrimidin-2-amine, semi-fumarate salt

Prepared as described for Example 129a using tert-butyl (2S)-2-acetylpyrrolidine-1-carboxylate (500 mg, 2.34 mmol) to afford the title compound (60 mg, 6%, 95.0% purity by HPLC) as a white powder. ¹H NMR (400 MHz, D₂O) 7.90 (s, 1H), 7.46-7.10 (m, 3H), 6.44 (s, 1H), 4.42-4.23 (m, 1H), 3.82-3.61 (m, 1H), 3.34-3.15 (m, 1H), 3.06 (td, J=7.4, 11.4 Hz, 1H), 2.20-1.69 (m, 7H), 1.21 (d, J=6.6 Hz, 3H). MS ES⁺: 369.1. SFC: 99.1%, 3.81 min.

### Example 131a/b: 5-(2-fluoro-4-(trifluoromethyl)phenyl)-4-methyl-N-(1-((2R)-1-methylpyrrolidin-2-yl)ethyl) pyrimidin-2-amine

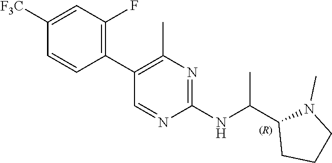

### Example 131a (isomer 1: 5-(2-fluoro-4-(trifluoromethyl)phenyl)-4-methyl-N-((1*R)-1-((2R)-1-methylpyrrolidin-2-yl)ethyl)pyrimidin-2-amine, 0.8 eq. fumarate salt

Step 1: A solution of 5-(2-fluoro-4-(trifluoromethyl)phenyl)-4-methyl-N-((1*R)-1-((2R)-pyrrolidin-2-yl)ethyl)pyrimidin-2-amine (150 mg, 0.41 mmol) and DIPEA (79 mg, 0.61 mmol) in THF (2 mL) was treated with Mel (75 mg, 0.53 mmol) and stirred at 0° C. for 2 h. The mixture was extracted with EtOAc (3×5 mL). The combined organic layers were washed with brine (10 mL), dried over Na₂SO₄, and concentrated under reduced pressure. The residue was purified by prep-HPLC (Phenomenex Luna C18 75×30 mm×3 μm, mobile phase A: 0.225% aq. HCOOH, mobile phase B: ACN; flow rate: 25 mL/min, gradient from 20% B to 50%). The product was dissolved in ACN (2 mL) and H₂O (10 mL) and lyophilized. The product was dissolved in H₂O (10 mL) and treated with sat. aq. NaHCO₃ to adjust to pH=8. The mixture was extracted with DCM (3×10 mL). The combined organic layers were washed with brine 10 mL (10 mL), dried over Na₂SO₄, filtered and evaporated to give 5-(2-fluoro-4-(trifluoromethyl)phenyl)-4-methyl-N-((1*R)-1-((2R)-1-methyl-pyrrolidin-2-yl)ethyl)pyrimidin-2-amine (120 mg, 77%) as a pale yellow gummy solid. MS ES⁺: 383.2.

Step 2: A solution of 5-(2-fluoro-4-(trifluoromethyl)phenyl)-4-methyl-N-((1*R)-1-((2R)-1-methylpyrrolidin-2-yl)ethyl)pyrimidin-2-amine (20 mg, 0.052 mmol) in EtOH (0.5 mL) was treated with fumaric acid (4.86 mg, 0.042 mmol), stirred at 25° C. for 1 h and concentrated. The resulting residue was dissolved in ACN (2 mL) and H₂O (10 mL) and lyophilized to give the title compound (22 mg, 87%, 97.1% purity by HPLC) as a white solid. ¹H NMR (400 MHz, D₂O): 8.22-8.13 (m, 1H), 7.65-7.54 (m, 2H), 7.54-7.40 (m, 1H), 6.62 (s, 1.6H), 4.34-4.17 (m, 1H), 3.80-3.51 (m, 2H), 3.40-3.12 (m, 1H), 3.10-2.67 (m, 3H), 2.43-2.29 (m, 1H), 2.27-2.21 (m, 3H), 2.20-1.84 (m, 3H), 1.30 (d, J=6.5 Hz, 3H). MS ES⁺: 383.2. SFC: 91.6%, 1.50 min.

### Example 131b (isomer 2): 5-(2-fluoro-4-(trifluoromethyl)phenyl)-4-methyl-N-((1*S)-1-((2R)-1-methylpyrrolidin-2-yl)ethyl)pyrimidin-2-amine, 1.1 eq. fumarate salt

Prepared as described for Example 131a using 5-(2-fluoro-4-(trifluoromethyl)phenyl)-4-methyl-N-((1*S)-1-((2R)-pyrrolidin-2-yl)ethyl)pyrimidin-2-amine (150 mg, 0.407 mmol) to afford the title compound (41 mg, 19%, 98.9% purity by HPLC) as a white solid. ¹H NMR (400 MHz, D₂O): 8.23-8.12 (m, 1H), 7.65-7.53 (m, 2H), 7.53-7.43 (m, 1H), 6.66 (s, 2.2H), 4.69-4.56 (m, 1H), 3.64-3.48 (m, 2H), 3.16-3.09 (m, 1H), 3.06 (s, 3H), 2.33-2.17 (m, 4H), 2.16-1.79 (m, 3H), 1.32 (d, J=6.9 Hz, 3H). MS ES⁺: 383.3. SFC: 99.4%, 1.63 min.

### Example 132a/b: 5-(2-fluoro-4-(trifluoromethyl)phenyl)-4-methyl-N-(1-((2S)-1-methylpyrrolidin-2-yl)ethyl)pyrimidin-2-amine

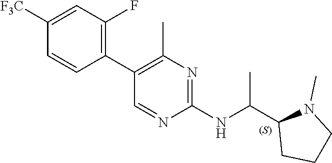

### Example 132a (isomer 1): 5-(2-fluoro-4-(trifluoromethyl)phenyl)-4-methyl-N-((1*R)-1-((2S)-1-methylpyrrolidin-2-yl)ethyl)pyrimidin-2-amine, fumarate salt

Prepared as described for Example 131a using 5-(2-fluoro-4-(trifluoromethyl)phenyl)-4-methyl-N-((1*R)-1-((2S)-pyrrolidin-2-yl)ethyl)pyrimidin-2-amine (150 mg, 0.407 mmol) to afford the title compound (46 mg, 29%) as a light yellow solid. ¹H NMR (400 MHz, D₂O) 8.16 (s, 1H), 7.62-7.51 (m, 2H), 7.49-7.38 (m, 1H), 6.66 (s, 2H), 4.41-4.14 (m, 1H), 3.79-3.47 (m, 2H), 3.35-3.08 (m, 1H), 2.91 (s, 3H), 2.41-2.27 (m, 1H), 2.26-2.17 (m, 3H), 2.16-1.97 (m, 2H), 1.96-1.81 (m, 1H), 1.34-1.18 (m, 3H). MS ES⁺: 383.1. SFC: 99.9%, 14.9 min.

### Example 132b (isomer 2): 5-(2-fluoro-4-(trifluoromethyl)phenyl)-4-methyl-N-((1*S)-1-((2S)-1-methylpyrrolidin-2-yl)ethyl)pyrimidin-2-amine, semi-fumarate salt

Prepared as described for Example 131a using 5-(2-fluoro-4-(trifluoromethyl)phenyl)-4-methyl-N-((1*S)-1-((2S)-pyrrolidin-2-yl)ethyl)pyrimidin-2-amine (150 mg, 0.407 mmol) to afford the title compound (35 mg, 18%, 94.7% purity by HPLC) as a light yellow solid. ¹H NMR (400 MHz, D₂O): 8.20 (s, 1H), 7.64-7.53 (m, 2H), 7.52-7.42 (m, 1H), 6.73 (s, 1H), 3.77-3.51 (m, 3H), 3.20-3.01 (m, 4H), 2.34-2.20 (m, 2H), 2.13-1.81 (m, 2H), 1.34-1.30 (m, 2H), 1.29-1.26 (m, 3H). MS ES⁺: 383.1. SFC: 96%, 15.2 min.

### Example 133a/b: 5-(2-fluoro-4-(trifluoromethyl)phenyl)-N-(((trans)-3-fluoropyrrolidin-2-yl)methyl)-4-methyl-pyrimidin-2-amine

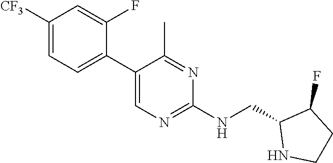 [SMILES: Cc1nc(NC[C@H]2NCC[C@@H]2F)ncc1-c1ccc(C(F)(F)F)cc1F]

### Example 133a (isomer 1): 5-(2-fluoro-4-(trifluoromethyl)phenyl)-N-(((2*S,3*R)-3-fluoropyrrolidin-2-yl)methyl)-4-methyl-pyrimidin-2-amine, 1.4 eq. fumarate salt

Prepared as described for Example 96 using 2-chloro-5-(2-fluoro-4-(trifluoromethyl) phenyl)-4-methyl-pyrimidine (1.1 g, 3.78 mmol) and trans-tert-butyl 2-(aminomethyl)-3-fluoro-pyrrolidine-1-carboxylate (826.08 mg, 3.78 mmol) to afford the title compound (123.41 mg, 6%, 96.70% purity by HPLC) as a light yellow solid. ¹H NMR (400 MHz, D₂O): 8.20 (s, 1H), 7.71-7.54 (m, 2H), 7.54-7.42 (m, 1H), 6.68 (s, 2.8H), 5.49-5.29 (m, 1H), 4.34-4.19 (m, 1H), 3.84-3.45 (m, 4H), 2.53-2.28 (m, 2H), 2.25 (s, 3H). MS ES⁺: 373.2. SFC: 100%, 3.09 min.

### Example 133b (isomer 2): 5-(2-fluoro-4-(trifluoromethyl)phenyl)-N-(((2*R,3*S)-3-fluoropyrrolidin-2-yl)methyl)-4-methyl-pyrimidin-2-amine, fumarate salt

Prepared as described for Example 96 using 2-chloro-5-(2-fluoro-4-(trifluoromethyl) phenyl)-4-methyl-pyrimidine (1.1 g, 3.78 mmol) and trans-tert-butyl 2-(aminomethyl)-3-fluoro-pyrrolidine-1-carboxylate (826.08 mg, 3.78 mmol) to afford the title compound (122.23 mg, 6%, 97.39% purity by HPLC) as a light yellow solid. ¹H NMR (400 MHz, D₂O): 8.18 (s, 1H), 7.65-7.53 (m, 2H), 7.52-7.38 (m, 1H), 6.68 (s, 3H), 5.50-5.23 (m, 1H), 4.36-4.14 (m, 1H), 3.82-3.45 (m, 4H), 2.50-2.27 (m, 2H), 2.24 (s, 3H). MS ES⁺: 373.2. SFC: 99.8%, 3.44 min.

### Example 134: (S)-5-(2-fluoro-4-(trifluoromethyl)phenyl)-4-methyl-N-((2-methyl-pyrrolidin-2-yl)methyl)pyrimidin-2-amine, fumarate salt

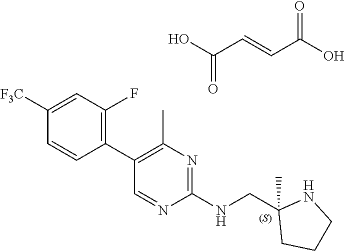

Prepared as described for Example 102 using 2-chloro-5-(2-fluoro-4-(trifluoromethyl) phenyl)-4-methyl-pyrimidine (300 mg, 1.03 mmol) and tert-butyl (2S)-2-(aminomethyl)-2-methyl-pyrrolidine-1-carboxylate (221 mg, 1.03 mmol) to afford the title compound (102 mg, 20%, 98.2% purity by HPLC) as a light yellow solid. ¹H NMR (400 MHz, D₂O): 8.20-8.04 (m, 11H), 7.63-7.34 (m, 3H), 6.53 (s, 2H), 3.82-3.47 (m, 3H), 3.39-3.21 (m, 2H), 2.28-1.90 (m, 6H), 1.88-1.78 (m, 1H), 1.51-1.31 (m, 3H). MS ES⁺: 369.1. SFC: 99.8%, 4.55 min.

### Example 135: (R)-5-(2-fluoro-4-(trifluoromethyl)phenyl)-4-methyl-N-((2-methyl-pyrrolidin-2-yl)methyl)pyrimidin-2-amine, fumarate salt

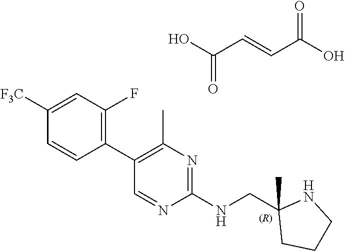

Prepared as described for Example 134 using 2-chloro-5-(2-fluoro-4-(trifluoromethyl) phenyl)-4-methyl-pyrimidine (136 mg, 0.467 mmol) and tert-butyl (2R)-2-(aminomethyl)-2-methyl-pyrrolidine-1-carboxylate (100 mg, 0.467 mmol) to afford the title compound (130 mg, 57%, 98.6% purity by HPLC) as a light yellow powder. ¹H NMR (400 MHz, D₂O): 7.91 (s, 1H), 7.41-7.15 (m, 3H), 6.55 (s, 2H), 3.75-3.42 (m, 3H), 3.40-3.19 (m, 2H), 2.27-1.88 (m, 6H), 1.85-1.74 (m, 1H), 1.27-1.27 (m, 1H), 1.35 (s, 3H). MS ES⁺: 369.1. SFC: 100%, 4.23 min.

### Example 136: (S)—N-((1,2-dimethylpyrrolidin-2-yl)methyl)-5-(2-fluoro-4-(trifluoromethyl)phenyl)-4-methyl-pyrimidin-2-amine, 1.5 eq. fumarate salt

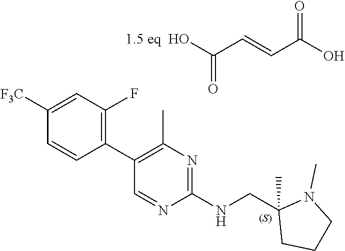

Step 1: A solution of (S)-5-(2-fluoro-4-(trifluoromethyl)phenyl)-4-methyl-N-((2-methylpyrrolidin-2-yl)methyl)pyrimidin-2-amine (100 mg, 0.271 mmol) and DIPEA (42 mg, 0.33 mmol) in THF (0.5 mL) was treated with Mel (46 mg, 0.33 mmol) at 0° C., stirred at 0° C. for 1.5 h, and extracted with EtOAc (3×5 mL). The combined organic layers were washed with brine (10 mL), dried over Na₂SO₄ and concentrated. The resulting yellow oil was purified by prep-TLC (DCM/MeOH=10:1) to give the free base product (52 mg, 48%, 96.28% purity by HPLC) as a light yellow gummy solid.

Step 2: A solution of (S)—N-((1,2-dimethylpyrrolidin-2-yl)methyl)-5-(2-fluoro-4-(trifluoromethyl)phenyl)-4-methyl-pyrimidin-2-amine (40 mg, 0.11 mmol) in EtOH (0.5 mL) was treated with fumaric acid (18 mg, 0.16 mmol), stirred at 25° C. for 1 h and concentrated. The resulting oil was dissolved in ACN (2 mL) and H₂O (5 mL) and lyophilized to give the title compound (22 mg, 37%, 95.1% purity by HPLC) as a light yellow solid. ¹H NMR (400 MHz, D₂O): 8.17 (s, 1H), 7.61-7.53 (m, 2H), 7.50-7.42 (m, 1H), 6.70-6.66 (m, 3H), 3.92-3.81 (m, 1H), 3.72-3.56 (m, 2H), 3.24-3.10 (m, 1H), 2.91-2.77 (m, 3H), 2.23 (s, 3H), 2.18-1.81 (m, 4H), 1.32 (s, 3H). MS ES⁺: 383.2. SFC: 99.2%, 11.4 min.

### Example 137: (R)—N-((1,2-dimethylpyrrolidin-2-yl)methyl)-5-(2-fluoro-4-(trifluoromethyl)phenyl)-4-methyl-pyrimidin-2-amine, di-fumarate salt

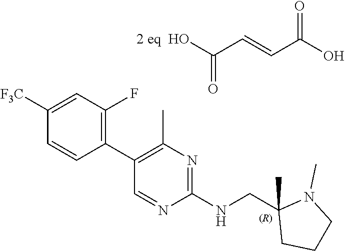

Prepared as described for Example 136 using (R)-5-(2-fluoro-4-(trifluoromethyl)phenyl)-4-methyl-N-((2-methylpyrrolidin-2-yl)methyl)pyrimidin-2-amine (150 mg, 0.407 mmol) to afford the title compound (50 mg, 20%, 93.9% purity by HPLC) as a white powder. ¹H NMR (400 MHz, D₂O): 8.24 (s, 1H), 7.68-7.54 (m, 2H), 7.48 (t, J=7.4 Hz, 1H), 6.74 (s, 4H), 3.95 (d, J=15.2 Hz, 1H), 3.66 (d, J=15.2 Hz, 2H), 3.29-3.13 (m, 1H), 2.84 (s, 3H), 2.28 (s, 3H), 2.21-2.05 (m, 2H), 2.04-1.83 (m, 2H), 1.33 (s, 3H). MS ES⁺: 383.2. SFC: 100%, 10.54 min.

### Example 138a/b/c/d: N-((1,5-dimethylpyrrolidin-2-yl)methyl)-5-(2-fluoro-4-(trifluoromethyl)phenyl)-4-methyl-pyrimidin-2-amine

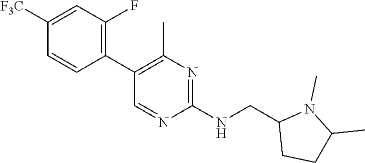 [SMILES: Cc1nc(NCC2CCC(C)N2C)ncc1-c1ccc(C(F)(F)F)cc1F]

Step 1: A suspension of LiAlH₄ (947 mg, 24.9 mmol) in THF (30 mL) was treated dropwise with a solution of 1,5-dimethylpyrrole-2-carbonitrile (3.00 g, 24.9 mmol) in THF (30 mL) under ice cooling, stirred at 85° C. for 1 h, cooled to 0° C. and treated dropwise with aq. NH₃ (28%) until pH >8 was reached. The mixture was filtered and extracted with DCM (3×100 mL). The combined organic layers were dried over Na₂SO₄ and evaporated to give (1,5-dimethylpyrrol-2-yl)methanamine (3.00 g, 91.9%, 95% purity by HPLC) as a yellow oil. ¹H NMR (400 MHz, CD₃Cl): 5.92 (d, J=3.3 Hz, 1H), 5.82 (d, J=3.3 Hz, 1H), 3.92-3.72 (m, 2H), 3.50 (s, 3H), 2.23 (s, 3H).

Step 2: A solution of (1,5-dimethylpyrrol-2-yl)methanamine (2.00 g, 16.1 mmol) in conc. aq. HCl (2 mL) and EtOH (20 mL) was treated with PtO₂ (731 mg, 3.22 mmol) under N₂. The suspension was purged with H₂ (3×) and stirred under H₂ (50 psi) at 25° C. for 12 h. The mixture was filtered and the filtrate concentrated. The pH was adjusted to 9-10 by addition of 5M aq. NaOH solution and extracted with DCM/MeOH (10:1, 3×50 mL). The combined organic layers were dried over Na₂SO₄ and evaporated to give (1,5-dimethylpyrrolidin-2-yl)methanamine (2.00 g, 97%) as a dark oil, which was used in the next step without further purification.

Step 3: A solution of 2-chloro-5-(2-fluoro-4-(trifluoromethyl)phenyl)-4-methyl-pyrimidine (0.7 0 g, 2.41 mmol), (1,5-dimethylpyrrolidin-2-yl)methanamine (617 mg, 4.82 mmol) and DIPEA (622 mg, 4.82 mmol) in dioxane (5 mL) was stirred at 110° C. for 8 h, cooled to RT and extracted with DCM (3×50 mL). The combined organic layers were dried over Na₂SO₄ and evaporated. The residue was purified by prep-HPLC (YMC-Triart Prep C18 (150 mm×40 mm, 7 μm); mobile phase A: 0.2% aq. HCOOH, mobile phase B: ACN; flow rate: 25 ml/min, gradient from 20% to 50% B). The product was dissolved in ACN (5 mL) and H₂O (20 mL), lyophilized and separated into diastereomeric pairs of enantiomers by SFC (DAICEL CHIRALPAK IC (250 mm×30 mm, 10 μm); mobile phase A: supercritical CO₂, B: 0.1% NH₃·H₂O in EtOH, A:B=70:30; flow rate=70 mL/min; T: 38° C.; UV detection at 220 nm) to give diastereomeric pair of enantiomers 1 (600 mg, 65%, 99.3% purity by HPLC) and diastereomeric pair of enantiomer 2 (65 mg, 6.91%, 97.9% purity by HPLC) as yellow gummy solids.

The diastereomeric pair of enantiomers 1 was separated into enantiomers using chiral HPLC (CHIRALPAK IG-3 (IG30CD-WE016); mobile phase: EtOH/DIPEA=100/0.1; flow rate 0.5 mL/min; T=25° C.; UV detection at 254 nm) to give isomer 1 (236 mg) and isomer 2 (223 mg).

### Example 138a (isomer U: N-(((2*R,5*R)-1,5-dimethylpyrrolidin-2-yl)methyl)-5-(2-fluoro-4-(trifluoromethyl)phenyl)-4-methyl-pyrimidin-2-amine, fumarate salt

A solution of isomer 1 (160 mg, 0.418 mmol) in EtOH (2 mL) was treated with fumaric acid (107 mg, 0.92 mmol), stirred at 25° C. for 1 h and concentrated. The resulting residue was dissolved in ACN (2 mL) and H₂O (10 mL) and lyophilized to give N-(((2*R,5*R)-1,5-dimethylpyrrolidin-2-yl)methyl)-5-(2-fluoro-4-(trifluoromethyl)phenyl)-4-methyl-pyrimidin-2-amine fumarate (214 mg, 79%, 98.3% purity by HPLC) as a white solid. ¹H NMR (400 MHz, D₂O): 8.17 (s, 1H), 7.63-7.50 (m, 2H), 7.49-7.38 (m, 1H), 6.88-6.52 (m, 4.4H), 3.91-3.79 (m, 1H), 3.79-3.66 (m, 2H), 3.50-3.36 (m, 1H), 2.96 (s, 3H), 2.34-2.15 (m, 5H), 2.03-1.86 (m, 1H), 1.75-1.57 (m, 1H), 1.35 (d, J=6.5 Hz, 3H). MS ES⁺: 383.2. SFC: 99.4%, 1.22 min.

### Example 138b (isomer 2): N-(((2*S,5*S)-1,5-dimethylpyrrolidin-2-yl)methyl)-5-(2-fluoro-4-(trifluoromethyl)phenyl)-4-methyl-pyrimidin-2-amine, fumarate salt

A solution of isomer 2 (222 mg, 0.581 mmol) and fumaric acid (134.77 mg, 1.16 mmol) in EtOH (2 mL) was stirred at 25° C. for 1 h and evaporated. The residue was dissolved in ACN (2 mL) and H₂O (10 mL) and lyophilized to give N-(((2*S,5*S)-1,5-dimethylpyrrolidin-2-yl)methyl)-5-(2-fluoro-4-(trifluoromethyl)phenyl)-4-methyl-pyrimidin-2-amine fumarate (260 mg, 72%, 99.3% purity by HPLC) as an off-white solid. ¹H NMR (400 MHz, D₂O): 7.97 (d, J=19.6 Hz, 1H), 7.44-7.11 (m, 3H), 6.63 (d, J=8.1 Hz, 4H), 3.68 (d, J=5.1 Hz, 3H), 3.37 (s, 1H), 3.09-2.84 (m, 3H), 2.30-1.96 (m, 5H), 1.85 (s, 1H), 1.55 (s, 1H), 1.38-1.22 (m, 3H). MS ES⁺: 383.3. SFC: 99.8%, 1.30 min.

The diastereomeric pair of enantiomers 2 was separated into enantiomers by SFC (DAICEL CHIRALPAK IC (250 mm×30 mm, 10 μm); mobile phase: A: supercritical CO₂, B: 0.1% NH₃H₂O in EtOH, A:B=90:10; flow rate=60 mL/min; T=38° C.; UV detection at 220 nm). The enantiomerically pure products were dissolved in ACN (2 mL) and H₂O (10 mL) and lyophilized to give isomer 3 (31 mg) and isomer 4 (31 mg).

### Example 138c (isomer 3): N-(((2*R,5*S)-1,5-dimethylpyrrolidin-2-yl)methyl)-5-(2-fluoro-4-(trifluoromethyl)phenyl)-4-methyl-pyrimidin-2-amine, fumarate salt

A solution of isomer 3 (31 mg, 0.08 mmol) and fumaric acid (9.4 mg, 0.08 mmol) in EtOH (1 mL) was stirred at 25° C. for 1 h and evaporated. The residue was dissolved in ACN (2 mL) and H₂O (10 mL) and lyophilized to give N-(((2*R,5*S)-1,5-dimethylpyrrolidin-2-yl)methyl)-5-(2-fluoro-4-(trifluoromethyl)phenyl)-4-methyl-pyrimidin-2-amine fumarate (22 mg, 47%, 97.7% purity by HPLC) as a yellow solid. ¹H NMR (400 MHz, D₂O): 8.19-8.01 (m, 1H), 7.61-7.31 (m, 3H), 6.69-6.59 (m, 3H), 3.95-3.51 (m, 4H), 2.89-2.74 (m, 3H), 2.35-2.01 (m, 5H), 1.97-1.72 (m, 2H), 1.35-1.20 (m, 3H). MS ES⁺: 383.3.

### Example 138d (isomer 4): N-(((2*S,5*R)-1,5-dimethylpyrrolidin-2-yl)methyl)-5-(2-fluoro-4-(trifluoromethyl)phenyl)-4-methyl-pyrimidin-2-amine, fumarate salt

A solution of isomer 4 (27 mg, 0.07 mmol) and fumaric acid (8.2 mg, 0.07 mmol) in EtOH (1 mL) was stirred at 25° C. for 1 h and evaporated. The residue was dissolved in ACN (2 mL) and H₂O (10 mL) and lyophilized to give N-(((2*S,5*R)-1,5-dimethylpyrrolidin-2-yl)methyl)-5-(2-fluoro-4-(trifluoromethyl)phenyl)-4-methyl-pyrimidin-2-amine fumarate (20 mg, 48%, 95.2% purity by HPLC) as a yellow solid. ¹H NMR (400 MHz, D₂O): 8.21-8.01 (m, 1H), 7.63-7.31 (m, 3H), 6.64 (d, J=3.1 Hz, 3H), 3.93-3.53 (m, 4H), 2.81 (s, 3H), 2.40-2.08 (m, 5H), 1.96-1.64 (m, 2H), 1.33-1.16 (m, 3H). MS ES⁺: 383.3.

### Example 139: (R)—N-(5-(2-fluoro-4-(trifluoromethyl)phenyl)-4-methyl-pyrimidin-2-yl)-1-methylpyrrolidine-2-carboxamide

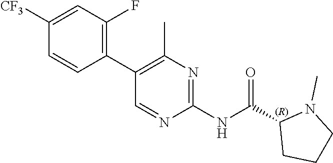

Step 1: A mixture of (2-fluoro-4-(trifluoromethyl)phenyl)boronic acid (1.00 g, 4.81 mmol), 5-bromo-4-methyl-pyrimidin-2-amine (904 mg, 4.81 mmol), K₂CO₃ (1.33 g, 9.62 mmol) and 4-di(tert-butyl)phosphanyl-N,N-dimethyl-aniline-dichloropalladium (681 mg, 961 μmol) in dioxane (10 mL) and H₂O (5 mL) was de-gassed (N₂), stirred at 110° C. for 10 h, cooled to RT and evaporated. The residue was purified by silica gel chromatography (EtOAc/petroleum ether 1:1) to afford 5-(2-fluoro-4-(trifluoromethyl)phenyl)-4-methyl-pyrimidin-2-amine (0.95 g, 69%, 95% purity by HPLC) as a white solid. MS ES⁺: 272.1.

Step 2: A solution of 5-(2-fluoro-4-(trifluoromethyl)phenyl)-4-methyl-pyrimidin-2-amine (300 mg, 1.11 mmol), (2R)-1-methylpyrrolidine-2-carboxylic acid (143 mg, 1.11 mmol) and pyridine (892 μL, 11.1 mmol) in DCM (1 mL) was treated dropwise with POCl₃ (170 mg, 1.11 mmol) at −40° C. and stirred for 1 h. The mixture was poured into ice H₂O (20 mL) and extracted with DCM (3×20 mL). The combined organic layers were dried over Na₂SO₄ and evaporated. Purification of the residue by prep-HPLC (Phenomenex Luna C18 (80 mm×40 mm, 3 μm); mobile phase A: H₂O (0.225% HCOOH), mobile phase B: ACN; flow rate: 25 mL/min, gradient from 12% B to 42%). The product was dissolved in ACN (2 mL) and H₂O (10 mL) and lyophilized to give the title compound (35 mg, 8%, 96.8% purity by HPLC) as an off-white solid. ¹H NMR (400 MHz, DMSO-d₆): 10.17 (s, 1H), 8.55 (s, 1H), 7.95-7.84 (m, 1H), 7.80-7.68 (m, 2H), 3.18-3.10 (m, 1H), 3.06-2.99 (m, 1H), 2.39 (s, 4H), 2.33-2.29 (m, 3H), 2.26-2.15 (m, 1H), 1.87-1.75 (m, 3H). MS ES⁺: 383.2. SFC: 99.6%, 1.59 min.

### Example 140: (S)—N-(5-(2-fluoro-4-(trifluoromethyl)phenyl)-4-methyl-pyrimidin-2-yl)-1-methylpyrrolidine-2-carboxamide

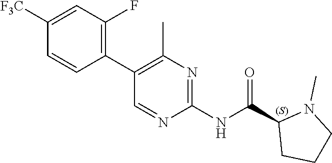

Prepared as described for Example 139 using (2S)-1-methylpyrrolidine-2-carboxylic acid (105 mg, 811 μmol) to afford the title compound (5.64 mg, 1.4%, 97.4% purity by HPLC) as a white solid. ¹H NMR (400 MHz, DMSO-de): δ 10.17 (s, 1H), 8.55 (s, 1H), 7.92-7.85 (m, 1H), 7.77-7.69 (m, 2H), 3.18-3.10 (m, 1H), 3.07-2.99 (m, 1H), 2.42-2.34 (m, 4H), 2.32-2.30 (m, 3H), 2.26-2.12 (m, 1H), 1.89-1.70 (m, 3H). MS ES⁺: 383.2. SFC: 96.8%, 1.40 min.

### 3. Biological Efficacy of Compounds of the Invention

The IC₅₀ values for the compounds of the examples is shown in Table 1.

The ability of the test compounds to inhibit nicotine stimulated nAChRα6 activity was determined in a fluorescence-based calcium assay. Compound activity was determined using a HEK cell line stably expressing a human nAChRα6-α3 chimera, in addition to β2 and β3^{V273S} subunits (as in Capelli et al, Br J Pharmacol 163(2): 313-29 2011). In this cell line, nicotine stimulated an increase in intracellular calcium concentration, measured as an increase in fluorescence when cells were incubated with a calcium sensitive dye. Test compounds were pre-incubated with cells prior to nicotine stimulation to detect any reduction in the magnitude of the nicotine response.

The day prior to the assay, compounds were serially diluted in DMSO (200× final assay concentration (FAC)), in 384-well plates which were then stored in the dark at room temperature (RT) until use. Cells were seeded at 20 k/well in black, poly-D-lysine coated, clear bottom 384-well plates and incubated for 4 hours at 37° C., followed by overnight incubation at 30° C. The following day, the DMSO compound plate was diluted 1:20 (10×FAC) in assay buffer (HEPES buffered saline solution with 10 mM HEPES, 0.1% bovine serum albumin, 1 mM probenecid). A nicotine solution was prepared in assay buffer (7×FAC) and dispensed into a 384-well plate. The growth media was removed from the cell plate, replaced with 53 μl calcium dye/well (Calcium 5; Molecular Devices) and the plate incubated at 30° C. for 45 minutes. Test compounds were then added to the cells (7 μl of 10×FAC) and incubated for 10 minutes, after which nicotine EC₈₀ (10 μl of 7×FAC) was added and changes in fluorescence measured using a FLIPR plate reader (Molecular Devices).

Compound activity was examined using a 10-point, half-log concentration-response range and each concentration was tested in duplicate wells. Responses were calculated as changes in relative fluorescence units (max-min) and IC₅₀ values were derived from this data using a four-parameter curve fit.

Results

TABLE 1

| Example number | IC₅₀ (μM) |
|---|---|
| 1 | 0.048 |
| 2 | 0.302 |
| 3 | 0.331 |
| 4 | 0.211 |
| 5 | 0.635 |
| 6 | 0.123 |
| 7 | 0.59 |
| 8 | 0.286 |
| 9 | 0.264 |
| 10 | 0.201 |
| 11 | 0.246 |
| 12 | 0.108 |
| 13 | 0.05 |
| 14 | 0.042 |
| 15 | 1.943 |
| 16 | 0.075 |
| 17 | 0.02 |
| 18 | 0.526 |
| 18a | 0.331 |
| 18b | 0.442 |
| 19 | 0.216 |
| 20 | 0.49 |
| 21 | 0.599 |
| 22 | 0.05 |
| 23 | 0.233 |
| 24a | 0.062 |
| 24b | 0.005 |
| 25 | 0.034 |
| 26 | 0.156 |
| 27 | 0.056 |
| 28a | 0.054 |
| 28b | 0.01 |
| 29 | 0.095 |
| 30 | 0.082 |
| 31 | 0.082 |
| 32 | 0.099 |
| 33 | 0.045 |
| 34 | 0.13 |
| 35 | 0.029 |
| 36 | 0.14 |
| 37 | 0.103 |
| 38a | 0.059 |
| 38b | 0.07 |
| 39a | 0.904 |
| 39b | 0.901 |
| 40 | 0.068 |
| 41 | 0.081 |
| 42 | 1.196 |
| 43 | 0.178 |
| 44a | 0.076 |
| 44b | 0.088 |
| 45 | 1.311 |
| 46 | 2.175 |
| 47 | 1.25 |
| 48 | 0.083 |
| 49 | 0.069 |
| 50 | 0.103 |
| 51 | 1.104 |
| 52 | 1.531 |
| 53a | 1.238 |
| 53b | 0.734 |
| 54a | 0.331 |
| 54b | 0.442 |
| 55a | 0.098 |
| 55b | 0.081 |
| 56a | 0.133 |
| 56b | 0.162 |
| 57a | 0.139 |
| 57b | 0.144 |
| 58a | 0.038 |
| 58b | 0.056 |
| 59a | 0.202 |
| 59b | 0.134 |
| 60 | 1.181 |
| 61 | 0.533 |
| 62 | 0.064 |
| 63 | 0.141 |
| 64a | 0.09 |
| 64b | 0.088 |
| 65 | 0.084 |
| 66 | 0.094 |
| 67 | 0.066 |
| 68a | 0.078 |
| 68b | 0.067 |
| 69a | 0.436 |
| 69b | 0.309 |
| 70 | 0.072 |
| 71 | 0.054 |
| 72 | 0.052 |
| 73 | nd |
| 74a | 0.693 |
| 74b | 1.474 |
| 75 | 0.094 |
| 76a | 0.118 |
| 76b | 0.107 |
| 77a | 0.07 |
| 77b | 0.093 |
| 78a | 0.061 |
| 78b | 0.06 |
| 79a | 0.42 |
| 79b | 0.296 |
| 80 | 1.469 |
| 81 | 0.098 |
| 82 | 0.018 |
| 83a | 1.151 |
| 83b | 0.456 |
| 84a | 1.528 |
| 84b | 1.646 |
| 85 | 0.051 |
| 86a | 0.122 |
| 86b | 0.126 |
| 87 | 0.045 |
| 88 | 0.149 |
| 89 | 0.18 |
| 90a | 0.211 |
| 90b | 0.186 |
| 91a | 0.008 |
| 91b | 0.028 |
| 92a | 0.033 |
| 92b | 0.073 |
| 93a | 0.05 |
| 93b | 0.011 |
| 94a | 0.005 |
| 94b | 0.005 |
| 95a | 0.048 |
| 95b | 0.07 |
| 96a | 0.151 |
| 96b | 0.099 |
| 97a | 0.184 |
| 97b | 0.152 |
| 97c | 0.18 |
| 97d | 0.299 |
| 98 | 0.067 |
| 99 | 0.083 |
| 100 | 0.11 |
| 101 | 0.109 |
| 102 | 0.005 |
| 103 | 0.04 |
| 104 | 0.027 |
| 104a | 0.104 |
| 104b | 0.045 |
| 105 | 0.05 |
| 106 | 0.054 |
| 106a | 0.105 |
| 106b | 0.138 |
| 107 | 0.154 |
| 108 | 0.043 |
| 108a | 0.086 |
| 108b | 0.077 |
| 109 | 0.096 |
| 110 | 0.054 |
| 111 | 0.09 |
| 112 | 0.107 |
| 113 | 0.045 |
| 114 | 0.142 |
| 115 | 0.121 |
| 116 | 0.127 |
| 117 | 0.025 |
| 118 | 0.075 |
| 119 | 0.155 |
| 120 | 0.179 |
| 121 | 0.191 |
| 122 | 0.039 |
| 123 | 0.15 |
| 124 | 0.903 |
| 125 | 0.223 |
| 126 | 0.259 |
| 127 | 0.119 |
| 128 | 0.18 |
| 129a | 0.065 |
| 129b | 0.105 |
| 130a | 0.118 |
| 130b | 0.049 |
| 131a | 0.172 |
| 131b | 0.291 |
| 132a | 0.092 |
| 132b | 0.097 |
| 133a | 0.305 |
| 133b | 0.30 |
| 134 | 0.101 |
| 135 | 0.07 |
| 136 | 0.096 |
| 137 | 0.11 |
| 138a | 0.207 |
| 138b | 0.085 |
| 138c | 0.069 |
| 138d | 0.025 |
| 139 | 0.086 |
| 140 | 0.079 |

### 4. In Vivo Efficacy: Tacrine Studies

Resting tremor is one of the classic symptoms of Parkinson's disease. These characteristic tremors can be modelled pharmacologically in preclinical models using cholinomimetics such as the acetylcholine esterase inhibitor, tacrine (5 mg/kg, i.p.) (Salamone et al, Prog Neurobiol 56: 591-611 1998) to induce tremulous jaw movement behaviours. These tremulous jaw movements are thought to arise from an imbalance between cholinergic and dopaminergic neurotransmission and are thought to mimic the imbalances in neurotransmission resulting from the dopamine loss seen in Parkinson's disease (Aosaki et al, Geriatr Gerontol Int 10: s148-s157 2010). These tremors can be reversed by agents increasing the dopamine tone, such as dopamine agonists (Salamone et al, Behav Brain Res 156: 173-179 2005).

The objective of these studies was to evaluate the effects of oral administration of Example Compounds on tremulous jaw movements induced by tacrine in male C57BL/6J mice or Sprague-Dawley rats. Animals were administered vehicle (0.5% methylcellulose, p.o.) or Example Compound (across a range of doses; 10 mg/kg, p.o. data shown) 15 to 60 minutes prior to behavioural testing (n=10/group). Tacrine (5 mg/kg, i.p.) was administered to all animals immediately before behavioural assessment commenced. For each animal the latency to the first tremulous jaw movement and the total duration of the tremulous jaw movements post tacrine administration were recorded.

| | % Decrease from vehicle | |
|---|---|---|
| | or percentage relative | |
| | to vehicle (100%), | |
| Example | (mean ± SEM) | Species |
| 24b | 23.9 ± 3.8*** | mouse |
| 78b | 23.8 ± 7.7*** | mouse |
| 94b | 79.9 ± 7.2 | mouse |
| 65 | 30.0 ± 12.8* | rat |

illustrative data shown is percentage attenuation of tacrine-induced tremulous jaw movements relative to vehicle (100%). Each compound shown was tested at 10 mg/kg p.o.

Statistical significance was assessed using an unpaired student's t-test vs vehicle * p<0.05, ***p<0.001.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 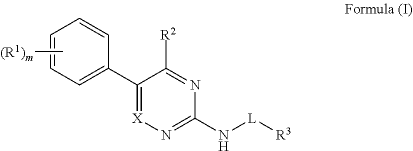
or a pharmaceutically acceptable salt, solvate or prodrug thereof, wherein:
X is N or CR⁴;
L is a bond, —C(O)—, —C(O)—C(R⁵)₂—, —C(R⁵)₂—, or —C(R⁵)₂C(R⁵)₂—;
each R¹ is independently selected from halo, cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, and C₃-C₆ halocycloalkoxy;
m is 2, 3, 4 or 5;
R² is selected from halo, cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, and C₃-C₆ halocycloalkoxy;
R³ is selected from a C₃-C₆ cycloalkyl and 4- to 10-membered heterocyclic group, wherein the cycloalkyl or heterocyclic group is optionally substituted with one, two, three, four or five substituents independently selected from halo, cyano, hydroxy, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, C₃-C₆ halocycloalkoxy, —N(R⁶)₂, and —SO₂(R⁶);
R⁴ is selected from hydrogen, halo, cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, and C₃-C₆ halocycloalkoxy;
each R⁵ is independently selected from hydrogen, CH₃, CH₂CH₃, and CF₃; and
each R⁶ is independently selected from hydrogen and C₁-C₃ alkyl, or two R⁶ together with the nitrogen to which they are attached form a 3- to 6-membered saturated heterocyclic group;
provided the compound is not:
4-methoxy-5-(2-methyl-4-(trifluoromethoxy)phenyl)-N-((1s,4s)-4-methylcyclohexyl)pyrimidin-2-amine;
4-methoxy-5-(2-methoxy-4-(trifluoromethoxy)phenyl)-N-((1s,4s)-4-methylcyclohexyl)pyrimidin-2-amine; or
N-cyclopropyl-5-(3,4-dimethoxyphenyl)-4-methyl-pyrimidin-2-amine.

2. The compound as claimed in claim 1, wherein X is N.

3. The compound as claimed in claim 1, wherein X is CR⁴.

4. The compound as claimed in claim 3, wherein R⁴ is selected from hydrogen, halo, cyano, C₁-C₃ alkyl, C₁-C₃ haloalkyl, cyclopropyl, C₁-C₃ alkoxy, C₁-C₃ haloalkoxy, and cyclopropoxy.

5. The compound as claimed in claim 1, wherein each R¹ is independently selected from halo, cyano, C₁-C₃ alkyl, C₁-C₃ haloalkyl, C₁-C₃ alkoxy, an C₁-C₃ haloalkoxy.

6. The compound as claimed in claim 1, wherein m is 2.

7. The compound as claimed in any one of the claim 1, wherein R² is selected from halo, cyano, C₁-C₃ alkyl, C₁-C₃ haloalkyl, cyclopropyl, C₁-C₃ alkoxy, C₁-C₃ haloalkoxy, and cyclopropoxy.

8. The compound as claimed in any one of the claim 1, wherein L is a bond, —C(O)—, —C(O)—CH₂—, —C(O)—CH(CH₃)—, —C(O)—CH(CF₃)—, —CH₂—, —CH(CH₃)—, —CH(CF₃)—, —CH₂CH₂—, —CH₂—CH(CH₃)—, —CH₂—CH(CF₃)—, —CH(CH₃)—CH₂—, or —CH(CF₃)—CH₂—.

9. The compound as claimed in claim 1, wherein R³ is selected from a C₃-C₆ cycloalkyl group, a 4-, 5- or 6-membered saturated monocyclic heterocyclic group, a 7-, 8-, 9- or 10-membered saturated bicyclic heterocyclic group, and a 5- or 6-membered heteroaryl group, each of which is optionally substituted.

10. The compound as claimed in any one of the claim 1, wherein the cycloalkyl or heterocyclic group of R³ is optionally substituted with one, two, three, four or five substituents independently selected from halo, cyano, hydroxy, C₁-C₃ alkyl, C₁-C₃ haloalkyl, C₁-C₃ alkoxy, C₁-C₃ haloalkoxy, —N(R⁶)₂, and —SO₂(R⁶), wherein each R⁶ is independently selected from hydrogen and C₁-C₃ alkyl.

11. The compound as claimed in claim 1, wherein the compound is selected from:
4-methyl-N-(2-(1-methylpyrrolidin-2-yl)ethyl)-5-phenylpyrimidin-2-amine;
N-(2-(1-methylpyrrolidin-2-yl)ethyl)-5-phenylpyrimidin-2-amine;
5-(4-fluorophenyl)-N-(2-(1-methylpyrrolidin-2-yl)ethyl)pyrimidin-2-amine;
5-(4-fluorophenyl)-N-((1-methylpiperidin-3-yl)methyl)pyrimidin-2-amine;
5-(4-fluorophenyl)-N-(1-methylpiperidin-3-yl)pyrimidin-2-amine;
5-(4-fluorophenyl)-N-(1-isopropylpiperidin-3-yl)pyrimidin-2-amine;
N¹-(5-(4-fluorophenyl)pyrimidin-2-yl)-N³,N³-dimethylcyclohexane-1,3-diamine;
5-(4-fluorophenyl)-N-((1-methylpiperidin-2-yl)methyl)pyrimidin-2-amine;
5-(4-fluorophenyl)-N-(1-isopropylpiperidin-3-yl)-4-methyl-pyrimidin-2-amine;
5-(2,4-difluorophenyl)-N-((1-ethylpiperidin-2-yl)methyl)-4-methyl-pyrimidin-2-amine;
5-(2,4-difluorophenyl)-N-((1-(dimethylamino)cyclopentyl)methyl)-4-methyl-pyrimidin-2-amine;
N-(5-(2,4-difluorophenyl)-4-methyl-pyrimidin-2-yl)-8-methyl-8-azabicyclo[3.2.1]octan-3-amine;
5-(2,4-difluorophenyl)-4-methyl-N-((1-methylpyrrolidin-3-yl)methyl)pyrimidin-2-amine;
5-(2,4-difluorophenyl)-4-methyl-N-(2-(1-methylpyrrolidin-2-yl)ethyl)pyrimidin-2-amine;
6-(2,4-difluorophenyl)-5-methyl-N-((1-methylpyrrolidin-2-yl)methyl)-1,2,4-triazin-3-amine;
5-(2-fluoro-4-methoxyphenyl)-4-methyl-N-(2-(1-methylpyrrolidin-2-yl)ethyl)pyrimidin-2-amine;
5-(2-fluoro-4-methoxyphenyl)-4-methyl-N-((1-methylpiperidin-2-yl)methyl)pyrimidin-2-amine;
6-(2-fluoro-4-methoxyphenyl)-5-methyl-N-((1-methylpyrrolidin-2-yl)methyl)-1,2,4-triazin-3-amine;
5-(2,4-difluorophenyl)-2-(((1-methylpyrrolidin-2-yl)methyl)amino)pyrimidine-4-carbonitrile;
6-(2,4-difluorophenyl)-5-methyl-N-((1-methylpiperidin-2-yl)methyl)-1,2,4-triazin-3-amine;
6-(2,4-difluorophenyl)-5-methyl-N-(2-(1-methylpyrrolidin-2-yl)ethyl)-1,2,4-triazin-3-amine;
5-(2,4-difluorophenyl)-N-(1-isopropylpyrrolidin-3-yl)-4-methyl-pyrimidin-2-amine;
6-(2-fluoro-4-methoxyphenyl)-5-methyl-N-(2-(1-methylpyrrolidin-2-yl)ethyl)-1,2,4-triazin-3-amine;
5-(2,4-difluorophenyl)-4-methyl-N-((1-methylpyrrolidin-2-yl)methyl)pyrimidin-2-amine;
5-(2-fluoro-4-methoxyphenyl)-2-(((1-methylpiperidin-2-yl)methyl)amino)pyrimidine-4-carbonitrile;
6-(2-fluoro-4-methoxyphenyl)-5-methyl-N-((1-methylpiperidin-2-yl)methyl)-1,2,4-triazin-3-amine;
5-(2-fluoro-4-methoxyphenyl)-2-((2-(1-methylpyrrolidin-2-yl)ethyl)amino)pyrimidine-4-carbonitrile;
5-(2-fluoro-4-methoxyphenyl)-4-methyl-N-((1-methylpyrrolidin-2-yl)methyl)pyrimidin-2-amine;
5-(2,4-difluorophenyl)-4-methyl-N-(1-methylpyrrolidin-3-yl)pyrimidin-2-amine;
5-(2-fluoro-4-methoxyphenyl)-4-methyl-N-((1-methylpiperidin-3-yl)methyl)pyrimidin-2-amine;
5-(2-fluoro-4-methoxyphenyl)-2-(((1-methylpyrrolidin-3-yl)methyl)amino)pyrimidine-4-carbonitrile;
5-(2-fluoro-4-methoxyphenyl)-4-methyl-N-((4-methylmorpholin-3-yl)methyl)pyrimidin-2-amine;
5-(2-fluoro-5-methoxyphenyl)-4-methyl-N-((1-methylpiperidin-2-yl)methyl)pyrimidin-2-amine;
5-(2-fluoro-5-methoxyphenyl)-4-methyl-N-((1-methylpiperidin-3-yl)methyl)pyrimidin-2-amine;
5-(2-chloro-4-methoxyphenyl)-4-methyl-N-((1-methylpiperidin-2-yl)methyl)pyrimidin-2-amine;
5-(2-fluoro-4-methoxyphenyl)-4-methyl-N-((4-methylmorpholin-2-yl)methyl)pyrimidin-2-amine;
4-cyclopropyl-5-(2-fluoro-4-methoxyphenyl)-N-((1-methylpyrrolidin-3-yl)methyl)pyrimidin-2-amine;
2-((3-(dimethylamino)cyclopentyl)amino)-5-(2-fluoro-4-methoxyphenyl)pyrimidine-4-carbonitrile;
N¹-(6-(2,4-difluorophenyl)-5-methyl-1,2,4-triazin-3-yl)-N³,N³-dimethylcyclohexane-1,3-diamine;
5-(2-fluoro-4-methoxyphenyl)-4-methyl-N-(1-methylpiperidin-4-yl)pyrimidin-2-amine;
5-(2-fluoro-4-methoxyphenyl)-N-(1-isopropylpiperidin-4-yl)-4-methyl-pyrimidin-2-amine;
5-(2,4-difluorophenyl)-4-methyl-N-(pyridin-3-ylmethyl)pyrimidin-2-amine;
N¹-(5-(2-fluoro-4-methoxyphenyl)-4-(trifluoromethyl)pyrimidin-2-yl)-N³,N³-dimethylcyclopentane-1,3-diamine;
N¹-(5-(2-fluoro-4-methoxyphenyl)-4-methyl-pyrimidin-2-yl)-N³,N³-dimethylcyclopentane-1,3-diamine;
5-(2-fluoro-4-methoxyphenyl)-4-methyl-N-(tetrahydro-2H-pyran-4-yl)pyrimidin-2-amine;
5-(2-fluoro-4-methoxyphenyl)-4-methyl-N-(tetrahydro-2H-pyran-3-yl)pyrimidin-2-amine;
5-(2-chloro-4-methoxyphenyl)-4-methyl-N-((tetrahydrofuran-2-yl)methyl)pyrimidin-2-amine;
5-(2-fluoro-4-methoxyphenyl)-N-(1-isopropylpiperidin-3-yl)-4-methyl-pyrimidin-2-amine;
5-(2-fluoro-4-methoxyphenyl)-N-((1-isopropylpiperidin-3-yl)methyl)-4-methyl-pyrimidin-2-amine;
5-(2-fluoro-4-methoxyphenyl)-N-((1-isopropylpiperidin-2-yl)methyl)-4-methyl-pyrimidin-2-amine;
5-(2-fluoro-4-methoxyphenyl)-4-methyl-N-((tetrahydro-2H-pyran-3-yl)methyl)pyrimidin-2-amine;
5-(2,4-difluorophenyl)-4-methyl-N-(3-(methylsulfonyl)cyclopentyl)pyrimidin-2-amine;
5-(2-fluoro-4-methoxyphenyl)-N-(3-methoxycyclohexyl)-4-methyl-pyrimidin-2-amine;
N¹-(5-(2,4-difluorophenyl)-4-methyl-pyrimidin-2-yl)-N³,N³-dimethylcyclohexane-1,3-diamine;
2-((3-(dimethylamino)cyclohexyl)amino)-5-(2-fluoro-4-methoxyphenyl)pyrimidine-4-carbonitrile;
5-(2,4-difluorophenyl)-2-((3-(dimethylamino)cyclopentyl)amino)pyrimidine-4-carbonitrile;
5-(2,4-difluorophenyl)-2-((3-(dimethylamino)cyclohexyl)amino)pyrimidine-4-carbonitrile;
5-(2,4-difluorophenyl)-4-methyl-N-(1-(2,2,2-trifluoroethyl)piperidin-3-yl)pyrimidin-2-amine;
5-(2-fluoro-5-methoxyphenyl)-4-methyl-N-((4-methylmorpholin-3-yl)methyl)pyrimidin-2-amine;
5-(2-chloro-4-methoxyphenyl)-4-methyl-N-((4-methylmorpholin-3-yl)methyl)pyrimidin-2-amine;
5-(2-chloro-4-methoxyphenyl)-4-methyl-N-((1-methyl-1H-imidazol-5-yl)methyl)pyrimidin-2-amine;
5-(2,4-difluorophenyl)-4-methyl-N-(1-(1-methylpyrrolidin-3-yl)ethyl)pyrimidin-2-amine;
5-(2,4-difluorophenyl)-2-((1-isopropylpiperidin-4-yl)amino)pyrimidine-4-carbonitrile;
5-(2,4-difluorophenyl)-2-((1-methylpiperidin-4-yl)amino)pyrimidine-4-carbonitrile;
5-(2,4-difluorophenyl)-N-(1-isopropylpiperidin-4-yl)-4-methyl-pyrimidin-2-amine;
N¹-(5-(2,4-difluorophenyl)-4-methyl-pyrimidin-2-yl)-N³,N³-dimethylcyclobutane-1,3-diamine;
6-(2-fluoro-4-methoxyphenyl)-5-methyl-N-(1-methylpiperidin-3-yl)-1,2,4-triazin-3-amine;
5-(2-fluoro-4-methoxyphenyl)-4,6-dimethyl-N-((1-methylpyrrolidin-2-yl)methyl)pyrimidin-2-amine;
5-(2,4-difluorophenyl)-4-methyl-N-(1-methylpiperidin-4-yl)pyrimidin-2-amine;
5-(2,4-difluorophenyl)-N-(1-isopropylpiperidin-3-yl)-4-methyl-pyrimidin-2-amine;
5-(2-fluoro-4-(trifluoromethoxy)phenyl)-4-methyl-N-(pyrrolidin-2-ylmethyl)pyrimidin-2-amine;
5-(2-fluoro-4-methoxyphenyl)-4-methoxy-6-methyl-N-((1-methylpyrrolidin-2-yl)methyl)pyrimidin-2-amine;
6-(2-fluoro-4-methoxyphenyl)-N-(1-isopropylpiperidin-3-yl)-5-methyl-1,2,4-triazin-3-amine;
5-(2,4-difluorophenyl)-4-methyl-N-(1-methylpiperidin-3-yl)pyrimidin-2-amine;
5-(2,4-difluorophenyl)-N-(1-ethylpiperidin-3-yl)-4-methyl-pyrimidin-2-amine;
5-(2,4-difluorophenyl)-4-methyl-N-((4-methylmorpholin-3-yl)methyl)pyrimidin-2-amine;
N-((4,4-difluoro-1-methylpyrrolidin-2-yl)methyl)-5-(2,4-difluorophenyl)-4-methyl-pyrimidin-2-amine;
5-(2-fluoro-4-methoxyphenyl)-2-((1-isopropylpiperidin-4-yl)amino)pyrimidine-4-carbonitrile;
5-(2,4-difluorophenyl)-4-methyl-N-(pyrrolidin-2-ylmethyl)pyrimidin-2-amine;
5-(2,4-difluorophenyl)-N-((3,3-difluoropyrrolidin-2-yl)methyl)-4-methyl-pyrimidin-2-amine;
N-((3,3-difluoro-1-methylpyrrolidin-2-yl)methyl)-5-(2,4-difluorophenyl)-4-methyl-pyrimidin-2-amine;
5-(2,4-difluorophenyl)-N-((1-ethylpyrrolidin-2-yl)methyl)-4-methyl-pyrimidin-2-amine;
N-(5-(2,4-difluorophenyl)-4-methyl-pyrimidin-2-yl)hexahydro-1H-pyrrolizin-1-amine;
5-(2,4-difluorophenyl)-4-methyl-N-((1-(trideuteriomethyl)pyrrolidin-2-yl)methyl)pyrimidin-2-amine;
5-(2-fluoro-4-(trifluoromethoxy)phenyl)-N-(1-isopropylpiperidin-4-yl)-4-methyl-pyrimidin-2-amine;
5-(2-fluoro-4-(trifluoromethoxy)phenyl)-4-methyl-N-(1-methylpiperidin-4-yl)pyrimidin-2-amine;
5-(2-fluoro-4-(trifluoromethyl)phenyl)-4-methyl-N-((1-methylpyrrolidin-2-yl)methyl)pyrimidin-2-amine;
5-(4-fluoro-2-(trifluoromethyl)phenyl)-4-methyl-N-((1-methylpyrrolidin-2-yl)methyl)pyrimidin-2-amine;
5-(2-fluoro-4-(trifluoromethoxy)phenyl)-4-methyl-N-((1-methylpyrrolidin-2-yl)methyl)pyrimidin-2-amine;
5-(2,4-difluorophenyl)-4-methyl-N-((1-(2,2,2-trifluoroethyl)pyrrolidin-2-yl)methyl)pyrimidin-2-amine;
5-(2,4-difluorophenyl)-N-((3-fluoro-1-methylpyrrolidin-2-yl)methyl)-4-methyl-pyrimidin-2-amine;
N-(5-(2-fluoro-4-(trifluoromethoxy)phenyl)-4-methyl-pyrimidin-2-yl)hexahydro-1H-pyrrolizin-1-amine;
N-((1-ethylpyrrolidin-2-yl)methyl)-5-(2-fluoro-4-(trifluoromethyl)phenyl)-4-methyl-pyrimidin-2-amine;
5-(2-fluoro-4-(trifluoromethyl)phenyl)-4-methyl-N-((1-methylpyrrolidin-3-yl)methyl)pyrimidin-2-amine;
5-(2-fluoro-4-(trifluoromethyl)phenyl)-N-(1-isopropylpyrrolidin-3-yl)-4-methyl-pyrimidin-2-amine;
5-(2-fluoro-4-(trifluoromethyl)phenyl)-4-methyl-N-((1-methyl-4-piperidinyl)methyl)pyrimidin-2-amine;
5-(2-fluoro-4-(trifluoromethyl)phenyl)-4-methyl-N-(pyrrolidin-2-ylmethyl)pyrimidin-2-amine;
5-(2-fluoro-4-(trifluoromethyl)phenyl)-4-methyl-N-(2-piperidinylmethyl)pyrimidin-2-amine;
5-(2-fluoro-4-(trifluoromethyl)phenyl)-4-methyl-N-(2-(1-methylpyrrolidin-2-yl)ethyl)pyrimidin-2-amine;
5-(2-fluoro-4-(trifluoromethyl)phenyl)-4-methyl-N-((1-methyl-3-piperidinyl)methyl)pyrimidin-2-amine;
5-(2-fluoro-4-(trifluoromethyl)phenyl)-4-methyl-N-((4-methylmorpholin-2-yl)methyl)pyrimidin-2-amine;
N¹-(5-(2-fluoro-4-(trifluoromethyl)phenyl)-4-methyl-pyrimidin-2-yl)-N²,N²-dimethyl-cyclohexane-1,2-diamine;
5-(2-fluoro-4-(trifluoromethyl)phenyl)-4-methyl-N-(pyrrolidin-3-yl)pyrimidin-2-amine;
N³-(5-(2-fluoro-4-(trifluoromethyl)phenyl)-4-methyl-pyrimidin-2-yl)cyclopentane-1,3-diamine;
N-(4-fluoropyrrolidin-3-yl)-5-(2-fluoro-4-(trifluoromethyl)phenyl)-4-methyl-pyrimidin-2-amine;
5-(2-fluoro-4-(trifluoromethyl)phenyl)-4-methyl-N-((3-piperidinyl)methyl)pyrimidin-2-amine;
4-((5-(2-fluoro-4-(trifluoromethyl)phenyl)-4-methyl-pyrimidin-2-yl)amino)pyrrolidin-3-ol;
5-(2-fluoro-4-(trifluoromethyl)phenyl)-4-methyl-N-(3-piperidinyl)pyrimidin-2-amine;
5-(2-fluoro-4-(trifluoromethyl)phenyl)-4-methyl-N-((1-(trideuteriomethyl)pyrrolidin-2-yl)methyl)pyrimidin-2-amine;
N-(5-(2-fluoro-4-(trifluoromethyl)phenyl)-4-methyl-pyrimidin-2-yl)quinuclidin-3-amine;
N-(5-(2-fluoro-4-(trifluoromethyl)phenyl)-4-methyl-pyrimidin-2-yl)-8-azabicyclo[3.2.1]octan-3-amine;
N-(5-(2-fluoro-4-(trifluoromethyl)phenyl)-4-methyl-pyrimidin-2-yl)-2-azaspiro[3.3]heptan-6-amine;
N-(5-(2-fluoro-4-(trifluoromethyl)phenyl)-4-methyl-pyrimidin-2-yl)-6-azaspiro[2.5]octan-1-amine;
5-(2-fluoro-4-(trifluoromethyl)phenyl)-N-(3-fluoropiperidin-4-yl)-4-methyl-pyrimidin-2-amine;
N¹-(5-(2-fluoro-4-(trifluoromethyl)phenyl)-4-methyl-pyrimidin-2-yl)cyclohexane-1,3-diamine;
5-(2-fluoro-4-(trifluoromethyl)phenyl)-4-methyl-N-(2,2,6,6-tetramethylpiperidin-4-yl)pyrimidin-2-amine;
5-(2-fluoro-4-(trifluoromethyl)phenyl)-4-methyl-N-(piperidin-3-ylmethyl)pyrimidin-2-amine;
5-(2-fluoro-4-(trifluoromethyl)phenyl)-4-methyl-N-(piperidin-3-yl)pyrimidin-2-amine;
5-(2-fluoro-4-(trifluoromethyl)phenyl)-4-methyl-N-(1-(pyrrolidin-2-yl)ethyl)pyrimidin-2-amine;
5-(2-fluoro-4-(trifluoromethyl)phenyl)-4-methyl-N-(1-(1-methylpyrrolidin-2-yl)ethyl)pyrimidin-2-amine;
5-(2-fluoro-4-(trifluoromethyl)phenyl)-N-((3-fluoropyrrolidin-2-yl)methyl)-4-methyl-pyrimidin-2-amine;
5-(2-fluoro-4-(trifluoromethyl)phenyl)-4-methyl-N-((2-methyl-pyrrolidin-2-yl)methyl)pyrimidin-2-amine;
N-((1,2-dimethylpyrrolidin-2-yl)methyl)-5-(2-fluoro-4-(trifluoromethyl)phenyl)-4-methyl-pyrimidin-2-amine;
N-((1,5-dimethylpyrrolidin-2-yl)methyl)-5-(2-fluoro-4-(trifluoromethyl)phenyl)-4-methyl-pyrimidin-2-amine; and
N-(5-(2-fluoro-4-(trifluoromethyl)phenyl)-4-methyl-pyrimidin-2-yl)-1-methylpyrrolidine-2-carboxamide;
or an enantiomer of any of the foregoing;
or a pharmaceutically acceptable salt, solvate or prodrug of any of the foregoing.

12. The compound as claimed in claim 1, wherein the compound is selected from:
(R)-5-(2-fluoro-4-(trifluoromethyl)phenyl)-4-methyl-N-((1-methylpyrrolidin-2-yl)methyl)pyrimidin-2-amine;
(R)-5-(2-fluoro-4-(trifluoromethyl)phenyl)-4-methyl-N-(pyrrolidin-2-ylmethyl)pyrimidin-2-amine;
(S)-5-(2-fluoro-4-(trifluoromethyl)phenyl)-4-methyl-N-(pyrrolidin-2-ylmethyl)pyrimidin-2-amine;
N-(5-(2-fluoro-4-(trifluoromethyl)phenyl)-4-methyl-pyrimidin-2-yl)-2-azaspiro[3.3]heptan-6-amine;
5-(2-fluoro-4-(trifluoromethyl)phenyl)-4-methyl-N-((1*S)-1-((2S)-pyrrolidin-2-yl)ethyl)pyrimidin-2-amine; and
N-(((2*S,5*R)-1,5-dimethylpyrrolidin-2-yl)methyl)-5-(2-fluoro-4-(trifluoromethyl)phenyl)-4-methyl-pyrimidin-2-amine;
or a pharmaceutically acceptable salt, solvate or prodrug of any of the foregoing.

13. The compound as claimed in claim 1, wherein the compound is selected from:
(S)-5-(2,4-difluorophenyl)-4-methyl-N-((1-methylpyrrolidin-2-yl)methyl)pyrimidin-2-amine;
(R)-5-(2,4-difluorophenyl)-4-methyl-N-((1-methylpyrrolidin-2-yl)methyl)pyrimidin-2-amine;
5-(2,4-difluorophenyl)-N-(1-isopropylpiperidin-4-yl)-4-methyl-pyrimidin-2-amine;
(R)-5-(2,4-difluorophenyl)-N-(1-ethylpiperidin-3-yl)-4-methyl-pyrimidin-2-amine;
(S)-5-(2,4-difluorophenyl)-N-(1-ethylpiperidin-3-yl)-4-methyl-pyrimidin-2-amine;
5-(2,4-difluorophenyl)-4-methyl-N-(pyrrolidin-2-ylmethyl)pyrimidin-2-amine;
5-(2,4-difluorophenyl)-4-methyl-N-((1-(trideuteriomethyl)pyrrolidin-2-yl)methyl)pyrimidin-2-amine;
(S)-5-(2,4-difluorophenyl)-4-methyl-N-((1-(trideuteriomethyl)pyrrolidin-2-yl)methyl)pyrimidin-2-amine;
(R)-5-(2,4-difluorophenyl)-4-methyl-N-((1-(trideuteriomethyl)pyrrolidin-2-yl)methyl)pyrimidin-2-amine;
(R)-5-(2,4-difluorophenyl)-4-methyl-N-(pyrrolidin-2-ylmethyl)pyrimidin-2-amine;
(S)-5-(2,4-difluorophenyl)-4-methyl-N-(pyrrolidin-2-ylmethyl)pyrimidin-2-amine; and
5-(2-fluoro-4-(trifluoromethyl)phenyl)-4-methyl-N-((3S)-3-piperidinyl)pyrimidin-2-amine;
or a pharmaceutically acceptable salt, solvate or prodrug of any of the foregoing.

14. A process for the preparation of a compound of formula (I) or a pharmaceutically acceptable salt, solvate or prodrug thereof, as claimed in claim 1, wherein the process comprises:
(a) reacting a compound of formula (II) or a salt thereof, with a compound of formula (III) or a salt thereof, to provide a compound of formula (IV) or a salt thereof: 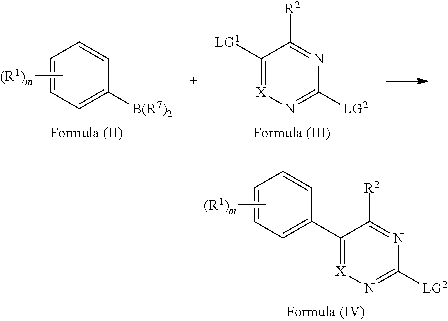
wherein m, R¹, R² and X are as defined in claim 1; R⁷ is independently selected from hydroxy, C₁-C₅ alkyl, and C₁-C₅ alkoxy, or two R⁷ together with the boron to which they are attached form an optionally substituted 3- to 12-membered heterocyclic group; and LG¹ and LG² are leaving groups, or a sulfonate group;
(b) reacting a compound of formula (IV) or a salt thereof, with a compound of formula (V) or a salt thereof, to provide a compound of formula (I) or a salt thereof: 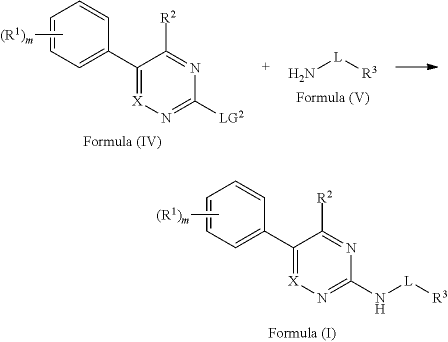
wherein R³ and L are as defined in claim 1;
and optionally thereafter carrying out one or more of the following procedures:
converting a compound of formula (I) into another compound of formula (I);
removing any protecting groups;
forming a pharmaceutically acceptable salt, solvate or prodrug.

15. A pharmaceutical composition comprising a compound of formula (I) or a pharmaceutically acceptable salt, solvate or prodrug thereof, as claimed in claim 1, in association with a pharmaceutically acceptable adjuvant, diluent or carrier, and optionally one or more other therapeutic agents.

16. (canceled)

17. A method of treating or preventing a disease, disorder or condition, wherein the method comprises administering to a patient in need thereof a therapeutically or prophylactically effective amount of a compound of formula (I): 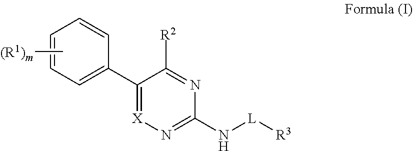
or a pharmaceutically acceptable salt, solvate or prodrug thereof, wherein:
X is N or CR⁴;
L is a bond, —C(O)—, —C(O)—C(R⁵)₂—, —C(R⁵)₂—, or —C(R⁵)₂C(R⁵)₂—;
each R¹ is independently selected from halo, cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, and C₃-C₆ halocycloalkoxy;
m is 0, 1, 2, 3, 4 or 5;
R² is selected from hydrogen, halo, cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, and C₃-C₆ halocycloalkoxy;
R³ is selected from a C₃-C₆ cycloalkyl and 4- to 10-membered heterocyclic group, wherein the cycloalkyl or heterocyclic group is optionally substituted with one, two, three, four or five substituents independently selected from halo, cyano, hydroxy, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, C₃-C₆ halocycloalkoxy, —N(R⁶)₂, and —SO₂(R⁶);
R⁴ is selected from hydrogen, halo, cyano, C₁-C₅ alkyl, C₁-C₅ haloalkyl, C₃-C₆ cycloalkyl, C₃-C₆ halocycloalkyl, C₁-C₅ alkoxy, C₁-C₅ haloalkoxy, C₃-C₆ cycloalkoxy, and C₃-C₆ halocycloalkoxy;
each R⁵ is independently selected from hydrogen, CH₃, CH₂CH₃, and CF₃; and
each R⁶ is independently selected from hydrogen and C₁-C₃ alkyl, or two R⁶ together with the nitrogen to which they are attached form a 3- to 6-membered saturated heterocyclic group;
wherein the disease, disorder or condition is associated with nicotinic acetylcholine receptor α6 (nAChRα6) activity: or wherein the disease, disorder or condition has dysregulation of dopamine, noradrenaline or serotonin as a key pathological mechanism; or wherein the disease, disorder or condition is selected from a movement disorder, dystonia, dyskinesia, Parkinson's disease, Huntington's disease, a psychiatric disorder, an addiction disorder, and a non-motor symptom of Parkinson's disease.

18-21. (canceled)

22. The method of claim 17, wherein the movement disorder is tremor; or wherein the dyskinesia is L-DOPA induced dyskinesia in Parkinson's disease; or wherein the psychiatric disorder is selected from schizophrenia, psychotic disorder, psychosis, schizoaffective disorder, bipolar disorder, attention deficit hyperactivity disorder (ADHD), autism spectrum disorder (ASD), and Tourettes syndrome; or wherein the addiction disorder is selected from substance or drug dependence, alcohol dependence, nicotine dependence, binge eating, and gambling disorder; or wherein the non-motor symptom of Parkinson's disease is selected from apathy, anhedonia, and depression.

23. The method of claim 22, wherein the tremor is selected from a resting tremor in Parkinson's disease and essential tremor including essential tremor in isolation, essential tremor in Parkinson's disease, essential tremor in Alzheimer's disease, and essential tremor in other neurodegenerative diseases; or wherein the bipolar disorder is selected from bipolar I, bipolar IL, bipolar mania, and bipolar depression; or wherein the autism spectrum disorder (ASD) is Fragile X syndrome.
